(19) United States
(12) Patent Application Publication
(10) Pub. No.: US 2024/0408099 A1
(43) Pub. Date: Dec. 12, 2024

(54) COMBINATION THERAPIES OF KRAS G12D INHIBITORS WITH Pan ErbB FAMILY INHIBITORS

(71) Applicant: MIRATI THERAPEUTICS, INC., San Diego, CA (US)

(72) Inventors: Jill Hallin, San Diego, CA (US); James Gail Christensen, San Diego, CA (US); Vickie Bowcut, San Diego, CA (US); Peter Olson, San Diego, CA (US)

(21) Appl. No.: 18/694,867

(22) PCT Filed: Oct. 4, 2022
(86) PCT No.: PCT/US2022/045621

§ 371 Date: Mar. 22, 2024

Related U.S. Application Data
(60) Provisional application No. 63/252,534, filed on Oct. 5, 2021.

(51) Int. Cl. A61K 31/519; A61K 31/517; A61K 39/395; A61P 35/00

(52) U.S. Cl. CPC A61K 31/519; A61K 31/517; A61K 39/3955; A61P 35/00

(57) ABSTRACT

The present invention relates to combination therapies for treating KRas G12D cancers. In particular, the present invention relates to methods of treating cancer in a subject in need thereof, comprising administering to the subject a therapeutically effective amount of a combination of a pan ErbB family inhibitor and a KRAS G12D inhibitor of Formula (I), pharmaceutical compositions comprising a therapeutically effective amounts of the inhibitors, kits comprising the compositions and methods of use therefor.

### FIELD OF THE INVENTION

The present invention relates to combination therapies useful for treating cancer. In particular, the present invention relates to therapeutically effective combinations of a pan ErbB family inhibitor and a KRas G12D inhibitor, pharmaceutical compositions comprising the inhibitors, kits comprising the compositions and methods of use therefor.

### BACKGROUND OF THE INVENTION

Kirsten Rat Sarcoma 2 Viral Oncogene Homolog (“KRas”) is a small GTPase and a member of the Ras family of oncogenes. KRas serves as a molecular switch cycling between inactive (GDP-bound) and active (GTP-bound) states to transduce upstream cellular signals received from multiple tyrosine kinases to downstream effectors regulating a wide variety of processes, including cellular proliferation (e.g., see Alamgeer et al., (2013) Current Opin Pharmcol. 13:394-401).

The role of activated KRas in malignancy was observed over thirty years ago (e.g., see Der et al., (1982) Proc. Natl Acad. Sci. USA 79(11):3637-3640). Aberrant expression of KRas accounts for up to 20% of all cancers and oncogenic KRas mutations that stabilize GTP binding and lead to constitutive activation of KRas and downstream signaling have been reported in 25-30% of lung adenocarcinomas. (e.g., see Samatar and Poulikakos (2014) Nat Rev Drug Disc 13(12): 928-942 doi: 10.1038/nrd428). Single nucleotide substitutions that result in missense mutations at codons 12 and 13 of the KRas primary amino acid sequence comprise approximately 33% of these KRas driver mutations in lung adenocarcinoma, with a G12D mutation being a common activating mutation (e.g., see Li, Balmain and Counter, (2018) Nat Rev Cancer December; 18(12):767-777; Sanchez-Vega, et al, (2018) Cell; 173, 321-337).

The well-known role of KRas in malignancy and the discovery of these frequent mutations in KRas in various tumor types made KRas a highly attractable target of the pharmaceutical industry for cancer therapy. Notwithstanding thirty years of large scale discovery efforts to develop inhibitors of KRas for treating cancer, only a single KRas G12C inhibitor (the KRas G12C inhibitor sotorasib) has demonstrated sufficient safety and/or efficacy to obtain regulatory approval (e.g., see: FDA Approves First KRAS Inhibitor: Sotorasib. [No authors listed] Cancer Discov. 2021 August; 11(8):OF4. doi: 10.1158/2159-8290.CD-NB2021-0362. Epub 2021 Jun. 22). To date, no KRas G12D inhibitors have demonstrated sufficient safety and/or efficacy to obtain regulatory approval.

Compounds that inhibit KRas activity are still highly desirable and under investigation, including those that disrupt effectors such as guanine nucleotide exchange factors (e.g., see Sun et al., (2012) Agnew Chem Int Ed Engl. 51(25):6140-6143 doi: 10.1002/anie201201358) as well as those that target KRas G12D (e.g., see K-Ras(G12D) Has a Potential Allosteric Small Molecule Binding Site, Feng H, Zhang Y, Bos P H, Chambers J M, Dupont M M, Stockwell B R, Biochemistry, 2019 May 28; 58(21):2542-2554. doi: 10.1021/acs.biochem.8b01300. Epub 2019 May 14; and Second harmonic generation detection of Ras conformational changes and discovery of a small molecule binder, Donohue E, Khorsand S, Mercado G, Varney K M, Wilder P T, Yu W, MacKerell AD Jr, Alexander P, Van Q N, Moree B, Stephen A G, Weber D J, Salafsky J, McCormick F., Proc Natl Acad Sci USA 2019 Aug. 27; 116(35):17290-17297, doi: 10.1073/pnas.1905516116. Epub 2019 Aug. 9). Clearly there remains a continued interest and effort to develop inhibitors of KRas, particularly inhibitors of activating KRas mutants, including KRas G12D.

While the KRas G12D inhibitors disclosed herein are potent inhibitors of KRas G12D signaling and exhibit single agent activity inhibiting the in vitro proliferation of cell lines harboring a KRas G12D mutation, the relative potency and/or observed maximal effect of any given KRas G12D inhibitor can vary between KRAS mutant cell lines. The reason or reasons for the range of potencies and observed maximal effect is not fully understood but certain cell lines appear to possess differing intrinsic resistance. Thus, there is a need to develop alternative approaches to maximize the potency, efficacy, therapeutic index and/or clinical benefit of KRas G12D inhibitors in vitro and in vivo.

The combination therapy of the present invention, in one aspect, synergistically increases the potency of KRas G12D inhibitors resulting in improved efficacy of KRas G12D inhibitors disclosed herein. The combination therapy of the present invention, in another aspect, provides improved clinical benefit to patients compared to treatment with KRas G12D inhibitors disclosed herein as a single agent.

### SUMMARY OF THE INVENTION

In one aspect of the invention, provided herein are methods of treating cancer in a subject in need thereof, comprising administering to the subject a therapeutically effective amount of a combination of a pan ErbB family inhibitor and a KRAS G12D inhibitor of formula (I):

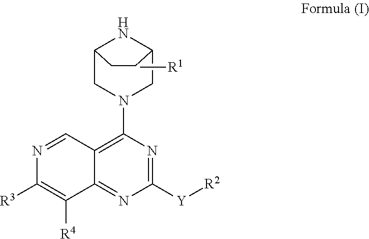

or a pharmaceutically acceptable salt thereof:
wherein:
R¹ is hydrogen, hydroxy, halogen, C1-C3 alkyl, C1-C3 cyanoalkyl, C1-C3 hydroxyalkyl, HC(═O)—, —CO₂R⁵, —CO₂N(R⁵)₂ or a 5-6 membered heteroaryl;
Y is a bond, O or NR⁵;
R² is hydrogen, —N(R⁵)₂, heterocyclyl, C1-C6 alkyl, -L-heterocyclyl, -L-aryl, -L-heteroaryl, -L-cycloalkyl, -L-N(R⁵)₂, -L-NHC(═NH)NH₂, -L-C(O)N(R⁵)₂, -L-C1-C6 haloalkyl, -L-OR⁵, -L-(CH₂OR⁵)(CH₂)ₙOR⁵, -L-NR⁵C(O)-aryl, -L-COOH, or -LC(═O)OC1-C6 alkyl, wherein the heterocyclyl and the aryl portion of -L-NR⁵C(O)-aryl and the heterocyclyl portion of -L-heterocyclyl and the cycloalkyl portion of the -L-cycloalkyl may be optionally substituted with one or more R⁶, and wherein the aryl or heteroaryl of the -L-aryl and the -L-heteroaryl may be optionally substituted with one or more R⁷;
each L is independently a C1-C4 alkylene optionally substituted with hydroxy, C1-C4 hydroxyalkyl or heteroaryl;
R³ is aryl or heteroaryl, wherein the aryl or the heteroaryl is optionally substituted with one or more R⁸;
R⁴ is hydrogen, halogen or C1-C3 alkyl;
each R⁵ is independently hydrogen or C1-C3 alkyl;
each R⁶ is independently halogen, hydroxy, C1-C3 hydroxyalkyl, C1-C3 alkyl, C1-C3 haloalkyl, C1-C3 alkoxy, cyano, -Q-phenyl, -Q-phenylSO₂F, —NHC(O)phenyl, —NHC(O)phenylSO₂F, C1-C3 alkyl substituted pyrazolyl, araC1-C3 alkyl-, tert-butyldimethylsilyloxyCH₂—, —N(R⁵)₂, (C1-C3 alkoxy)C1-C3 alkyl-, (C1-C3 alkyl)C(═O), oxo, (C1-C3 haloalkyl)C(═O)—, —SO₂F, (C1-C3 alkoxy)C1-C3 alkoxy, —CH₂OC(O)N(R⁵)₂, —CH₂NHC(O)OC1-C6 alkyl, —CH₂NHC(O)N(R⁵)₂, —CH₂NHC(O)C1-C6 alkyl, —CH₂(pyrazolyl), —CH₂NHSO₂C1-C6 alkyl, —CH₂OC(O)heterocyclyl, —OC(O)N(R⁵)₂, —OC(O)NH(C1-C3 alkyl)O(C1-C3 alkyl), —OC(O)NH(C1-C3 alkyl)O(C1-C3 alkyl)phenyl(C1-C3 alkyl)N(CH₃)₂, —OC(O)NH(C1-C3 alkyl)O(C1-C3 alkyl)phenyl or —OC(O)heterocyclyl, —CH₂heterocyclyl, wherein the phenyl of —NHC(O)phenyl or —OC(O)NH(C1-C3 alkyl)O(C1-C3 alkyl)phenyl is optionally substituted with —C(O)H or OH and wherein the heterocyclyl of —CH₂heterocyclyl is optionally substituted with oxo;
Q is a bond or O;
each R⁷ is independently halogen, hydroxy, HC(═O)—, C1-C4 alkyl, C1-C4 alkoxy, C1-C4 haloalkyl, C1-C4 hydroxyalkyl, or —N(R⁵)₂; and
each R⁸ is independently halogen, cyano, hydroxy, C1-C4 alkyl, —S—C1-C3 alkyl, C2-C4 alkenyl, C2-C4 alkynyl, C2-C4 hydroxyalkynyl, C1-C3 cyanoalkyl, triazolyl, C1-C3 haloalkyl, —O—C1-C3 haloalkyl, —S—C1-C3 haloalkyl, C1-C3 alkoxy, hydroxyC1-C3 alkyl, —CH₂C(═O)N(R⁵)₂, —C3-C4 alkynyl(NR⁵)₂, —N(R⁵)₂, deuteroC2-C4 alkynyl, (C1-C3 alkoxy)haloC1-C3 alkyl-, or C3-C6 cycloalkyl wherein said C3-C6 cycloalkyl is optionally substituted with halogen or C1-C3 alkyl.

In one aspect of the invention, KRas G12D inhibitors comprise compound MRTX1133 or MRTX1133 analogs and related compounds such as any of the compounds disclosed and described in WIPO publication WO2021/041671, including but not limited to: Ex. 252 (MRTX1133), 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-ethynyl-6-fluoronaphthalen-2-ol; Ex. 243, 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-ethynylnaphthalen-2-ol; Ex. 246, 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5,6-difluoronaphthalen-2-ol; Ex. 251, 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-chloronaphthalen-2-ol; Ex. 253, 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-ethyl-6-fluoronaphthalen-2-ol; Ex. 259, 4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethylnaphthalen-2-ol; and Ex. 282, 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-fluoronaphthalen-2-ol; or a pharmaceutically acceptable salt thereof and a pharmaceutically acceptable excipient.

In another aspect of the invention, pharmaceutical compositions are provided for use in the methods comprising a therapeutically effective amount of a combination of a pan ErbB family inhibitor and a KRas G12D inhibitor compound of Formula I, or a pharmaceutically acceptable salt thereof and a pharmaceutically acceptable excipient.

In one aspect of the invention, provided herein are methods of treating cancer in a subject in need thereof, comprising administering to the subject a therapeutically effective amount of a combination of a pan ErbB family inhibitor or a pharmaceutically acceptable salt or a pharmaceutical composition thereof and a KRAS G12D inhibitor of Formula (I), or a pharmaceutically acceptable salt or a pharmaceutical composition thereof. In one embodiment, the cancer is a KRas G12D-associated cancer. In one embodiment, the KRas G12D-associated cancer is pancreatic, colorectal, endometrial, and non-small cell lung cancer.

In some aspects of the invention, KRas G12D inhibitor compounds and pan ErbB family inhibitors are the only active agents in the provided compositions and methods.

In one embodiment, the pan ErbB family inhibitor is an irreversible inhibitor. Examples of irreversible pan ErbB family inhibitors suitable for the provided compositions and methods include, but are not limited to, Afatinib; Dacomitinib; Canertinib; Poziotinib, AV 412; PF 6274484 and HKI 357.

In one embodiment, the pan ErbB family inhibitor is a reversible inhibitor. Examples of reversible pan ErbB family inhibitors suitable for the provided compositions and methods include, but are not limited to erlotinib, gefitinib, sapitinib; varlitinib; TAK-285 (N-[2-[4-[3-chloro-4-[3-(trifluoromethyl)phenoxy]phenylamino]-5H-pyrrolo[3,2-d]pyrimidin-5-yl]ethyl]-3-hydroxy-3-methylbutyramide); AEE788 (6-[4-(4-Ethylpiperazin-1-ylmethyl)phenyl]-N-[1(R)-phenylethyl]-7H-pyrrolo[2,3-d]pyrimidin-4-amine);tarloxotinib 3-[N-[4-(3-Bromo-4-chlorophenylamino)pyrido[3,4-d]pyrimidin-6-yl]carbamoyl]-N,N-dimethyl-N-(1-methyl-4-nitro-1H-imidazol-5-ylmethyl)-2(E)-propen-1-aminium bromide); BMS 599626/AC-480 (N-[4-[1-(3-Fluorobenzyl)-1H-indazol-5-ylamino]-5-methylpyrrolo[2,1-f][1,2,4]triazin-6-yl]carbamic acid morpholin-3(S)-ylmethyl ester hydrochloride); and GW 583340 HCl (N-[3-chloro-4-(3-fluorobenzyloxy)phenyl]-6-[2-[2-(methylsulfonyl)ethylaminomethyl]thiazol-4-yl]quinazolin-4-amine).

In one embodiment, the pan ErbB family inhibitor is a combination of an EGFR inhibitor and a HER2 inhibitor, wherein the EGFR inhibitor and the HER2 inhibitor are a combination of two of: AG 1478 (N-(3-chlorophenyl)-6-methoxy-7-[11C]methoxyquinazolin-4-amine); AG 555 (2-cyano-3-(3,4-dihydroxyphenyl)-N-(3-phenylpropyl)-2(E)-propenamide); AG 556 ((E)-2-cyano-3-(3,4-dihydroxyphenyl)-N-(4-phenylbutyl)acrylamide; AG 825 (3-[3-(benzothiazol-2-ylsulfanylmethyl)-4-hydroxy-5-methoxyphenyl]-2-cyano-2-propenamide); CP 724714 (2-methoxy-N-[3-[4-[3-methyl-4-(6-methylpyridin-3-yloxy)phenylamino]quinazolin-6-yl]-2(E)-propenyl]acetamide; BIBU 1361 (N-(3-chloro-4-fluorophenyl)-6-[4-(diethylaminomethyl)piperidin-1-yl]pyrimido[5,4-d]pyrimidin-4-amine); BIBU 1382; JNJ 28871063 ((E)-4-amino-6-[4-(benzyloxy)-3-chlorophenylamino]pyrimidine-5-carbaldehyde 0-[2-(4-morpholinyl)ethyl]oxime); PD 153035 (4-(3-bromophenylamino)-6,7-dimethoxyquinazoline); and PD 158780 (N4-(3-bromophenyl)-N6-methyl-pyrido[3,4-d]pyrimidine-4,6-diamine).

In one embodiment, the pan ErbB family inhibitor is an anti-EGFR antibody, a anti-HER2 antibody or combination of an anti-EGFR antibody and anti-HER2 antibody. Antibodies, including monoclonal antibodies, antibody conjugates and bispecific antibodies, targeting EGFR and/or HER2 are well known and a number of antibodies are commercially available for research and human clinical use.

Examples of anti-EGFR antibodies suitable for the provided compositions and methods include necitumumab, panitumumab and cetuximab. Examples of anti-HER2 antibodies suitable for the provided compositions and methods include, pertuzumab, trastuzumab, and trastuzumab emtansine.

In yet another aspect, the invention provides for methods for increasing the sensitivity of a cancer cell to a KRas G12D inhibitor, comprising contacting the cancer cell with a therapeutically effective amount of a combination of a KRas G12D inhibitor compound of Formula (I), or a pharmaceutically acceptable salt or a pharmaceutical composition thereof, and a pan ErbB family inhibitor, or a pharmaceutically acceptable salt or a pharmaceutical composition thereof, wherein the pan ErbB family inhibitor synergistically increases the sensitivity of the cancer cell to the KRas G12D inhibitor. In one embodiment, the contacting is in vitro. In one embodiment, the contacting is in vivo.

Also provided herein are methods for treating cancer in a subject in need thereof, the method comprising (a) determining that cancer is associated with a KRas G12D mutation (e.g., a KRas G12D-associated cancer) (e.g., as determined using a regulatory agency-approved, e.g., FDA-approved, assay or kit); and (b) administering to the patient a therapeutically effective amount of a combination of a pan ErbB family inhibitor, or a pharmaceutically acceptable salt or a pharmaceutical composition thereof, and a KRas G12D inhibitor compound of Formula I, or a pharmaceutically acceptable salt or a pharmaceutical composition thereof, wherein the pan ErbB family inhibitor synergistically increases the sensitivity of the KRas G12D-associated cancer to the KRas G12D inhibitor.

Also provided herein are kits comprising a pan ErbB family inhibitor, or a pharmaceutically acceptable salt or a pharmaceutical composition thereof and a KRas G12D inhibitor compound of Formula (I), or a pharmaceutically acceptable salt or a pharmaceutical composition thereof. Also provided is a kit comprising a pan ErbB family inhibitor, or a pharmaceutically acceptable salt or a pharmaceutical composition thereof, and a KRas G12D inhibitor compound of Formula (I), or a pharmaceutically acceptable salt or a pharmaceutical composition thereof, for use in treating a KRas G12D cancer.

In a related aspect, the invention provides a kit containing a dose of a pan ErbB family inhibitor, or a pharmaceutically acceptable salt or a pharmaceutical composition thereof and a KRas G12D inhibitor compound of Formula (I), or a pharmaceutically acceptable salt or a pharmaceutical composition thereof in an amount effective to inhibit proliferation of cancer cells in a subject. The kit in some cases includes an insert with instructions for administration of a pan ErbB family inhibitor, or a pharmaceutically acceptable salt or a pharmaceutical composition thereof and a KRas G12D inhibitor compound of Formula (I), or a pharmaceutically acceptable salt or a pharmaceutical composition thereof. The insert may provide a user with one set of instructions for using a pan ErbB family inhibitor, or a pharmaceutically acceptable salt or a pharmaceutical composition thereof in combination with a KRas G12D inhibitor compound of Formula (I), or a pharmaceutically acceptable salt or a pharmaceutical composition thereof.

In some embodiments of any of the methods described herein, before treatment with the compositions or methods of the invention, the patient was treated with one or more of a chemotherapy, a targeted anticancer agent, radiation therapy, and surgery, and optionally, the prior treatment was unsuccessful; and/or the patient has been administered surgery and optionally, the surgery was unsuccessful; and/or the patient has been treated with a platinum-based chemotherapeutic agent, and optionally, the patient has been previously determined to be non-responsive to treatment with the platinum-based chemotherapeutic agent; and/or the patient has been treated with a kinase inhibitor, and optionally, the prior treatment with the kinase inhibitor was unsuccessful; and/or the patient was treated with one or more other therapeutic agent(s).

### BRIEF DESCRIPTION OF THE DRAWINGS

FIG. 1 depicts the average tumor volumes in mouse xenografts for MRTX1133, alone and in combination with cetuximab (LS180 colon cancer cell line).

FIG. 2 depicts the average tumor volumes in mouse xenografts for MRTX1133, alone and in combination with afatinib (AsPC-1 pancreatic cancer cell line).

FIG. 3 depicts the average tumor volumes in mouse xenografts for MRTX1133, alone and in combination with cetuximab (GP2D colon cancer cell line).

FIG. 4 depicts the average tumor volumes in mouse xenografts for MRTX1133, alone and in combination with afatinib or cetuximab (Panc0203 pancreatic cancer cell line).

FIG. 5 depicts the average tumor volumes in mouse xenografts for MRTX1133, alone and in combination with afatinib or cetuximab (SW1990 pancreatic cancer cell line).

FIG. 6 depicts the average tumor volumes in mouse xenografts for MRTX1133, alone and in combination with cetuximab (SNU1033 rectal cancer cell line).

FIG. 7 depicts the average tumor volumes in mouse xenografts for MRTX1133, alone and in combination with cetuximab (AsPC-1 pancreatic cancer cell line).

FIG. 8 depicts the average tumor volumes in mouse xenografts for MRTX1133, alone and in combination with erlotinib (HPAC pancreatic cancer cell line).

### DETAILED DESCRIPTION OF THE INVENTION

The present invention relates to combination therapies for treating KRas G12D cancers. In particular, the present invention relates to methods of treating cancer in a subject in need thereof, comprising administering to the subject a therapeutically effective amount of a combination of a pan ErbB family inhibitor, or a pharmaceutically acceptable salt or a pharmaceutical composition thereof, and a KRAS G12D inhibitor of Formula (I), or a pharmaceutically acceptable salt or a pharmaceutical composition thereof, pharmaceutical compositions comprising therapeutically effective amounts of the inhibitors, kits comprising the compositions and methods of use therefor.

Combinations of a pan ErbB family inhibitor, or a pharmaceutically acceptable salt or a pharmaceutical composition thereof, with a KRas G12D inhibitor, or a pharmaceutically acceptable salt or a pharmaceutical composition thereof, synergistically increase the potency of KRas G12D inhibitor compounds of Formula (I), against cancer cells that express KRas G12D thereby increasing the efficacy and therapeutic index of KRas G12D inhibitor compounds of Formula (I), or pharmaceutically acceptable salts thereof.

### Definitions

Unless defined otherwise, all technical and scientific terms used herein have the same meaning as is commonly understood by one of skill in the art to which this invention belongs. All patents, patent applications, and publications referred to herein are incorporated by reference.

As used herein, “KRas G12D” refers to a mutant form of a mammalian KRas protein that contains an amino acid substitution of an aspartic acid for a glycine at amino acid position 12. The assignment of amino acid codon and residue positions for human KRas is based on the amino acid sequence identified by UniProtKB/Swiss-Prot P01116: Variant p.Gly12Asp.

As used herein, a “KRas G12D inhibitor” refers to compounds of the present invention that are represented by Formula (I), as described herein. These compounds are capable of negatively modulating or inhibiting all or a portion of the enzymatic activity of KRas G12D. In one embodiment, the KRas G12D inhibitor is a compound selected from compound Nos 1-458 (as numbered in WO2021/041671), or pharmaceutically acceptable salts thereof.

A “KRas G12D-associated disease or disorder” as used herein refers to diseases or disorders associated with or mediated by or having a KRas G12D mutation. A non-limiting example of a KRas G12D-associated disease or disorder is a KRas G12D-associated cancer.

As used herein, an “ErbB family” or “ErbB family member” refers to a member of a mammalian transmembrane protein tyrosine kinase family including: EGFR, ErbB2 (HER2), ErbB3 (HER3), and ErbB4 (HER4).

As used herein, a “pan ErbB family inhibitor” refers to an agent, e.g., a compound or antibody, that is capable of negatively modulating or inhibiting all or a portion of the activity of at least one member of the ErbB family. The modulation or inhibition of one or more ErbB family members may occur through modulating or inhibiting kinase enzymatic activity of one or more ErbB family member or by blocking homodimerization or heterodimerization of ErbB family members. In some embodiments of the methods herein, the term “pan ErbB inhibitor” refers to the use of a single pan ErbB inhibitor. In some embodiments of the methods herein, the term “pan ErbB inhibitor” refers to the use of two pan ErbB inhibitors.

As used herein, the term “subject,” “individual,” or “patient,” used interchangeably, refers to any animal, including mammals such as mice, rats, other rodents, rabbits, dogs, cats, swine, cattle, sheep, horses, primates, and humans. In some embodiments, the patient is a human. In some embodiments, the subject has experienced and/or exhibited at least one symptom of the disease or disorder to be treated and/or prevented. In some embodiments, the subject has been identified or diagnosed as having a cancer having a KRas G12D mutation (e.g., as determined using a regulatory agency-approved, e.g., FDA-approved, assay or kit). In some embodiments, the subject has a tumor that is positive for a KRas G12D mutation (e.g., as determined using a regulatory agency-approved assay or kit). The subject can be a subject with a tumor(s) that is positive for a KRas G12D mutation (e.g., identified as positive using a regulatory agency-approved, e.g., FDA-approved, assay or kit). The subject can be a subject whose tumors have a KRas G12D mutation (e.g., where the tumor is identified as such using a regulatory agency-approved, e.g., FDA-approved, kit or assay). In some embodiments, the subject is suspected of having a KRas G12D gene-associated cancer. In some embodiments, the subject has a clinical record indicating that the subject has a tumor that has a KRas G12D mutation (and optionally the clinical record indicates that the subject should be treated with any of the compositions provided herein).

The term “pediatric patient” as used herein refers to a patient under the age of 16 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W.B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994.

In some embodiments of any of the methods or uses described herein, an assay is used to determine whether the patient has KRas G12D mutation using a sample (e.g., a biological sample or a biopsy sample such as a paraffin-embedded biopsy sample) from a patient (e.g., a patient suspected of having a KRas G12D-associated cancer, a patient having one or more symptoms of a KRas G12D-associated cancer, and/or a patient that has an increased risk of developing a KRas G12D-associated cancer) can include, for example, next generation sequencing, immunohistochemistry, fluorescence microscopy, break apart FISH analysis, Southern blotting, Western blotting, FACS analysis, Northern blotting, and PCR-based amplification (e.g., RT-PCR, quantitative real-time RT-PCR, allele-specific genotyping or ddPCR). As is well-known in the art, the assays are typically performed, e.g., with at least one labelled nucleic acid probe or at least one labelled antibody or antigen-binding fragment thereof.

The term “regulatory agency” is a country's agency for the approval of the medical use of pharmaceutical agents with the country. For example, a non-limiting example of a regulatory agency is the U.S. Food and Drug Administration (FDA).

The term “amino” refers to —NH₂;

The term “acyl” refers to —C(O)CH₃.

The term “alkyl” as employed herein refers to straight and branched chain aliphatic groups having from 1 to 12 carbon atoms, 1-8 carbon atoms 1-6 carbon atoms, or 1-3 carbon atoms which is optionally substituted with one, two or three substituents. Examples of alkyl groups include, without limitation, methyl, ethyl, propyl, isopropyl, butyl, isobutyl, sec-butyl, tert-butyl, pentyl, and hexyl.

The term “haloalkyl” refers to an alkyl chain in which one or more hydrogen has been replaced by a halogen. Examples of haloalkyls are trifluoromethyl, difluoromethyl and fluoromethyl.

The term “haloalkyloxy” refers to —O-haloalkyl.

An “alkylene,” group is an alkyl group, as defined hereinabove, that is positioned between and serves to connect two other chemical groups. Exemplary alkylene groups include, without limitation, methylene, ethylene, propylene, and butylene.

The term “alkoxy” refers to —OC1-C6 alkyl.

The term “cycloalkyl” as employed herein includes saturated and partially unsaturated cyclic hydrocarbon groups having 3 to 12 carbons, for example 3 to 8 carbons, and as a further example 3 to 6 carbons, wherein the cycloalkyl group additionally is optionally substituted. Examples of cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroalkyl” refers to an alkyl group, as defined hereinabove, wherein one or more carbon atoms in the chain are replaced by a heteroatom selected from the group consisting of O, S, and N.

As used herein, the term “hydroxyalkyl” refers to -alkyl-OH.

The term “dihydroxyalkyl” refers to an alkyl group as defined herein wherein two carbon atoms are each substituted with a hydroxyl group.

The term “alkylaminyl” refers to —NR^{x}-alkyl, wherein R^{x} is hydrogen. In one embodiment, R^{x} is hydrogen.

The term “dialkylaminylalkyl” refers to -alkyl-N(R^{y})₂, wherein each R^{y} is C1-C4 alkyl, wherein the alkyl of the -alkyl-N(R^{y})₂ may be optionally substituted with hydroxy or hydroxyalkyl.

An “aryl” group is a C₆-C₁₄ aromatic moiety comprising one to three aromatic rings, which is optionally substituted. As one embodiment, the aryl group is a C₆-C₁₀ aryl group. Examples of aryl groups include, without limitation, phenyl, naphthyl, anthracenyl, fluorenyl, and dihydrobenzofuranyl.

An “aralkyl” or “arylalkyl” group comprises an aryl group covalently linked to an alkyl group, either of which may independently be optionally substituted or unsubstituted. An example of an aralkyl group is (C₁-C₆)alkyl(C₆-C₁₀)aryl, including, without limitation, benzyl, phenethyl, and naphthylmethyl. An example of a substituted aralkyl is wherein the alkyl group is substituted with hydroxyalkyl.

An “aryl” group is a C₆-C₁₄ aromatic moiety comprising one to three aromatic rings, which is optionally substituted. As one embodiment, the aryl group is a C₆-C₁₀ aryl group. Examples of aryl groups include, without limitation, phenyl, naphthyl, anthracenyl, fluorenyl, and dihydrobenzofuranyl.

An “aralkyl” or “arylalkyl” group comprises an aryl group covalently linked to an alkyl group, either of which may independently be optionally substituted or unsubstituted. An example of an aralkyl group is (C₁-C₆)alkyl(C₆-C₁₀)aryl, including, without limitation, benzyl, phenethyl, and naphthylmethyl. An example of a substituted aralkyl is wherein the alkyl group is substituted with hydroxyalkyl.

A “heterocyclyl” or “heterocyclic” group is a ring structure having from about 3 to about 12 atoms, for example 4 to 8 atoms, wherein one or more atoms are selected from the group consisting of N, O, and S, the remainder of the ring atoms being carbon. The heterocyclyl may be a monocyclic, a bicyclic, a spirocyclic or a bridged ring system. The heterocyclic group is optionally substituted with R⁷ on carbon or nitrogen at one or more positions, wherein R⁷ is as defined for Formula I. The heterocyclic group is also independently optionally substituted on nitrogen with alkyl, aryl, aralkyl, alkylcarbonyl, alkylsulfonyl, arylcarbonyl, arylsulfonyl, alkoxycarbonyl, aralkoxycarbonyl, or on sulfur with oxo or lower alkyl. Examples of heterocyclic groups include, without limitation, epoxy, azetidinyl, aziridinyl, tetrahydrofuranyl, tetrahydropyranyl, pyrrolidinyl, pyrrolidinonyl, piperidinyl, piperazinyl, imidazolidinyl, thiazolidinyl, dithianyl, trithianyl, dioxolanyl, oxazolidinyl, oxazolidinonyl, decahydroquinolinyl, piperidonyl, 4-piperidinonyl, thiomorpholinyl, thiomorpholinyl 1,1 dioxide, morpholinyl, oxazepanyl, azabicyclohexanes, azabicycloheptanes and oxa azabiocycloheptanes. Specifically excluded from the scope of this term are compounds having adjacent annular O and/or S atoms.

The term “heterocyclylalkyl” refers to a heterocyclyl group as defined herein linked to the remaining portion of the molecule via an alkyl linker, wherein the alkyl linker of the heterocyclylalkyl may be optionally substituted with hydroxy or hydroxyalkyl.

As used herein, the term “heteroaryl” refers to groups having 5 to 14 ring atoms, preferably 5, 6, 9, or 10 ring atoms; having 6, 10, or 14 π electrons shared in a cyclic array; and having, in addition to carbon atoms, from one to three heteroatoms per ring selected from the group consisting of N, O, and S. Examples of heteroaryl groups include acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, furanyl, furazanyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, methylenedioxyphenyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolinyl, 2H-pyrrolyl, pyrrolyl, quinazolinyl, quinolinyl, 4H-quinolizinyl, quinoxalinyl, quinuclidinyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

A “heteroarylalkyl” group comprises a heteroaryl group covalently linked to an alkyl group, wherein the radical is on the alkyl group, either of which is independently optionally substituted or unsubstituted. Examples of heteroarylalkyl groups include a heteroaryl group having 5, 6, 9, or 10 ring atoms bonded to a C1-C6 alkyl group. Examples of heteroaralkyl groups include pyridylmethyl, pyridylethyl, pyrrolylmethyl, pyrrolylethyl, imidazolylmethyl, imidazolylethyl, thiazolylmethyl, thiazolylethyl, benzimidazolylmethyl, benzimidazolylethyl quinazolinylmethyl, quinolinylmethyl, quinolinylethyl, benzofuranylmethyl, indolinylethyl isoquinolinylmethyl, isoinodylmethyl, cinnolinylmethyl, and benzothiophenylethyl. Specifically excluded from the scope of this term are compounds having adjacent annular O and/or S atoms.

As used herein, “an effective amount” of a compound is an amount that is sufficient to negatively modulate or inhibit the activity of the desired target, i.e., a ErbB family member or KRas G12D. Such amount may be administered as a single dosage or may be administered according to a regimen, whereby it is effective.

As used herein, a “therapeutically effective amount” of a compound is an amount that is sufficient to ameliorate, or in some manner reduce a symptom or stop or reverse progression of a condition, or negatively modulate or inhibit the activity of ErbB family member or KRas G12D. Such amount may be administered as a single dosage or may be administered according to a regimen, whereby it is effective.

As used herein, a “therapeutically effective amount of a combination” of two compounds is an amount that together synergistically increases the activity of the combination in comparison to the therapeutically effective amount of each compound in the combination, i.e., more than merely additive. Alternatively, in vivo, the therapeutically effective amount of the combination of a pan ErbB family member inhibitor, or a pharmaceutically acceptable salt or a pharmaceutical composition thereof, and a KRas G12D inhibitor compound of Formula (I), or a pharmaceutically acceptable salt or a pharmaceutical composition thereof, results in an increased duration of overall survival (“OS”) in subjects relative to treatment with only the KRas G12D inhibitor. In one embodiment, the therapeutically effective amount of the combination of a pan ErbB family member inhibitor, or a pharmaceutically acceptable salt or a pharmaceutical composition thereof, and a KRas G12D inhibitor compound of Formula (I), or a pharmaceutically acceptable salt or a pharmaceutical composition thereof, results in an increased duration of progression-free survival (“PF S”) in subjects relative to treatment with only the KRas G12D inhibitor. In one embodiment, the therapeutically effective amount of the combination of a pan ErbB inhibitor, or a pharmaceutically acceptable salt or a pharmaceutical composition thereof, and a KRas G12D inhibitor compound of Formula (I), or a pharmaceutically acceptable salt or a pharmaceutical composition thereof, results in increased tumor regression in subjects relative to treatment with only the KRas G12D inhibitor. In one embodiment, the therapeutically effective amount of the combination of a pan ErbB inhibitor, or a pharmaceutically acceptable salt or a pharmaceutical composition thereof, and a KRas G12D inhibitor compound of Formula (I), or a pharmaceutically acceptable salt or a pharmaceutical composition thereof, results in increased tumor growth inhibition in subjects relative to treatment with only the KRas G12D inhibitor. In one embodiment, the therapeutically effective amount of the combination of a pan ErbB inhibitor, or a pharmaceutically acceptable salt or a pharmaceutical composition thereof, and a KRas G12D inhibitor compound of Formula (I), or a pharmaceutically acceptable salt or a pharmaceutical composition thereof, results in an improvement in the duration of stable disease in subjects compared to treatment with only the KRas G12D inhibitor. The amount of each compound in the combination may be the same or different than the therapeutically effective amount of each compound when administered alone as a monotherapy as long as the combination is synergistic. Such amounts may be administered as a single dosage or may be administered according to a regimen, whereby it is effective.

As used herein, treatment means any manner in which the symptoms or pathology of a condition, disorder or disease are ameliorated or otherwise beneficially altered. Treatment also encompasses any pharmaceutical use of the compositions herein.

As used herein, amelioration of the symptoms of a particular disorder by administration of a particular pharmaceutical composition refers to any lessening, whether permanent or temporary, lasting or transient that can be attributed to or associated with administration of the composition.

As used herein, the term “about” when used to modify a numerically defined parameter (e.g., the dose of a KRAS inhibitor or a pan ErbB family inhibitor or a pharmaceutically acceptable salt thereof, or the length of treatment time with a combination therapy described herein) means that the parameter may vary by as much as 10% below or above the stated numerical value for that parameter. For example, a dose of about 5 mg/kg may vary between 4.5 mg/kg and 5.5 mg/kg. “About” when used at the beginning of a listing of parameters is meant to modify each parameter. For example, about 0.5 mg, 0.75 mg or 1.0 mg means about 0.5 mg, about 0.75 mg or about 1.0 mg. Likewise, about 5% or more, 10% or more, 15% or more, 20% or more, and 25% or more means about 5% or more, about 10% or more, about 15% or more, about 20% or more, and about 25% or more.

### Inhibitor Compounds

In one aspect of the invention, provided herein are methods of treating cancer in a subject in need thereof, comprising administering to the subject a therapeutically effective amount of a combination of a pan ErbB family inhibitor, or a pharmaceutically acceptable salt or a pharmaceutical composition thereof, and a KRAS G12D inhibitor of Formula (I), or a pharmaceutically acceptable salt or a pharmaceutical composition thereof.

### 1. ErbB Family

Epidermal Growth Factor Receptor (EGFR) is a transmembrane protein tyrosine kinase of the ErbB receptor family. Upon binding epidermal growth factor (EGF), the EGFR receptor can homo-dimerize with another EGFR molecule or hetero-dimerize with another family member such as ErbB2 (HER2), ErbB3 (HER3), or ErbB4 (HER4). Homo- and/or hetero-dimerization of ErbB receptors results in the phosphorylation of key tyrosine residues in the intracellular domain and leads to the stimulation of numerous intracellular signal transduction pathways involved in cell proliferation and survival.

Overexpression of the EGFR gene has been identified in a variety of cancers including bladder, brain, head and neck, pancreas, lung, breast, ovary, colon, prostate, and kidney. In addition to overexpression, EGFR activating mutations have been detected in a subset of non-small cell lung cancers (NSCLCs) tumors. These mutations tend to occur within EGFR exons 18-21, which encodes a portion of the EGFR kinase domain. Approximately 90% of these mutations are exon 19 deletions or exon 21 L858R point mutations (Ladanyi and Pao (2008) Mod Path. May; 21 Suppl 2:S16-22. doi: 10.1038/modpathol.3801018). These mutations increase the kinase activity of EGFR, leading to hyperactivation of downstream pro-survival signaling pathways.

The frequency of overexpression and/or activating mutations of EGFR has made it a desired target for anticancer therapies and a number of EGFR inhibitors have been developed and are clinically available.

First generation erlotinib and gefitinib inhibit EGFR activity by competitively binding to the ATP binding site of the EGFR kinase domain; however additional mutations in the EGFR gene, e.g., the T790M mutation, produces mutant EGFR proteins to which drugs like erlotinib and gefitinib bind less well. Those mutations are associated with resistance to the drugs and to relapse in cancer patients bearing such mutation leading to the development of second generation EGFR inhibitors targeting the T790M mutant.

Furthermore, inhibition of the pathway-related enzyme MEK results in increased expression of ErbB family members, especially EGFR, that can lead to adaptive and acquired resistance to ErbB family inhibitors (Sun et al., (2014) Cell Reports 7:86-93).

2. Pan ErbB family Inhibitors

The pan ErbB family inhibitors used in the methods of the present invention may be reversible or irreversible ErbB family inhibitors. In one embodiment, the pan ErbB family inhibitor inhibits the activity of more than one ErbB family member.

In one embodiment, the pan ErbB family inhibitor is an irreversible inhibitor. Irreversible pan ErbB family inhibitors inhibit the activity of EGFR and HER2 by forming a covalent bond with the sulfhydryl group of cysteine 797 and cysteine 773, respectively, that blocks the binding of ATP to the intracellular catalytic domain. As such, these inhibitors are active against, for example, cell lines harboring EGFR exon 19 deletions/insertions, and L858R and T790M resistant mutations.

Exemplary irreversible pan ErbB family inhibitors for use in the methods include afatinib ((E)-N-(4-((3-chloro-4-fluorophenyl)amino)-7-((tetrahydrofuran-3-yl)oxy)quinazolin-6-yl)-4-(dimethylamino)but-2-enamide); dacomitinib ((2E)-N-{4-[(3-Chloro-4-fluorophenyl)amino]-7-methoxy-6-quinazolinyl}-4-(1-piperidinyl)-2-butenamide); canertinib (N-(4-((3-chloro-4-fluorophenyl)amino)-7-(3-morpholinopropoxy)quinazolin-6-yl)acrylamide); poziotinib (1-(4-((4-((3,4-dichloro-2-fluorophenyl)amino)-7-methoxyquinazolin-6-yl)oxy)piperidin-1-yl)prop-2-en-1-one); AV 412 (N-[4-[(3-Chloro-4-fluorophenyl)amino]-7-[3-methyl-3-(4-methyl-1-piperazinyl)-1-butyn-1-yl]-6-quinazolinyl]-2-propenamide); PF 6274484 (N-[4-[(3-Chloro-4-fluorophenyl)amino]-7-methoxy-6-quinazolinyl]-2-propenamide) and HKI 357 ((2E)-N-[[4-[[(3-Chloro-4-[(3-fluorophenyl)methoxy]phenyl]amino]-3-cyano-7-ethoxy-6-quinolinyl]-4-(dimethylamino)-2-butenamide), and pharmaceutically acceptable salts or pharmaceutical compositions thereof. In one embodiment, the irreversible pan ErbB family inhibitor is afatinib. In one embodiment, the irreversible pan ErbB family inhibitor is dacomitinib.

In one embodiment, the pan ErbB family inhibitor is a reversible inhibitor. Exemplary reversible pan EGFR family inhibitors include erlotinib ([6,7-Bis-(2-methoxy-ethoxy)-quinazolin-4-yl]-(3-ethynyl-phenyl)-amine)), gefitinib (4-(3′-chloro-4′-fluoroanilino)-7-methoxy-6-(3-morpholinopropoxy)quinazoline, sapitinib (2-(4-((4-((3-chloro-2-fluorophenyl)amino)-7-methoxyquinazolin-6-yl)oxy)piperidin-1-yl)-N-methylacetamide); varlitinib ((R)—N4-(3-chloro-4-(thiazol-2-ylmethoxy)phenyl)-N6-(4-methyl-4,5-dihydrooxazol-2-yl)quinazoline-4,6-diamine); TAK-285 (N-(2-(4-((3-chloro-4-(3-(trifluoromethyl)phenoxy)phenyl)amino)-5H-pyrrolo[3,2-d]pyrimidin-5-yl)ethyl)-3-hydroxy-3-methylbutanamide); AEE788 ((S)-6-(4-((4-ethylpiperazin-1-yl)methyl)phenyl)-N-(1-phenylethyl)-7H-pyrrolo[2,3-d]pyrimidin-4-amine); tarloxotinib 3-[N-[4-(3-Bromo-4-chlorophenylamino)pyrido[3,4-d]pyrimidin-6-yl]carbamoyl]-N,N-dimethyl-N-(1-methyl-4-nitro-1H-imidazol-5-ylmethyl)-2(E)-propen-1-aminium bromide); BMS 599626 ((3S)-3-Morpholinylmethyl-[4-[[1-[(3-fluorophenyl)methyl]-1H-indazol-5-yl]amino]-5-methylpyrrolo[2,1-f][1,2,4]triazin-6-yl]-carbamate dihydrochloride); and GW 583340 HCl (N-[3-Chloro-4-[(3-fluorophenyl)methoxy]phenyl]-6-[2-[[[2-(methylsulfonyl)ethyl]amino]methyl]-4-thiazolyl]-4-quinazolinamine dihydrochloride), and pharmaceutically acceptable salts or pharmaceutical compositions thereof. In one embodiment, the reversible pan ErbB family inhibitor is sapitinib. In one embodiment, the reversible pan ErbB family inhibitor is tarloxotinib.

In one embodiment, the pan ErbB family inhibitor is a combination of an EGFR inhibitor and a HER2 inhibitor, wherein the EGFR inhibitor and the HER2 inhibitor are a combination of two of: AG 1478 HCl (N-(3-Chlorophenyl)-6,7-dimethoxy-4-quinazolinanine hydrochloride); AG 494 (E)-2-Cyano-3-(3,4-dihydroxyphenyl)-N-phenyl-2-propenamide; AG 555 (E)-2-Cyano-3-(3,4-dihydroxyphenyl)-N-(3-phenylpropyl)-2-propenamide; AG 556 (E)-2-Cyano-3-(3,4-dihydroxyphenyl)-N-(4-phenylbutyl)-2-propenamide; AG 825 (E)-3-[3-[2-Benzothiazolythio)methyl]-4-hydroxy-5-methoxyphenyl]-2-cyano-2-propenamide; CP 724714 (2-Methoxy-N-[(2E)-3-[4-[[3-methyl-4-[(6-methyl-3-pyridinyl)oxy]phenyl]amino]-6-quinazolinyl]-2-propen-1-yl]acetamide; BIBU 1361 diHCl (N-(3-Chloro-4-fluorophenyl)-6-[4-[(diethylamino)methyl]-1-piperidinyl]-pyrimido[5,4-d]pyrimidin-4-amine dihydrochloride); BIBU 1382 (N⁸-(3-Chloro-4-fluorophenyl)-N²-(1-methyl-4-piperidinyl)-pyrimido[5,4-d]pyrimidine-2,8-diamine dihydrochloride); JNJ 28871063 HCl (5E-4-Amino-6-(4-benzyloxy-3-chlorophenylamino)pyrimidine-5-carboxaldehyde N-(2-morpholin-4-ylethyl) oxime hydrochloride); PD 153035 (4-[(3-Bromophenyl)amino]-6,7-dimethoxyquinazoline hydrochloride); PD 158780 (N⁴-(3-Bromophenyl)-N⁶-methyl-pyrido[3,4-d]pyrimidine-4,6-diamine), and pharmaceutically acceptable salts or a pharmaceutical compositions thereof.

Methods for manufacturing reversible and irreversible pan ErbB family inhibitors that target wild type and mutant ErbB family members are well known to those skilled in the art and pan ErbB family inhibitors may be obtained from a wide-variety of commercial suppliers, in forms suitable for both research or human use. In addition, suitable reversible and irreversible pan ErbB family inhibitors for use in the compositions and methods disclosed herein, and methods for preparing such inhibitors are disclosed in US Patent Application Publication Nos: US20180050993; US20180016268; US20180008607; US20170362204; US 20170362203; US20170355683; US20170342055; US20170267671; US20170183330; US20170174697; 20170008856; US20160375148; US20160332994; US20160257682; US 20160244469; US 20160137610; US20160102076; US20160016948; US20150284340; US20150274678; US20150250778; US 20150246047; US20150126508; US20150025055; US 20140221403; US 20140178412; US20140161722; US20140155606; US20140038981; US20140038940; US20140005391; US 20130296348; US20130209461; US20130137709; US 20120316135L US 20120094999; US 20110295004; US 20110033453; US 20100196365; US20100143295; US 20100120678; US 20100034689; US 20090209758; US 20090111772; US20090029968; US20080194578; US 20080139590; US 2000125448; US 20080051395; US 20070232607; US 20060235046 and US20040023957.

In one embodiment, the pan ErbB family inhibitor is an anti-EGFR antibody, an anti-HER2 antibody or a combination of an anti-EGFR antibody and anti-HER2 antibody, or pharmaceutical compositions thereof. Antibodies, including monoclonal antibodies, antibody drug conjugates and bispecific antibodies, targeting EGFR and/or HER-2 are well known and a number of antibodies are commercially available for research and human clinical use.

Exemplary anti-EGFR monoclonal antibodies approved for human clinical use include, but are not limited to, necitumumab (Eli Lilly), panitumumab (Amgen) and cetuximab (ImClone). Other anti-EGFR antibodies suitable for use in the methods include EP384, H11, 11.6, 225 and 199.12 (Thermo Fisher), GT133 (GeneTex) and those disclosed in United States Patent Application Publication Nos: US 20080274114; US 20100166755; US 20100117110; US 20120034211; US 20120308576; US 20130273033; US 20130344093; US 20140286969; US 20150337042; US 20170218073; US 20170267765, US 20180036405, US 20180066066, US 20180094062, US 20180155433, US 20180306049, US 20180362443, US 20190040143, US 20190151328, US 20190194347, US 20190194350, US 20190209704, US 20190216924, and US 20190263930.

In one embodiment, the anti-EGFR monoclonal antibody is cetuximab.

Exemplary anti-HER-2 monoclonal antibodies approved for human clinical use include, but are not limited to, pertuzumab (Roche), trastuzumab (Roche) and trastuzumab emtansine (Roche). Other anti-Her2 antibodies, antibody drug conjugates and bispecific antibodies suitable for use in the methods include those disclosed in United States Patent Application Publication Nos: US 20030228663; US 20060018899; US 20090187007; US 20090285837; US 20110159014; US 20110177095; US 20110313137; US 20120309942; US 20150166664; US 20150352225; US 20160051695; US 20160096893, US 20180022816, US 20180022820, US 20180057608, US 20180118837, US 20180258173, US 20190177428, and US 20190248918.

### 3. KRas G12D Inhibitors

In one embodiment, the KRas G12D inhibitors used in the methods are compounds of Formula (I):

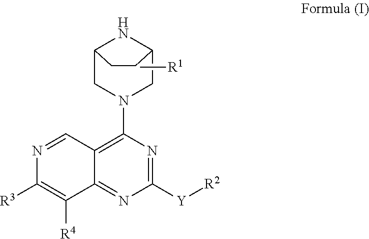

or a pharmaceutically acceptable salt thereof:
wherein:
R¹ is hydrogen, hydroxy, halogen, C1-C3 alkyl, C1-C3 cyanoalkyl, C1-C3 hydroxyalkyl, HC(═O)—, —CO₂R⁵, —CO₂N(R⁵)₂ or a 5-6 membered heteroaryl;
Y is a bond, O or NR⁵;
R² is hydrogen, —N(R⁵)₂, heterocyclyl, C1-C6 alkyl, -L-heterocyclyl, -L-aryl, -L-heteroaryl, -L-cycloalkyl, -L-N(R⁵)₂, -L-NHC(═NH)NH₂, -L-C(O)N(R⁵)₂, -L-C1-C6 haloalkyl, -L-OR⁵, -L-(CH₂OR⁵)(CH₂)ₙOR⁵, -L-NR⁵C(O)-aryl, -L-COOH, or -LC(═O)OC1-C6 alkyl, wherein the heterocyclyl and the aryl portion of -L-NR⁵C(O)-aryl and the heterocyclyl portion of -L-heterocyclyl and the cycloalkyl portion of the -L-cycloalkyl may be optionally substituted with one or more R⁶, and wherein the aryl or heteroaryl of the -L-aryl and the -L-heteroaryl may be optionally substituted with one or more R⁷;
each L is independently a C1-C4 alkylene optionally substituted with hydroxy, C1-C4 hydroxyalkyl or heteroaryl;
R³ is aryl or heteroaryl, wherein the aryl or the heteroaryl is optionally substituted with one or more R⁸;
R⁴ is hydrogen, halogen or C1-C3 alkyl;
each R⁵ is independently hydrogen or C1-C3 alkyl;
each R⁶ is independently halogen, hydroxy, C1-C3 hydroxyalkyl, C1-C3 alkyl, C1-C3 haloalkyl, C1-C3 alkoxy, cyano, -Q-phenyl, -Q-phenylSO₂F, —NHC(O)phenyl, —NHC(O)phenylSO₂F, C1-C3 alkyl substituted pyrazolyl, araC1-C3 alkyl-, tert-butyldimethylsilyloxyCH₂-, —N(R⁵)₂, (C1-C3 alkoxy)C1-C3 alkyl-, (C1-C3 alkyl)C(═O), oxo, (C1-C3 haloalkyl)C(═O)—, —SO₂F, (C1-C3 alkoxy)C1-C3 alkoxy, —CH₂OC(O)N(R⁵)₂, —CH₂NHC(O)OC1-C6 alkyl, —CH₂NHC(O)N(R⁵)₂, —CH₂NHC(O)C1-C6 alkyl, —CH₂(pyrazolyl), —CH₂NHSO₂C1-C6 alkyl, —CH₂OC(O)heterocyclyl, —OC(O)N(R⁵)₂, —OC(O)NH(C1-C3 alkyl)O(C1-C3 alkyl), —OC(O)NH(C1-C3 alkyl)O(C1-C3 alkyl)phenyl(C1-C3 alkyl)N(CH₃)₂, —OC(O)NH(C1-C3 alkyl)O(C1-C3 alkyl)phenyl or —OC(O)heterocyclyl, —CH₂heterocyclyl, wherein the phenyl of —NHC(O)phenyl or —OC(O)NH(C1-C3 alkyl)O(C1-C3 alkyl)phenyl is optionally substituted with —C(O)H or OH and wherein the heterocyclyl of —CH₂heterocyclyl is optionally substituted with oxo;
Q is a bond or O;
each R⁷ is independently halogen, hydroxy, HC(═O)—, C1-C4 alkyl, C1-C4 alkoxy, C1-C4 haloalkyl, C1-C4 hydroxyalkyl, or —N(R⁵)₂; and
each R⁸ is independently halogen, cyano, hydroxy, C1-C4 alkyl, —S—C1-C3 alkyl, C2-C4 alkenyl, C2-C4 alkynyl, C2-C4 hydroxyalkynyl, C1-C3 cyanoalkyl, triazolyl, C1-C3 haloalkyl, —O—C1-C3 haloalkyl, —S—C1-C3 haloalkyl, C1-C3 alkoxy, hydroxyC1-C3 alkyl, —CH₂C(═O)N(R⁵)₂, —C3-C4 alkynyl(NR⁵)₂, —N(R⁵)₂, deuteroC2-C4 alkynyl, (C1-C3 alkoxy)haloC1-C3 alkyl-, or C3-C6 cycloalkyl wherein said C3-C6 cycloalkyl is optionally substituted with halogen or C1-C3 alkyl.

Nonlimiting examples of KRas G12D inhibitor compounds of Formula (I), useful in the methods disclosed herein are are selected from the group consisting of compound Nos 1-458 (as numbered in WO2021/041671), or pharmaceutically acceptable salts thereof, including the following structures: In one embodiment, the KRas G12D inhibitor is selected from:

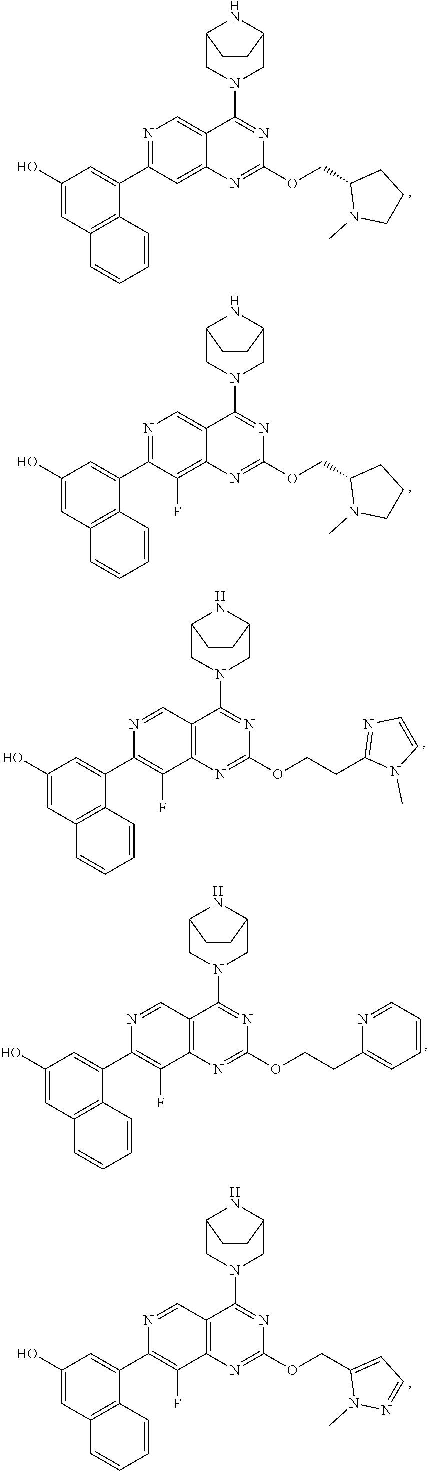 [SMILES: CN1CCC[C@H]1COc1nc(N2CC3CCC(C2)N3)c2cnc(-c3cc(O)cc4ccccc34)cc2n1 ; CN1CCC[C@H]1COc1nc(N2CC3CCC(C2)N3)c2cnc(-c3cc(O)cc4ccccc34)c(F)c2n1 ; Cn1ccnc1CCOc1nc(N2CC3CCC(C2)N3)c2cnc(-c3cc(O)cc4ccccc34)c(F)c2n1 ; Oc1cc(-c2ncc3c(N4CC5CCC(C4)N5)nc(OCCc4ccccn4)nc3c2F)c2ccccc2c1 ; Cn1nccc1COc1nc(N2CC3CCC(C2)N3)c2cnc(-c3cc(O)cc4ccccc34)c(F)c2n1]

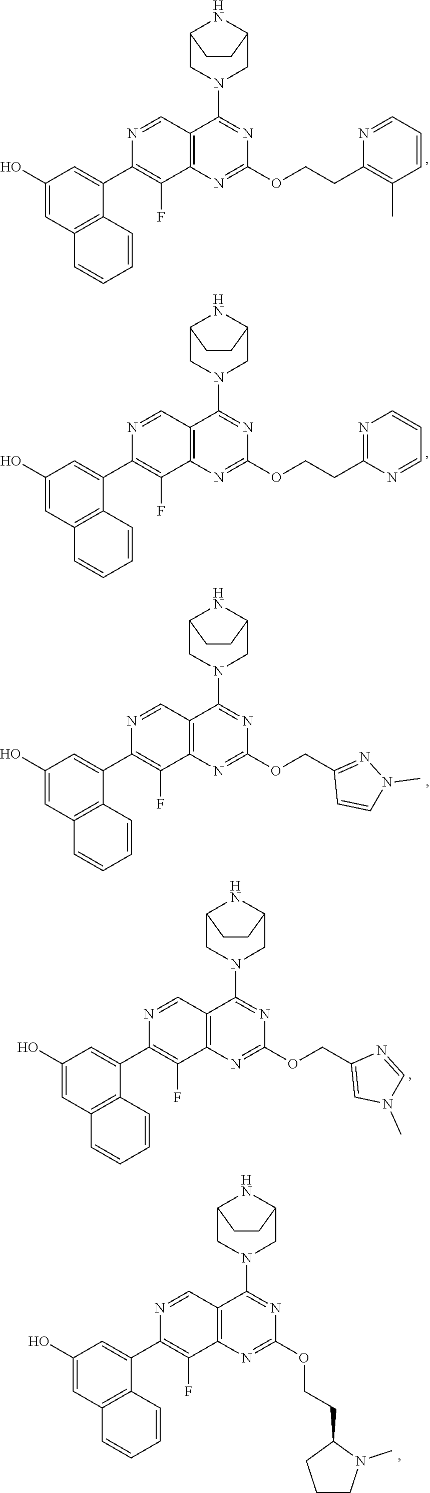 [SMILES: Cc1cccnc1CCOc1nc(N2CC3CCC(C2)N3)c2cnc(-c3cc(O)cc4ccccc34)c(F)c2n1 ; Oc1cc(-c2ncc3c(N4CC5CCC(C4)N5)nc(OCCc4ncccn4)nc3c2F)c2ccccc2c1 ; Cn1ccc(COc2nc(N3CC4CCC(C3)N4)c3cnc(-c4cc(O)cc5ccccc45)c(F)c3n2)n1 ; Cn1cnc(COc2nc(N3CC4CCC(C3)N4)c3cnc(-c4cc(O)cc5ccccc45)c(F)c3n2)c1 ; CN1CCC[C@H]1CCOc1nc(N2CC3CCC(C2)N3)c2cnc(-c3cc(O)cc4ccccc34)c(F)c2n1]

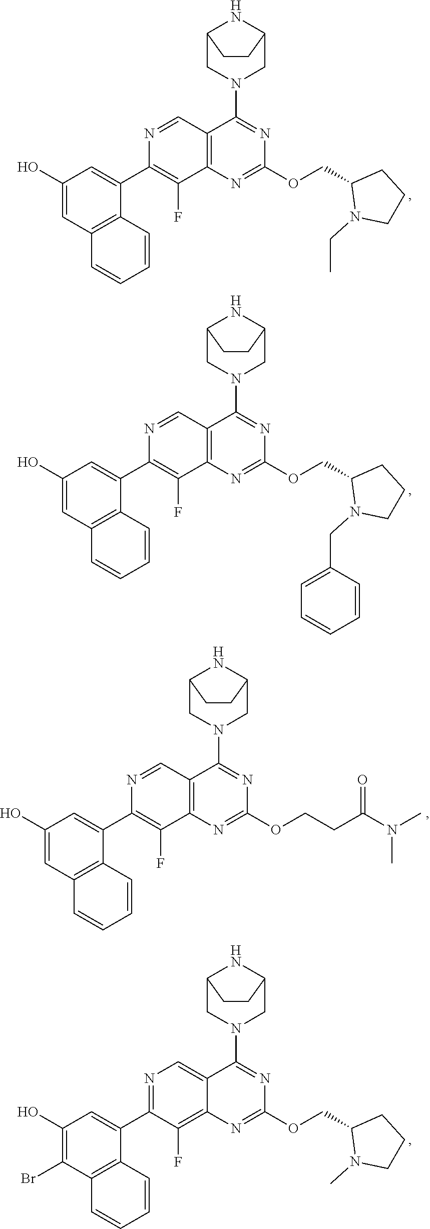 [SMILES: CCN1CCC[C@H]1COc1nc(N2CC3CCC(C2)N3)c2cnc(-c3cc(O)cc4ccccc34)c(F)c2n1 ; Oc1cc(-c2ncc3c(N4CC5CCC(C4)N5)nc(OC[C@@H]4CCCN4Cc4ccccc4)nc3c2F)c2ccccc2c1 ; CN(C)C(=O)CCOc1nc(N2CC3CCC(C2)N3)c2cnc(-c3cc(O)cc4ccccc34)c(F)c2n1 ; CN1CCC[C@H]1COc1nc(N2CC3CCC(C2)N3)c2cnc(-c3cc(O)c(Br)c4ccccc34)c(F)c2n1]

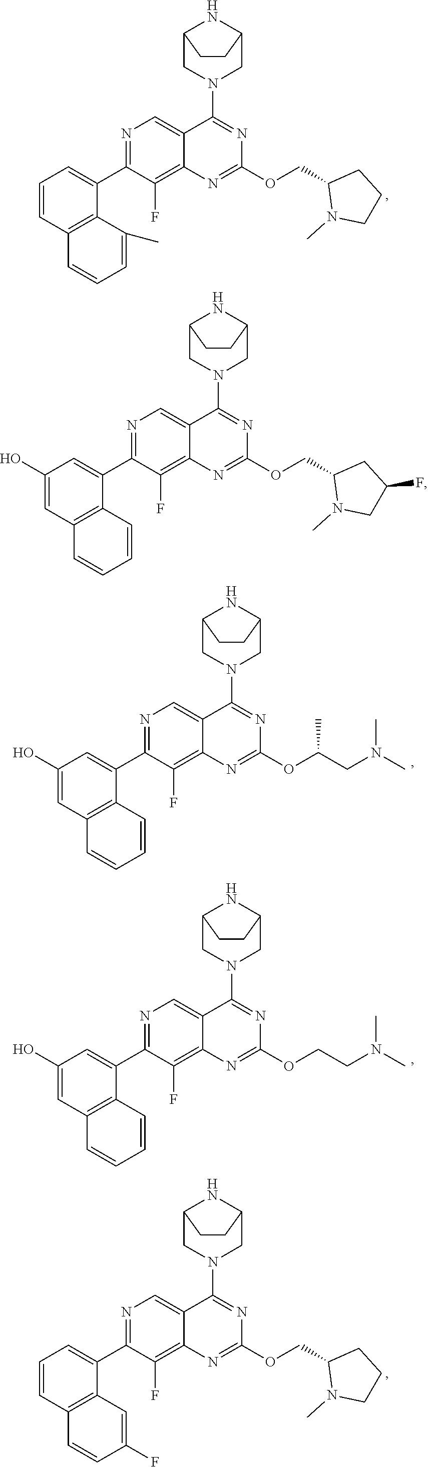 [SMILES: Cc1cccc2cccc(-c3ncc4c(N5CC6CCC(C5)N6)nc(OC[C@@H]5CCCN5C)nc4c3F)c12 ; CN1C[C@H](F)C[C@H]1COc1nc(N2CC3CCC(C2)N3)c2cnc(-c3cc(O)cc4ccccc34)c(F)c2n1 ; C[C@H](CN(C)C)Oc1nc(N2CC3CCC(C2)N3)c2cnc(-c3cc(O)cc4ccccc34)c(F)c2n1 ; CN(C)CCOc1nc(N2CC3CCC(C2)N3)c2cnc(-c3cc(O)cc4ccccc34)c(F)c2n1 ; CN1CCC[C@H]1COc1nc(N2CC3CCC(C2)N3)c2cnc(-c3cccc4ccc(F)cc34)c(F)c2n1]

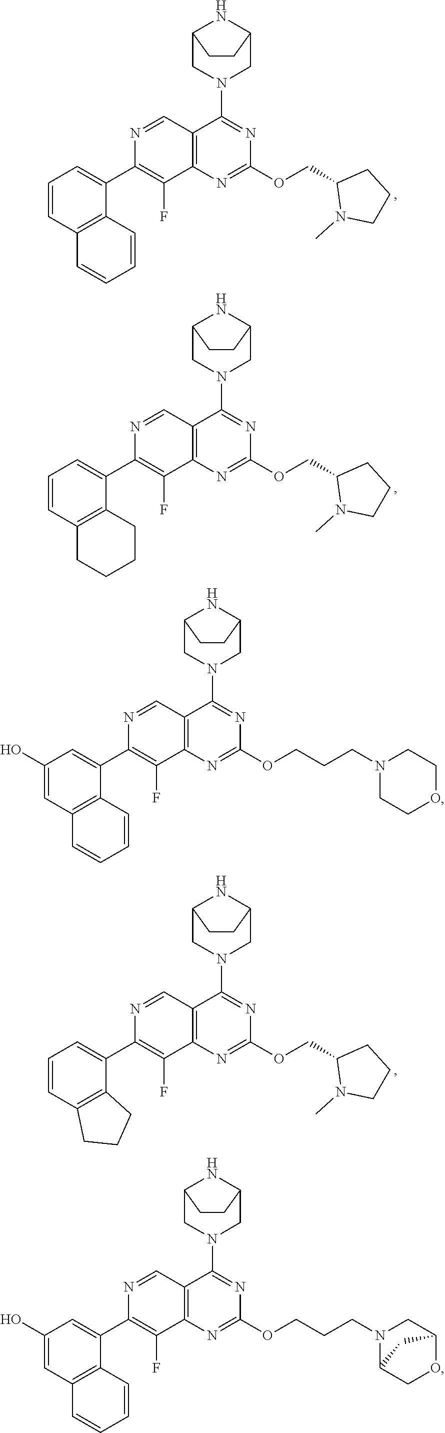

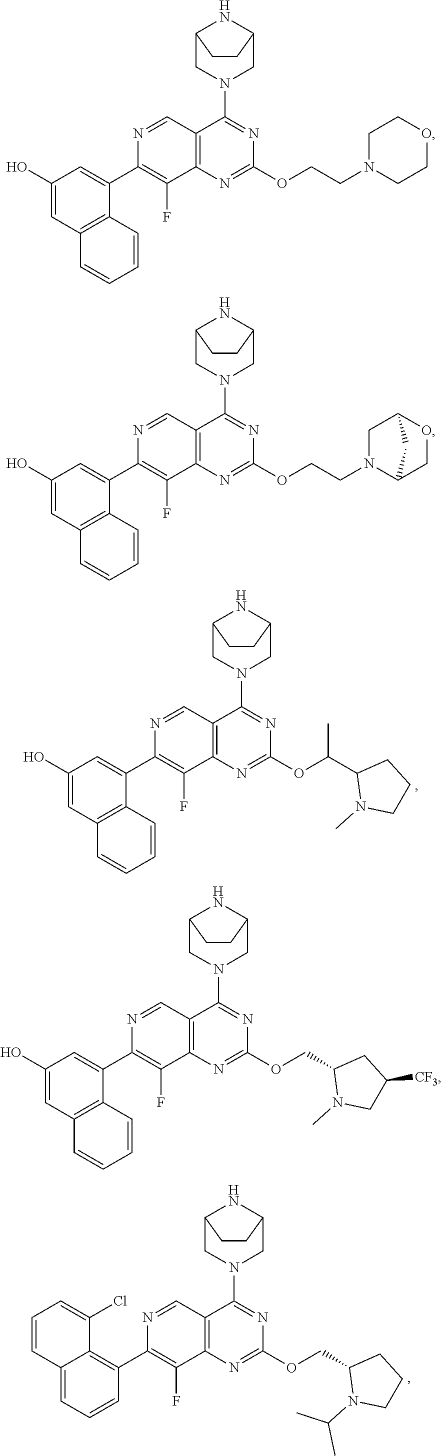

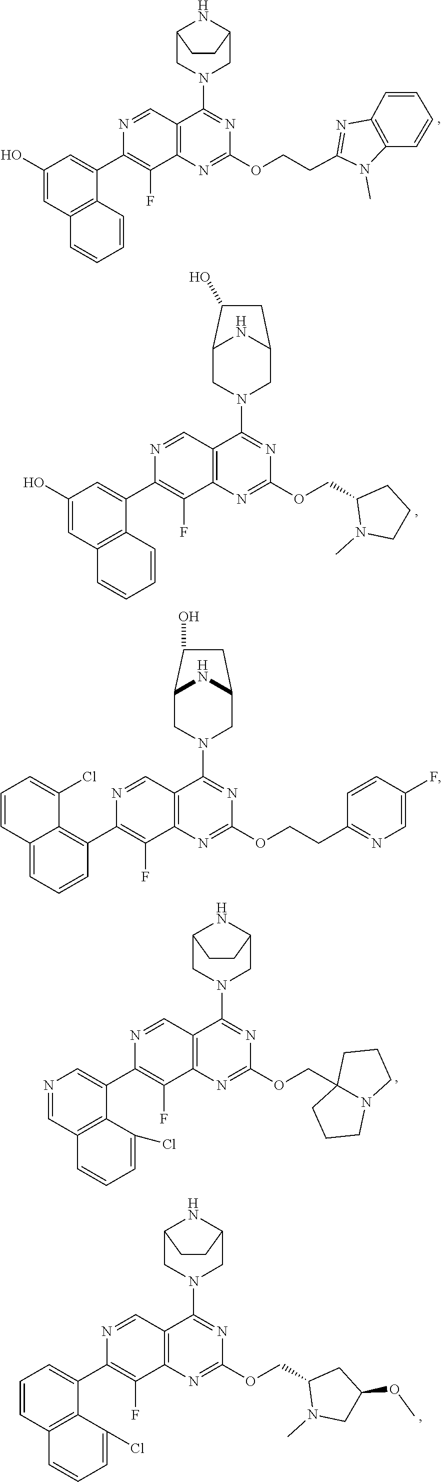

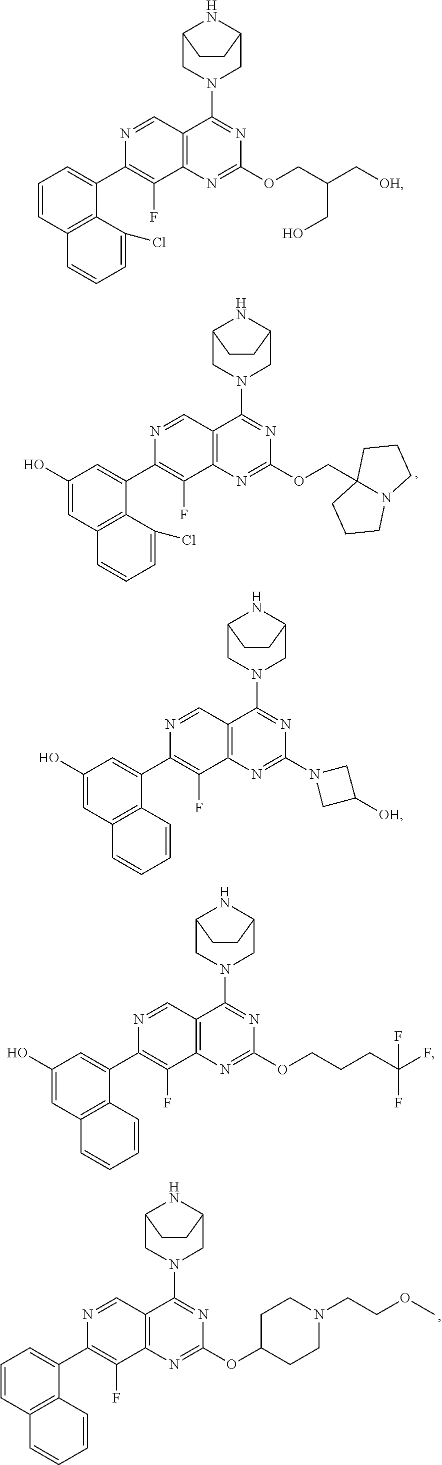 [SMILES: OCC(CO)COc1nc(N2CC3CCC(C2)N3)c2cnc(-c3cccc4cccc(Cl)c34)c(F)c2n1 ; Oc1cc(-c2ncc3c(N4CC5CCC(C4)N5)nc(OCC45CCCN4CCC5)nc3c2F)c2c(Cl)cccc2c1 ; Oc1cc(-c2ncc3c(N4CC5CCC(C4)N5)nc(N4CC(O)C4)nc3c2F)c2ccccc2c1 ; Oc1cc(-c2ncc3c(N4CC5CCC(C4)N5)nc(OCCCC(F)(F)F)nc3c2F)c2ccccc2c1 ; COCCN1CCC(Oc2nc(N3CC4CCC(C3)N4)c3cnc(-c4cccc5ccccc45)c(F)c3n2)CC1]

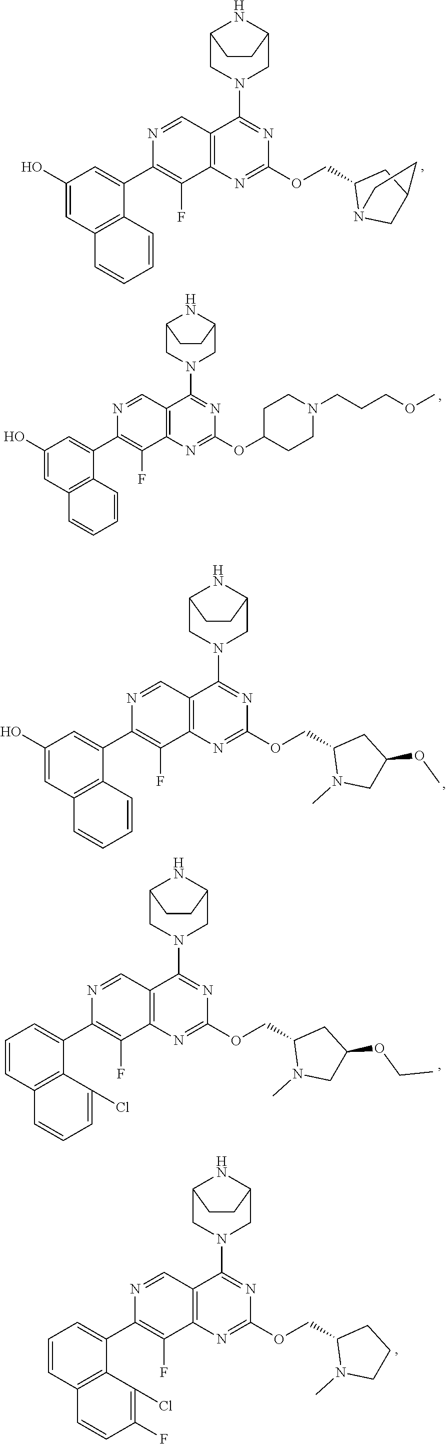 [SMILES: Oc1cc(-c2ncc3c(N4CC5CCC(C4)N5)nc(OC[C@@H]4CC5CCN4C5)nc3c2F)c2ccccc2c1 ; COCCCN1CCC(Oc2nc(N3CC4CCC(C3)N4)c3cnc(-c4cc(O)cc5ccccc45)c(F)c3n2)CC1 ; CO[C@@H]1C[C@@H](COc2nc(N3CC4CCC(C3)N4)c3cnc(-c4cc(O)cc5ccccc45)c(F)c3n2)N(C)C1 ; CCO[C@@H]1C[C@@H](COc2nc(N3CC4CCC(C3)N4)c3cnc(-c4cccc5cccc(Cl)c45)c(F)c3n2)N(C)C1 ; CN1CCC[C@H]1COc1nc(N2CC3CCC(C2)N3)c2cnc(-c3cccc4ccc(F)c(Cl)c34)c(F)c2n1]

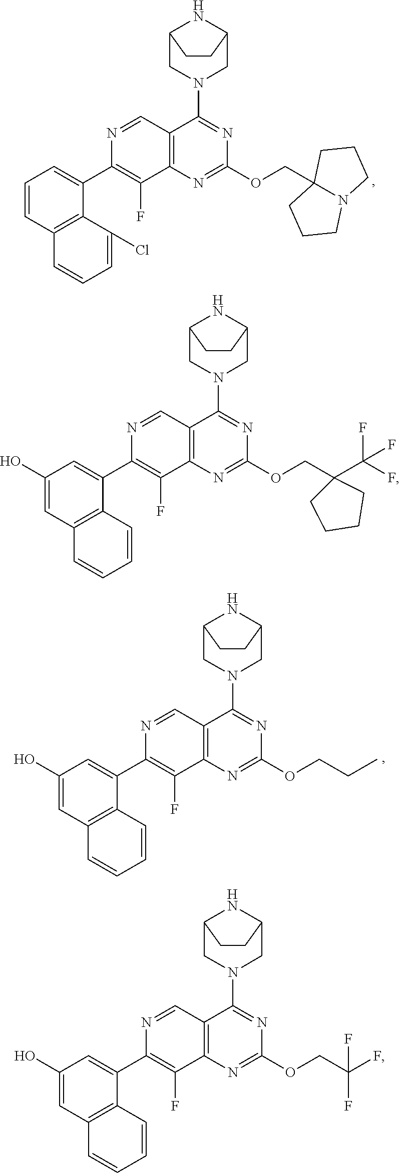 [SMILES: Fc1c(-c2cccc3cccc(Cl)c23)ncc2c(N3CC4CCC(C3)N4)nc(OCC34CCCN3CCC4)nc12 ; Oc1cc(-c2ncc3c(N4CC5CCC(C4)N5)nc(OCC4(C(F)(F)F)CCCC4)nc3c2F)c2ccccc2c1 ; CCCOc1nc(N2CC3CCC(C2)N3)c2cnc(-c3cc(O)cc4ccccc34)c(F)c2n1 ; Oc1cc(-c2ncc3c(N4CC5CCC(C4)N5)nc(OCC(F)(F)F)nc3c2F)c2ccccc2c1]

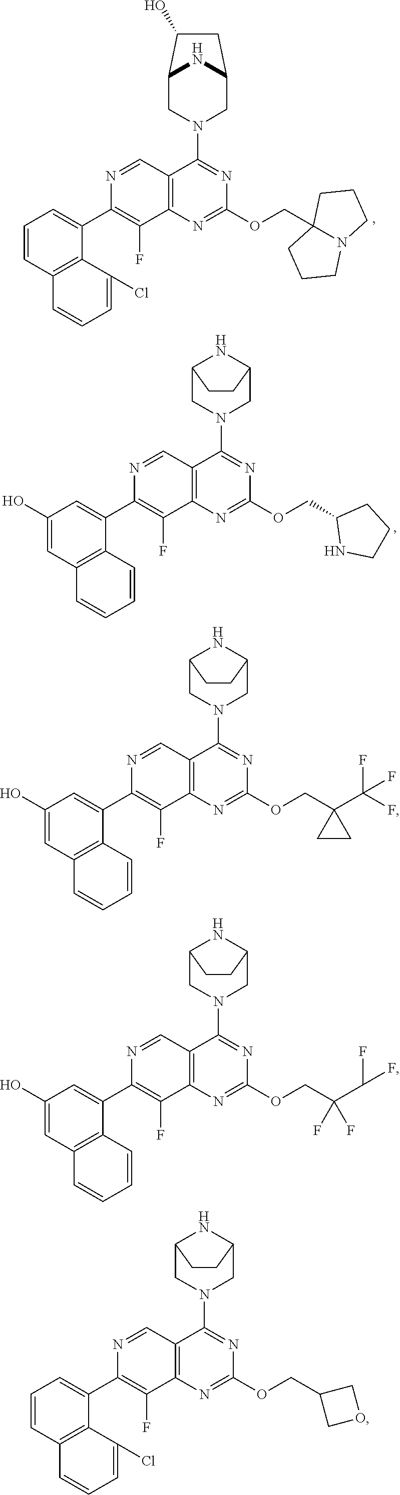

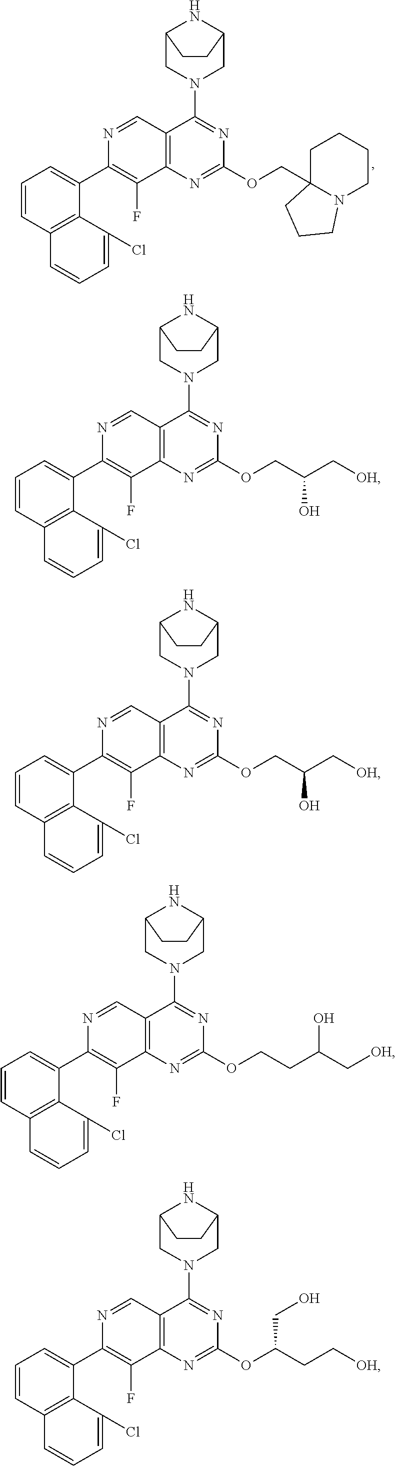 [SMILES: Fc1c(-c2cccc3cccc(Cl)c23)ncc2c(N3CC4CCC(C3)N4)nc(OCC34CCCCN3CCC4)nc12 ; OC[C@H](O)COc1nc(N2CC3CCC(C2)N3)c2cnc(-c3cccc4cccc(Cl)c34)c(F)c2n1 ; OC[C@@H](O)COc1nc(N2CC3CCC(C2)N3)c2cnc(-c3cccc4cccc(Cl)c34)c(F)c2n1 ; OCC(O)CCOc1nc(N2CC3CCC(C2)N3)c2cnc(-c3cccc4cccc(Cl)c34)c(F)c2n1 ; OCC[C@@H](CO)Oc1nc(N2CC3CCC(C2)N3)c2cnc(-c3cccc4cccc(Cl)c34)c(F)c2n1]

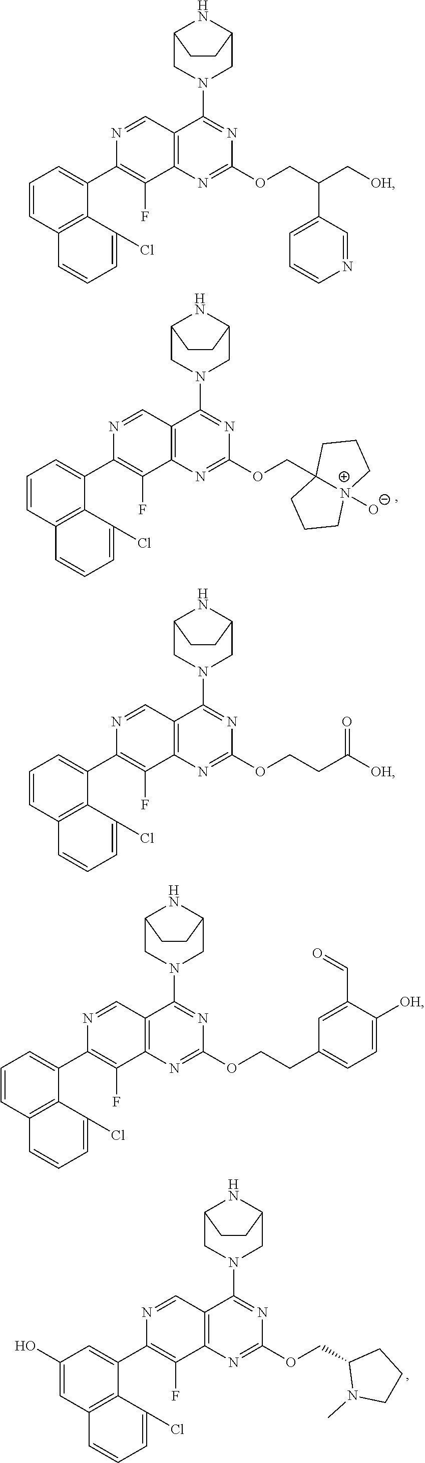 [SMILES: OCC(COc1nc(N2CC3CCC(C2)N3)c2cnc(-c3cccc4cccc(Cl)c34)c(F)c2n1)c1cccnc1 ; [O-][N+]12CCCC1(COc1nc(N3CC4CCC(C3)N4)c3cnc(-c4cccc5cccc(Cl)c45)c(F)c3n1)CCC2 ; O=C(O)CCOc1nc(N2CC3CCC(C2)N3)c2cnc(-c3cccc4cccc(Cl)c34)c(F)c2n1 ; O=Cc1cc(CCOc2nc(N3CC4CCC(C3)N4)c3cnc(-c4cccc5cccc(Cl)c45)c(F)c3n2)ccc1O ; CN1CCC[C@H]1COc1nc(N2CC3CCC(C2)N3)c2cnc(-c3cc(O)cc4cccc(Cl)c34)c(F)c2n1]

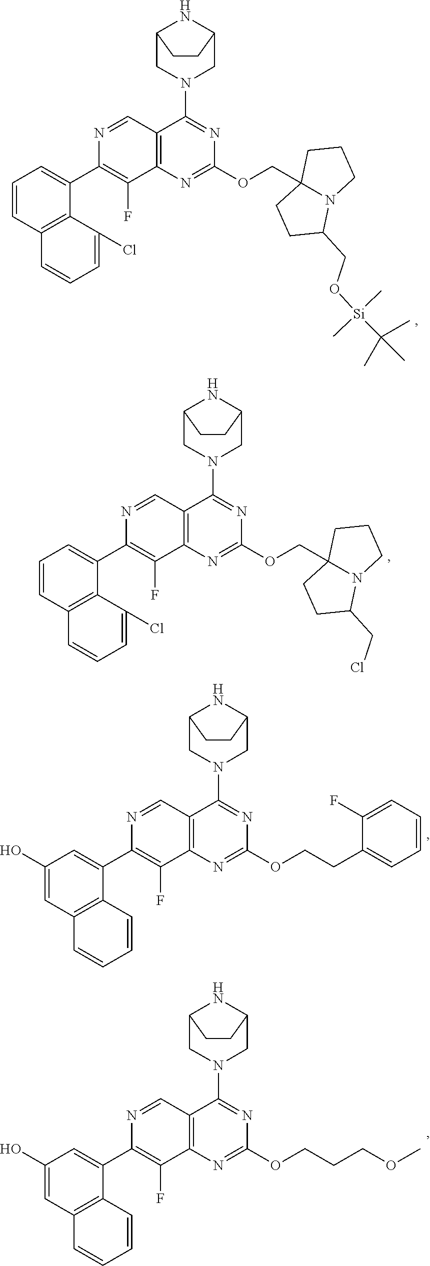 [SMILES: CC(C)(C)[Si](C)(C)OCC1CCC2(COc3nc(N4CC5CCC(C4)N5)c4cnc(-c5cccc6cccc(Cl)c56)c(F)c4n3)CCCN12 ; Fc1c(-c2cccc3cccc(Cl)c23)ncc2c(N3CC4CCC(C3)N4)nc(OCC34CCCN3C(CCl)CC4)nc12 ; Oc1cc(-c2ncc3c(N4CC5CCC(C4)N5)nc(OCCc4ccccc4F)nc3c2F)c2ccccc2c1 ; COCCCOc1nc(N2CC3CCC(C2)N3)c2cnc(-c3cc(O)cc4ccccc34)c(F)c2n1]

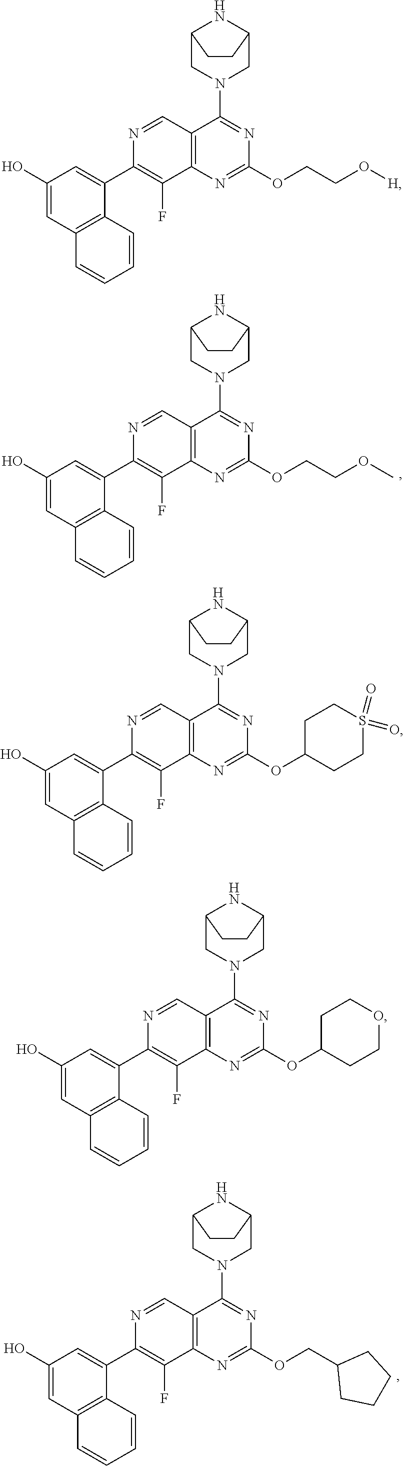

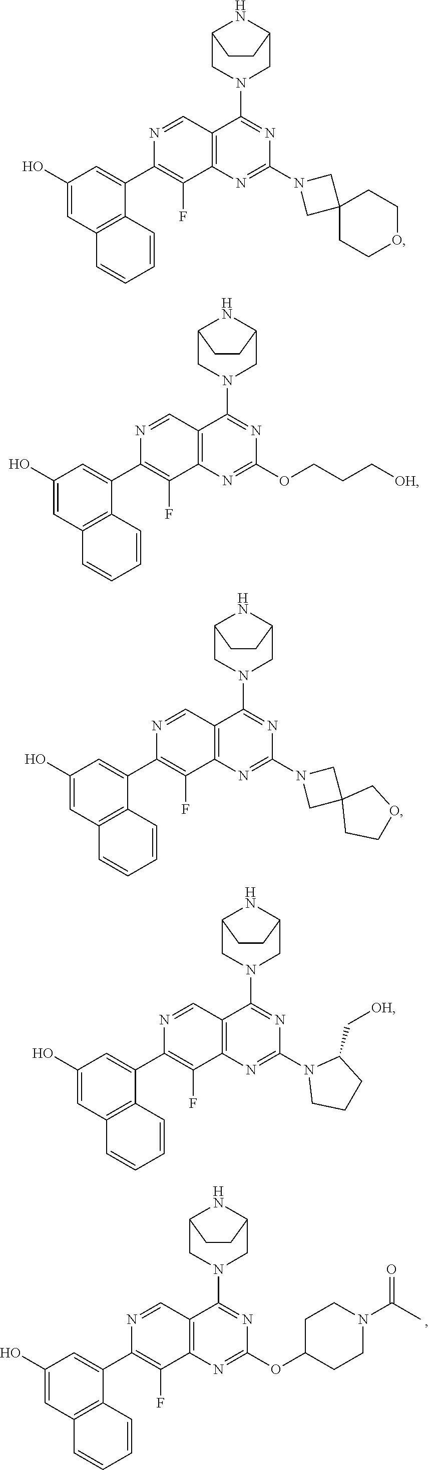 [SMILES: Oc1cc(-c2ncc3c(N4CC5CCC(C4)N5)nc(N4CC5(CCOCC5)C4)nc3c2F)c2ccccc2c1 ; OCCCOc1nc(N2CC3CCC(C2)N3)c2cnc(-c3cc(O)cc4ccccc34)c(F)c2n1 ; Oc1cc(-c2ncc3c(N4CC5CCC(C4)N5)nc(N4CC5(CCOC5)C4)nc3c2F)c2ccccc2c1 ; OC[C@@H]1CCCN1c1nc(N2CC3CCC(C2)N3)c2cnc(-c3cc(O)cc4ccccc34)c(F)c2n1 ; CC(=O)N1CCC(Oc2nc(N3CC4CCC(C3)N4)c3cnc(-c4cc(O)cc5ccccc45)c(F)c3n2)CC1]

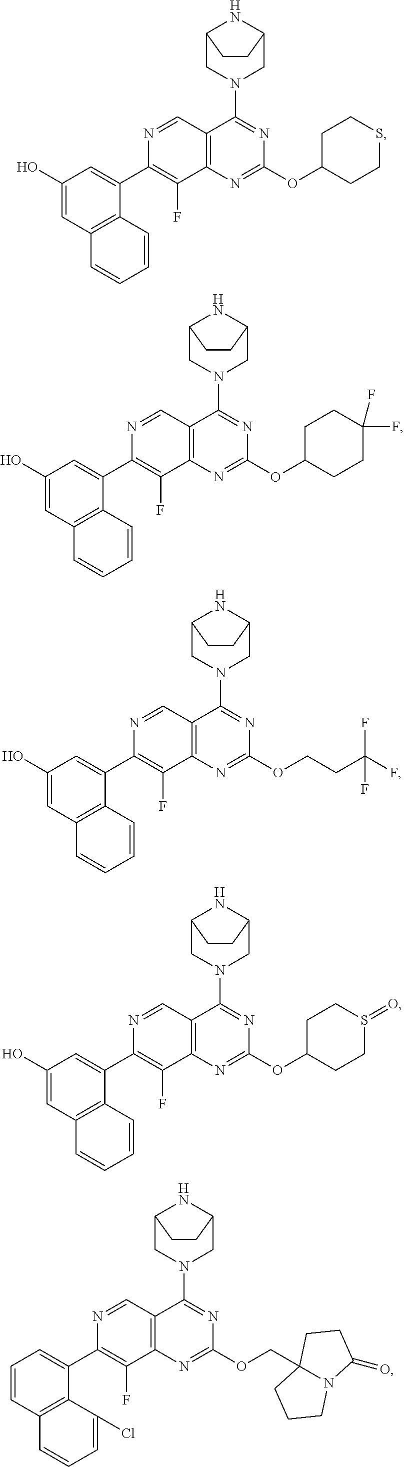 [SMILES: Oc1cc(-c2ncc3c(N4CC5CCC(C4)N5)nc(OC4CCSCC4)nc3c2F)c2ccccc2c1 ; Oc1cc(-c2ncc3c(N4CC5CCC(C4)N5)nc(OC4CCC(F)(F)CC4)nc3c2F)c2ccccc2c1 ; Oc1cc(-c2ncc3c(N4CC5CCC(C4)N5)nc(OCCC(F)(F)F)nc3c2F)c2ccccc2c1 ; O=S1CCC(Oc2nc(N3CC4CCC(C3)N4)c3cnc(-c4cc(O)cc5ccccc45)c(F)c3n2)CC1 ; O=C1CCC2(COc3nc(N4CC5CCC(C4)N5)c4cnc(-c5cccc6cccc(Cl)c56)c(F)c4n3)CCCN12]

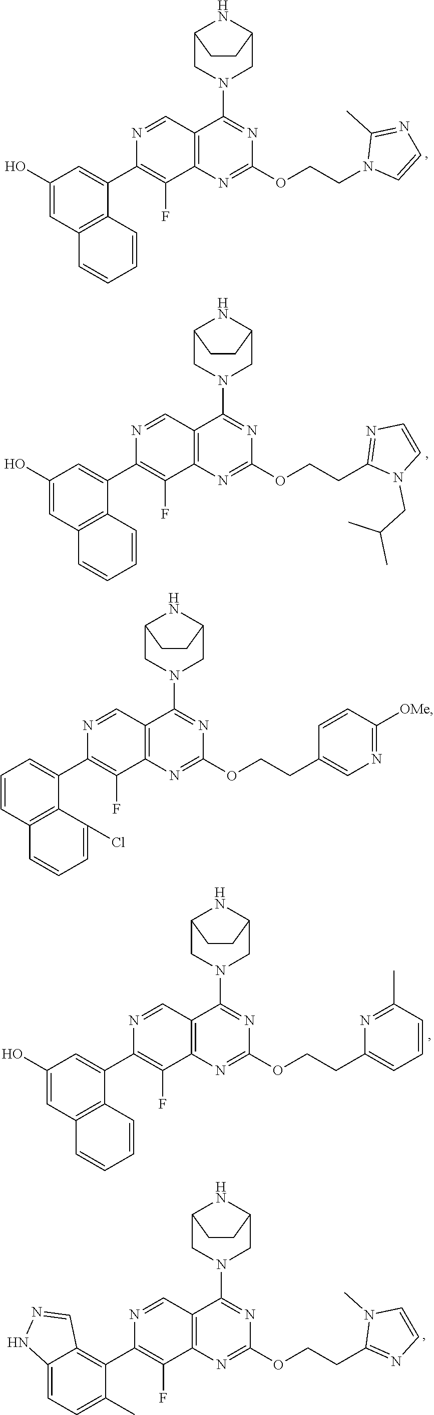 [SMILES: Cc1nccn1CCOc1nc(N2CC3CCC(C2)N3)c2cnc(-c3cc(O)cc4ccccc34)c(F)c2n1 ; CC(C)Cn1ccnc1CCOc1nc(N2CC3CCC(C2)N3)c2cnc(-c3cc(O)cc4ccccc34)c(F)c2n1 ; COc1ccc(CCOc2nc(N3CC4CCC(C3)N4)c3cnc(-c4cccc5cccc(Cl)c45)c(F)c3n2)cn1 ; Cc1cccc(CCOc2nc(N3CC4CCC(C3)N4)c3cnc(-c4cc(O)cc5ccccc45)c(F)c3n2)n1 ; Cc1ccc2[nH]ncc2c1-c1ncc2c(N3CC4CCC(C3)N4)nc(OCCc3nccn3C)nc2c1F]

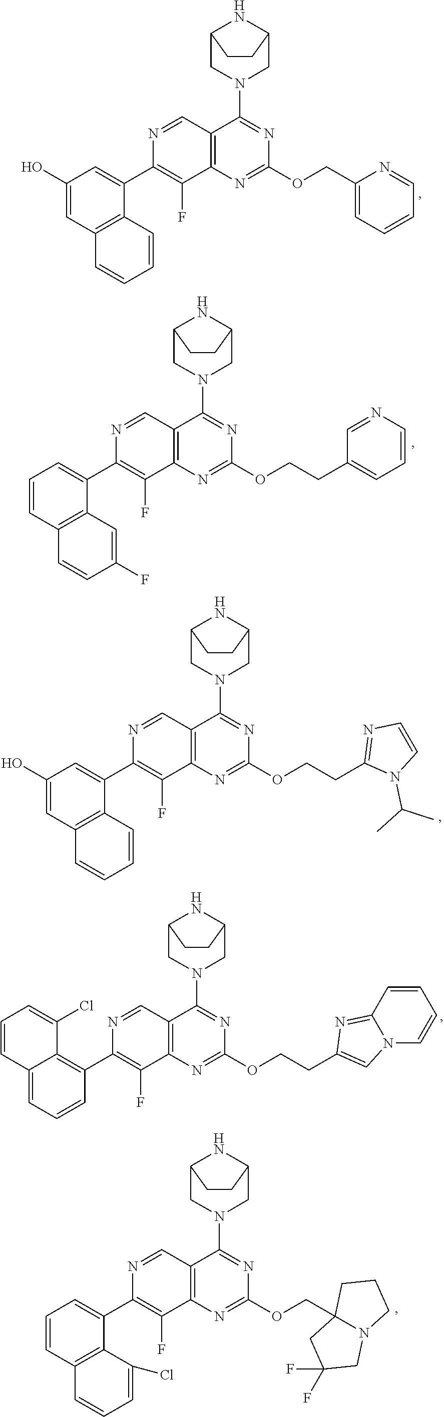 [SMILES: Oc1cc(-c2ncc3c(N4CC5CCC(C4)N5)nc(OCc4ccccn4)nc3c2F)c2ccccc2c1 ; Fc1ccc2cccc(-c3ncc4c(N5CC6CCC(C5)N6)nc(OCCc5cccnc5)nc4c3F)c2c1 ; CC(C)n1ccnc1CCOc1nc(N2CC3CCC(C2)N3)c2cnc(-c3cc(O)cc4ccccc34)c(F)c2n1 ; Fc1c(-c2cccc3cccc(Cl)c23)ncc2c(N3CC4CCC(C3)N4)nc(OCCc3cn4ccccc4n3)nc12 ; Fc1c(-c2cccc3cccc(Cl)c23)ncc2c(N3CC4CCC(C3)N4)nc(OCC34CCCN3CC(F)(F)C4)nc12]

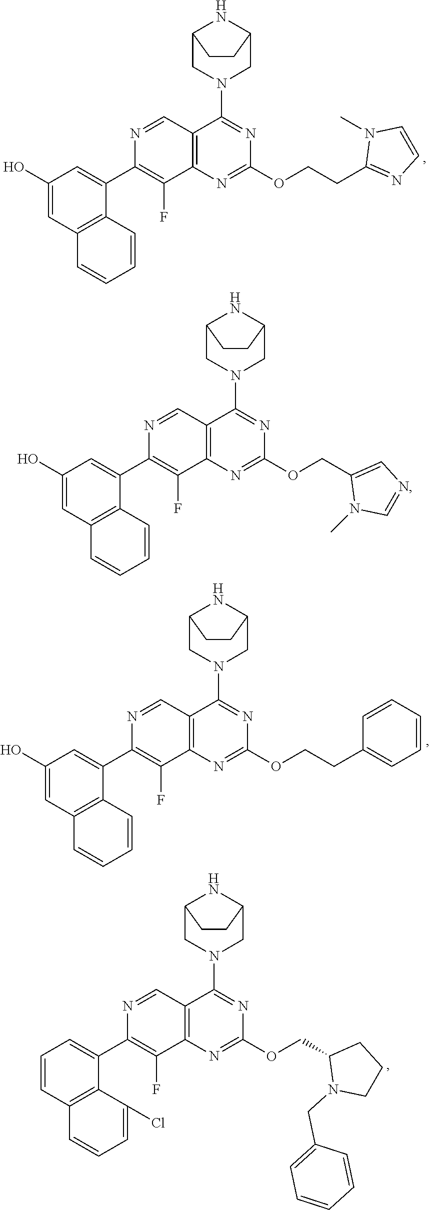 [SMILES: Cn1ccnc1CCOc1nc(N2CC3CCC(C2)N3)c2cnc(-c3cc(O)cc4ccccc34)c(F)c2n1 ; Cn1cncc1COc1nc(N2CC3CCC(C2)N3)c2cnc(-c3cc(O)cc4ccccc34)c(F)c2n1 ; Oc1cc(-c2ncc3c(N4CC5CCC(C4)N5)nc(OCCc4ccccc4)nc3c2F)c2ccccc2c1 ; Fc1c(-c2cccc3cccc(Cl)c23)ncc2c(N3CC4CCC(C3)N4)nc(OC[C@@H]3CCCN3Cc3ccccc3)nc12]

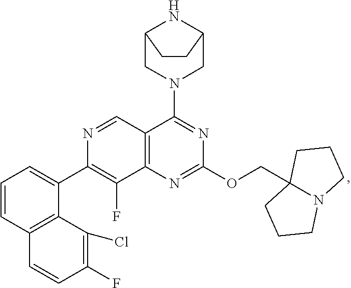 [SMILES: Fc1ccc2cccc(-c3ncc4c(N5CC6CCC(C5)N6)nc(OCC56CCCN5CCC6)nc4c3F)c2c1Cl]

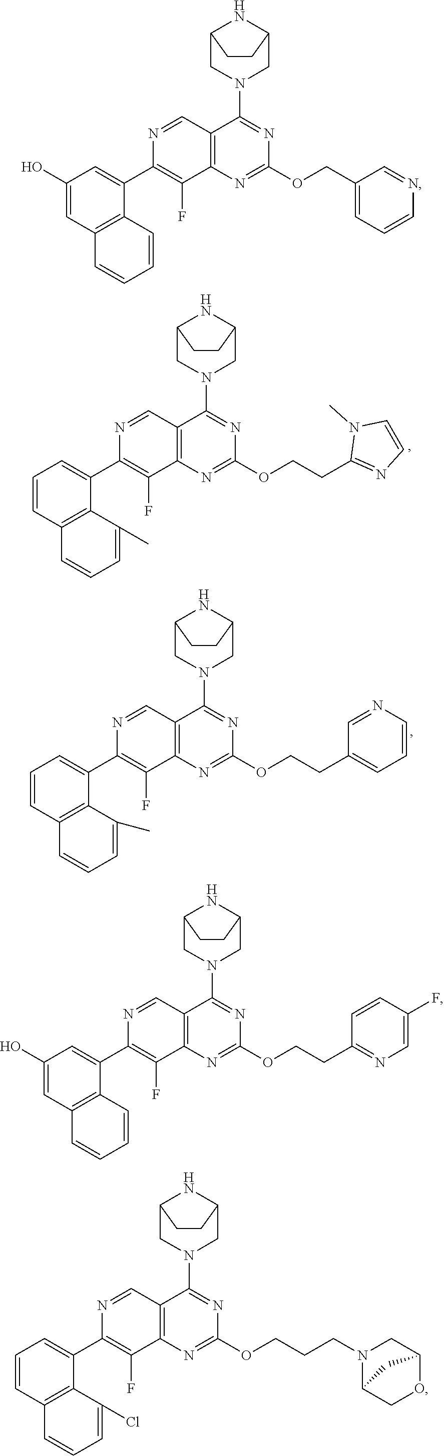 [SMILES: Oc1cc(-c2ncc3c(N4CC5CCC(C4)N5)nc(OCc4cccnc4)nc3c2F)c2ccccc2c1 ; Cc1cccc2cccc(-c3ncc4c(N5CC6CCC(C5)N6)nc(OCCc5nccn5C)nc4c3F)c12 ; Cc1cccc2cccc(-c3ncc4c(N5CC6CCC(C5)N6)nc(OCCc5cccnc5)nc4c3F)c12 ; Oc1cc(-c2ncc3c(N4CC5CCC(C4)N5)nc(OCCc4ccc(F)cn4)nc3c2F)c2ccccc2c1 ; Fc1c(-c2cccc3cccc(Cl)c23)ncc2c(N3CC4CCC(C3)N4)nc(OCCCN3C[C@@H]4C[C@H]3CO4)nc12]

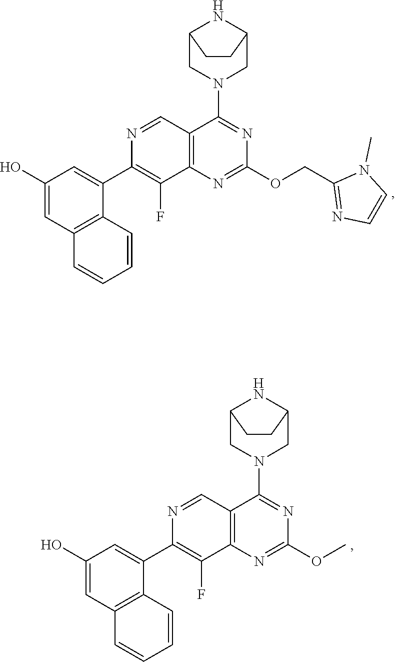 [SMILES: Cn1ccnc1COc1nc(N2CC3CCC(C2)N3)c2cnc(-c3cc(O)cc4ccccc34)c(F)c2n1 ; COc1nc(N2CC3CCC(C2)N3)c2cnc(-c3cc(O)cc4ccccc34)c(F)c2n1]

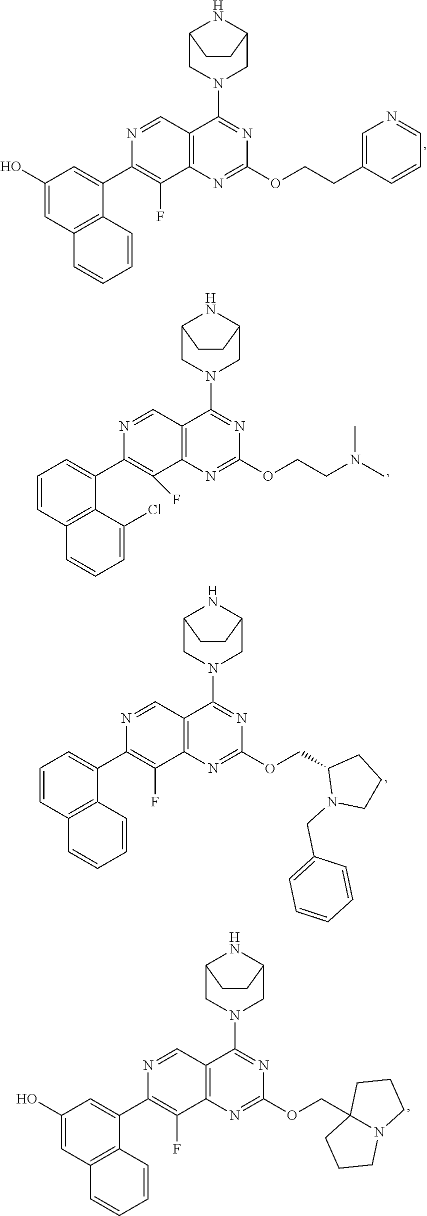 [SMILES: Oc1cc(-c2ncc3c(N4CC5CCC(C4)N5)nc(OCCc4cccnc4)nc3c2F)c2ccccc2c1 ; CN(C)CCOc1nc(N2CC3CCC(C2)N3)c2cnc(-c3cccc4cccc(Cl)c34)c(F)c2n1 ; Fc1c(-c2cccc3ccccc23)ncc2c(N3CC4CCC(C3)N4)nc(OC[C@@H]3CCCN3Cc3ccccc3)nc12 ; Oc1cc(-c2ncc3c(N4CC5CCC(C4)N5)nc(OCC45CCCN4CCC5)nc3c2F)c2ccccc2c1]

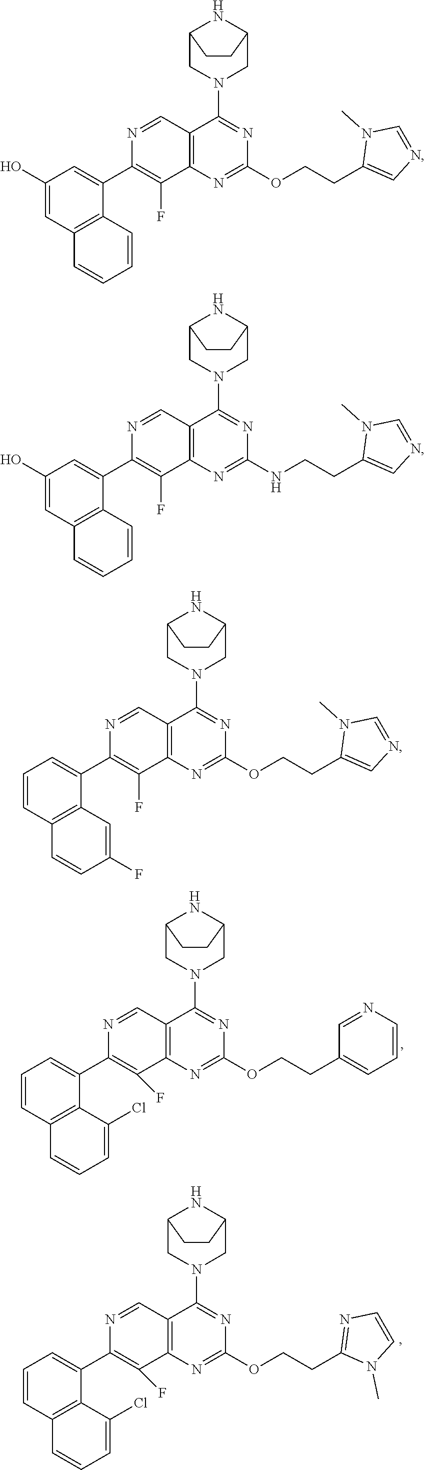 [SMILES: Cn1cncc1CCOc1nc(N2CC3CCC(C2)N3)c2cnc(-c3cc(O)cc4ccccc34)c(F)c2n1 ; Cn1cncc1CCNc1nc(N2CC3CCC(C2)N3)c2cnc(-c3cc(O)cc4ccccc34)c(F)c2n1 ; Cn1cncc1CCOc1nc(N2CC3CCC(C2)N3)c2cnc(-c3cccc4ccc(F)cc34)c(F)c2n1 ; Fc1c(-c2cccc3cccc(Cl)c23)ncc2c(N3CC4CCC(C3)N4)nc(OCCc3cccnc3)nc12 ; Cn1ccnc1CCOc1nc(N2CC3CCC(C2)N3)c2cnc(-c3cccc4cccc(Cl)c34)c(F)c2n1]

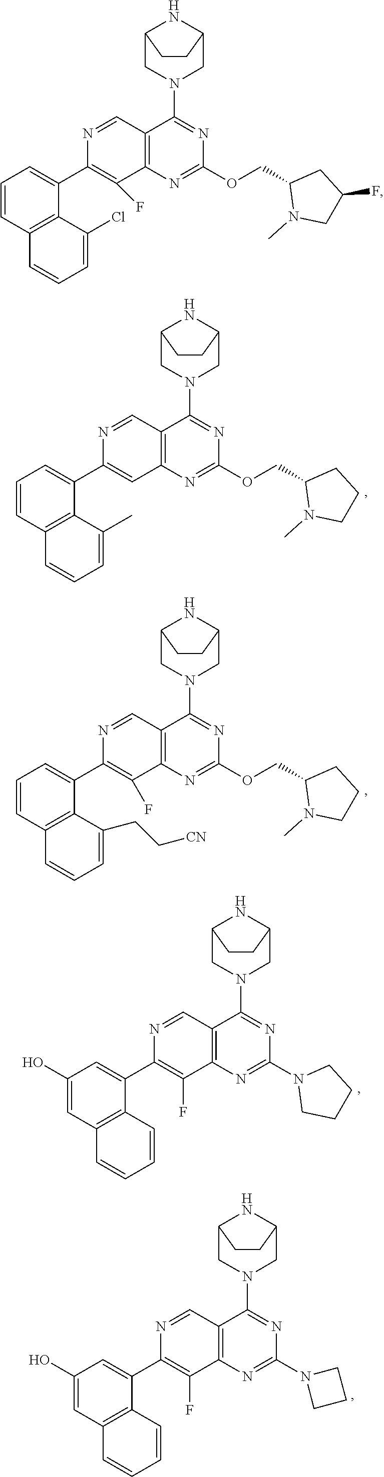 [SMILES: CN1C[C@H](F)C[C@H]1COc1nc(N2CC3CCC(C2)N3)c2cnc(-c3cccc4cccc(Cl)c34)c(F)c2n1 ; Cc1cccc2cccc(-c3cc4nc(OC[C@@H]5CCCN5C)nc(N5CC6CCC(C5)N6)c4cn3)c12 ; CN1CCC[C@H]1COc1nc(N2CC3CCC(C2)N3)c2cnc(-c3cccc4cccc(CCC#N)c34)c(F)c2n1 ; Oc1cc(-c2ncc3c(N4CC5CCC(C4)N5)nc(N4CCCC4)nc3c2F)c2ccccc2c1 ; Oc1cc(-c2ncc3c(N4CC5CCC(C4)N5)nc(N4CCC4)nc3c2F)c2ccccc2c1]

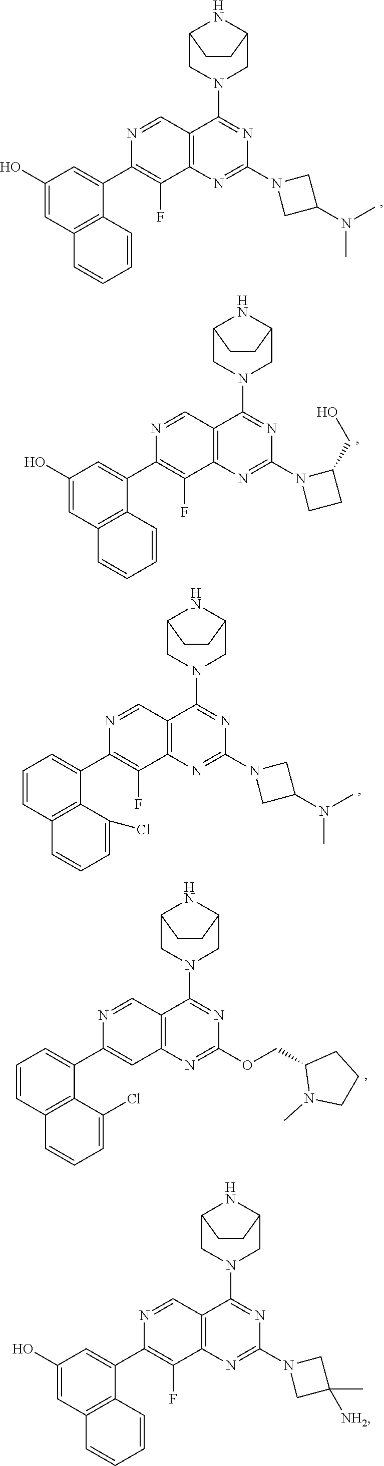 [SMILES: CN(C)C1CN(c2nc(N3CC4CCC(C3)N4)c3cnc(-c4cc(O)cc5ccccc45)c(F)c3n2)C1 ; OC[C@@H]1CCN1c1nc(N2CC3CCC(C2)N3)c2cnc(-c3cc(O)cc4ccccc34)c(F)c2n1 ; CN(C)C1CN(c2nc(N3CC4CCC(C3)N4)c3cnc(-c4cccc5cccc(Cl)c45)c(F)c3n2)C1 ; CN1CCC[C@H]1COc1nc(N2CC3CCC(C2)N3)c2cnc(-c3cccc4cccc(Cl)c34)cc2n1 ; CC1(N)CN(c2nc(N3CC4CCC(C3)N4)c3cnc(-c4cc(O)cc5ccccc45)c(F)c3n2)C1]

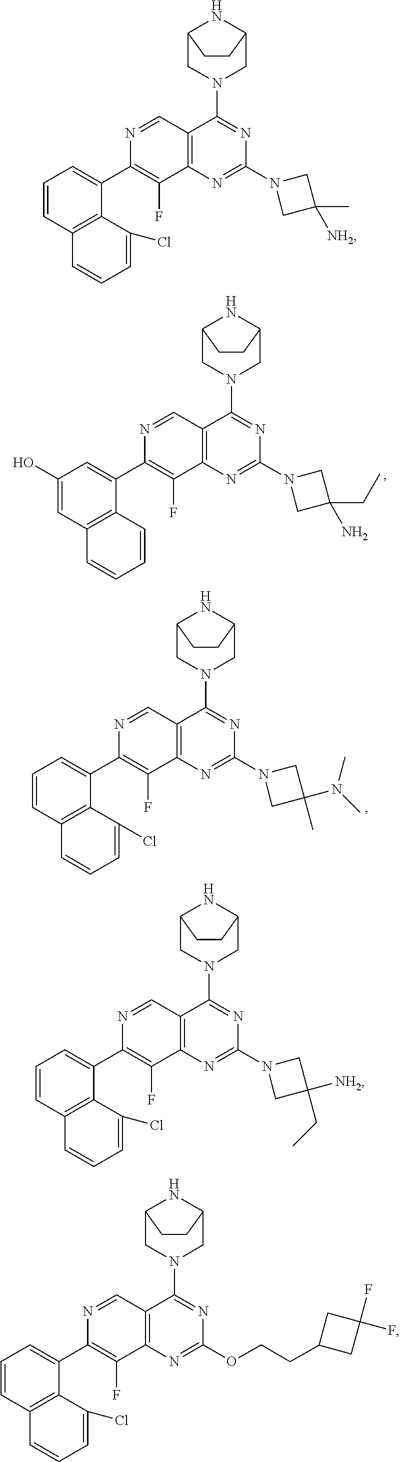 [SMILES: CC1(N)CN(c2nc(N3CC4CCC(C3)N4)c3cnc(-c4cccc5cccc(Cl)c45)c(F)c3n2)C1 ; CCC1(N)CN(c2nc(N3CC4CCC(C3)N4)c3cnc(-c4cc(O)cc5ccccc45)c(F)c3n2)C1 ; CN(C)C1(C)CN(c2nc(N3CC4CCC(C3)N4)c3cnc(-c4cccc5cccc(Cl)c45)c(F)c3n2)C1 ; CCC1(N)CN(c2nc(N3CC4CCC(C3)N4)c3cnc(-c4cccc5cccc(Cl)c45)c(F)c3n2)C1 ; Fc1c(-c2cccc3cccc(Cl)c23)ncc2c(N3CC4CCC(C3)N4)nc(OCCC3CC(F)(F)C3)nc12]

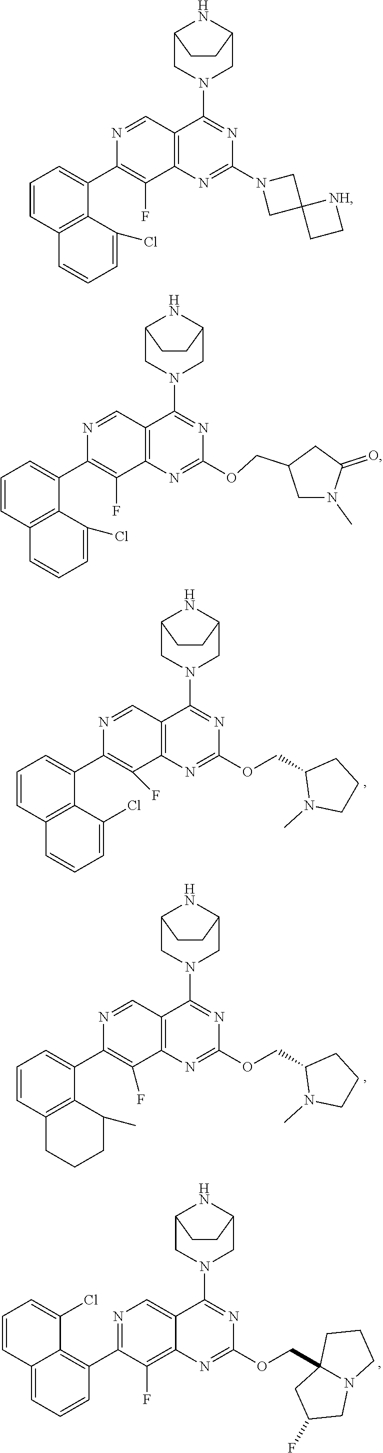

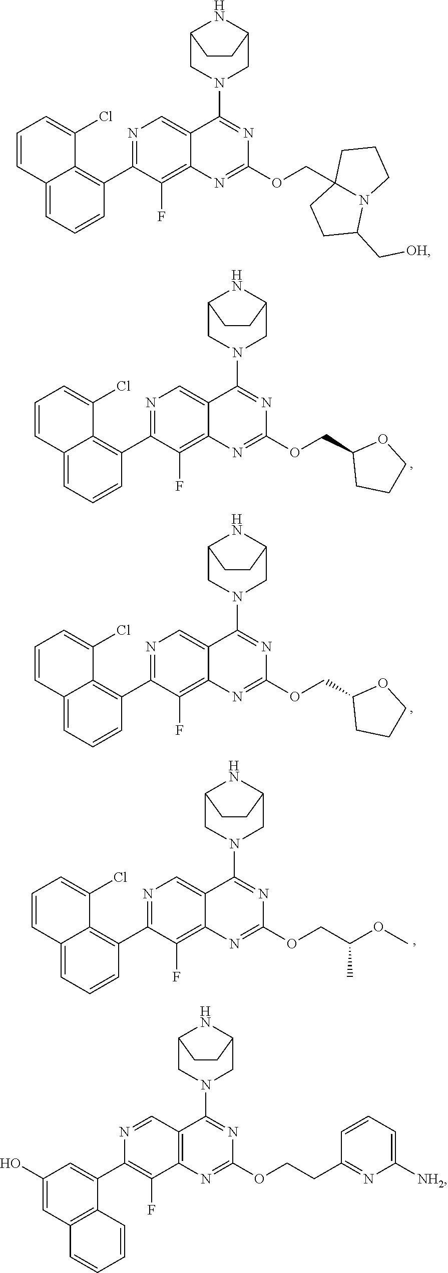 [SMILES: OCC1CCC2(COc3nc(N4CC5CCC(C4)N5)c4cnc(-c5cccc6cccc(Cl)c56)c(F)c4n3)CCCN12 ; Fc1c(-c2cccc3cccc(Cl)c23)ncc2c(N3CC4CCC(C3)N4)nc(OC[C@@H]3CCCO3)nc12 ; Fc1c(-c2cccc3cccc(Cl)c23)ncc2c(N3CC4CCC(C3)N4)nc(OC[C@H]3CCCO3)nc12 ; CO[C@H](C)COc1nc(N2CC3CCC(C2)N3)c2cnc(-c3cccc4cccc(Cl)c34)c(F)c2n1 ; Nc1cccc(CCOc2nc(N3CC4CCC(C3)N4)c3cnc(-c4cc(O)cc5ccccc45)c(F)c3n2)n1]

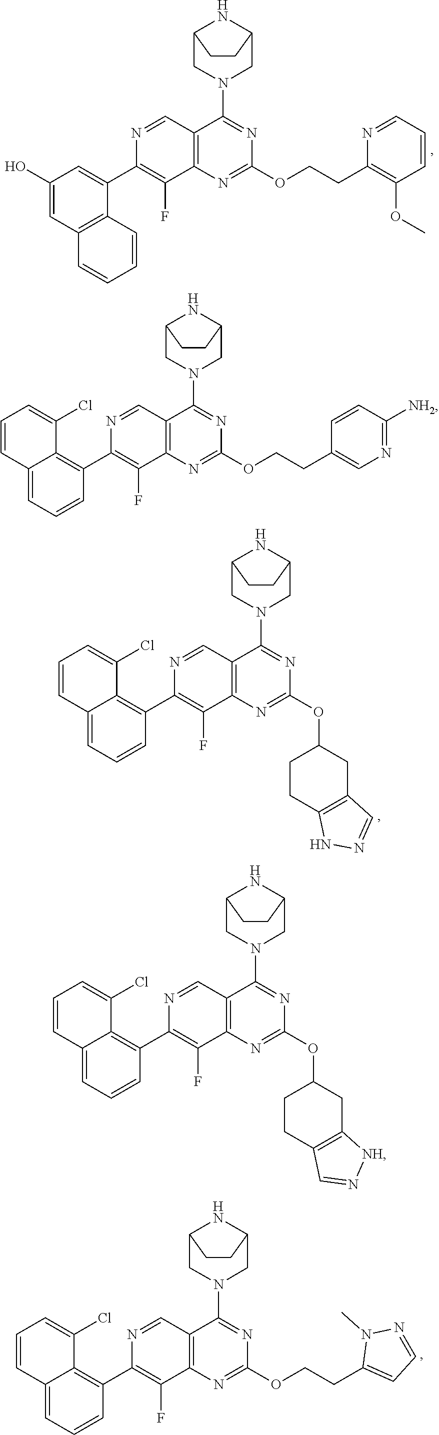 [SMILES: COc1cccnc1CCOc1nc(N2CC3CCC(C2)N3)c2cnc(-c3cc(O)cc4ccccc34)c(F)c2n1 ; Nc1ccc(CCOc2nc(N3CC4CCC(C3)N4)c3cnc(-c4cccc5cccc(Cl)c45)c(F)c3n2)cn1 ; Fc1c(-c2cccc3cccc(Cl)c23)ncc2c(N3CC4CCC(C3)N4)nc(OC3CCc4[nH]ncc4C3)nc12 ; Fc1c(-c2cccc3cccc(Cl)c23)ncc2c(N3CC4CCC(C3)N4)nc(OC3CCc4cn[nH]c4C3)nc12 ; Cn1nccc1CCOc1nc(N2CC3CCC(C2)N3)c2cnc(-c3cccc4cccc(Cl)c34)c(F)c2n1]

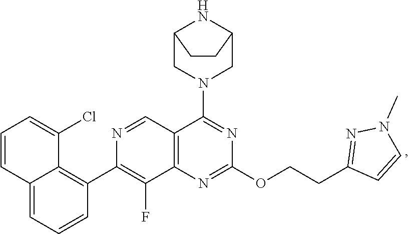 [SMILES: Cn1ccc(CCOc2nc(N3CC4CCC(C3)N4)c3cnc(-c4cccc5cccc(Cl)c45)c(F)c3n2)n1]

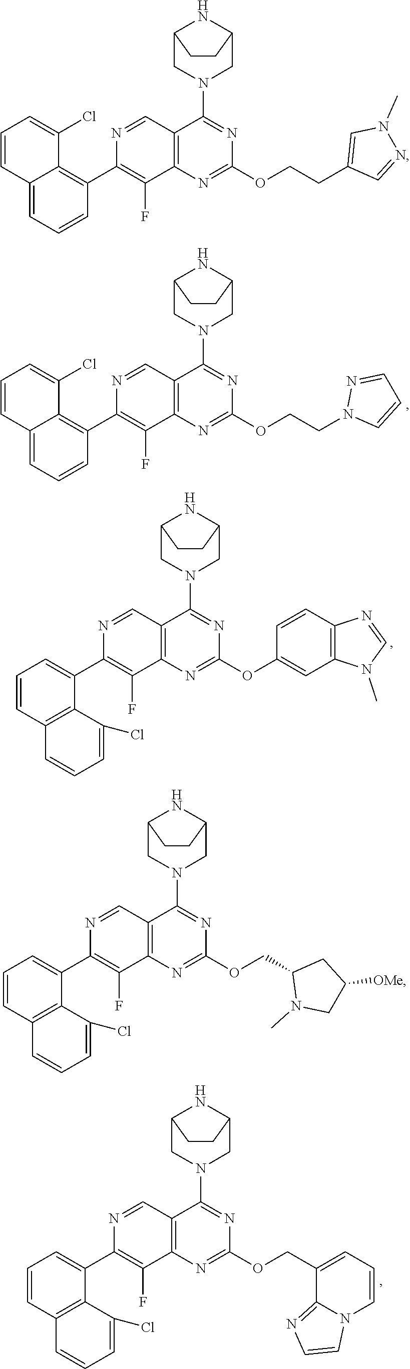 [SMILES: Cn1cc(CCOc2nc(N3CC4CCC(C3)N4)c3cnc(-c4cccc5cccc(Cl)c45)c(F)c3n2)cn1 ; Fc1c(-c2cccc3cccc(Cl)c23)ncc2c(N3CC4CCC(C3)N4)nc(OCCn3cccn3)nc12 ; Cn1cnc2ccc(Oc3nc(N4CC5CCC(C4)N5)c4cnc(-c5cccc6cccc(Cl)c56)c(F)c4n3)cc21 ; CO[C@H]1C[C@@H](COc2nc(N3CC4CCC(C3)N4)c3cnc(-c4cccc5cccc(Cl)c45)c(F)c3n2)N(C)C1 ; Fc1c(-c2cccc3cccc(Cl)c23)ncc2c(N3CC4CCC(C3)N4)nc(OCc3cccn4ccnc34)nc12]

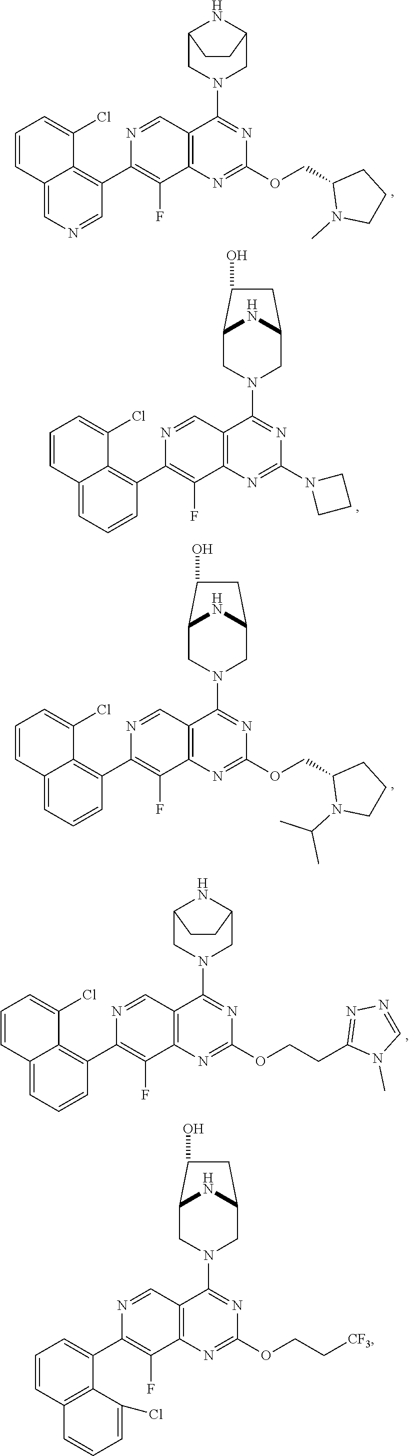

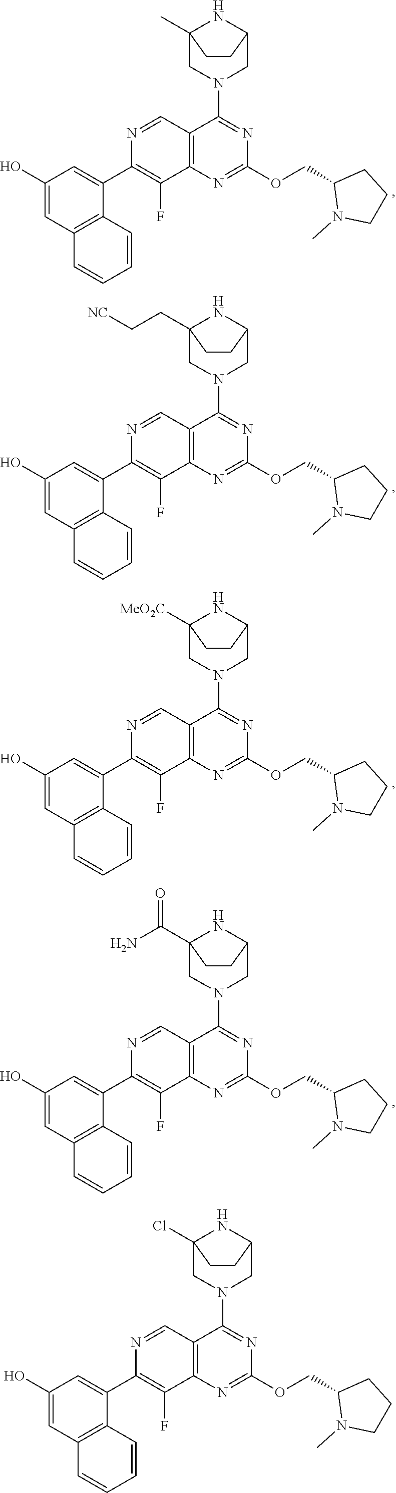

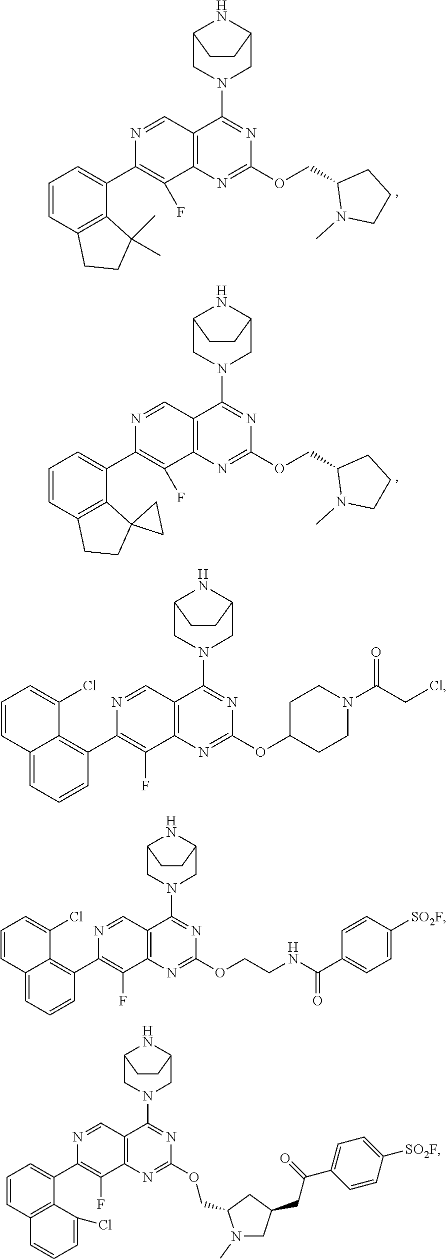 [SMILES: CN1CCC[C@H]1COc1nc(N2CC3CCC(C2)N3)c2cnc(-c3cccc4c3C(C)(C)CC4)c(F)c2n1 ; CN1CCC[C@H]1COc1nc(N2CC3CCC(C2)N3)c2cnc(-c3cccc4c3C3(CC4)CC3)c(F)c2n1 ; O=C(CCl)N1CCC(Oc2nc(N3CC4CCC(C3)N4)c3cnc(-c4cccc5cccc(Cl)c45)c(F)c3n2)CC1 ; O=C(NCCOc1nc(N2CC3CCC(C2)N3)c2cnc(-c3cccc4cccc(Cl)c34)c(F)c2n1)c1ccc(S(=O)(=O)F)cc1 ; CN1C[C@H](CC(=O)c2ccc(S(=O)(=O)F)cc2)C[C@H]1COc1nc(N2CC3CCC(C2)N3)c2cnc(-c3cccc4cccc(Cl)c34)c(F)c2n1]

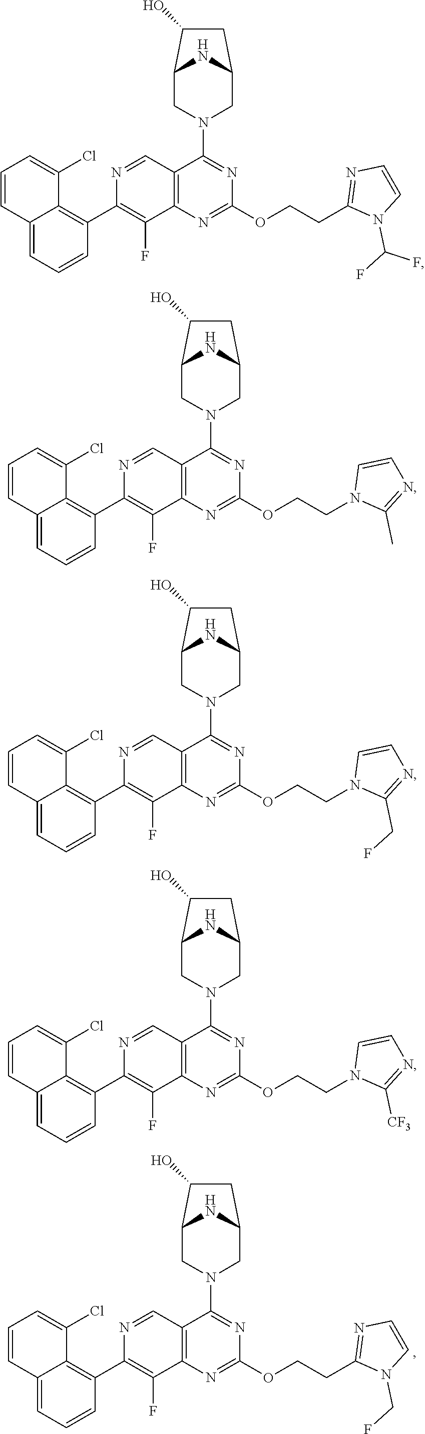

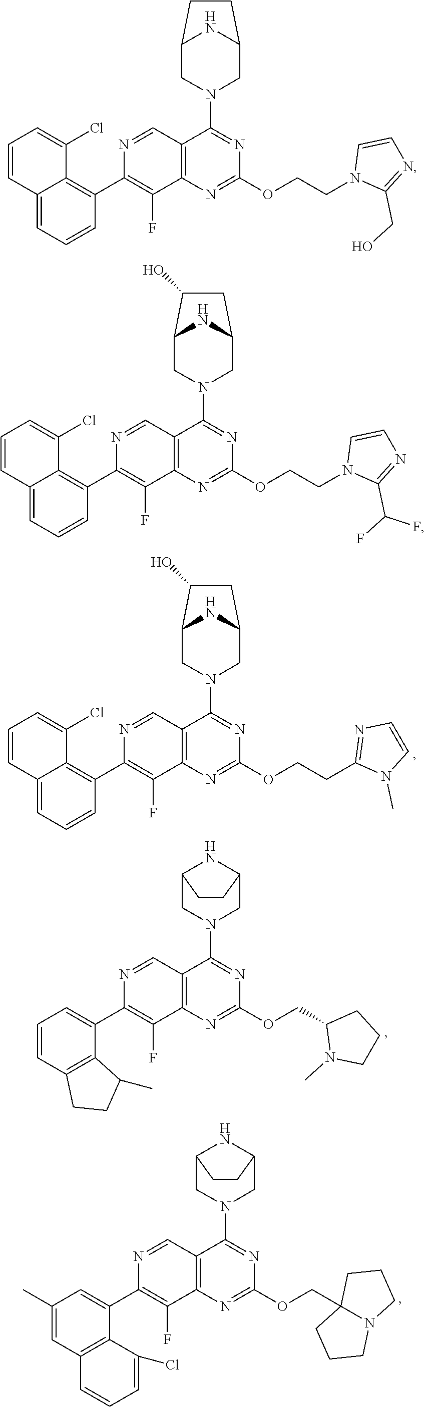

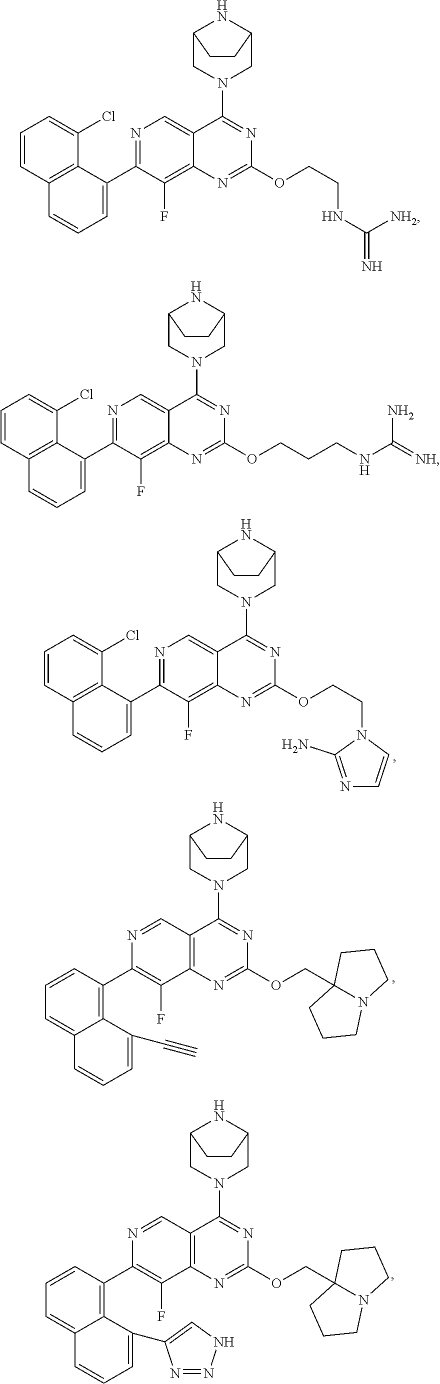 [SMILES: N=C(N)NCCOc1nc(N2CC3CCC(C2)N3)c2cnc(-c3cccc4cccc(Cl)c34)c(F)c2n1 ; N=C(N)NCCCOc1nc(N2CC3CCC(C2)N3)c2cnc(-c3cccc4cccc(Cl)c34)c(F)c2n1 ; Nc1nccn1CCOc1nc(N2CC3CCC(C2)N3)c2cnc(-c3cccc4cccc(Cl)c34)c(F)c2n1 ; C#Cc1cccc2cccc(-c3ncc4c(N5CC6CCC(C5)N6)nc(OCC56CCCN5CCC6)nc4c3F)c12 ; Fc1c(-c2cccc3cccc(-c4c[nH]nn4)c23)ncc2c(N3CC4CCC(C3)N4)nc(OCC34CCCN3CCC4)nc12]

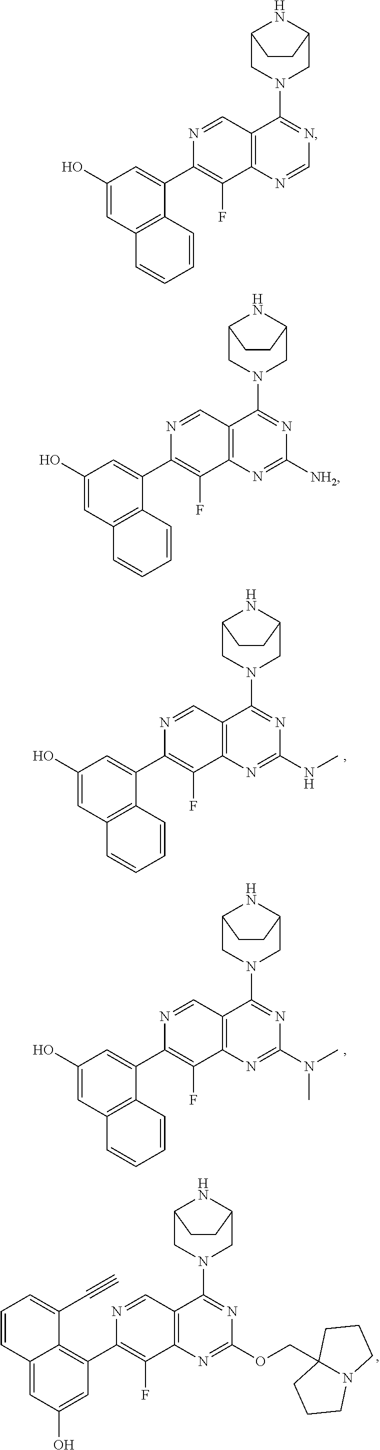 [SMILES: Oc1cc(-c2ncc3c(N4CC5CCC(C4)N5)ncnc3c2F)c2ccccc2c1 ; Nc1nc(N2CC3CCC(C2)N3)c2cnc(-c3cc(O)cc4ccccc34)c(F)c2n1 ; CNc1nc(N2CC3CCC(C2)N3)c2cnc(-c3cc(O)cc4ccccc34)c(F)c2n1 ; CN(C)c1nc(N2CC3CCC(C2)N3)c2cnc(-c3cc(O)cc4ccccc34)c(F)c2n1 ; C#Cc1cccc2cc(O)cc(-c3ncc4c(N5CC6CCC(C5)N6)nc(OCC56CCCN5CCC6)nc4c3F)c12]

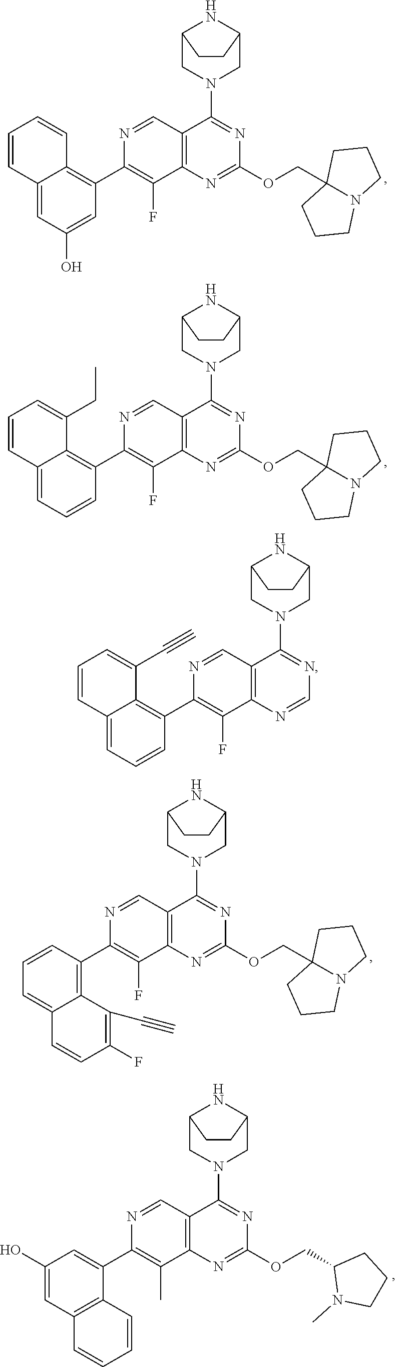

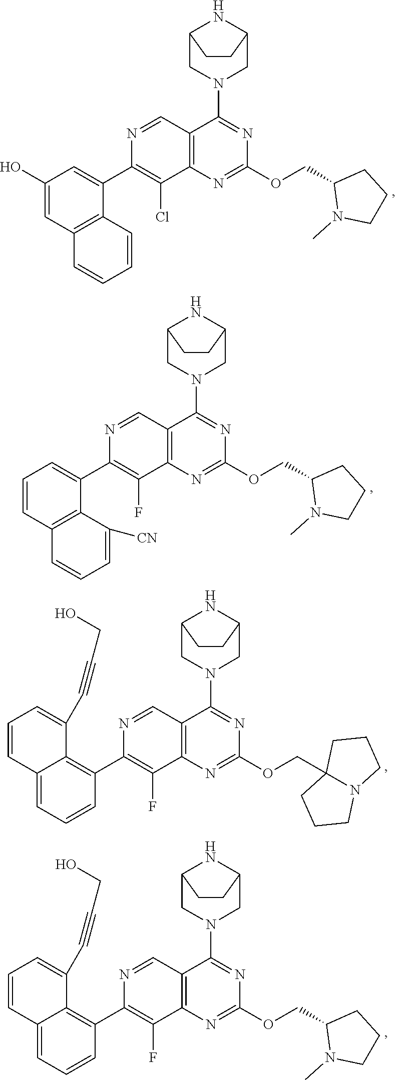

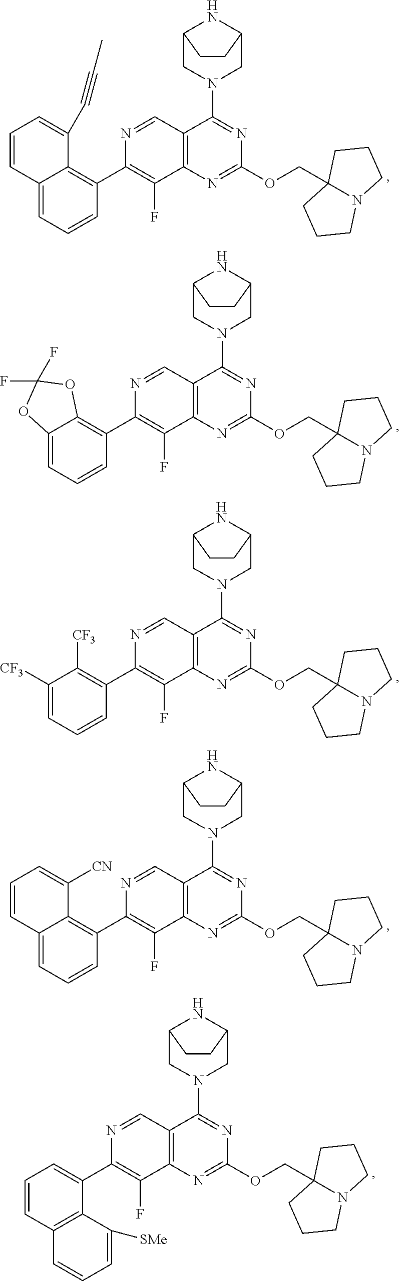 [SMILES: CC#Cc1cccc2cccc(-c3ncc4c(N5CC6CCC(C5)N6)nc(OCC56CCCN5CCC6)nc4c3F)c12 ; Fc1c(-c2cccc3c2OC(F)(F)O3)ncc2c(N3CC4CCC(C3)N4)nc(OCC34CCCN3CCC4)nc12 ; Fc1c(-c2cccc(C(F)(F)F)c2C(F)(F)F)ncc2c(N3CC4CCC(C3)N4)nc(OCC34CCCN3CCC4)nc12 ; N#Cc1cccc2cccc(-c3ncc4c(N5CC6CCC(C5)N6)nc(OCC56CCCN5CCC6)nc4c3F)c12 ; CSc1cccc2cccc(-c3ncc4c(N5CC6CCC(C5)N6)nc(OCC56CCCN5CCC6)nc4c3F)c12]

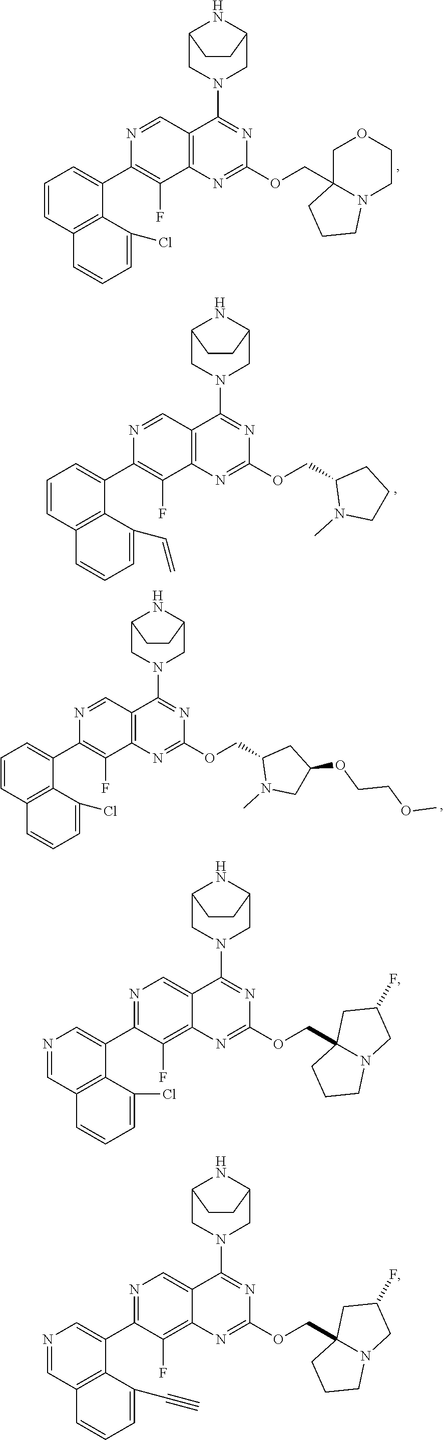

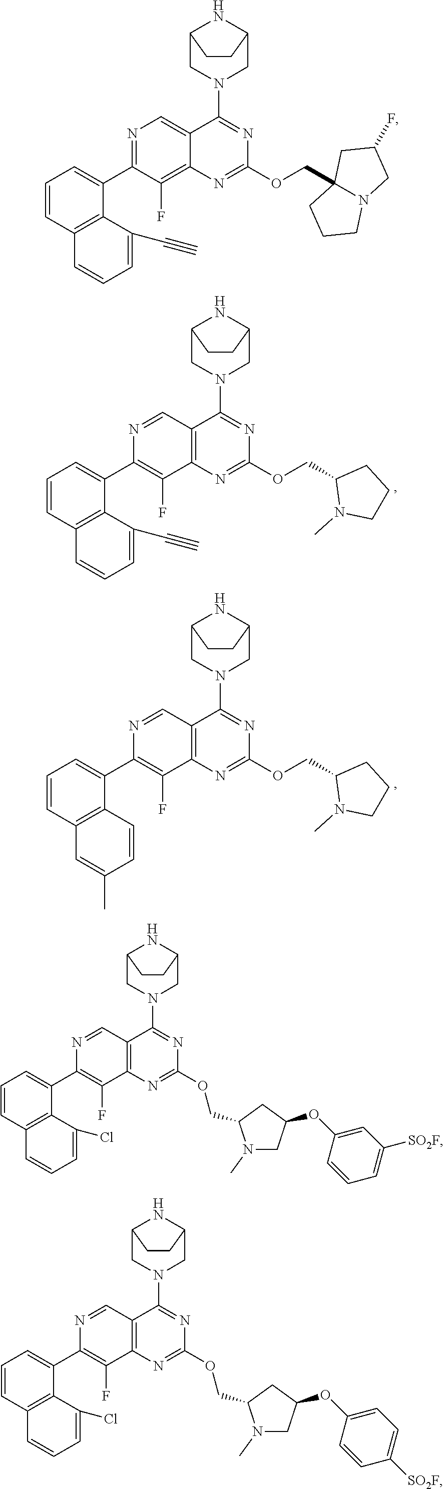

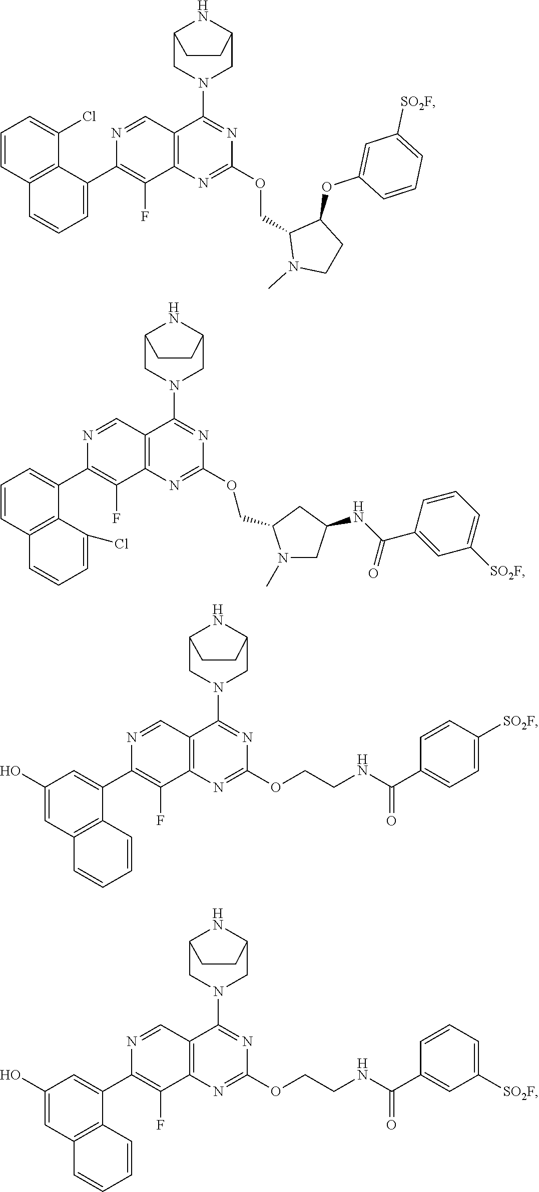 [SMILES: CN1CC[C@H](Oc2cccc(S(=O)(=O)F)c2)[C@H]1COc1nc(N2CC3CCC(C2)N3)c2cnc(-c3cccc4cccc(Cl)c34)c(F)c2n1 ; CN1C[C@H](NC(=O)c2cccc(S(=O)(=O)F)c2)C[C@H]1COc1nc(N2CC3CCC(C2)N3)c2cnc(-c3cccc4cccc(Cl)c34)c(F)c2n1 ; O=C(NCCOc1nc(N2CC3CCC(C2)N3)c2cnc(-c3cc(O)cc4ccccc34)c(F)c2n1)c1ccc(S(=O)(=O)F)cc1 ; O=C(NCCOc1nc(N2CC3CCC(C2)N3)c2cnc(-c3cc(O)cc4ccccc34)c(F)c2n1)c1cccc(S(=O)(=O)F)c1]

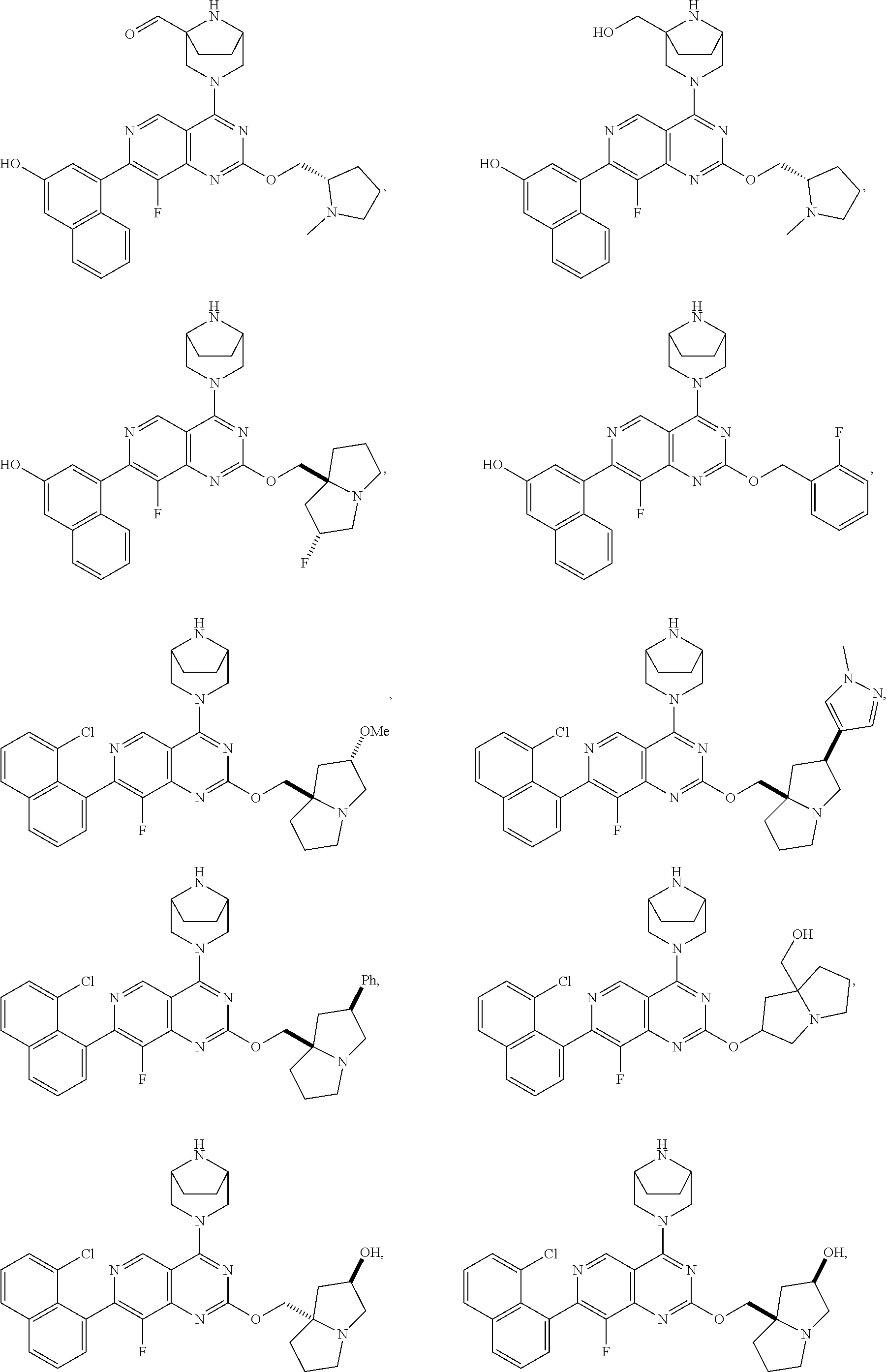

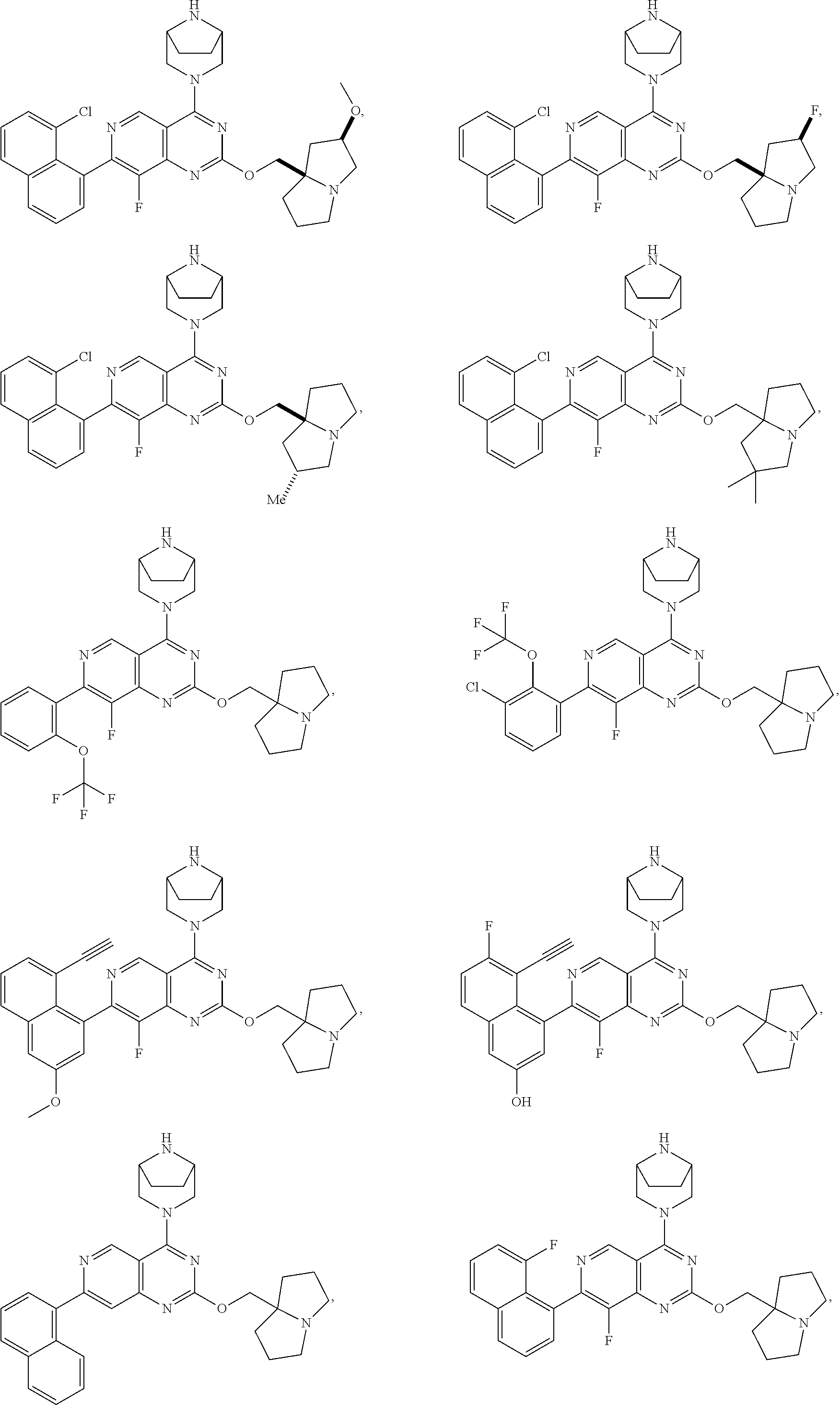

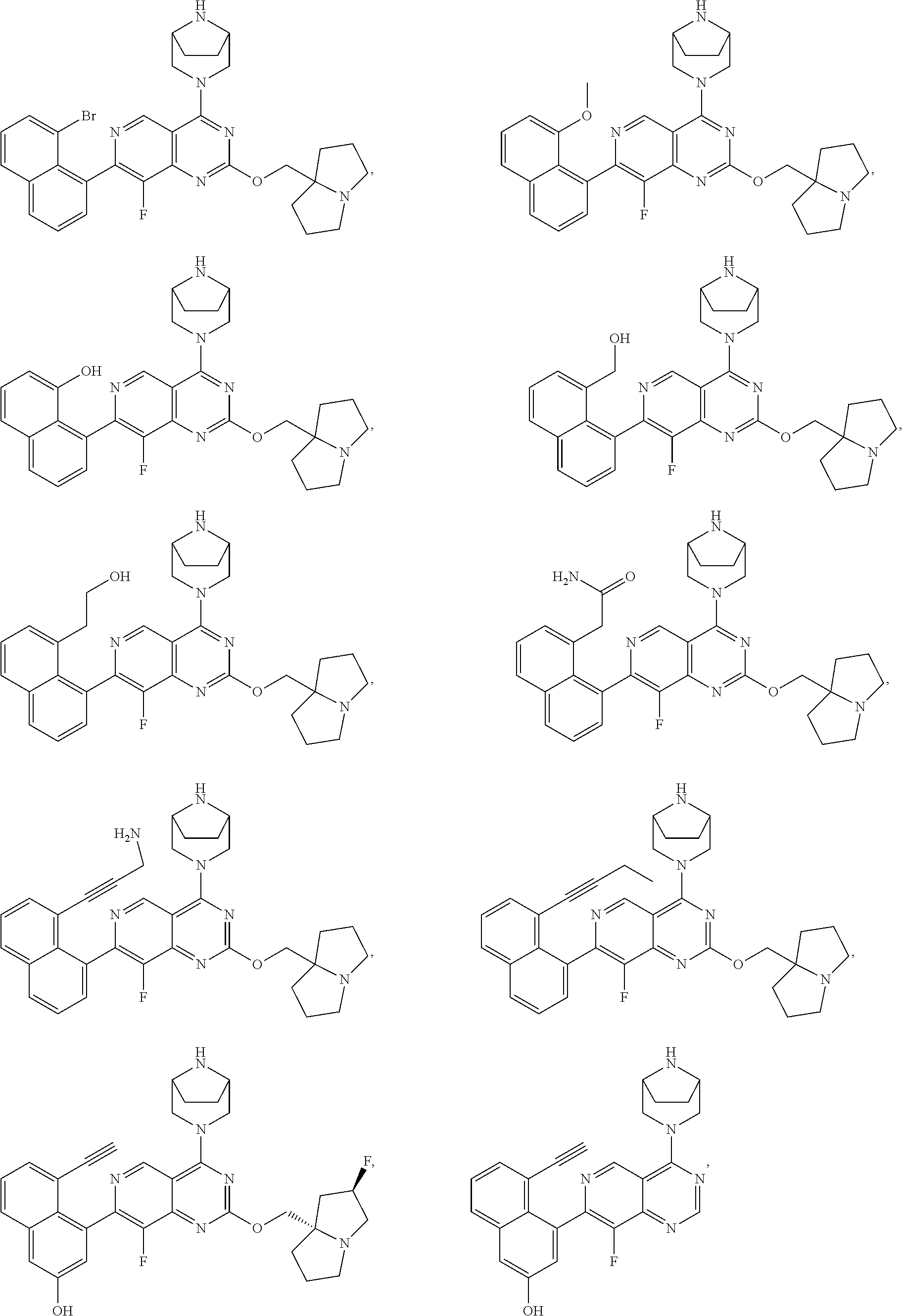

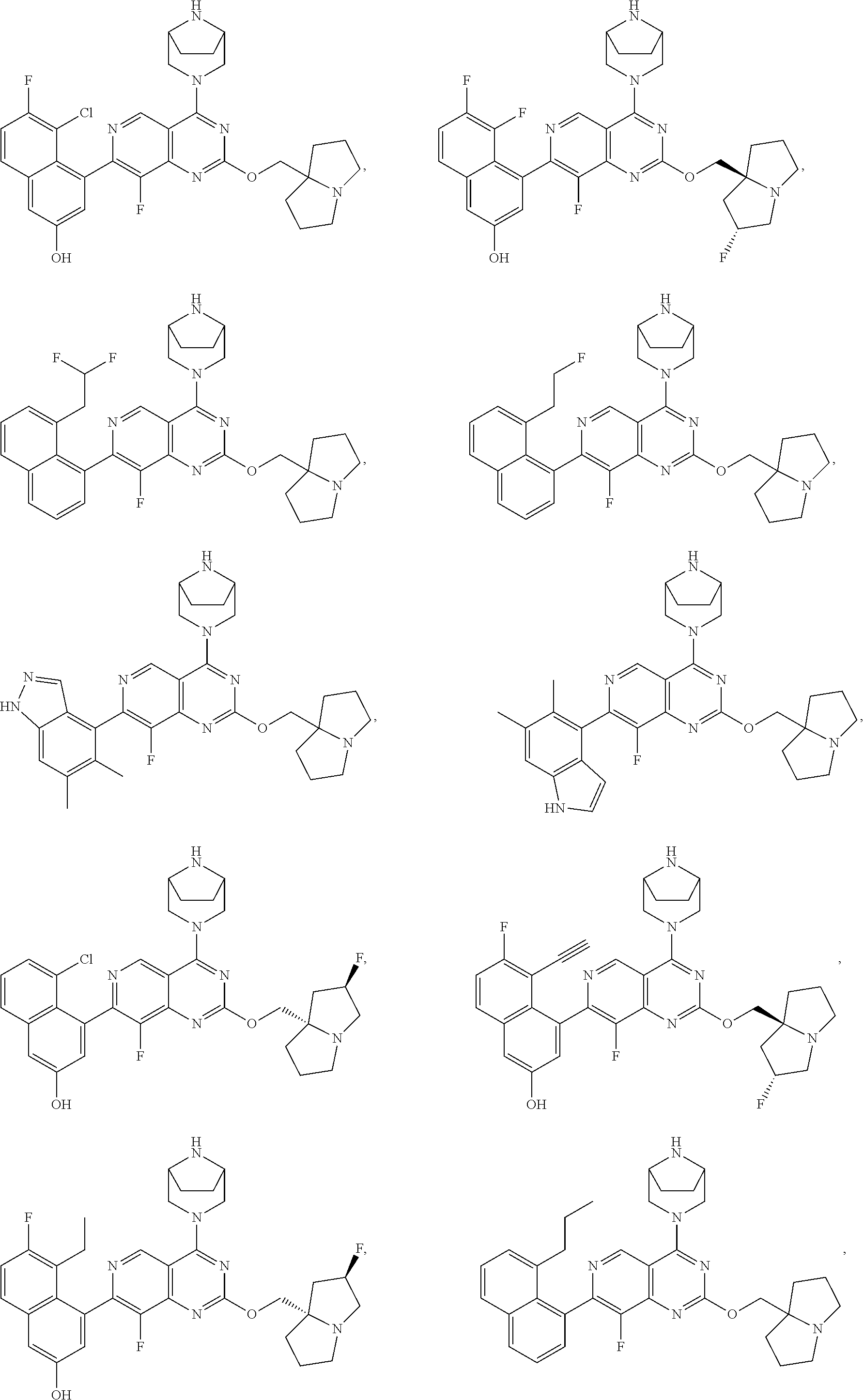

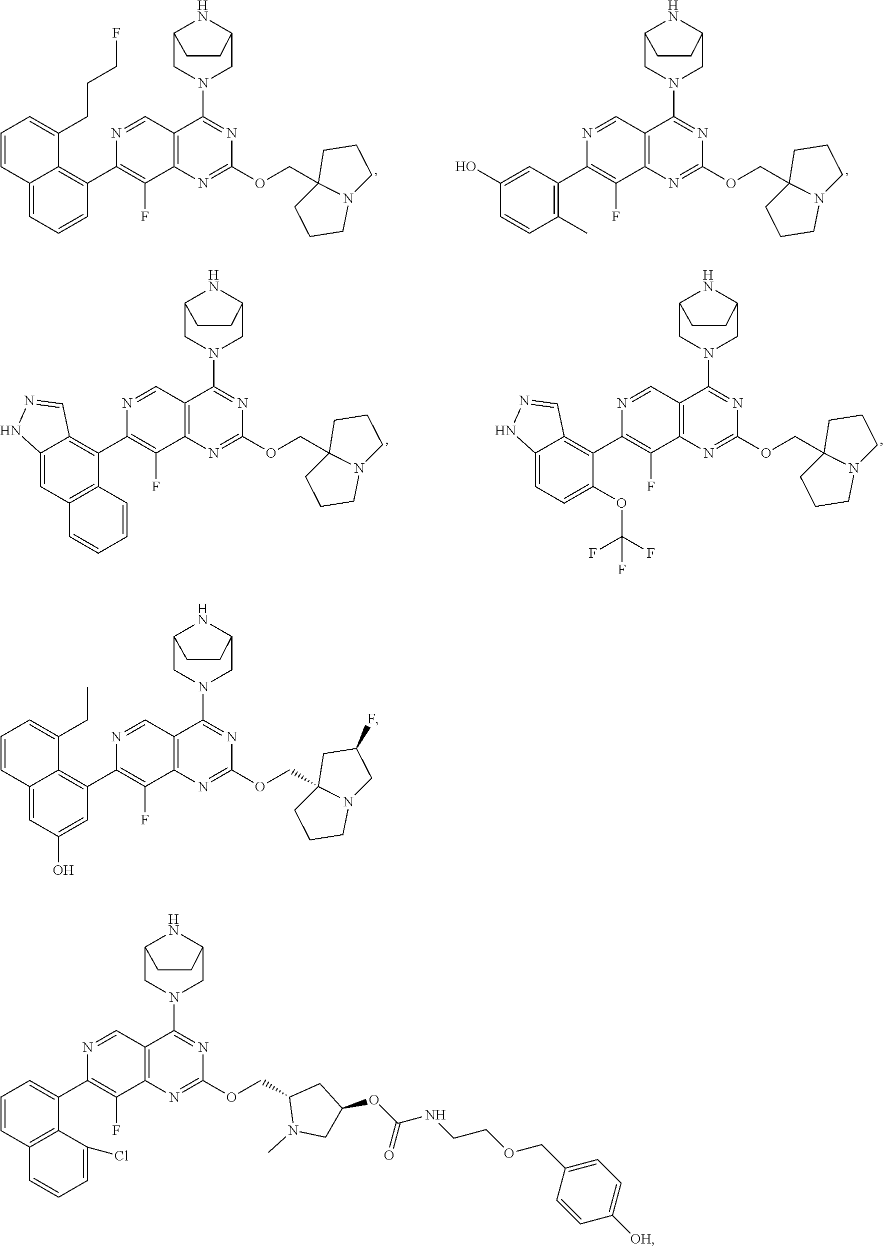 [SMILES: FCCCc1cccc2cccc(-c3ncc4c(N5CC6CCC(C5)N6)nc(OCC56CCCN5CCC6)nc4c3F)c12 ; Cc1ccc(O)cc1-c1ncc2c(N3CC4CCC(C3)N4)nc(OCC34CCCN3CCC4)nc2c1F ; Fc1c(-c2c3ccccc3cc3[nH]ncc23)ncc2c(N3CC4CCC(C3)N4)nc(OCC34CCCN3CCC4)nc12 ; Fc1c(-c2c(OC(F)(F)F)ccc3[nH]ncc23)ncc2c(N3CC4CCC(C3)N4)nc(OCC34CCCN3CCC4)nc12 ; CCc1cccc2cc(O)cc(-c3ncc4c(N5CC6CCC(C5)N6)nc(OC[C@@]56CCCN5C[C@H](F)C6)nc4c3F)c12 ; CN1C[C@H](OC(=O)NCCOCc2ccc(O)cc2)C[C@H]1COc1nc(N2CC3CCC(C2)N3)c2cnc(-c3cccc4cccc(Cl)c34)c(F)c2n1]

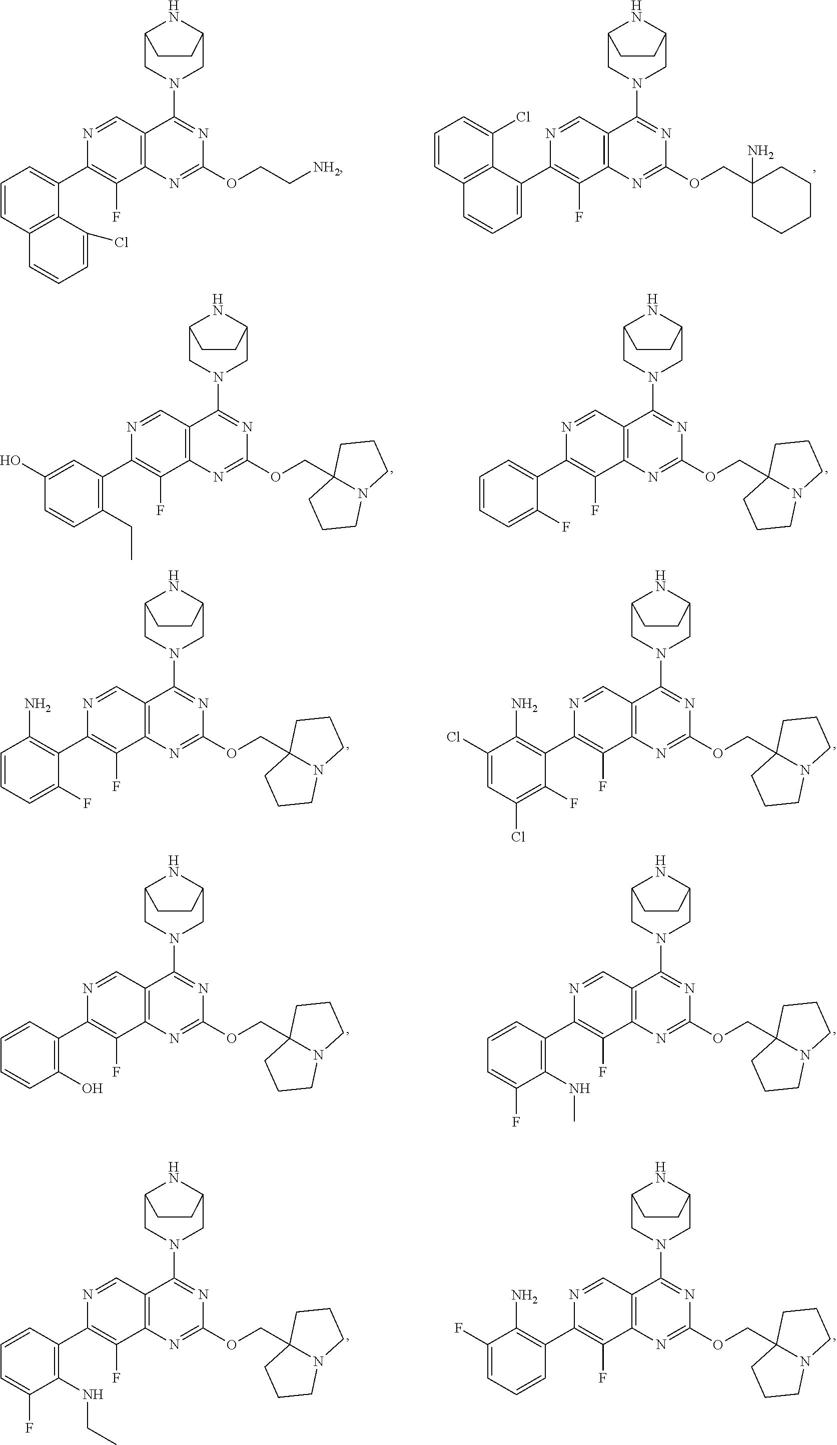

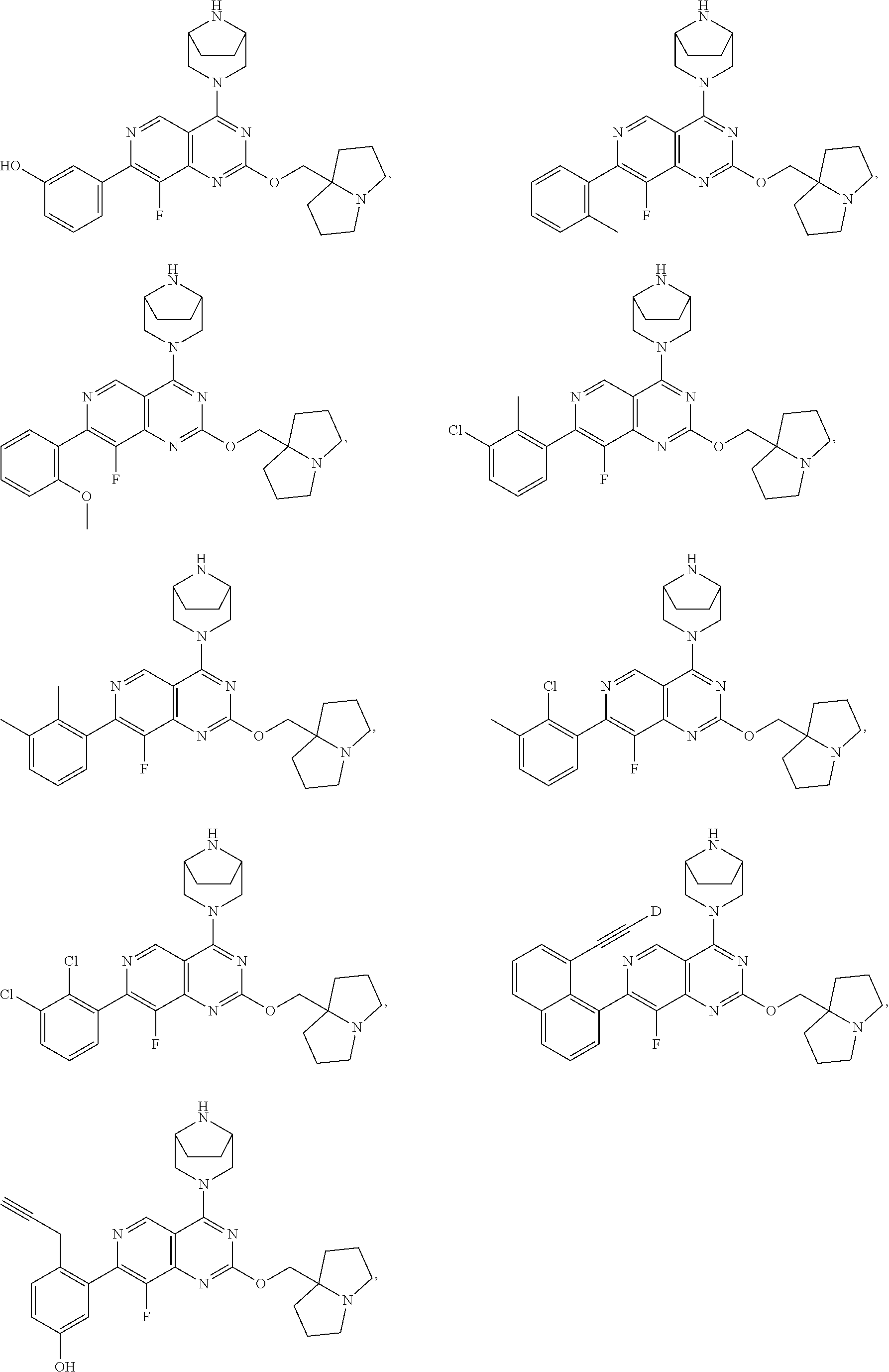

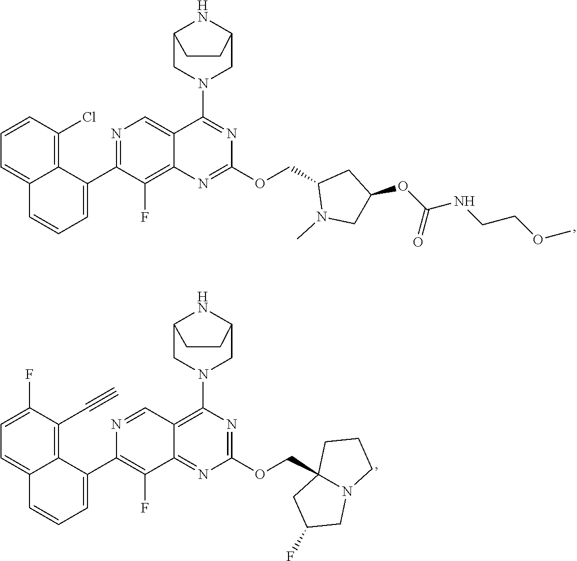

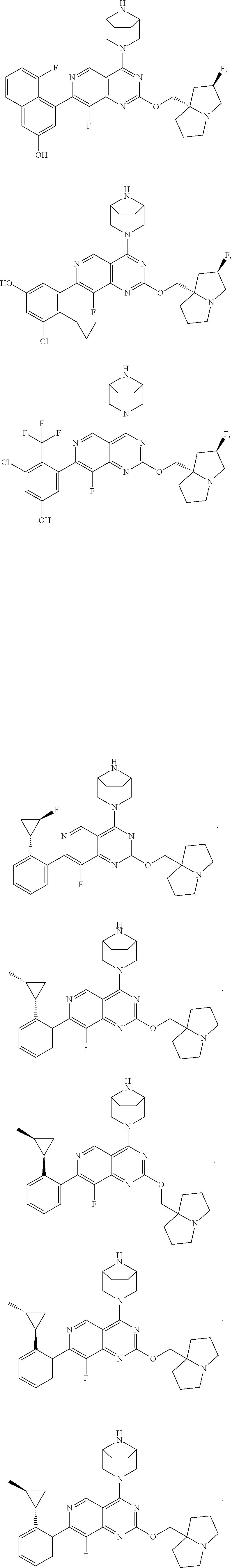

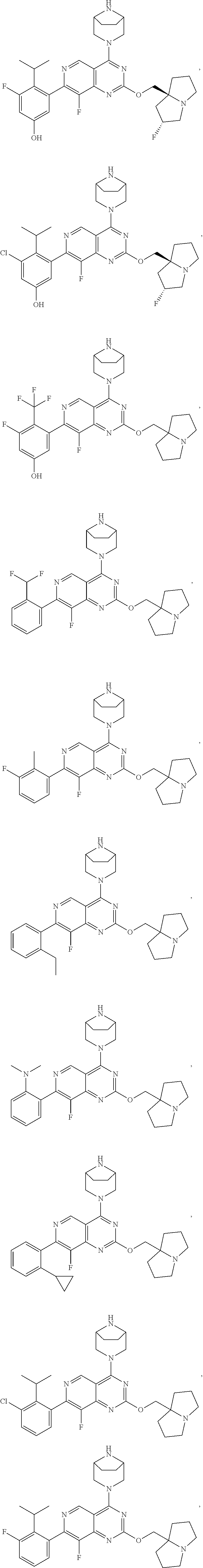

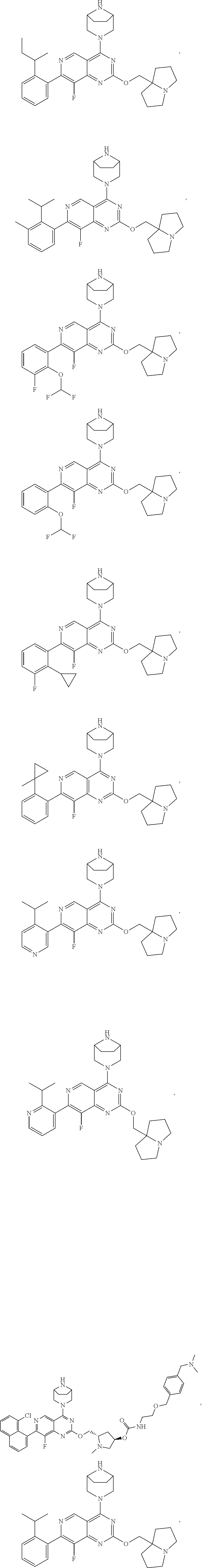

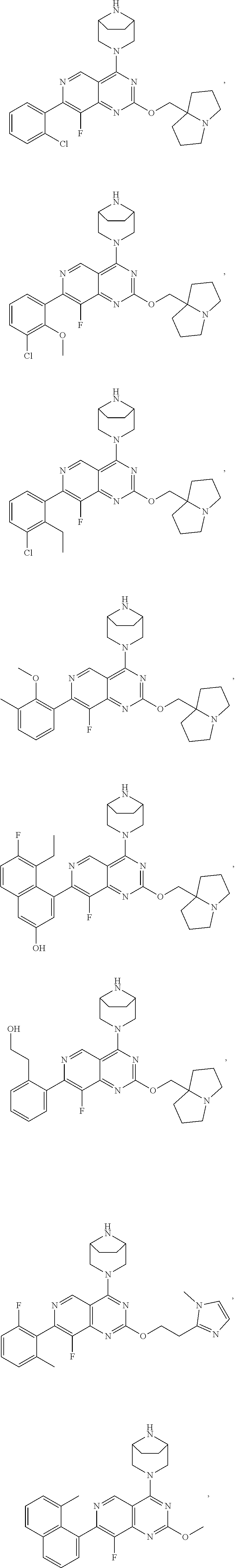

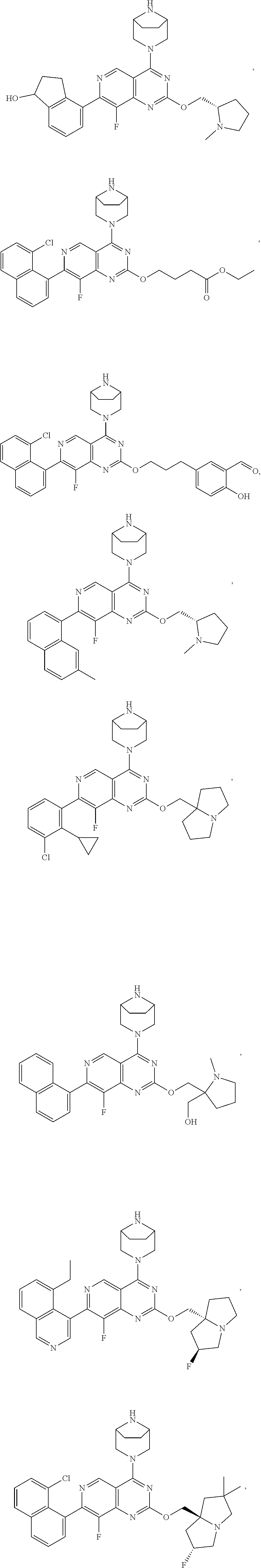

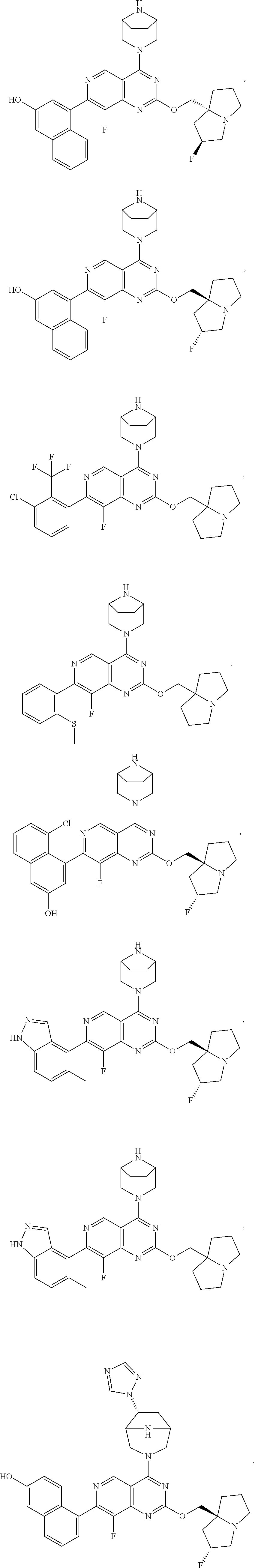

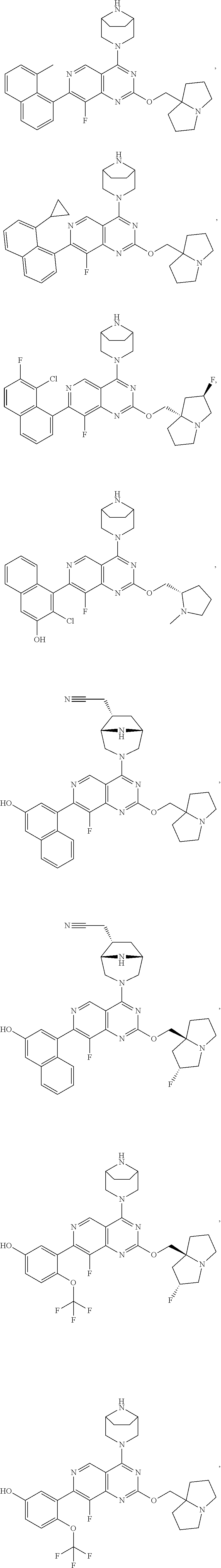

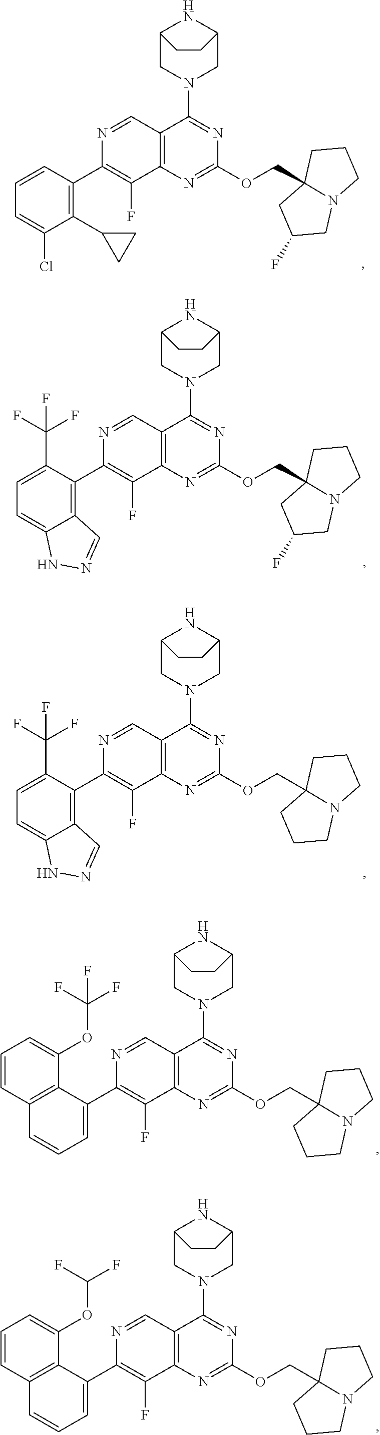 [SMILES: Fc1c(-c2cccc(Cl)c2C2CC2)ncc2c(N3CC4CCC(C3)N4)nc(OC[C@@]34CCCN3C[C@H](F)C4)nc12 ; Fc1c(-c2c(C(F)(F)F)ccc3[nH]ncc23)ncc2c(N3CC4CCC(C3)N4)nc(OC[C@@]34CCCN3C[C@H](F)C4)nc12 ; Fc1c(-c2c(C(F)(F)F)ccc3[nH]ncc23)ncc2c(N3CC4CCC(C3)N4)nc(OCC34CCCN3CCC4)nc12 ; Fc1c(-c2cccc3cccc(OC(F)(F)F)c23)ncc2c(N3CC4CCC(C3)N4)nc(OCC34CCCN3CCC4)nc12 ; Fc1c(-c2cccc3cccc(OC(F)F)c23)ncc2c(N3CC4CCC(C3)N4)nc(OCC34CCCN3CCC4)nc12]

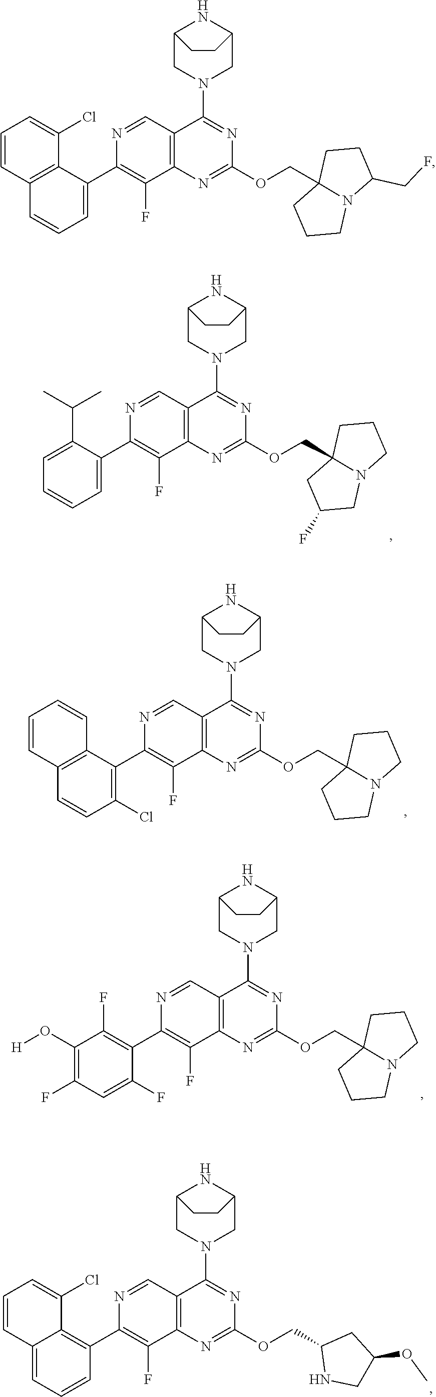 [SMILES: FCC1CCC2(COc3nc(N4CC5CCC(C4)N5)c4cnc(-c5cccc6cccc(Cl)c56)c(F)c4n3)CCCN12 ; CC(C)c1ccccc1-c1ncc2c(N3CC4CCC(C3)N4)nc(OC[C@@]34CCCN3C[C@H](F)C4)nc2c1F ; Fc1c(-c2c(Cl)ccc3ccccc23)ncc2c(N3CC4CCC(C3)N4)nc(OCC34CCCN3CCC4)nc12 ; Oc1c(F)cc(F)c(-c2ncc3c(N4CC5CCC(C4)N5)nc(OCC45CCCN4CCC5)nc3c2F)c1F ; CO[C@H]1CN[C@H](COc2nc(N3CC4CCC(C3)N4)c3cnc(-c4cccc5cccc(Cl)c45)c(F)c3n2)C1]

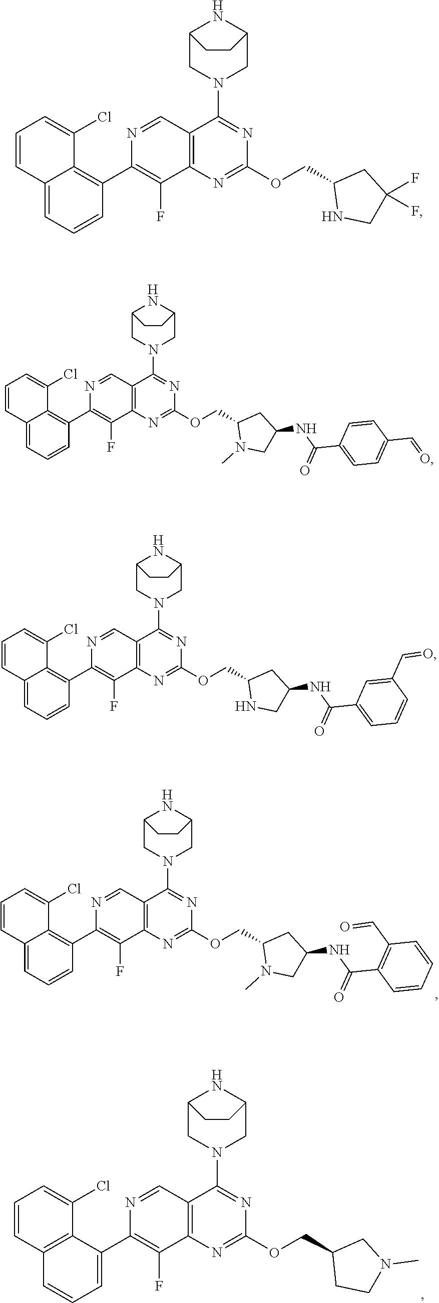 [SMILES: Fc1c(-c2cccc3cccc(Cl)c23)ncc2c(N3CC4CCC(C3)N4)nc(OC[C@@H]3CC(F)(F)CN3)nc12 ; CN1C[C@H](NC(=O)c2ccc(C=O)cc2)C[C@H]1COc1nc(N2CC3CCC(C2)N3)c2cnc(-c3cccc4cccc(Cl)c34)c(F)c2n1 ; O=Cc1cccc(C(=O)N[C@H]2CN[C@H](COc3nc(N4CC5CCC(C4)N5)c4cnc(-c5cccc6cccc(Cl)c56)c(F)c4n3)C2)c1 ; CN1C[C@H](NC(=O)c2ccccc2C=O)C[C@H]1COc1nc(N2CC3CCC(C2)N3)c2cnc(-c3cccc4cccc(Cl)c34)c(F)c2n1 ; CN1CC[C@@H](COc2nc(N3CC4CCC(C3)N4)c3cnc(-c4cccc5cccc(Cl)c45)c(F)c3n2)C1]

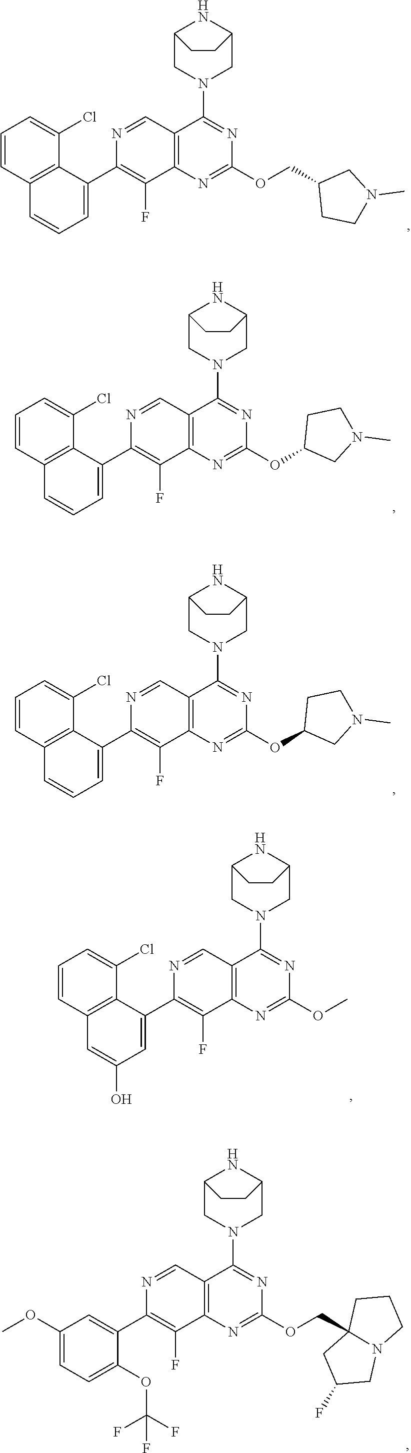

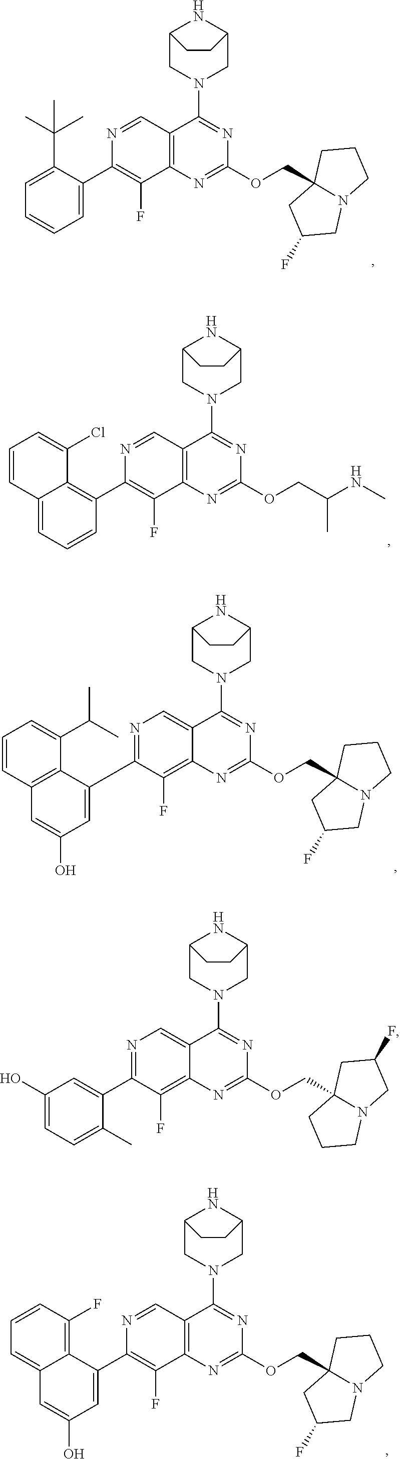 [SMILES: CC(C)(C)c1ccccc1-c1ncc2c(N3CC4CCC(C3)N4)nc(OC[C@@]34CCCN3C[C@H](F)C4)nc2c1F ; CNC(C)COc1nc(N2CC3CCC(C2)N3)c2cnc(-c3cccc4cccc(Cl)c34)c(F)c2n1 ; CC(C)c1cccc2cc(O)cc(-c3ncc4c(N5CC6CCC(C5)N6)nc(OC[C@@]56CCCN5C[C@H](F)C6)nc4c3F)c12 ; Cc1ccc(O)cc1-c1ncc2c(N3CC4CCC(C3)N4)nc(OC[C@@]34CCCN3C[C@H](F)C4)nc2c1F ; Oc1cc(-c2ncc3c(N4CC5CCC(C4)N5)nc(OC[C@@]45CCCN4C[C@H](F)C5)nc3c2F)c2c(F)cccc2c1]

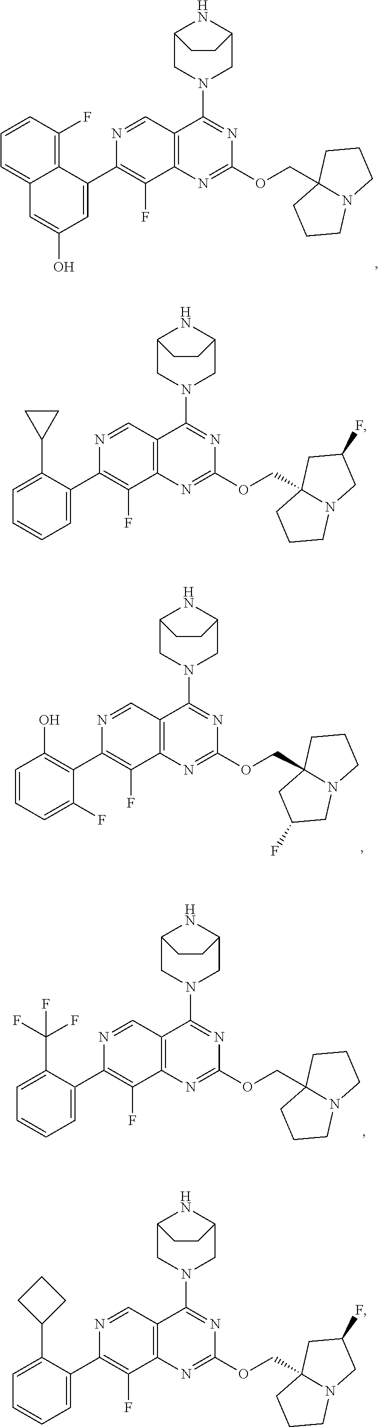 [SMILES: Oc1cc(-c2ncc3c(N4CC5CCC(C4)N5)nc(OCC45CCCN4CCC5)nc3c2F)c2c(F)cccc2c1 ; Fc1c(-c2ccccc2C2CC2)ncc2c(N3CC4CCC(C3)N4)nc(OC[C@@]34CCCN3C[C@H](F)C4)nc12 ; Oc1cccc(F)c1-c1ncc2c(N3CC4CCC(C3)N4)nc(OC[C@@]34CCCN3C[C@H](F)C4)nc2c1F ; Fc1c(-c2ccccc2C(F)(F)F)ncc2c(N3CC4CCC(C3)N4)nc(OCC34CCCN3CCC4)nc12 ; Fc1c(-c2ccccc2C2CCC2)ncc2c(N3CC4CCC(C3)N4)nc(OC[C@@]34CCCN3C[C@H](F)C4)nc12]

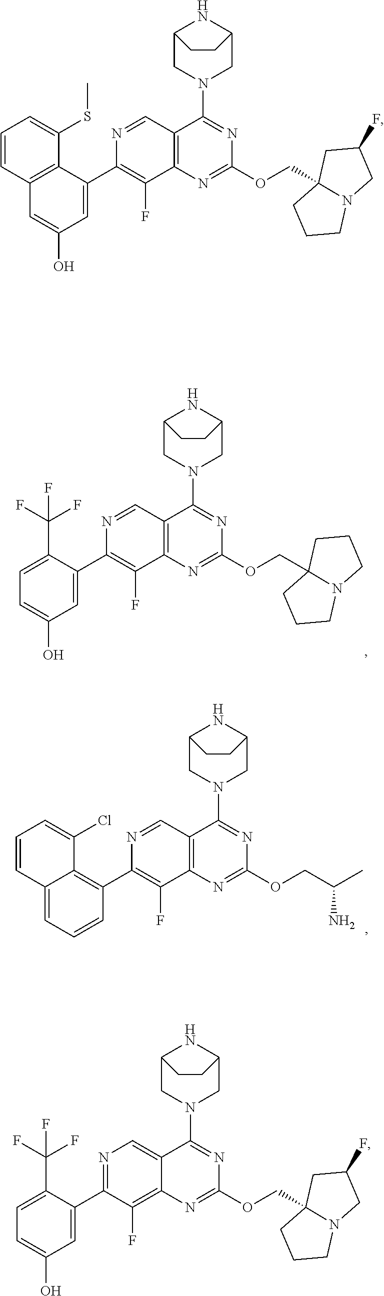 [SMILES: CSc1cccc2cc(O)cc(-c3ncc4c(N5CC6CCC(C5)N6)nc(OC[C@@]56CCCN5C[C@H](F)C6)nc4c3F)c12 ; Oc1ccc(C(F)(F)F)c(-c2ncc3c(N4CC5CCC(C4)N5)nc(OCC45CCCN4CCC5)nc3c2F)c1 ; C[C@H](N)COc1nc(N2CC3CCC(C2)N3)c2cnc(-c3cccc4cccc(Cl)c34)c(F)c2n1 ; Oc1ccc(C(F)(F)F)c(-c2ncc3c(N4CC5CCC(C4)N5)nc(OC[C@@]45CCCN4C[C@H](F)C5)nc3c2F)c1]

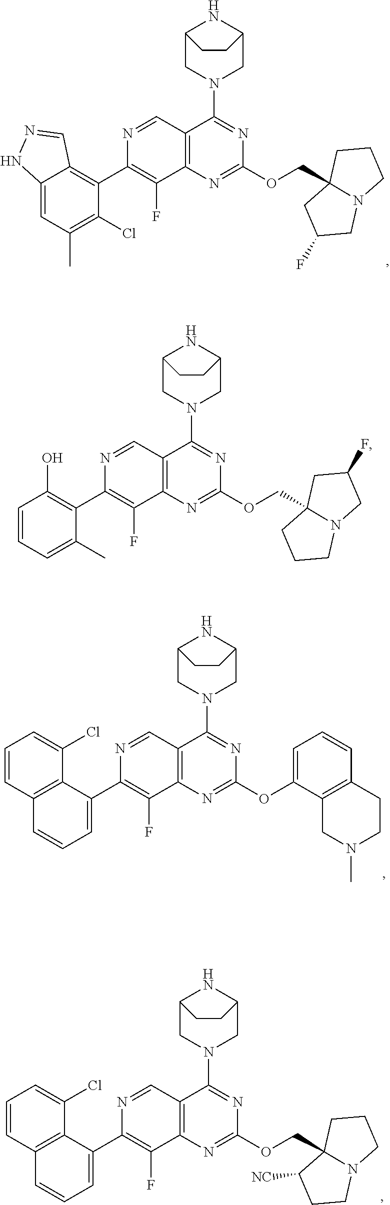 [SMILES: Cc1cc2[nH]ncc2c(-c2ncc3c(N4CC5CCC(C4)N5)nc(OC[C@@]45CCCN4C[C@H](F)C5)nc3c2F)c1Cl ; Cc1cccc(O)c1-c1ncc2c(N3CC4CCC(C3)N4)nc(OC[C@@]34CCCN3C[C@H](F)C4)nc2c1F ; CN1CCc2cccc(Oc3nc(N4CC5CCC(C4)N5)c4cnc(-c5cccc6cccc(Cl)c56)c(F)c4n3)c2C1 ; N#C[C@H]1CCN2CCC[C@@]12COc1nc(N2CC3CCC(C2)N3)c2cnc(-c3cccc4cccc(Cl)c34)c(F)c2n1]

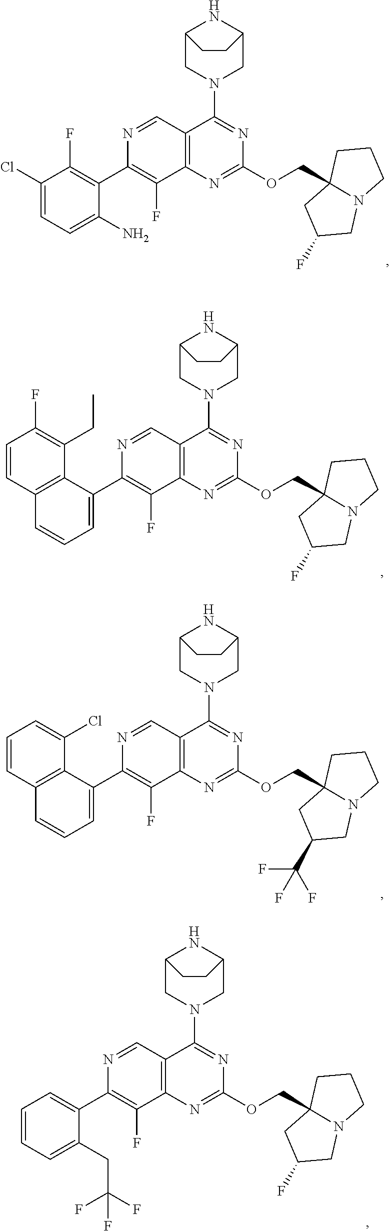 [SMILES: Nc1ccc(Cl)c(F)c1-c1ncc2c(N3CC4CCC(C3)N4)nc(OC[C@@]34CCCN3C[C@H](F)C4)nc2c1F ; CCc1c(F)ccc2cccc(-c3ncc4c(N5CC6CCC(C5)N6)nc(OC[C@@]56CCCN5C[C@H](F)C6)nc4c3F)c12 ; Fc1c(-c2cccc3cccc(Cl)c23)ncc2c(N3CC4CCC(C3)N4)nc(OC[C@@]34CCCN3C[C@@H](C(F)(F)F)C4)nc12 ; Fc1c(-c2ccccc2CC(F)(F)F)ncc2c(N3CC4CCC(C3)N4)nc(OC[C@@]34CCCN3C[C@H](F)C4)nc12]

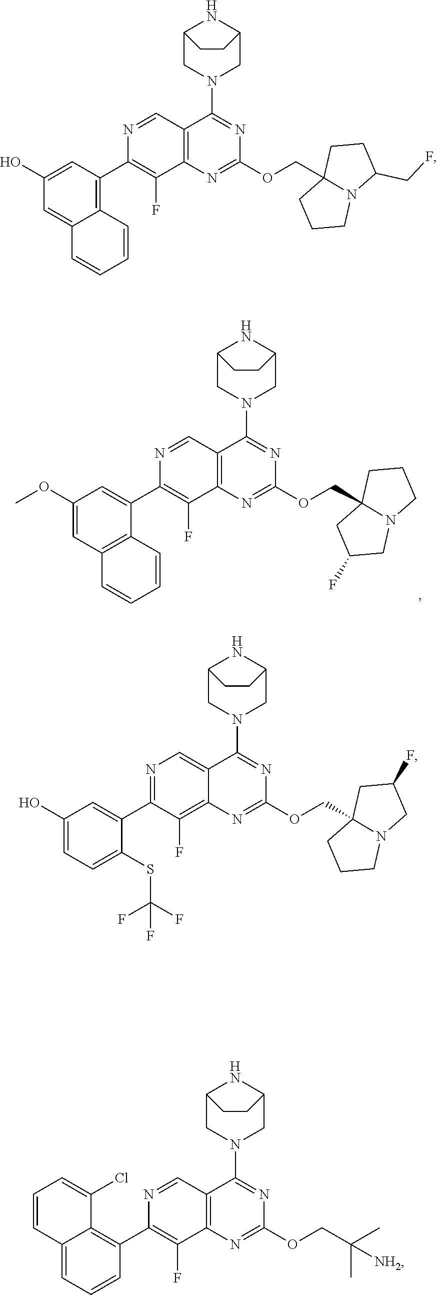 [SMILES: Oc1cc(-c2ncc3c(N4CC5CCC(C4)N5)nc(OCC45CCCN4C(CF)CC5)nc3c2F)c2ccccc2c1 ; COc1cc(-c2ncc3c(N4CC5CCC(C4)N5)nc(OC[C@@]45CCCN4C[C@H](F)C5)nc3c2F)c2ccccc2c1 ; Oc1ccc(SC(F)(F)F)c(-c2ncc3c(N4CC5CCC(C4)N5)nc(OC[C@@]45CCCN4C[C@H](F)C5)nc3c2F)c1 ; CC(C)(N)COc1nc(N2CC3CCC(C2)N3)c2cnc(-c3cccc4cccc(Cl)c34)c(F)c2n1]

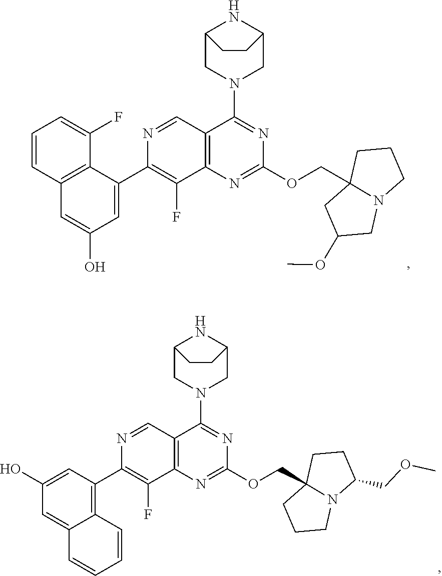 [SMILES: COC1CN2CCCC2(COc2nc(N3CC4CCC(C3)N4)c3cnc(-c4cc(O)cc5cccc(F)c45)c(F)c3n2)C1 ; COC[C@H]1CC[C@@]2(COc3nc(N4CC5CCC(C4)N5)c4cnc(-c5cc(O)cc6ccccc56)c(F)c4n3)CCCN12]

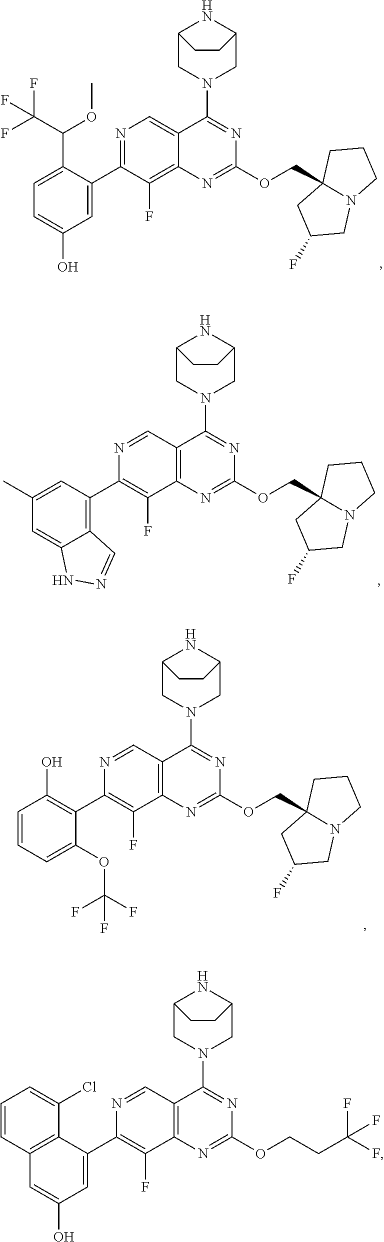 [SMILES: COC(c1ccc(O)cc1-c1ncc2c(N3CC4CCC(C3)N4)nc(OC[C@@]34CCCN3C[C@H](F)C4)nc2c1F)C(F)(F)F ; Cc1cc(-c2ncc3c(N4CC5CCC(C4)N5)nc(OC[C@@]45CCCN4C[C@H](F)C5)nc3c2F)c2cn[nH]c2c1 ; Oc1cccc(OC(F)(F)F)c1-c1ncc2c(N3CC4CCC(C3)N4)nc(OC[C@@]34CCCN3C[C@H](F)C4)nc2c1F ; Oc1cc(-c2ncc3c(N4CC5CCC(C4)N5)nc(OCCC(F)(F)F)nc3c2F)c2c(Cl)cccc2c1]

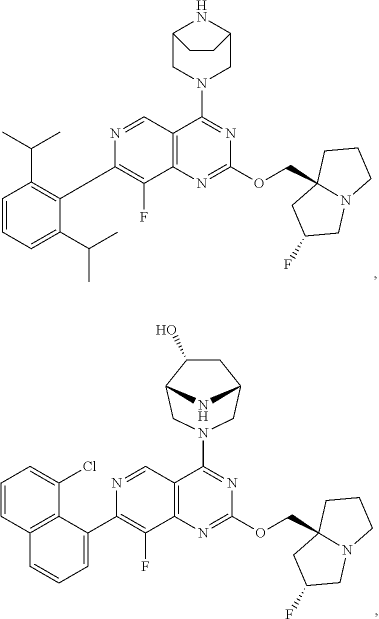 [SMILES: CC(C)c1cccc(C(C)C)c1-c1ncc2c(N3CC4CCC(C3)N4)nc(OC[C@@]34CCCN3C[C@H](F)C4)nc2c1F ; O[C@@H]1C[C@@H]2CN(c3nc(OC[C@@]45CCCN4C[C@H](F)C5)nc4c(F)c(-c5cccc6cccc(Cl)c56)ncc34)C[C@H]1N2]

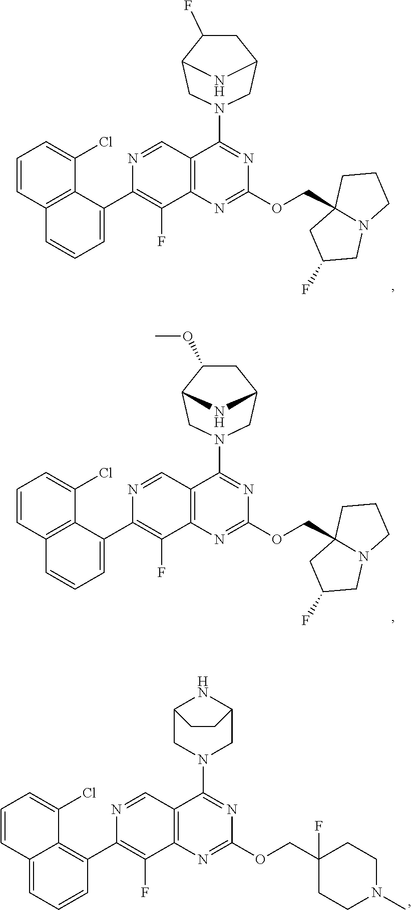 [SMILES: Fc1c(-c2cccc3cccc(Cl)c23)ncc2c(N3CC4CC(F)C(C3)N4)nc(OC[C@@]34CCCN3C[C@H](F)C4)nc12 ; CO[C@@H]1C[C@@H]2CN(c3nc(OC[C@@]45CCCN4C[C@H](F)C5)nc4c(F)c(-c5cccc6cccc(Cl)c56)ncc34)C[C@H]1N2 ; CN1CCC(F)(COc2nc(N3CC4CCC(C3)N4)c3cnc(-c4cccc5cccc(Cl)c45)c(F)c3n2)CC1]

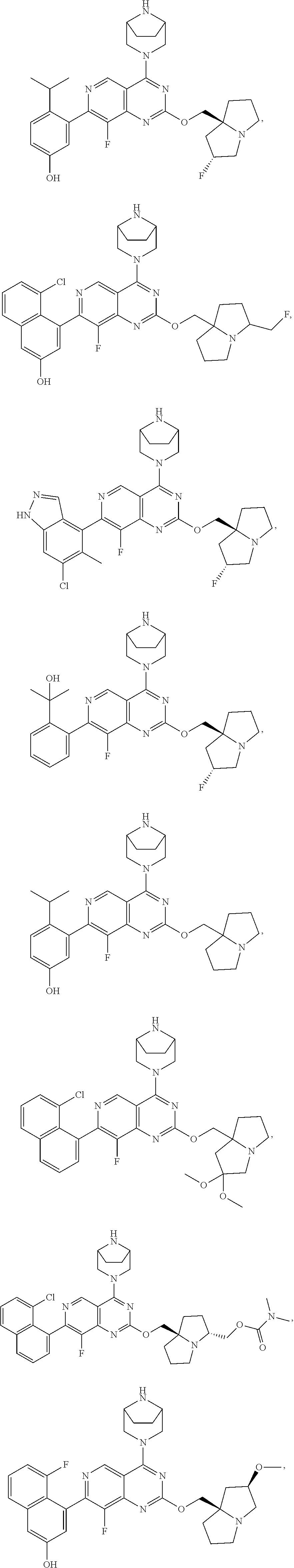

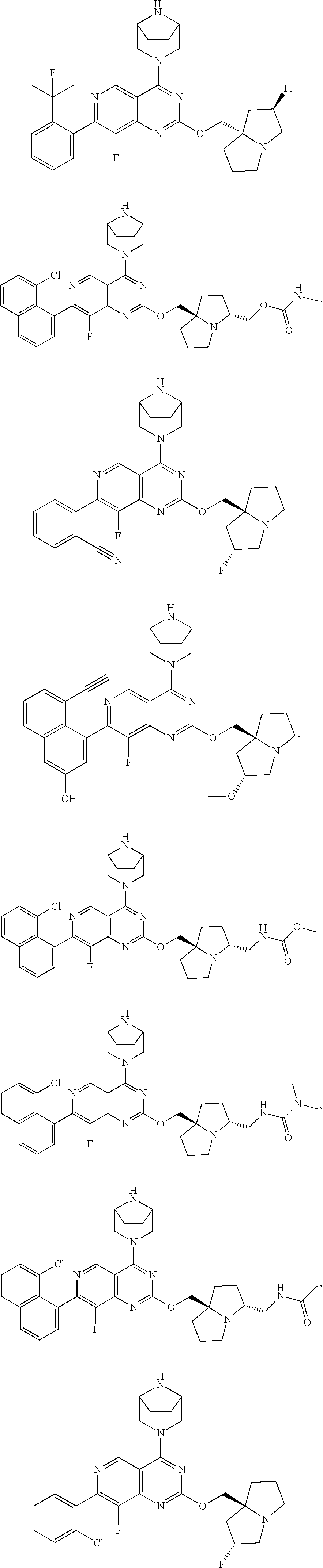

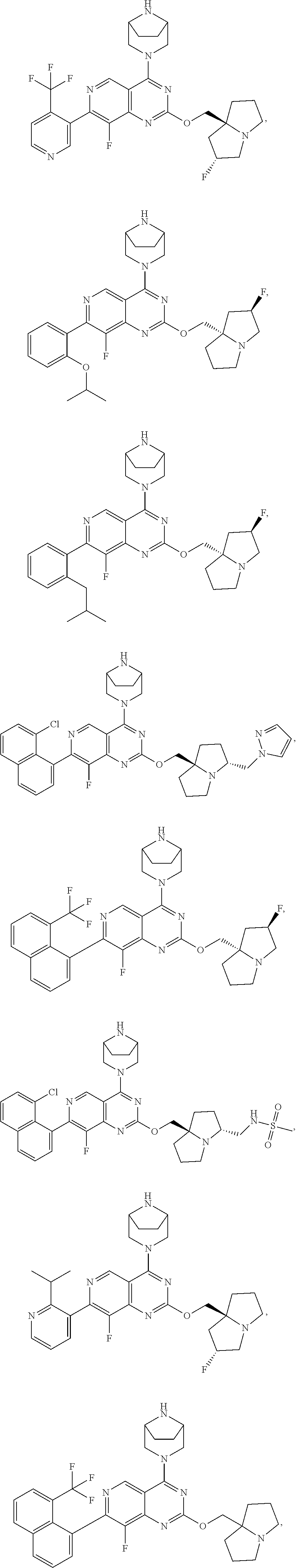

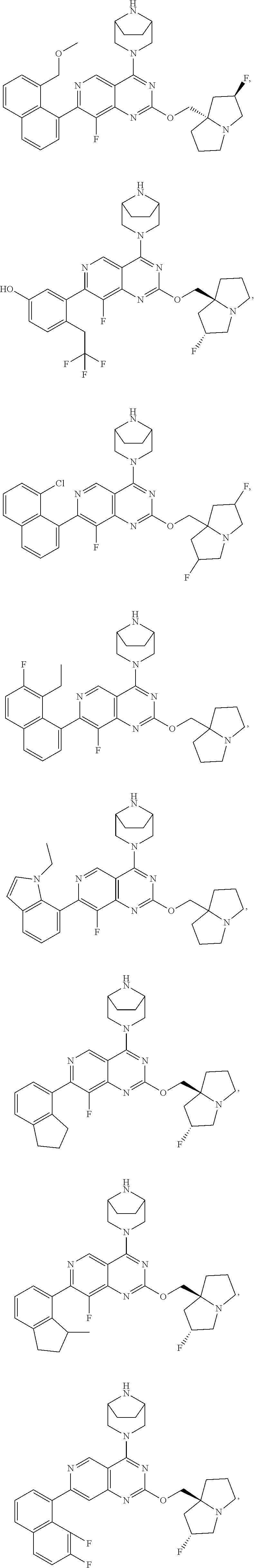

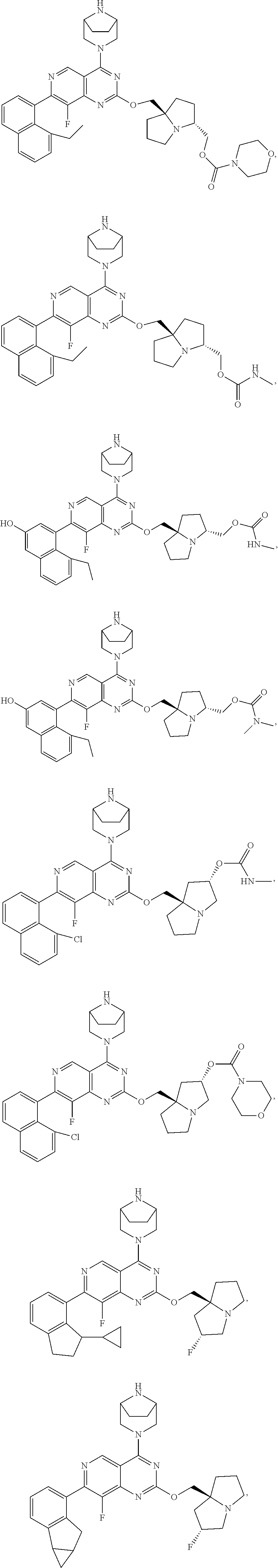

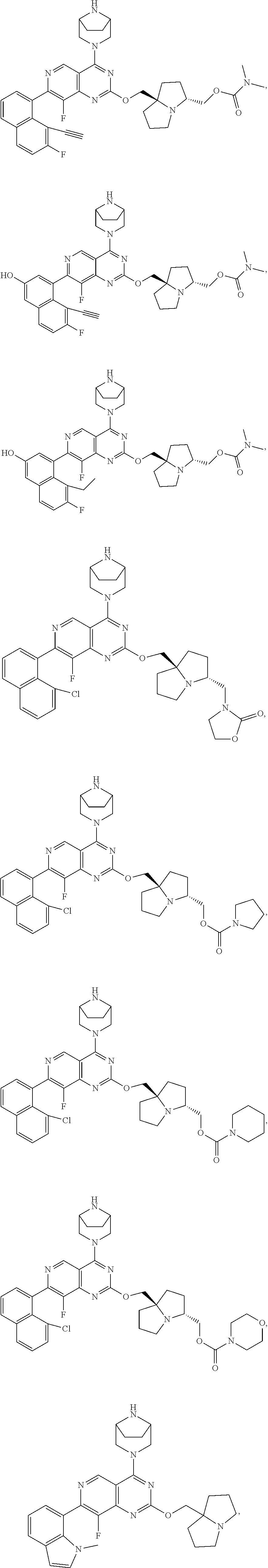

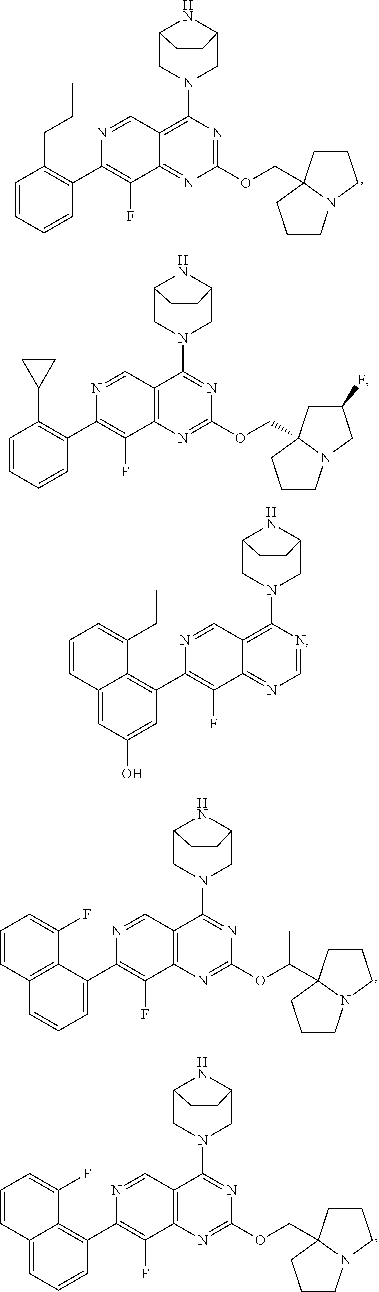

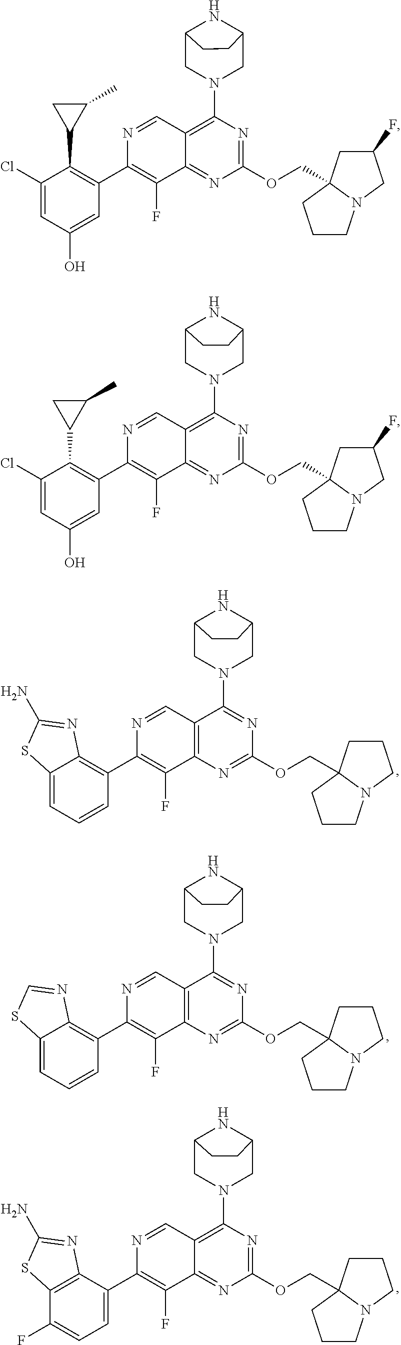

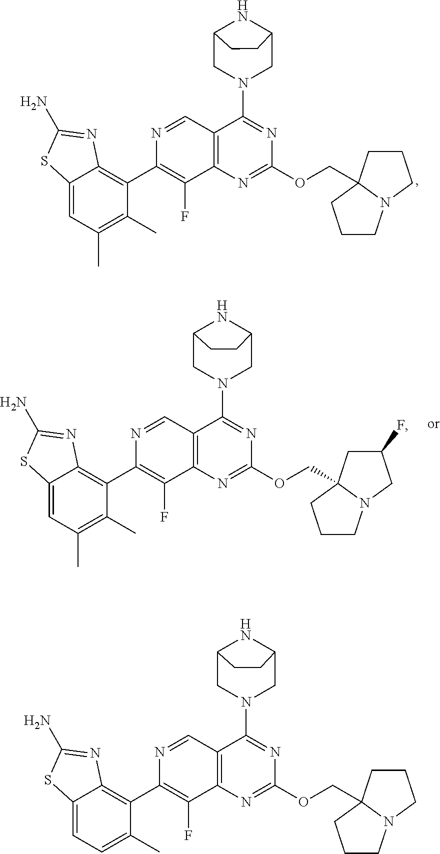 [SMILES: Cc1cc2sc(N)nc2c(-c2ncc3c(N4CC5CCC(C4)N5)nc(OCC45CCCN4CCC5)nc3c2F)c1C ; Cc1cc2sc(N)nc2c(-c2ncc3c(N4CC5CCC(C4)N5)nc(OC[C@@]45CCCN4C[C@H](F)C5)nc3c2F)c1C ; "or" ; Cc1ccc2sc(N)nc2c1-c1ncc2c(N3CC4CCC(C3)N4)nc(OCC34CCCN3CCC4)nc2c1F]

or pharmaceutically acceptable salts thereof.

In one aspect of the invention, KRas G12D inhibitors comprise compound MRTX1133 or MRTX1133 analogs and related compounds such as any of the compounds disclosed and described in WIPO publication WO2021/041671, including but not limited to: Ex. 252 (MRTX1133), 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-ethynyl-6-fluoronaphthalen-2-ol; Ex. 243, 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-ethynylnaphthalen-2-ol; Ex. 246, 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5,6-difluoronaphthalen-2-ol; Ex. 251, 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-chloronaphthalen-2-ol; Ex. 253, 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-ethyl-6-fluoronaphthalen-2-ol; Ex. 259, 4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethylnaphthalen-2-ol; and Ex. 282, 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-fluoronaphthalen-2-ol; or a pharmaceutically acceptable salt thereof and a pharmaceutically acceptable excipient

In one embodiment, the KRas G12D inhibitor is:

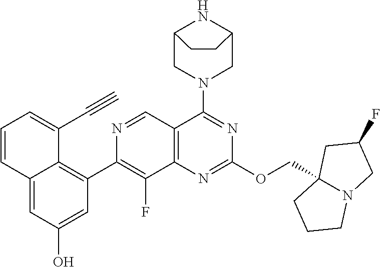 [SMILES: C#Cc1cccc2cc(O)cc(-c3ncc4c(N5CC6CCC(C5)N6)nc(OC[C@@]56CCCN5C[C@H](F)C6)nc4c3F)c12]

(also referred to as Example 243 in WO 2021/041671) or a pharmaceutically acceptable salt thereof.

In one embodiment, the KRas G12D inhibitor is:

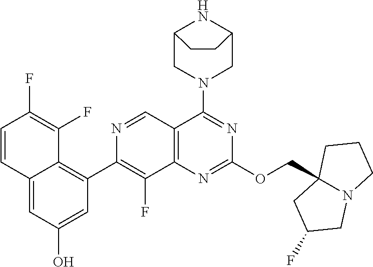 [SMILES: Oc1cc(-c2ncc3c(N4CC5CCC(C4)N5)nc(OC[C@@]45CCCN4C[C@H](F)C5)nc3c2F)c2c(F)c(F)ccc2c1]

(also referred to as Example 246 in WO 2021/041671) or a pharmaceutically acceptable salt thereof.

In one embodiment, the KRas G12D inhibitor is:

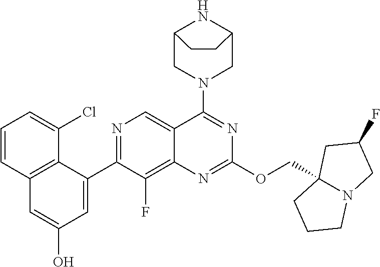 [SMILES: Oc1cc(-c2ncc3c(N4CC5CCC(C4)N5)nc(OC[C@@]45CCCN4C[C@H](F)C5)nc3c2F)c2c(Cl)cccc2c1]

(also referred to as Example 251 in WO 2021/041671) or a pharmaceutically acceptable salt thereof.

In one embodiment, the KRas G12D inhibitor is:

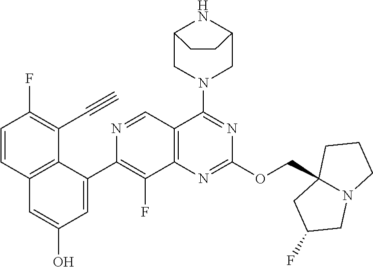 [SMILES: C#Cc1c(F)ccc2cc(O)cc(-c3ncc4c(N5CC6CCC(C5)N6)nc(OC[C@@]56CCCN5C[C@H](F)C6)nc4c3F)c12]

(also referred to as Example 252 in WO 2021/041671) or a pharmaceutically acceptable salt thereof. This compound is also known as MRTX1133 and may be referred to as “MRTX1133” in this application.

In one embodiment, the KRas G12D inhibitor is:

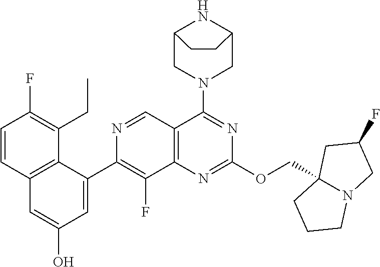 [SMILES: CCc1c(F)ccc2cc(O)cc(-c3ncc4c(N5CC6CCC(C5)N6)nc(OC[C@@]56CCCN5C[C@H](F)C6)nc4c3F)c12]

(also referred to as Example 253 in WO 2021/041671) or a pharmaceutically acceptable salt thereof.

In one embodiment, the KRas G12D inhibitor is:

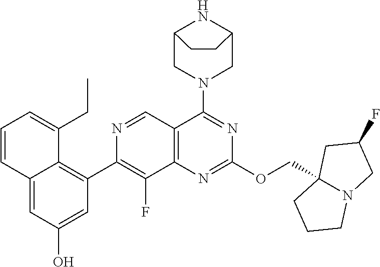 [SMILES: CCc1cccc2cc(O)cc(-c3ncc4c(N5CC6CCC(C5)N6)nc(OC[C@@]56CCCN5C[C@H](F)C6)nc4c3F)c12]

(also referred to as Example 259 in WO 2021/041671) or a pharmaceutically acceptable salt thereof.

In one embodiment, the KRas G12D inhibitor is:

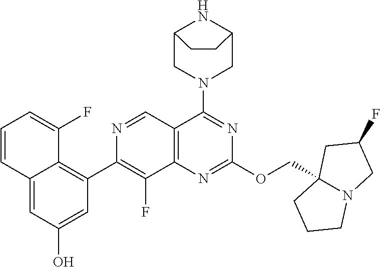 [SMILES: Oc1cc(-c2ncc3c(N4CC5CCC(C4)N5)nc(OC[C@@]45CCCN4C[C@H](F)C5)nc3c2F)c2c(F)cccc2c1]

(also referred to as Example 282 in WO 2021/041671) or a pharmaceutically acceptable salt thereof.

The KRas G12D inhibitors used in the methods of the present invention may have one or more chiral center and may be synthesized as stereoisomeric mixtures, isomers of identical constitution that differ in the arrangement of their atoms in space. The compounds may be used as mixtures or the individual components/isomers may be separated using commercially available reagents and conventional methods for isolation of stereoisomers and enantiomers well-known to those skilled in the art, e.g., using CHIRALPAK® (Sigma-Aldrich) or CHIRALCEL® (Diacel Corp) chiral chromatographic HPLC columns according to the manufacturer's instructions. Alternatively, compounds of the present invention may be synthesized using optically pure, chiral reagents and intermediates to prepare individual isomers or enantiomers. Unless otherwise indicated, all chiral (enantiomeric and diastereomeric) and racemic forms are within the scope of the invention. Unless otherwise indicated, whenever the specification, including the claims, refers to compounds of the invention, the term “compound” is to be understood to encompass all chiral (enantiomeric and diastereomeric) and racemic forms.

In one embodiment, the KRas G12D inhibitor compounds of Formula I used in the methods include trifluoroacetic acid salts of the above compounds.

Methods for manufacturing the KRas G12D inhibitors disclosed herein are known. For example, commonly owned published international PCT application number WO2021/041671 describes general reaction schemes for preparing compounds of Formula I and also provides detailed synthetic routes for the preparation of each KRas G12D inhibitor disclosed herein.

The pan ErbB family inhibitors and the KRas G12D compounds of Formula (I) or pharmaceutically acceptable salts thereof may be formulated into pharmaceutical compositions.

### Pharmaceutical Compositions

In another aspect, the invention provides pharmaceutical compositions comprising a pan ErbB family inhibitor, or a pharmaceutically acceptable salt thereof, and KRas G12D inhibitor, or a pharmaceutically acceptable salt thereof according to the invention and a pharmaceutically acceptable carrier, excipient, or diluent that may be used in the methods disclosed herein. The pan ErbB family inhibitor, or a pharmaceutically acceptable salt thereof, and KRas G12D inhibitor, or a pharmaceutically acceptable salt thereof may be independently formulated by any method well known in the art and may be prepared for administration by any route, including, without limitation, parenteral, oral, sublingual, transdermal, topical, intranasal, intratracheal, or intrarectal. In certain embodiments, a pan ErbB family inhibitor, or a pharmaceutically acceptable salt thereof, and KRas G12D inhibitor, or a pharmaceutically acceptable salt thereof, are administered intravenously in a hospital setting. In one embodiment, administration may be by the oral route.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions may contain, in addition to the inhibitor, diluents, fillers, salts, buffers, stabilizers, solubilizers, and other materials well known in the art. The preparation of pharmaceutically acceptable formulations is described in, e.g., Remington's Pharmaceutical Sciences, 18th Edition, ed. A. Gennaro, Mack Publishing Co., Easton, Pa., 1990.

As used herein, the term pharmaceutically acceptable salt refers to salts that retain the desired biological activity of the above-identified compounds and exhibit minimal or no undesired toxicological effects. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

The active compound is included in the pharmaceutically acceptable carrier or diluent in an amount sufficient to deliver to a patient a therapeutically effective amount without causing serious toxic effects in the patient treated. In one embodiment, a dose of the active compound for all of the above-mentioned conditions is in the range from about 0.01 to 300 mg/kg, for example 0.1 to 100 mg/kg per day, and as a further example 0.5 to about 25 mg per kilogram body weight of the recipient per day. A typical topical dosage will range from 0.01-3% wt/wt in a suitable carrier. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art.

The pharmaceutical compositions comprising a pan ErbB family inhibitor, or a pharmaceutically acceptable salt or a pharmaceutical composition thereof and a KRas G12D inhibitor, or a pharmaceutically acceptable salt or a pharmaceutical composition thereof, may be used in the methods of use described herein.

### Co-Administration

The pan ErbB family inhibitor, or a pharmaceutically acceptable salt thereof, and the KRas G12D inhibitor, or a pharmaceutically acceptable salt thereof, can be formulated into separate or individual dosage forms which can be co-administered one after the other. Another option is that if the route of administration is the same (e.g. oral) two active compounds can be formulated into a single form for co-administration, both methods of co-administration, however, being part of the same therapeutic treatment or regimen.

The pharmaceutical compositions comprising a pan ErbB family inhibitor, or a pharmaceutically acceptable salt thereof, and/or a KRas G12D inhibitor, or a pharmaceutically acceptable salt thereof, for use in the methods may be for simultaneous, separate or sequential use. In one embodiment, the pan ErbB family inhibitor, or a pharmaceutically acceptable salt thereof, is administered prior to administration of the KRas G12D inhibitor compound of Formula (I), or a pharmaceutically acceptable salt thereof. In another embodiment, the pan ErbB family inhibitor, or a pharmaceutically acceptable salt thereof, is administered after administration of the KRas G12D inhibitor compound of Formula (I), or a pharmaceutically acceptable salt thereof. In another embodiment, the pan ErbB family inhibitor, or a pharmaceutically acceptable salt thereof, is administered at about the same time as administration of the KRas G12D inhibitor compound of Formula (I), or a pharmaceutically acceptable salt thereof.

Separate administration of each inhibitor, at different times and by different routes, in some cases would be advantageous. Thus, the components in the combination i.e. the KRas G12D inhibitor compound of Formula (I), or a pharmaceutically acceptable salt thereof and the pan ErbB family inhibitor, or a pharmaceutically acceptable salt thereof, need not be necessarily administered at essentially the same time or in any order.

Oncology drugs are typically administered at the maximum tolerated dose (“MTD”), which is the highest dose of drug that does not cause unacceptable side effects. In one embodiment, the KRas G12D inhibitor, or a pharmaceutically acceptable salt or a pharmaceutical composition thereof and the pan ErbB family inhibitor, or a pharmaceutically acceptable salt or a pharmaceutical composition thereof, are each dosed at their respective MTDs. In one embodiment, the KRas G12D inhibitor, or a pharmaceutically acceptable salt or a pharmaceutical composition thereof, is dosed at its MTD and the pan ErbB family inhibitor, or a pharmaceutically acceptable salt or a pharmaceutical composition thereof, is dosed in an amount less than its MTD. In one embodiment, the KRas G12D inhibitor, or a pharmaceutically acceptable salt or a pharmaceutical composition thereof, is dosed at an amount less than its MTD and the pan ErbB family inhibitor, or a pharmaceutically acceptable salt or a pharmaceutical composition thereof, is dosed at its MTD. In one embodiment, the KRas G12D inhibitor, or a pharmaceutically acceptable salt or a pharmaceutical composition thereof and the pan ErbB family inhibitor, or a pharmaceutically acceptable salt or a pharmaceutical composition thereof are each dosed at less than their respective MTDs. The administration can be so timed that the peak pharmacokinetic effect of one compound coincides with the peak pharmacokinetic effect of the other.

In one embodiment, a single dose of KRas G12D inhibitor compound of Formula (I), or a pharmaceutically acceptable salt or a pharmaceutical composition thereof, is administered per day (i.e., in about 24 hour intervals) (i.e., QD). In another embodiment, two doses of the KRas G12D inhibitor compound of Formula (I), or a pharmaceutically acceptable salt or a pharmaceutical composition thereof, are administered per day (i.e., BID). In another embodiment, three doses of the KRas G12D inhibitor compound of Formula (I), or a pharmaceutically acceptable salt or a pharmaceutical composition thereof, are administered per day (i.e., TID).

In one embodiment, the pan ErbB family inhibitor, or a pharmaceutically acceptable salt or a pharmaceutical composition thereof, is administered QD. In another embodiment the pan ErbB family inhibitor, or a pharmaceutically acceptable salt or a pharmaceutical composition thereof, are administered BID. In another embodiment, the pan ErbB family inhibitor, or a pharmaceutically acceptable salt or a pharmaceutical composition thereof, of the invention are administered TID.

In one embodiment, a single dose of KRas G12D inhibitor compound of Formula (I), or a pharmaceutically acceptable salt or a pharmaceutical composition thereof, and pan ErbB family inhibitor, or a pharmaceutically acceptable salt or a pharmaceutical composition thereof are each administered once daily.

In one embodiment, the pan ErbB family inhibitor is an irreversible inhibitor. Exemplary irreversible pan ErbB family inhibitors for use in the methods herein include afatinib ((E)-N-(4-((3-chloro-4-fluorophenyl)amino)-7-((tetrahydrofuran-3-yl)oxy)quinazolin-6-yl)-4-(dimethylamino)but-2-enamide); dacomitinib ((2E)-N-{4-[(3-Chloro-4-fluorophenyl)amino]-7-methoxy-6-quinazolinyl}-4-(1-piperidinyl)-2-butenamide); canertinib (N-(4-((3-chloro-4-fluorophenyl)amino)-7-(3-morpholinopropoxy)quinazolin-6-yl)acrylamide); poziotinib (1-(4-((4-((3,4-dichloro-2-fluorophenyl)amino)-7-methoxyquinazolin-6-yl)oxy)piperidin-1-yl)prop-2-en-1-one); AV 412 (N-[4-[(3-Chloro-4-fluorophenyl)amino]-7-[3-methyl-3-(4-methyl-1-piperazinyl)-1-butyn-1-yl]-6-quinazolinyl]-2-propenamide); PF 6274484 (N-[4-[(3-Chloro-4-fluorophenyl)amino]-7-methoxy-6-quinazolinyl]-2-propenamide) and HKI 357 ((2E)-N-[[4-[[(3-Chloro-4-[(3-fluorophenyl)methoxy]phenyl]amino]-3-cyano-7-ethoxy-6-quinolinyl]-4-(dimethylamino)-2-butenamide), or pharmaceutically acceptable salts or pharmaceutically compositions thereof.

In one embodiment, the pan ErbB family inhibitor is a reversible inhibitor. Exemplary reversible pan EGFR family inhibitors include erlotinib ([6,7-Bis-(2-methoxy-ethoxy)-quinazolin-4-yl]-(3-ethynyl-phenyl)-amine)), gefitinib ((4-(3′-chloro-4′-fluoroanilino)-7-methoxy-6-(3-morpholinopropoxy)quinazoline), sapitinib (2-(4-((4-((3-chloro-2-fluorophenyl)amino)-7-methoxyquinazolin-6-yl)oxy)piperidin-1-yl)-N-methylacetamide); varlitinib ((R)—N4-(3-chloro-4-(thiazol-2-ylmethoxy)phenyl)-N6-(4-methyl-4,5-dihydrooxazol-2-yl)quinazoline-4,6-diamine); TAK-285 (N-(2-(4-((3-chloro-4-(3-(trifluoromethyl)phenoxy)phenyl)amino)-5H-pyrrolo[3,2-d]pyrimidin-5-yl)ethyl)-3-hydroxy-3-methylbutanamide); AEE788 ((S)-6-(4-((4-ethylpiperazin-1-yl)methyl)phenyl)-N-(1-phenylethyl)-7H-pyrrolo[2,3-d]pyrimidin-4-amine); tarloxotinib ([(E)-4-[[4-(3-bromo-4-chloroanilino)pyrido[3,4-d]pyrimidin-6-yl]amino]-4-oxobut-2-enyl]-dimethyl-[(3-methyl-5-nitroimidazol-4-yl)methyl]azanium); BMS 599626 ((3S)-3-Morpholinylmethyl-[4-[[1-[(3-fluorophenyl)methyl]-1H-indazol-5-yl]amino]-5-methylpyrrolo[2,1-f][1,2,4]triazin-6-yl]-carbamate dihydrochloride); and GW 583340 HCl (N-[3-Chloro-4-[(3-fluorophenyl)methoxy]phenyl]-6-[2-[[[2-(methylsulfonyl)ethyl]amino]methyl]-4-thiazolyl]-4-quinazolinamine dihydrochloride), or pharmaceutically acceptable salts or pharmaceutically compositions thereof.

In one embodiment, the pan ErbB family inhibitor is an anti-EGFR antibody, an anti-HER2 antibody or a combination of an anti-EGFR antibody and anti-HER2 antibody, or pharmaceutical compositions thereof. In one embodiment, the anti-EGFR antibody is necitumumab, panitumumab or cetuximab. In one embodiment, the anti-EGFR antibody is cetuximab. In one embodiment, the anti-HER2 antibodies suitable for use in the methods herein is pertuzumab, trastuzumab, or trastuzumab emtansine.

In one embodiment, the pan ErbB family inhibitor is a an EGFR inhibitor and a HER2 inhibitor, wherein the EGFR inhibitor and the HER2 inhibitor are independently selected from two agents selected from the group consisting of: AG 1478 HCl (N-(3-Chlorophenyl)-6,7-dimethoxy-4-quinazolinanine hydrochloride); AG 494 (E)-2-Cyano-3-(3,4-dihydroxyphenyl)-N-phenyl-2-propenamide; AG 555 (E)-2-Cyano-3-(3,4-dihydroxyphenyl)-N-(3-phenylpropyl)-2-propenamide; AG 556 (E)-2-Cyano-3-(3,4-dihydroxyphenyl)-N-(4-phenylbutyl)-2-propenamide; AG 825 (E)-3-[3-[2-Benzothiazolythio)methyl]-4-hydroxy-5-methoxyphenyl]-2-cyano-2-propenamide; CP 724714 (2-Methoxy-N-[(2E)-3-[4-[[3-methyl-4-[(6-methyl-3-pyridinyl)oxy]phenyl]amino]-6-quinazolinyl]-2-propen-1-yl]acetamide; BIBU 1361 diHCl (N-(3-Chloro-4-fluorophenyl)-6-[4-[(diethylamino)methyl]-1-piperidinyl]-pyrimido[5,4-d]pyrimidin-4-amine dihydrochloride); BIBU 1382 (N⁸-(3-Chloro-4-fluorophenyl)-N²-(1-methyl-4-piperidinyl)-pyrimido[5,4-d]pyrimidine-2,8-diamine dihydrochloride); JNJ 28871063 HCl (5E-4-Amino-6-(4-benzyloxy-3-chlorophenylamino)pyrimidine-5-carboxaldehyde N-(2-morpholin-4-ylethyl) oxime hydrochloride); PD 153035 (4-[(3-Bromophenyl)amino]-6,7-dimethoxyquinazoline hydrochloride); PD 158780 (N⁴-(3-Bromophenyl)-N⁶-methyl-pyrido[3,4-d]pyrimidine-4,6-diamine) or pharmaceutically acceptable salts or pharmaceutically compositions thereof.

### Combination Therapies

In one aspect of the invention, provided herein are methods of treating cancer in a subject in need thereof, comprising administering to the subject a therapeutically effective amount of a combination of a pan ErbB family inhibitor, or a pharmaceutically acceptable salt or a pharmaceutical composition thereof, and a KRAS G12D inhibitor of Formula (I), or a pharmaceutically acceptable salt or a pharmaceutical composition thereof. In one embodiment, the cancer is a KRas G12D-associated cancer. In one embodiment, the KRas G12D-associated cancer is pancreatic, colorectal, endometrial, and non-small cell lung cancer.

In yet another aspect, the invention provides for methods for increasing the sensitivity of a cancer cell to a KRas G12D inhibitor, comprising contacting the cancer cell with an effective amount of a combination of a KRas G12D inhibitor compound of Formula (I), or a pharmaceutically acceptable salt or a pharmaceutical composition thereof, and a pan ErbB family inhibitor, or a pharmaceutically acceptable salt or a pharmaceutical composition thereof, wherein the pan ErbB family inhibitor synergistically increases the sensitivity of the cancer cell to the KRas G12D inhibitor. In one embodiment, the contacting is in vitro. In one embodiment, the contacting is in vivo.

In one embodiment, the combination therapy comprises a combination of a compound having the formula:

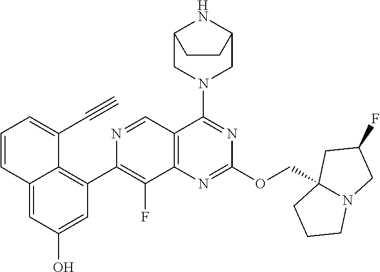 [SMILES: C#Cc1cccc2cc(O)cc(-c3ncc4c(N5CC6CCC(C5)N6)nc(OC[C@@]56CCCN5C[C@H](F)C6)nc4c3F)c12]

or a pharmaceutically acceptable salt thereof, and a pan ErbB family inhibitor. In one embodiment, the pan ErbB family inhibitor is afatinib. In one embodiment, the pan ErbB family inhibitor is dacomitinib. In one embodiment, the pan ErbB family inhibitor is poziotinib. In one embodiment, the pan ErbB family inhibitor is erlotinib. In one embodiment, the pan ErbB family inhibitor is Gefitinib. In one embodiment, the pan ErbB family inhibitor is sapitinib. In one embodiment, the pan ErbB family inhibitor is tarloxotinib. In one embodiment, the pan ErbB family inhibitor is an anti-EGFR antibody, wherein the anti-EGFR antibody is cetuximab.

In one embodiment, the combination therapy comprises a combination of a compound having the formula:

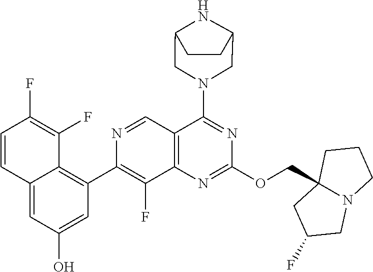 [SMILES: Oc1cc(-c2ncc3c(N4CC5CCC(C4)N5)nc(OC[C@@]45CCCN4C[C@H](F)C5)nc3c2F)c2c(F)c(F)ccc2c1]

or a pharmaceutically acceptable salt thereof, and a pan ErbB family inhibitor. In one embodiment, the pan ErbB family inhibitor is afatinib. In one embodiment, the pan ErbB family inhibitor is dacomitinib. In one embodiment, the pan ErbB family inhibitor is poziotinib. In one embodiment, the pan ErbB family inhibitor is erlotinib. In one embodiment, the pan ErbB family inhibitor is Gefitinib. In one embodiment, the pan ErbB family inhibitor is sapitinib. In one embodiment, the pan ErbB family inhibitor is tarloxotinib. In one embodiment, the pan ErbB family inhibitor is an anti-EGFR antibody, wherein the anti-EGFR antibody is cetuximab.

In one embodiment, the combination therapy comprises a combination of a compound having the formula:

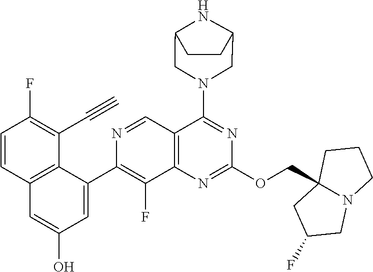 [SMILES: C#Cc1c(F)ccc2cc(O)cc(-c3ncc4c(N5CC6CCC(C5)N6)nc(OC[C@@]56CCCN5C[C@H](F)C6)nc4c3F)c12]

or a pharmaceutically acceptable salt thereof, and a pan ErbB family inhibitor. In one embodiment, the pan ErbB family inhibitor is afatinib. In one embodiment, the pan ErbB family inhibitor is dacomitinib. In one embodiment, the pan ErbB family inhibitor is poziotinib. In one embodiment, the pan ErbB family inhibitor is erlotinib. In one embodiment, the pan ErbB family inhibitor is Gefitinib. In one embodiment, the pan ErbB family inhibitor is sapitinib. In one embodiment, the pan ErbB family inhibitor is tarloxotinib. In one embodiment, the pan ErbB family inhibitor is an anti-EGFR antibody, wherein the anti-EGFR antibody is cetuximab.

In one embodiment, the combination therapy comprises a combination of a compound having the formula:

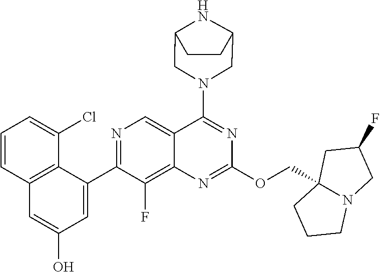 [SMILES: Oc1cc(-c2ncc3c(N4CC5CCC(C4)N5)nc(OC[C@@]45CCCN4C[C@H](F)C5)nc3c2F)c2c(Cl)cccc2c1]

or a pharmaceutically acceptable salt thereof, and a pan ErbB family inhibitor. In one embodiment, the pan ErbB family inhibitor is afatinib. In one embodiment, the pan ErbB family inhibitor is dacomitinib. In one embodiment, the pan ErbB family inhibitor is poziotinib. In one embodiment, the pan ErbB family inhibitor is erlotinib. In one embodiment, the pan ErbB family inhibitor is Gefitinib. In one embodiment, the pan ErbB family inhibitor is sapitinib. In one embodiment, the pan ErbB family inhibitor is tarloxotinib. In one embodiment, the pan ErbB family inhibitor is an anti-EGFR antibody, wherein the anti-EGFR antibody is cetuximab.

In one embodiment, the combination therapy comprises a combination of a compound having the formula:

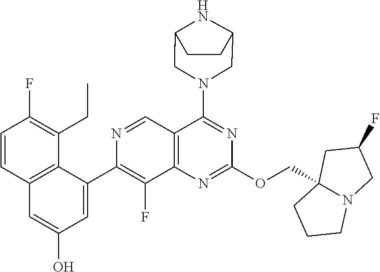 [SMILES: CCc1c(F)ccc2cc(O)cc(-c3ncc4c(N5CC6CCC(C5)N6)nc(OC[C@@]56CCCN5C[C@H](F)C6)nc4c3F)c12]

or a pharmaceutically acceptable salt thereof, and a pan ErbB family inhibitor. In one embodiment, the pan ErbB family inhibitor is afatinib. In one embodiment, the pan ErbB family inhibitor is dacomitinib. In one embodiment, the pan ErbB family inhibitor is poziotinib. In one embodiment, the pan ErbB family inhibitor is erlotinib. In one embodiment, the pan ErbB family inhibitor is Gefitinib. In one embodiment, the pan ErbB family inhibitor is sapitinib. In one embodiment, the pan ErbB family inhibitor is tarloxotinib. In one embodiment, the pan ErbB family inhibitor is an anti-EGFR antibody, wherein the anti-EGFR antibody is cetuximab.

In one embodiment, the combination therapy comprises a combination of a compound having the formula:

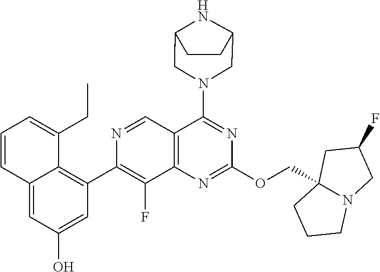 [SMILES: CCc1cccc2cc(O)cc(-c3ncc4c(N5CC6CCC(C5)N6)nc(OC[C@@]56CCCN5C[C@H](F)C6)nc4c3F)c12]

or a pharmaceutically acceptable salt thereof, and a pan ErbB family inhibitor. In one embodiment, the pan ErbB family inhibitor is afatinib. In one embodiment, the pan ErbB family inhibitor is dacomitinib. In one embodiment, the pan ErbB family inhibitor is poziotinib. In one embodiment, the pan ErbB family inhibitor is erlotinib. In one embodiment, the pan ErbB family inhibitor is Gefitinib. In one embodiment, the pan ErbB family inhibitor is sapitinib. In one embodiment, the pan ErbB family inhibitor is tarloxotinib. In one embodiment, the pan ErbB family inhibitor is an anti-EGFR antibody, wherein the anti-EGFR antibody is cetuximab.

In one embodiment, the combination therapy comprises a combination of a compound having the formula:

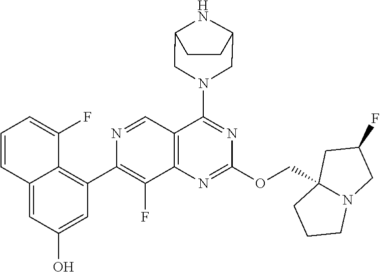 [SMILES: Oc1cc(-c2ncc3c(N4CC5CCC(C4)N5)nc(OC[C@@]45CCCN4C[C@H](F)C5)nc3c2F)c2c(F)cccc2c1]

or a pharmaceutically acceptable salt thereof, and a pan ErbB family inhibitor. In one embodiment, the pan ErbB family inhibitor is afatinib. In one embodiment, the pan ErbB family inhibitor is dacomitinib. In one embodiment, the pan ErbB family inhibitor is poziotinib. In one embodiment, the pan ErbB family inhibitor is erlotinib. In one embodiment, the pan ErbB family inhibitor is Gefitinib. In one embodiment, the pan ErbB family inhibitor is sapitinib. In one embodiment, the pan ErbB family inhibitor is tarloxotinib. In one embodiment, the pan ErbB family inhibitor is an anti-EGFR antibody, wherein the anti-EGFR antibody is cetuximab.

As used herein, the term “contacting” refers to the bringing together of indicated moieties in an in vitro system or an in vivo system. For example, “contacting” a cancer cell includes the administration of a combination provided herein to an individual or subject, such as a human, having KRas G12D, as well as, for example, introducing a combination provided herein into a sample containing a cellular or purified preparation containing KRas G12D.

By negatively modulating the activity of KRas G12D, the methods described herein are designed to inhibit undesired cellular proliferation resulting from enhanced KRas G12D activity within the cell. The ability of a compound to inhibit KRas G12D may be monitored in vitro using well known methods, including those described in published international PCT application number WO 2021/041671. Likewise, the inhibitory activity of combination in cells may be monitored, for example, by measuring the inhibition of KRas G12D activity of the amount of phosphorylated ERK to assess the effectiveness of treatment and dosages may be adjusted accordingly by the attending medical practitioner.

The compositions and methods provided herein may be used for the treatment of a KRas G12D-associated cancer in a subject in need thereof, comprising administering to said subject a therapeutically effective amount of a combination of a pan ErbB family inhibitor, or a pharmaceutically acceptable salt or a pharmaceutical composition thereof, and a KRas G12D inhibitor compound of Formula (I), or a pharmaceutically acceptable salt or a pharmaceutical composition thereof, wherein the pan ErbB family inhibitor synergistically increases the sensitivity of the KRas G12D-associated cancer to the KRas G12D inhibitor. In one embodiment, the KRas G12D-associated cancer is pancreatic, colorectal, endometrial, and non-small cell lung cancer.

In one embodiment, the therapeutically effective amount of the combination of a pan ErbB family inhibitor, or a pharmaceutically acceptable salt or a pharmaceutical composition thereof, and a KRas G12D inhibitor compound of Formula (I), or a pharmaceutically acceptable salt or a pharmaceutical composition thereof, results in an increased duration of overall survival (“OS”) in subjects relative to treatment with only the KRas G12D inhibitor. In one embodiment, the therapeutically effective amount of the combination of a pan ErbB family inhibitor, or a pharmaceutically acceptable salt or a pharmaceutical composition thereof, and a KRas G12D inhibitor compound of Formula (I), or a pharmaceutically acceptable salt or a pharmaceutical composition thereof, results in an increased duration of progression-free survival (“PFS”) in subjects relative to treatment with only the KRas G12D inhibitor. In one embodiment, the therapeutically effective amount of the combination of a pan ErbB family inhibitor, or a pharmaceutically acceptable salt or a pharmaceutical composition thereof, and a KRas G12D inhibitor compound of Formula (I), or a pharmaceutically acceptable salt or a pharmaceutical composition thereof, results in increased tumor regression in subjects relative to treatment with only the KRas G12D inhibitor. In one embodiment, the therapeutically effective amount of the combination of a pan ErbB family inhibitor, or a pharmaceutically acceptable salt or a pharmaceutical composition thereof, and a KRas G12D inhibitor compound of Formula (I), or a pharmaceutically acceptable salt or a pharmaceutical composition thereof, results in increased tumor growth inhibition in subjects relative to treatment with only the KRas G12D inhibitor. In one embodiment, the therapeutically effective amount of the combination of a pan ErbB family inhibitor, or a pharmaceutically acceptable salt or a pharmaceutical composition thereof, and a KRas G12D inhibitor compound of Formula (I), or a pharmaceutically acceptable salt or a pharmaceutical composition thereof, results in an improvement in the duration of stable disease in subjects compared to treatment with only the KRas G12D inhibitor. In one embodiment, the KRas G12D inhibitor is a compound selected from compound Nos. 1-458 (as numbered in WO2021/041671), or a pharmaceutically acceptable salt thereof (e.g., Example Nos. 252, 243, 246, 251, 253, 259 or 282 or a pharmaceutically acceptable salt thereof). In one embodiment, the pan ErbB family inhibitor is selected from afatinib, dacomitinib, poziotinib, erlotinib, gefitinib, sapitinib, tarloxotinib, and cetuximab. In one embodiment, the therapeutic combination comprises therapeutically effective amounts of Example No. 252 and afatinib. In one embodiment, the therapeutic combination comprises therapeutically effective amounts of Example No. 252 and dacomitinib. In one embodiment, the therapeutic combination comprises therapeutically effective amounts of Example No. 252 and poziotinib. In one embodiment, the therapeutic combination comprises therapeutically effective amounts of Example No. 252 and erlotinib. In one embodiment, the therapeutic combination comprises therapeutically effective amounts of Example No. 252 and gefitinib. In one embodiment, the therapeutic combination comprises therapeutically effective amounts of Example No. 252 and sapitinib. In one embodiment, the therapeutic combination comprises therapeutically effective amounts of Example No. 252 and tarloxotinib. In one embodiment, the therapeutic combination comprises therapeutically effective amounts of Example No. 252 and cetuximab. In one embodiment, the therapeutic combination comprises therapeutically effective amounts of Example No. 243 and afatinib. In one embodiment, the therapeutic combination comprises therapeutically effective amounts of Example No. 243 and dacomitinib. In one embodiment, the therapeutic combination comprises therapeutically effective amounts of Example No. 243 and poziotinib. In one embodiment, the therapeutic combination comprises therapeutically effective amounts of Example No. 243 and erlotinib. In one embodiment, the therapeutic combination comprises therapeutically effective amounts of Example No. 243 and gefitinib. In one embodiment, the therapeutic combination comprises therapeutically effective amounts of Example No. 243 and sapitinib. In one embodiment, the therapeutic combination comprises therapeutically effective amounts of Example No. 243 and tarloxotinib. In one embodiment, the therapeutic combination comprises therapeutically effective amounts of Example No. 243 and cetuximab. In one embodiment, the therapeutic combination comprises therapeutically effective amounts of Example No. 246 and afatinib. In one embodiment, the therapeutic combination comprises therapeutically effective amounts of Example No. 246 and dacomitinib. In one embodiment, the therapeutic combination comprises therapeutically effective amounts of Example No. 246 and poziotinib. In one embodiment, the therapeutic combination comprises therapeutically effective amounts of Example No. 246 and erlotinib. In one embodiment, the therapeutic combination comprises therapeutically effective amounts of Example No. 246 and gefitinib. In one embodiment, the therapeutic combination comprises therapeutically effective amounts of Example No. 246 and sapitinib. In one embodiment, the therapeutic combination comprises therapeutically effective amounts of Example No. 246 and tarloxotinib. In one embodiment, the therapeutic combination comprises therapeutically effective amounts of Example No. 246 and cetuximab. In one embodiment, the therapeutic combination comprises therapeutically effective amounts of Example No. 251 and afatinib. In one embodiment, the therapeutic combination comprises therapeutically effective amounts of Example No. 251 and dacomitinib. In one embodiment, the therapeutic combination comprises therapeutically effective amounts of Example No. 251 and poziotinib. In one embodiment, the therapeutic combination comprises therapeutically effective amounts of Example No. 251 and erlotinib. In one embodiment, the therapeutic combination comprises therapeutically effective amounts of Example No. 251 and gefitinib. In one embodiment, the therapeutic combination comprises therapeutically effective amounts of Example No. 251 and sapitinib. In one embodiment, the therapeutic combination comprises therapeutically effective amounts of Example No. 251 and tarloxotinib. In one embodiment, the therapeutic combination comprises therapeutically effective amounts of Example No. 251 and cetuximab. In one embodiment, the therapeutic combination comprises therapeutically effective amounts of Example No. 253 and afatinib. In one embodiment, the therapeutic combination comprises therapeutically effective amounts of Example No. 253 and dacomitinib. In one embodiment, the therapeutic combination comprises therapeutically effective amounts of Example No. 253 and poziotinib. In one embodiment, the therapeutic combination comprises therapeutically effective amounts of Example No. 253 and erlotinib. In one embodiment, the therapeutic combination comprises therapeutically effective amounts of Example No. 253 and gefitinib. In one embodiment, the therapeutic combination comprises therapeutically effective amounts of Example No. 253 and sapitinib. In one embodiment, the therapeutic combination comprises therapeutically effective amounts of Example No. 253 and tarloxotinib. In one embodiment, the therapeutic combination comprises therapeutically effective amounts of Example No. 253 and cetuximab. In one embodiment, the therapeutic combination comprises therapeutically effective amounts of Example No. 259 and afatinib. In one embodiment, the therapeutic combination comprises therapeutically effective amounts of Example No. 259 and dacomitinib. In one embodiment, the therapeutic combination comprises therapeutically effective amounts of Example No. 259 and poziotinib. In one embodiment, the therapeutic combination comprises therapeutically effective amounts of Example No. 259 and erlotinib. In one embodiment, the therapeutic combination comprises therapeutically effective amounts of Example No. 259 and gefitinib. In one embodiment, the therapeutic combination comprises therapeutically effective amounts of Example No. 259 and sapitinib. In one embodiment, the therapeutic combination comprises therapeutically effective amounts of Example No. 259 and tarloxotinib. In one embodiment, the therapeutic combination comprises therapeutically effective amounts of Example No. 259 and cetuximab. In one embodiment, the therapeutic combination comprises therapeutically effective amounts of Example No. 282 and afatinib. In one embodiment, the therapeutic combination comprises therapeutically effective amounts of Example No. 282 and dacomitinib. In one embodiment, the therapeutic combination comprises therapeutically effective amounts of Example No. 282 and poziotinib. In one embodiment, the therapeutic combination comprises therapeutically effective amounts of Example No. 282 and erlotinib. In one embodiment, the therapeutic combination comprises therapeutically effective amounts of Example No. 282 and gefitinib. In one embodiment, the therapeutic combination comprises therapeutically effective amounts of Example No. 282 and sapitinib. In one embodiment, the therapeutic combination comprises therapeutically effective amounts of Example No. 282 and tarloxotinib. In one embodiment, the therapeutic combination comprises therapeutically effective amounts of Example No. 282 and cetuximab.

In another embodiment, the pan ErbB family inhibitor, or a pharmaceutically acceptable salt or a pharmaceutical composition thereof, is administered in combination with the KRas G12D inhibitor, or a pharmaceutically acceptable salt or a pharmaceutical composition thereof, once disease progression has been observed for KRas G12D monotherapy, in which the combination therapy results in enhanced clinical benefit or time of survival for the patient by increasing OS, PFS, tumor regression, tumor growth inhibition or the duration of stable disease in the patient. In one embodiment, the KRas G12D inhibitor is a compound selected from compound Nos. 1-458 (as numbered in WO2021/041671), or a pharmaceutically acceptable salt thereof (e.g., Example Nos. 252, 243, 246, 251, 253, 259 or 282 or a pharmaceutically acceptable salt thereof). In one embodiment, the pan ErbB family inhibitor is selected from afatinib, dacomitinib, poziotinib, erlotinib, gefitinib, sapitinib, tarloxotinib, and cetuximab. In one embodiment, the therapeutic combination comprises therapeutically effective amounts of Example No. 252 and afatinib. In one embodiment, the therapeutic combination comprises therapeutically effective amounts of Example No. 252 and dacomitinib. In one embodiment, the therapeutic combination comprises therapeutically effective amounts of Example No. 252 and poziotinib. In one embodiment, the therapeutic combination comprises therapeutically effective amounts of Example No. 252 and erlotinib. In one embodiment, the therapeutic combination comprises therapeutically effective amounts of Example No. 252 and gefitinib. In one embodiment, the therapeutic combination comprises therapeutically effective amounts of Example No. 252 and sapitinib. In one embodiment, the therapeutic combination comprises therapeutically effective amounts of Example No. 252 and tarloxotinib. In one embodiment, the therapeutic combination comprises therapeutically effective amounts of Example No. 252 and cetuximab. In one embodiment, the therapeutic combination comprises therapeutically effective amounts of Example No. 243 and afatinib. In one embodiment, the therapeutic combination comprises therapeutically effective amounts of Example No. 243 and dacomitinib. In one embodiment, the therapeutic combination comprises therapeutically effective amounts of Example No. 243 and poziotinib. In one embodiment, the therapeutic combination comprises therapeutically effective amounts of Example No. 243 and erlotinib. In one embodiment, the therapeutic combination comprises therapeutically effective amounts of Example No. 243 and gefitinib. In one embodiment, the therapeutic combination comprises therapeutically effective amounts of Example No. 243 and sapitinib. In one embodiment, the therapeutic combination comprises therapeutically effective amounts of Example No. 243 and tarloxotinib. In one embodiment, the therapeutic combination comprises therapeutically effective amounts of Example No. 243 and cetuximab. In one embodiment, the therapeutic combination comprises therapeutically effective amounts of Example No. 246 and afatinib. In one embodiment, the therapeutic combination comprises therapeutically effective amounts of Example No. 246 and dacomitinib. In one embodiment, the therapeutic combination comprises therapeutically effective amounts of Example No. 246 and poziotinib. In one embodiment, the therapeutic combination comprises therapeutically effective amounts of Example No. 246 and erlotinib. In one embodiment, the therapeutic combination comprises therapeutically effective amounts of Example No. 246 and gefitinib. In one embodiment, the therapeutic combination comprises therapeutically effective amounts of Example No. 246 and sapitinib. In one embodiment, the therapeutic combination comprises therapeutically effective amounts of Example No. 246 and tarloxotinib. In one embodiment, the therapeutic combination comprises therapeutically effective amounts of Example No. 246 and cetuximab. In one embodiment, the therapeutic combination comprises therapeutically effective amounts of Example No. 251 and afatinib. In one embodiment, the therapeutic combination comprises therapeutically effective amounts of Example No. 251 and dacomitinib. In one embodiment, the therapeutic combination comprises therapeutically effective amounts of Example No. 251 and poziotinib. In one embodiment, the therapeutic combination comprises therapeutically effective amounts of Example No. 251 and erlotinib. In one embodiment, the therapeutic combination comprises therapeutically effective amounts of Example No. 251 and gefitinib. In one embodiment, the therapeutic combination comprises therapeutically effective amounts of Example No. 251 and sapitinib. In one embodiment, the therapeutic combination comprises therapeutically effective amounts of Example No. 251 and tarloxotinib. In one embodiment, the therapeutic combination comprises therapeutically effective amounts of Example No. 251 and cetuximab. In one embodiment, the therapeutic combination comprises therapeutically effective amounts of Example No. 253 and afatinib. In one embodiment, the therapeutic combination comprises therapeutically effective amounts of Example No. 253 and dacomitinib. In one embodiment, the therapeutic combination comprises therapeutically effective amounts of Example No. 253 and poziotinib. In one embodiment, the therapeutic combination comprises therapeutically effective amounts of Example No. 253 and erlotinib. In one embodiment, the therapeutic combination comprises therapeutically effective amounts of Example No. 253 and gefitinib. In one embodiment, the therapeutic combination comprises therapeutically effective amounts of Example No. 253 and sapitinib. In one embodiment, the therapeutic combination comprises therapeutically effective amounts of Example No. 253 and tarloxotinib. In one embodiment, the therapeutic combination comprises therapeutically effective amounts of Example No. 253 and cetuximab. In one embodiment, the therapeutic combination comprises therapeutically effective amounts of Example No. 259 and afatinib. In one embodiment, the therapeutic combination comprises therapeutically effective amounts of Example No. 259 and dacomitinib. In one embodiment, the therapeutic combination comprises therapeutically effective amounts of Example No. 259 and poziotinib. In one embodiment, the therapeutic combination comprises therapeutically effective amounts of Example No. 259 and erlotinib. In one embodiment, the therapeutic combination comprises therapeutically effective amounts of Example No. 259 and gefitinib. In one embodiment, the therapeutic combination comprises therapeutically effective amounts of Example No. 259 and sapitinib. In one embodiment, the therapeutic combination comprises therapeutically effective amounts of Example No. 259 and tarloxotinib. In one embodiment, the therapeutic combination comprises therapeutically effective amounts of Example No. 259 and cetuximab. In one embodiment, the therapeutic combination comprises therapeutically effective amounts of Example No. 282 and afatinib. In one embodiment, the therapeutic combination comprises therapeutically effective amounts of Example No. 282 and dacomitinib. In one embodiment, the therapeutic combination comprises therapeutically effective amounts of Example No. 282 and poziotinib. In one embodiment, the therapeutic combination comprises therapeutically effective amounts of Example No. 282 and erlotinib. In one embodiment, the therapeutic combination comprises therapeutically effective amounts of Example No. 282 and gefitinib. In one embodiment, the therapeutic combination comprises therapeutically effective amounts of Example No. 282 and sapitinib. In one embodiment, the therapeutic combination comprises therapeutically effective amounts of Example No. 282 and tarloxotinib. In one embodiment, the therapeutic combination comprises therapeutically effective amounts of Example No. 282 and cetuximab. In one embodiment of any of said combination therapies, the combination is useful for treating a KRas G12D-associated cancer. In one embodiment, the KRas G12D-associated cancer is pancreatic, colorectal, endometrial, and non-small cell lung cancer.

In one embodiment of any of the methods herein, the pan ErbB family inhibitor and the KRAS G12D inhibitor are administered on the same day.

In one embodiment of any of the methods herein, the pan ErbB family inhibitor and the KRAS G12D inhibitor are administered on different days.

The compositions and methods provided herein may be used for the treatment of a wide variety of cancers including tumors such as lung, colorectal, pancreas, prostate, breast, brain, skin, cervical carcinomas, testicular carcinomas, etc. More particularly, cancers that may be treated by the compositions and methods of the invention include, but are not limited to, tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, these compounds can be used to treat: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma. In certain embodiments, the cancer is non-small cell lung cancer.

Also provided herein is a method for treating cancer in a subject in need thereof, the method comprising (a) determining that cancer is associated with a KRas G12D mutation (e.g., a KRas G12D-associated cancer) (e.g., as determined using a regulatory agency-approved, e.g., FDA-approved, assay or kit); and (b) administering to the patient a therapeutically effective amount of a combination of a pan ErbB family inhibitor, or a pharmaceutically acceptable salt or a pharmaceutical composition thereof, and a KRas G12D inhibitor compound of Formula I, or a pharmaceutically acceptable salt or a pharmaceutical composition thereof, wherein the pan ErbB family inhibitor synergistically increases the sensitivity of the KRas G12D-associated cancer to the KRas G12D inhibitor. In one embodiment, the KRas G12D inhibitor is a compound selected from compound Nos. 1-458 (as numbered in WO2021/041671), or a pharmaceutically acceptable salt thereof (e.g., Example Nos. 252, 243, 246, 251, 253, 259 or 282 or a pharmaceutically acceptable salt thereof). In one embodiment, the pan ErbB family inhibitor is selected from afatinib, dacomitinib, poziotinib, erlotinib, gefitinib, sapitinib, tarloxotinib, and cetuximab. In one embodiment, the therapeutic combination comprises therapeutically effective amounts of Example No. 252 and afatinib. In one embodiment, the therapeutic combination comprises therapeutically effective amounts of Example No. 252 and dacomitinib. In one embodiment, the therapeutic combination comprises therapeutically effective amounts of Example No. 252 and poziotinib. In one embodiment, the therapeutic combination comprises therapeutically effective amounts of Example No. 252 and erlotinib. In one embodiment, the therapeutic combination comprises therapeutically effective amounts of Example No. 252 and gefitinib. In one embodiment, the therapeutic combination comprises therapeutically effective amounts of Example No. 252 and sapitinib. In one embodiment, the therapeutic combination comprises therapeutically effective amounts of Example No. 252 and tarloxotinib. In one embodiment, the therapeutic combination comprises therapeutically effective amounts of Example No. 252 and cetuximab. In one embodiment, the therapeutic combination comprises therapeutically effective amounts of Example No. 243 and afatinib. In one embodiment, the therapeutic combination comprises therapeutically effective amounts of Example No. 243 and dacomitinib. In one embodiment, the therapeutic combination comprises therapeutically effective amounts of Example No. 243 and poziotinib. In one embodiment, the therapeutic combination comprises therapeutically effective amounts of Example No. 243 and erlotinib. In one embodiment, the therapeutic combination comprises therapeutically effective amounts of Example No. 243 and gefitinib. In one embodiment, the therapeutic combination comprises therapeutically effective amounts of Example No. 243 and sapitinib. In one embodiment, the therapeutic combination comprises therapeutically effective amounts of Example No. 243 and tarloxotinib. In one embodiment, the therapeutic combination comprises therapeutically effective amounts of Example No. 243 and cetuximab. In one embodiment, the therapeutic combination comprises therapeutically effective amounts of Example No. 246 and afatinib. In one embodiment, the therapeutic combination comprises therapeutically effective amounts of Example No. 246 and dacomitinib. In one embodiment, the therapeutic combination comprises therapeutically effective amounts of Example No. 246 and poziotinib. In one embodiment, the therapeutic combination comprises therapeutically effective amounts of Example No. 246 and erlotinib. In one embodiment, the therapeutic combination comprises therapeutically effective amounts of Example No. 246 and gefitinib. In one embodiment, the therapeutic combination comprises therapeutically effective amounts of Example No. 246 and sapitinib. In one embodiment, the therapeutic combination comprises therapeutically effective amounts of Example No. 246 and tarloxotinib. In one embodiment, the therapeutic combination comprises therapeutically effective amounts of Example No. 246 and cetuximab. In one embodiment, the therapeutic combination comprises therapeutically effective amounts of Example No. 251 and afatinib. In one embodiment, the therapeutic combination comprises therapeutically effective amounts of Example No. 251 and dacomitinib. In one embodiment, the therapeutic combination comprises therapeutically effective amounts of Example No. 251 and poziotinib. In one embodiment, the therapeutic combination comprises therapeutically effective amounts of Example No. 251 and erlotinib. In one embodiment, the therapeutic combination comprises therapeutically effective amounts of Example No. 251 and gefitinib. In one embodiment, the therapeutic combination comprises therapeutically effective amounts of Example No. 251 and sapitinib. In one embodiment, the therapeutic combination comprises therapeutically effective amounts of Example No. 251 and tarloxotinib. In one embodiment, the therapeutic combination comprises therapeutically effective amounts of Example No. 251 and cetuximab. In one embodiment, the therapeutic combination comprises therapeutically effective amounts of Example No. 253 and afatinib. In one embodiment, the therapeutic combination comprises therapeutically effective amounts of Example No. 253 and dacomitinib. In one embodiment, the therapeutic combination comprises therapeutically effective amounts of Example No. 253 and poziotinib. In one embodiment, the therapeutic combination comprises therapeutically effective amounts of Example No. 253 and erlotinib. In one embodiment, the therapeutic combination comprises therapeutically effective amounts of Example No. 253 and gefitinib. In one embodiment, the therapeutic combination comprises therapeutically effective amounts of Example No. 253 and sapitinib. In one embodiment, the therapeutic combination comprises therapeutically effective amounts of Example No. 253 and tarloxotinib. In one embodiment, the therapeutic combination comprises therapeutically effective amounts of Example No. 253 and cetuximab. In one embodiment, the therapeutic combination comprises therapeutically effective amounts of Example No. 259 and afatinib. In one embodiment, the therapeutic combination comprises therapeutically effective amounts of Example No. 259 and dacomitinib. In one embodiment, the therapeutic combination comprises therapeutically effective amounts of Example No. 259 and poziotinib. In one embodiment, the therapeutic combination comprises therapeutically effective amounts of Example No. 259 and erlotinib. In one embodiment, the therapeutic combination comprises therapeutically effective amounts of Example No. 259 and gefitinib. In one embodiment, the therapeutic combination comprises therapeutically effective amounts of Example No. 259 and sapitinib. In one embodiment, the therapeutic combination comprises therapeutically effective amounts of Example No. 259 and tarloxotinib. In one embodiment, the therapeutic combination comprises therapeutically effective amounts of Example No. 259 and cetuximab. In one embodiment, the therapeutic combination comprises therapeutically effective amounts of Example No. 282 and afatinib. In one embodiment, the therapeutic combination comprises therapeutically effective amounts of Example No. 282 and dacomitinib. In one embodiment, the therapeutic combination comprises therapeutically effective amounts of Example No. 282 and poziotinib. In one embodiment, the therapeutic combination comprises therapeutically effective amounts of Example No. 282 and erlotinib. In one embodiment, the therapeutic combination comprises therapeutically effective amounts of Example No. 282 and gefitinib. In one embodiment, the therapeutic combination comprises therapeutically effective amounts of Example No. 282 and sapitinib. In one embodiment, the therapeutic combination comprises therapeutically effective amounts of Example No. 282 and tarloxotinib. In one embodiment, the therapeutic combination comprises therapeutically effective amounts of Example No. 282 and cetuximab.

In one embodiment, a compound of Formula I is administered as a capsule during the period of time. In one embodiment, a tablet or capsule formulation of a compound of Formula I comprises about 10 mg to about 100 mg (e.g., about 10 mg to about 95 mg, about mg to about 90 mg, about 10 mg to about 85 mg, about 10 mg to about 80 mg, about 10 mg to about 75 mg, about 10 mg to about 70 mg, about 10 mg to about 65 mg, about 10 mg to about 60 mg, about 10 mg to about 55 mg, about 10 mg to about 50 mg, about 10 mg to about 45 mg, about 10 mg to about 40 mg, about 10 mg to about 35 mg, about 10 mg to about 30 mg, about 10 mg to about 25 mg, about 10 mg to about 20 mg, about 10 mg to about 15 mg, about mg to about 100 mg, about 15 mg to about 95 mg, about 15 mg to about 90 mg, about 15 mg to about 85 mg, about 15 mg to about 80 mg, about 15 mg to about 75 mg, about 15 mg to about 70 mg, about 15 mg to about 65 mg, about 15 mg to about 60 mg, about 15 mg to about 55 mg, about 15 mg to about 50 mg, about 15 mg to about 45 mg, about 15 mg to about 40 mg, about 15 mg to about 35 mg, about 15 mg to about 30 mg, about 15 mg to about 25 mg, about 15 mg to about 20 mg, about 20 mg to about 100 mg, about 20 mg to about 95 mg, about 20 mg to about 90 mg, about 20 mg to about 85 mg, about 20 mg to about 80 mg, about 20 mg to about 75 mg, about 20 mg to about 70 mg, about 20 mg to about 65 mg, about 20 mg to about 60 mg, about 20 mg to about 55 mg, about 20 mg to about 50 mg, about 20 mg to about 45 mg, about 20 mg to about 40 mg, about 20 mg to about 35 mg, about 20 mg to about 30 mg, about 20 mg to about 25 mg, about 25 mg to about 100 mg, about 25 mg to about 95 mg, about 25 mg to about 90 mg, about 25 mg to about 85 mg, about 25 mg to about 80 mg, about 25 mg to about 75 mg, about 25 mg to about 70 mg, about 25 mg to about 65 mg, about 25 mg to about 60 mg, about 25 mg to about 55 mg, about 25 mg to about 50 mg, about 25 mg to about 45 mg, about 25 mg to about 40 mg, about 25 mg to about 35 mg, about 25 mg to about 30 mg, about mg to about 100 mg, about 30 mg to about 95 mg, about 30 mg to about 90 mg, about 30 mg to about 85 mg, about 30 mg to about 80 mg, about 30 mg to about 75 mg, about 30 mg to about 70 mg, about 30 mg to about 65 mg, about 30 mg to about 60 mg, about 30 mg to about 55 mg, about 30 mg to about 50 mg, about 30 mg to about 45 mg, about 30 mg to about 40 mg, about 30 mg to about 35 mg, about 35 mg to about 100 mg, about 35 mg to about 95 mg, about mg to about 90 mg, about 35 mg to about 85 mg, about 35 mg to about 80 mg, about 35 mg to about 75 mg, about 35 mg to about 70 mg, about 35 mg to about 65 mg, about 35 mg to about 60 mg, about 35 mg to about 55 mg, about 35 mg to about 50 mg, about 35 mg to about 45 mg, about 35 mg to about 40 mg, about 40 mg to about 100 mg, about 40 mg to about 95 mg, about 40 mg to about 90 mg, about 40 mg to about 85 mg, about 40 mg to about 80 mg, about 40 mg to about 75 mg, about 40 mg to about 70 mg, about 40 mg to about 65 mg, about 40 mg to about 60 mg, about 40 mg to about 55 mg, about 40 mg to about 50 mg, about 40 mg to about 45 mg, about 45 mg to about 100 mg, about 45 mg to about 95 mg, about 45 mg to about 90 mg, about 45 mg to about 85 mg, about 45 mg to about 80 mg, about 45 mg to about 75 mg, about 45 mg to about 70 mg, about 45 mg to about 65 mg, about 45 mg to about 60 mg, about 45 mg to about 55 mg, about 45 mg to about 50 mg, about 50 mg to about 100 mg, about 50 mg to about 95 mg, about 50 mg to about 90 mg, about 50 mg to about 85 mg, about 50 mg to about 80 mg, about 50 mg to about 75 mg, about 50 mg to about 70 mg, about 50 mg to about 65 mg, about 50 mg to about 60 mg, about 50 mg to about 55 mg, about 55 mg to about 100 mg, about 55 mg to about 95 mg, about 55 mg to about 90 mg, about 55 mg to about 85 mg, about 55 mg to about 80 mg, about 55 mg to about 75 mg, about 55 mg to about 70 mg, about 55 mg to about 65 mg, about 55 mg to about 60 mg, about 60 mg to about 100 mg, about 60 mg to about 95 mg, about 60 mg to about 90 mg, about 60 mg to about 85 mg, about 60 mg to about 80 mg, about 60 mg to about 75 mg, about 60 mg to about 70 mg, about 60 mg to about 65 mg, about 65 mg to about 100 mg, about 65 mg to about 95 mg, about 65 mg to about 90 mg, about 65 mg to about 85 mg, about 65 mg to about 80 mg, about 65 mg to about 75 mg, about 65 mg to about 70 mg, about 70 mg to about 100 mg, about 70 mg to about 95 mg, about 70 mg to about 90 mg, about 70 mg to about 85 mg, about 70 mg to about 80 mg, about 70 mg to about 75 mg, about 75 mg to about 100 mg, about 75 mg to about 95 mg, about 75 mg to about 90 mg, about 75 mg to about 85 mg, about 75 mg to about 80 mg, about 80 mg to about 100 mg, about 80 mg to about 95 mg, about 80 mg to about 90 mg, about 80 mg to about 85 mg, about 85 mg to about 100 mg, about 85 mg to about 95 mg, about 85 mg to about 90 mg, about 90 mg to about 100 mg, about 90 mg to about 95 mg, about 95 mg to about 100 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg) of a compound of Formula I (e.g., a compound selected from compound Nos. 1-458 (as numbered in WO2021/041671), or a pharmaceutically acceptable salt thereof (e.g., Example Nos. 252, 243, 246, 251, 253, 259, and 282 or a pharmaceutically acceptable salt thereof). In one embodiment, a compound of Formula I is orally administered once a day (QD) on a daily basis during a period of time. In one embodiment, a compound of Formula I is orally administered twice a day (BID) on a daily basis during a period of time. In one embodiment, a compound of Formula I is orally administered in the amount of about 20 mg to about 500 mg (e.g., about 20 mg to about 480 mg, about 20 mg to about 460 mg, about 20 mg to about 440 mg, about 20 mg to about 420 mg, about 20 mg to about 400 mg, about 20 mg to about 380 mg, about 20 mg to about 360 mg, about 20 mg to about 340 mg, about 20 mg to about 320 mg, about 20 mg to about 300 mg, about 20 mg to about 280 mg, about 20 mg to about 260 mg, about 20 mg to about 240 mg, about 20 mg to about 220 mg, about 20 mg to about 200 mg, about 20 mg to about 180 mg, about 20 mg to about 160 mg, about 20 mg to about 140 mg, about 20 mg to about 120 mg, about 20 mg to about 100 mg, about 20 mg to about 80 mg, about 20 mg to about 60 mg, about 20 mg to about 40 mg, about 40 mg to about 500 mg, about 40 mg to about 480 mg, about 40 mg to about 460 mg, about 40 mg to about 440 mg, about 40 mg to about 420 mg, about 40 mg to about 400 mg, about 40 mg to about 380 mg, about 40 mg to about 360 mg, about 40 mg to about 340 mg, about 40 mg to about 320 mg, about 40 mg to about 300 mg, about 40 mg to about 280 mg, about 40 mg to about 260 mg, about 40 mg to about 240 mg, about 40 mg to about 220 mg, about 40 mg to about 200 mg, about 40 mg to about 180 mg, about 40 mg to about 160 mg, about 40 mg to about 140 mg, about 40 mg to about 120 mg, about 40 mg to about 100 mg, about 40 mg to about 80 mg, about 40 mg to about 60 mg, about 60 mg to about 500 mg, about 60 mg to about 480 mg, about 60 mg to about 460 mg, about 60 mg to about 440 mg, about 60 mg to about 420 mg, about 60 mg to about 400 mg, about 60 mg to about 380 mg, about 60 mg to about 360 mg, about 60 mg to about 340 mg, about 60 mg to about 320 mg, about 60 mg to about 300 mg, about 60 mg to about 280 mg, about 60 mg to about 260 mg, about 60 mg to about 240 mg, about 60 mg to about 220 mg, about 60 mg to about 200 mg, about 60 mg to about 180 mg, about 60 mg to about 160 mg, about 60 mg to about 140 mg, about 60 mg to about 120 mg, about 60 mg to about 100 mg, about 60 mg to about 80 mg, about 80 mg to about 500 mg, about 80 mg to about 480 mg, about 80 mg to about 460 mg, about 80 mg to about 440 mg, about 80 mg to about 420 mg, about 80 mg to about 400 mg, about 80 mg to about 380 mg, about 80 mg to about 360 mg, about 80 mg to about 340 mg, about 80 mg to about 320 mg, about 80 mg to about 300 mg, about 80 mg to about 280 mg, about 80 mg to about 260 mg, about 80 mg to about 240 mg, about 80 mg to about 220 mg, about 80 mg to about 200 mg, about 80 mg to about 180 mg, about 80 mg to about 160 mg, about 80 mg to about 140 mg, about 80 mg to about 120 mg, about 80 mg to about 100 mg, about 100 mg to about 500 mg, about 100 mg to about 480 mg, about 100 mg to about 460 mg, about 100 mg to about 440 mg, about 100 mg to about 420 mg, about 100 mg to about 400 mg, about 100 mg to about 380 mg, about 100 mg to about 360 mg, about 100 mg to about 340 mg, about 100 mg to about 320 mg, about 100 mg to about 300 mg, about 100 mg to about 280 mg, about 100 mg to about 260 mg, about 100 mg to about 240 mg, about 100 mg to about 220 mg, about 100 mg to about 200 mg, about 100 mg to about 180 mg, about 100 mg to about 160 mg, about 100 mg to about 140 mg, about 100 mg to about 120 mg, about 120 mg to about 500 mg, about 120 mg to about 480 mg, about 120 mg to about 460 mg, about 120 mg to about 440 mg, about 120 mg to about 420 mg, about 120 mg to about 400 mg, about 120 mg to about 380 mg, about 120 mg to about 360 mg, about 120 mg to about 340 mg, about 120 mg to about 320 mg, about 120 mg to about 300 mg, about 120 mg to about 280 mg, about 120 mg to about 260 mg, about 120 mg to about 240 mg, about 120 mg to about 220 mg, about 120 mg to about 200 mg, about 120 mg to about 180 mg, about 120 mg to about 160 mg, about 120 mg to about 140 mg, about 140 mg to about 500 mg, about 140 mg to about 480 mg, about 140 mg to about 460 mg, about 140 mg to about 440 mg, about 140 mg to about 420 mg, about 140 mg to about 400 mg, about 140 mg to about 380 mg, about 140 mg to about 360 mg, about 140 mg to about 340 mg, about 140 mg to about 320 mg, about 140 mg to about 300 mg, about 140 mg to about 280 mg, about 140 mg to about 260 mg, about 140 mg to about 240 mg, about 140 mg to about 220 mg, about 140 mg to about 200 mg, about 140 mg to about 180 mg, about 140 mg to about 160 mg, about 160 mg to about 500 mg, about 160 mg to about 480 mg, about 160 mg to about 460 mg, about 160 mg to about 440 mg, about 160 mg to about 420 mg, about 160 mg to about 400 mg, about 160 mg to about 380 mg, about 160 mg to about 360 mg, about 160 mg to about 340 mg, about 160 mg to about 320 mg, about 160 mg to about 300 mg, about 160 mg to about 280 mg, about 160 mg to about 260 mg, about 160 mg to about 240 mg, about 160 mg to about 220 mg, about 160 mg to about 200 mg, about 160 mg to about 180 mg, about 180 mg to about 500 mg, about 180 mg to about 480 mg, about 180 mg to about 460 mg, about 180 mg to about 440 mg, about 180 mg to about 420 mg, about 180 mg to about 400 mg, about 180 mg to about 380 mg, about 180 mg to about 360 mg, about 180 mg to about 340 mg, about 180 mg to about 320 mg, about 180 mg to about 300 mg, about 180 mg to about 280 mg, about 180 mg to about 260 mg, about 180 mg to about 240 mg, about 180 mg to about 220 mg, about 180 mg to about 200 mg, about 200 mg to about 500 mg, about 200 mg to about 480 mg, about 200 mg to about 460 mg, about 200 mg to about 440 mg, about 200 mg to about 420 mg, about 200 mg to about 400 mg, about 200 mg to about 380 mg, about 200 mg to about 360 mg, about 200 mg to about 340 mg, about 200 mg to about 320 mg, about 200 mg to about 300 mg, about 200 mg to about 280 mg, about 200 mg to about 260 mg, about 200 mg to about 240 mg, about 200 mg to about 220 mg, about 220 mg to about 500 mg, about 220 mg to about 480 mg, about 220 mg to about 460 mg, about 220 mg to about 440 mg, about 220 mg to about 420 mg, about 220 mg to about 400 mg, about 220 mg to about 380 mg, about 220 mg to about 360 mg, about 220 mg to about 340 mg, about 220 mg to about 320 mg, about 220 mg to about 300 mg, about 220 mg to about 280 mg, about 220 mg to about 260 mg, about 220 mg to about 240 mg, about 240 mg to about 500 mg, about 240 mg to about 480 mg, about 240 mg to about 460 mg, about 240 mg to about 440 mg, about 240 mg to about 420 mg, about 240 mg to about 400 mg, about 240 mg to about 380 mg, about 240 mg to about 360 mg, about 240 mg to about 340 mg, about 240 mg to about 320 mg, about 240 mg to about 300 mg, about 240 mg to about 280 mg, about 240 mg to about 260 mg, about 260 mg to about 500 mg, about 260 mg to about 480 mg, about 260 mg to about 460 mg, about 260 mg to about 440 mg, about 260 mg to about 420 mg, about 260 mg to about 400 mg, about 260 mg to about 380 mg, about 260 mg to about 360 mg, about 260 mg to about 340 mg, about 260 mg to about 320 mg, about 260 mg to about 300 mg, about 260 mg to about 280 mg, about 280 mg to about 500 mg, about 280 mg to about 480 mg, about 280 mg to about 460 mg, about 280 mg to about 440 mg, about 280 mg to about 420 mg, about 280 mg to about 400 mg, about 280 mg to about 380 mg, about 280 mg to about 360 mg, about 280 mg to about 340 mg, about 280 mg to about 320 mg, about 280 mg to about 300 mg, about 300 mg to about 500 mg, about 300 mg to about 480 mg, about 300 mg to about 460 mg, about 300 mg to about 440 mg, about 300 mg to about 420 mg, about 300 mg to about 400 mg, about 300 mg to about 380 mg, about 300 mg to about 360 mg, about 300 mg to about 340 mg, about 300 mg to about 320 mg, about 320 mg to about 500 mg, about 320 mg to about 480 mg, about 320 mg to about 460 mg, about 320 mg to about 440 mg, about 320 mg to about 420 mg, about 320 mg to about 400 mg, about 320 mg to about 380 mg, about 320 mg to about 360 mg, about 320 mg to about 340 mg, about 340 mg to about 500 mg, about 340 mg to about 480 mg, about 340 mg to about 460 mg, about 340 mg to about 440 mg, about 340 mg to about 420 mg, about 340 mg to about 400 mg, about 340 mg to about 380 mg, about 340 mg to about 360 mg, about 360 mg to about 500 mg, about 360 mg to about 480 mg, about 360 mg to about 460 mg, about 360 mg to about 440 mg, about 360 mg to about 420 mg, about 360 mg to about 400 mg, about 360 mg to about 380 mg, about 380 mg to about 500 mg, about 380 mg to about 480 mg, about 380 mg to about 460 mg, about 380 mg to about 440 mg, about 380 mg to about 420 mg, about 380 mg to about 400 mg, about 400 mg to about 500 mg, about 400 mg to about 480 mg, about 400 mg to about 460 mg, about 400 mg to about 440 mg, about 400 mg to about 420 mg, about 420 mg to about 500 mg, about 420 mg to about 480 mg, about 420 mg to about 460 mg, about 420 mg to about 440 mg, about 440 mg to about 500 mg, about 440 mg to about 480 mg, about 440 mg to about 460 mg, about 460 mg to about 500 mg, about 460 mg to about 480 mg, about 480 mg to about 500 mg, about 25, about 50, about 75, about 100, about 150, about 200, about 250, about 300, about 350, about 400, about 450, or about 500 mg), during a period of time.

In one embodiment, the combination therapy comprises oral administration of a compound of Formula I once or twice a day on a daily basis (during a period of time), e.g., in an amount of about 10 mg to about 400 mg (e.g., about 10 mg to about 380 mg, about 10 mg to about 360 mg, about 10 mg to about 340 mg, about 10 mg to about 320 mg, about 10 mg to about 300 mg, about 10 mg to about 280 mg, about 10 mg to about 260 mg, about 10 mg to about 240 mg, about 10 mg to about 220 mg, about 10 mg to about 200 mg, about 10 mg to about 180 mg, about 10 mg to about 160 mg, about 10 mg to about 140 mg, about 10 mg to about 120 mg, about 10 mg to about 100 mg, about 10 mg to about 80 mg, about 10 mg to about 60 mg, about 10 mg to about 40 mg, about 10 mg to about 20 mg, about 20 mg to about 400 mg, about 20 mg to about 380 mg, about 20 mg to about 360 mg, about 20 mg to about 340 mg, about 20 mg to about 320 mg, about 20 mg to about 300 mg, about 20 mg to about 280 mg, about 20 mg to about 260 mg, about 20 mg to about 240 mg, about 20 mg to about 220 mg, about 20 mg to about 200 mg, about 20 mg to about 180 mg, about 20 mg to about 160 mg, about 20 mg to about 140 mg, about 20 mg to about 120 mg, about 20 mg to about 100 mg, about 20 mg to about 80 mg, about 20 mg to about 60 mg, about 20 mg to about 40 mg, about 40 mg to about 400 mg, about 40 mg to about 380 mg, about 40 mg to about 360 mg, about 40 mg to about 340 mg, about 40 mg to about 320 mg, about 40 mg to about 300 mg, about 40 mg to about 280 mg, about 40 mg to about 260 mg, about 40 mg to about 240 mg, about 40 mg to about 220 mg, about 40 mg to about 200 mg, about 40 mg to about 180 mg, about 40 mg to about 160 mg, about 40 mg to about 140 mg, about 40 mg to about 120 mg, about 40 mg to about 100 mg, about 40 mg to about 80 mg, about 40 mg to about 60 mg, about 60 mg to about 400 mg, about 60 mg to about 380 mg, about 60 mg to about 360 mg, about 60 mg to about 340 mg, about 60 mg to about 320 mg, about 60 mg to about 300 mg, about 60 mg to about 280 mg, about 60 mg to about 260 mg, about 60 mg to about 240 mg, about 60 mg to about 220 mg, about 60 mg to about 200 mg, about 60 mg to about 180 mg, about 60 mg to about 160 mg, about 60 mg to about 140 mg, about 60 mg to about 120 mg, about 60 mg to about 100 mg, about 60 mg to about 80 mg, about 80 mg to about 400 mg, about 80 mg to about 380 mg, about 80 mg to about 360 mg, about 80 mg to about 340 mg, about 80 mg to about 320 mg, about 80 mg to about 300 mg, about 80 mg to about 280 mg, about 80 mg to about 260 mg, about 80 mg to about 240 mg, about 80 mg to about 220 mg, about 80 mg to about 200 mg, about 80 mg to about 180 mg, about 80 mg to about 160 mg, about 80 mg to about 140 mg, about 80 mg to about 120 mg, about 80 mg to about 100 mg, about 100 mg to about 400 mg, about 100 mg to about 380 mg, about 100 mg to about 360 mg, about 100 mg to about 340 mg, about 100 mg to about 320 mg, about 100 mg to about 300 mg, about 100 mg to about 280 mg, about 100 mg to about 260 mg, about 100 mg to about 240 mg, about 100 mg to about 220 mg, about 100 mg to about 200 mg, about 100 mg to about 180 mg, about 100 mg to about 160 mg, about 100 mg to about 140 mg, about 100 mg to about 120 mg, about 120 mg to about 400 mg, about 120 mg to about 380 mg, about 120 mg to about 360 mg, about 120 mg to about 340 mg, about 120 mg to about 320 mg, about 120 mg to about 300 mg, about 120 mg to about 280 mg, about 120 mg to about 260 mg, about 120 mg to about 240 mg, about 120 mg to about 220 mg, about 120 mg to about 200 mg, about 120 mg to about 180 mg, about 120 mg to about 160 mg, about 120 mg to about 140 mg, about 140 mg to about 400 mg, about 140 mg to about 380 mg, about 140 mg to about 360 mg, about 140 mg to about 340 mg, about 140 mg to about 320 mg, about 140 mg to about 300 mg, about 140 mg to about 280 mg, about 140 mg to about 260 mg, about 140 mg to about 240 mg, about 140 mg to about 220 mg, about 140 mg to about 200 mg, about 140 mg to about 180 mg, about 140 mg to about 160 mg, about 160 mg to about 400 mg, about 160 mg to about 380 mg, about 160 mg to about 360 mg, about 160 mg to about 360 mg, about 160 mg to about 340 mg, about 160 mg to about 320 mg, about 160 mg to about 300 mg, about 160 mg to about 280 mg, about 160 mg to about 260 mg, about 160 mg to about 240 mg, about 160 mg to about 220 mg, about 160 mg to about 200 mg, about 160 mg to about 180 mg, about 180 mg to about 400 mg, about 180 mg to about 380 mg, about 180 mg to about 360 mg, about 180 mg to about 340 mg, about 180 mg to about 320 mg, about 180 mg to about 300 mg, about 180 mg to about 280 mg, about 180 mg to about 260 mg, about 180 mg to about 240 mg, about 180 mg to about 220 mg, about 180 mg to about 200 mg, about 200 mg to about 400 mg, about 200 mg to about 380 mg, about 200 mg to about 360 mg, about 200 mg to about 340 mg, about 200 mg to about 320 mg, about 200 mg to about 300 mg, about 200 mg to about 280 mg, about 200 mg to about 260 mg, about 200 mg to about 240 mg, about 200 mg to about 220 mg, about 220 mg to about 400 mg, about 220 mg to about 380 mg, about 220 mg to about 360 mg, about 220 mg to about 340 mg, about 220 mg to about 320 mg, about 220 mg to about 300 mg, about 220 mg to about 280 mg, about 220 mg to about 260 mg, about 220 mg to about 240 mg, about 240 mg to about 400 mg, about 240 mg to about 380 mg, about 240 mg to about 360 mg, about 240 mg to about 340 mg, about 240 mg to about 320 mg, about 240 mg to about 300 mg, about 240 mg to about 280 mg, about 240 mg to about 260 mg, about 260 mg to about 400 mg, about 260 mg to about 380 mg, about 260 mg to about 360 mg, about 260 mg to about 340 mg, about 260 mg to about 320 mg, about 260 mg to about 300 mg, about 260 mg to about 280 mg, about 280 mg to about 400 mg, about 280 mg to about 380 mg, about 280 mg to about 360 mg, about 280 mg to about 340 mg, about 280 mg to about 320 mg, about 280 mg to about 300 mg, about 300 mg to about 400 mg, about 300 mg to about 380 mg, about 300 mg to about 360 mg, about 300 mg to about 340 mg, about 300 mg to about 320 mg, about 320 mg to about 400 mg, about 320 mg to about 380 mg, about 320 mg to about 360 mg, about 340 mg to about 360 mg, about 340 mg to about 400 mg, about 340 mg to about 380 mg, about 340 mg to about 360 mg, about 360 mg to about 400 mg, about 360 mg to about 380 mg, about 380 mg to about 400 mg, about 100 mg, about 200 mg, about 300 mg, or about 400 mg), and oral administration of a pan ErbB family inhibitor, or a pharmaceutically acceptable salt or a pharmaceutical composition thereof which is administered, for example once a day on a daily basis (during a period of time). In one embodiment, the KRAS G12D inhibitor, or a pharmaceutically acceptable salt or a pharmaceutical composition thereof, is orally administered once daily. In one embodiment, the KRAS G12D inhibitor, or a pharmaceutically acceptable salt or a pharmaceutical composition thereof, is orally administered twice daily.

One skilled in the art will recognize that, both in vivo and in vitro trials using suitable, known and generally accepted cell and/or animal models are predictive of the ability of a test compound of the combination or the combination to treat or prevent a given disorder.

One skilled in the art will further recognize that human clinical trials including first-in-human, dose ranging and efficacy trials, in healthy patients and/or those suffering from a given disorder, may be completed according to methods well known in the clinical and medical arts.

### Synergy

In one embodiment, the addition of a pan ErbB family inhibitor, or a pharmaceutically acceptable salt or a pharmaceutical composition thereof, synergistically increases the activity of KRas G12D inhibitor compound of Formula (I), or a pharmaceutically acceptable salt or a pharmaceutical composition thereof against cancer or cancer cell lines expressing KRas G12D. Any method for determining whether two compounds exhibit synergy may be used for determining the synergistic effect of the combination.

Several mathematical models have been developed to determine whether two compounds act synergistically, i.e., beyond a mere additive effect. For instance, Loewe Additivity (Loewe (1928) Physiol. 27: 47-187), Bliss Independence (Bliss (1939) Ann. Appl. Biol. 26: 585-615), Highest Single Agent, ZIP (Yadav et al (2015) Comput Struct Biotech J 13: 504-513) and other models (Chou & Talalay (1984) Adv Enzyme Regul 22: 27-55. #6382953; and Greco et al. (1995) Pharmacol Rev 47(2): 331-85. #7568331) are well known models in the pharmaceutical industry and may be used to calculate a “synergy score” that indicates whether synergy was detected and the magnitude of such synergy. Combining these synergy scores produces a composite synergy score which may be used to evaluate and characterize the KRas G12D inhibitor compounds of Formula (I) in combination with a pan ErbB family inhibitor.

In general, the mathematical models use data obtained from single agent values to determine the predicted additive effect of the combination which is compared to the observed effect for the combination. If the observed effect is greater than the predicted effect, the combination is deemed to be synergistic. For example, the Bliss independence model compares the observed combination response (Y_{O}) with the predicted combination response (Y_{P}), which was obtained based on the assumption that there is no effect from drug-drug interactions. Typically, the combination effect is declared synergistic if Y_{O} is greater than Y_{P}.

In some embodiments, “synergistic effect” as used herein refers to combination of a KRAS inhibitor or a pharmaceutically acceptable salt thereof, and a pan ErbB family inhibitor or a pharmaceutically acceptable salt thereof producing an effect, for example, any of the beneficial or desired results including clinical results or endpoints as described herein, which is greater than the sum of the effect observed when a compound of Formula I or a pharmaceutically acceptable salt thereof (e.g., a compound selected from compound Nos. 1-458 as numbered in WO2021/041671) and a pan ErbB family inhibitor or a pharmaceutically acceptable salt thereof are administered alone. In one embodiment, the KRas G12D inhibitor is a compound selected from compound Nos. 1-458 (as numbered in WO2021/041671), or a pharmaceutically acceptable salt thereof (e.g., Example Nos. 252, 243, 246, 251, 253, 259 or 282 or a pharmaceutically acceptable salt thereof). In one embodiment, the pan ErbB family inhibitor is selected from afatinib, dacomitinib, poziotinib, erlotinib, gefitinib, sapitinib, tarloxotinib, and cetuximab. In one embodiment, the therapeutic combination comprises therapeutically effective amounts of Example No. 252 and afatinib. In one embodiment, the therapeutic combination comprises therapeutically effective amounts of Example No. 252 and dacomitinib. In one embodiment, the therapeutic combination comprises therapeutically effective amounts of Example No. 252 and poziotinib. In one embodiment, the therapeutic combination comprises therapeutically effective amounts of Example No. 252 and erlotinib. In one embodiment, the therapeutic combination comprises therapeutically effective amounts of Example No. 252 and gefitinib. In one embodiment, the therapeutic combination comprises therapeutically effective amounts of Example No. 252 and sapitinib. In one embodiment, the therapeutic combination comprises therapeutically effective amounts of Example No. 252 and tarloxotinib. In one embodiment, the therapeutic combination comprises therapeutically effective amounts of Example No. 252 and cetuximab. In one embodiment, the therapeutic combination comprises therapeutically effective amounts of Example No. 243 and afatinib. In one embodiment, the therapeutic combination comprises therapeutically effective amounts of Example No. 243 and dacomitinib. In one embodiment, the therapeutic combination comprises therapeutically effective amounts of Example No. 243 and poziotinib. In one embodiment, the therapeutic combination comprises therapeutically effective amounts of Example No. 243 and erlotinib. In one embodiment, the therapeutic combination comprises therapeutically effective amounts of Example No. 243 and gefitinib. In one embodiment, the therapeutic combination comprises therapeutically effective amounts of Example No. 243 and sapitinib. In one embodiment, the therapeutic combination comprises therapeutically effective amounts of Example No. 243 and tarloxotinib. In one embodiment, the therapeutic combination comprises therapeutically effective amounts of Example No. 243 and cetuximab. In one embodiment, the therapeutic combination comprises therapeutically effective amounts of Example No. 246 and afatinib. In one embodiment, the therapeutic combination comprises therapeutically effective amounts of Example No. 246 and dacomitinib. In one embodiment, the therapeutic combination comprises therapeutically effective amounts of Example No. 246 and poziotinib. In one embodiment, the therapeutic combination comprises therapeutically effective amounts of Example No. 246 and erlotinib. In one embodiment, the therapeutic combination comprises therapeutically effective amounts of Example No. 246 and gefitinib. In one embodiment, the therapeutic combination comprises therapeutically effective amounts of Example No. 246 and sapitinib. In one embodiment, the therapeutic combination comprises therapeutically effective amounts of Example No. 246 and tarloxotinib. In one embodiment, the therapeutic combination comprises therapeutically effective amounts of Example No. 246 and cetuximab. In one embodiment, the therapeutic combination comprises therapeutically effective amounts of Example No. 251 and afatinib. In one embodiment, the therapeutic combination comprises therapeutically effective amounts of Example No. 251 and dacomitinib. In one embodiment, the therapeutic combination comprises therapeutically effective amounts of Example No. 251 and poziotinib. In one embodiment, the therapeutic combination comprises therapeutically effective amounts of Example No. 251 and erlotinib. In one embodiment, the therapeutic combination comprises therapeutically effective amounts of Example No. 251 and gefitinib. In one embodiment, the therapeutic combination comprises therapeutically effective amounts of Example No. 251 and sapitinib. In one embodiment, the therapeutic combination comprises therapeutically effective amounts of Example No. 251 and tarloxotinib. In one embodiment, the therapeutic combination comprises therapeutically effective amounts of Example No. 251 and cetuximab. In one embodiment, the therapeutic combination comprises therapeutically effective amounts of Example No. 253 and afatinib. In one embodiment, the therapeutic combination comprises therapeutically effective amounts of Example No. 253 and dacomitinib. In one embodiment, the therapeutic combination comprises therapeutically effective amounts of Example No. 253 and poziotinib. In one embodiment, the therapeutic combination comprises therapeutically effective amounts of Example No. 253 and erlotinib. In one embodiment, the therapeutic combination comprises therapeutically effective amounts of Example No. 253 and gefitinib. In one embodiment, the therapeutic combination comprises therapeutically effective amounts of Example No. 253 and sapitinib. In one embodiment, the therapeutic combination comprises therapeutically effective amounts of Example No. 253 and tarloxotinib. In one embodiment, the therapeutic combination comprises therapeutically effective amounts of Example No. 253 and cetuximab. In one embodiment, the therapeutic combination comprises therapeutically effective amounts of Example No. 259 and afatinib. In one embodiment, the therapeutic combination comprises therapeutically effective amounts of Example No. 259 and dacomitinib. In one embodiment, the therapeutic combination comprises therapeutically effective amounts of Example No. 259 and poziotinib. In one embodiment, the therapeutic combination comprises therapeutically effective amounts of Example No. 259 and erlotinib. In one embodiment, the therapeutic combination comprises therapeutically effective amounts of Example No. 259 and gefitinib. In one embodiment, the therapeutic combination comprises therapeutically effective amounts of Example No. 259 and sapitinib. In one embodiment, the therapeutic combination comprises therapeutically effective amounts of Example No. 259 and tarloxotinib. In one embodiment, the therapeutic combination comprises therapeutically effective amounts of Example No. 259 and cetuximab. In one embodiment, the therapeutic combination comprises therapeutically effective amounts of Example No. 282 and afatinib. In one embodiment, the therapeutic combination comprises therapeutically effective amounts of Example No. 282 and dacomitinib. In one embodiment, the therapeutic combination comprises therapeutically effective amounts of Example No. 282 and poziotinib. In one embodiment, the therapeutic combination comprises therapeutically effective amounts of Example No. 282 and erlotinib. In one embodiment, the therapeutic combination comprises therapeutically effective amounts of Example No. 282 and gefitinib. In one embodiment, the therapeutic combination comprises therapeutically effective amounts of Example No. 282 and sapitinib. In one embodiment, the therapeutic combination comprises therapeutically effective amounts of Example No. 282 and tarloxotinib. In one embodiment, the therapeutic combination comprises therapeutically effective amounts of Example No. 282 and cetuximab.

In some embodiments, the methods provided herein can result in a 1% to 99% (e.g., 1% to 98%, 1% to 95%, 1% to 90%, 1 to 85%, 1 to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 2% to 99%, 2% to 90%, 2% to 85%, 2% to 80%, 2% to 75%, 2% to 70%, 2% to 65%, 2% to 60%, 2% to 55%, 2% to 50%, 2% to 45%, 2% to 40%, 2% to 35%, 2% to 30%, 2% to 25%, 2% to 20%, 2% to 15%, 2% to 10%, 2% to 5%, 4% to 99%, 4% to 95%, 4% to 90%, 4% to 85%, 4% to 80%, 4% to 75%, 4% to 70%, 4% to 65%, 4% to 60%, 4% to 55%, 4% to 50%, 4% to 45%, 4% to 40%, 4% to 35%, 4% to 30%, 4% to 25%, 4% to 20%, 4% to 15%, 4% to 10%, 6% to 99%, 6% to 95%, 6% to 90%, 6% to 85%, 6% to 80%, 6% to 75%, 6% to 70%, 6% to 65%, 6% to 60%, 6% to 55%, 6% to 50%, 6% to 45%, 6% to 40%, 6% to 35%, 6% to 30%, 6% to 25%, 6% to 20%, 6% to 15%, 6% to 10%, 8% to 99%, 8% to 95%, 8% to 90%, 8% to 85%, 8% to 80%, 8% to 75%, 8% to 70%, 8% to 65%, 8% to 60%, 8% to 55%, 8% to 50%, 8% to 45%, 8% to 40%, 8% to 35%, 8% to 30%, 8% to 25%, 8% to 20%, 8% to 15%, 10% to 99%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, 10% to 40%, 10% to 35%, 10% to 30%, 10% to 25%, 10% to 20%, 10% to 15%, 15% to 99%, 15% to 95%, 15% to 90%, 15% to 85%, 15% to 80%, 15% to 75%, 15% to 70%, 15% to 65%, 15% to 60%, 15% to 55%, 15% to 50%, 15% to 55%, 15% to 50%, 15% to 45%, 15% to 40%, 15% to 35%, 15% to 30%, 15% to 25%, 15% to 20%, 20% to 99%, 20% to 95%, 20% to 90%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 25% to 99%, 25% to 95%, 25% to 90%, 25% to 85%, 25% to 80%, 25% to 75%, 25% to 70%, 25% to 65%, 25% to 60%, 25% to 55%, 25% to 50%, 25% to 45%, 25% to 40%, 25% to 35%, 25% to 30%, 30% to 99%, 30% to 95%, 30% to 90%, 30% to 85%, 30% to 80%, 30% to 75%, 30% to 70%, 30% to 65%, 30% to 60%, 30% to 55%, 30% to 50%, 30% to 45%, 30% to 40%, 30% to 35%, 35% to 99%, 35% to 95%, 35% to 90%, 35% to 85%, 35% to 80%, 35% to 75%, 35% to 70%, 35% to 65%, 35% to 60%, 35% to 55%, 35% to 50%, 35% to 45%, 35% to 40%, 40% to 99%, 40% to 95%, 40% to 90%, 40% to 85%, 40% to 80%, 40% to 75%, 40% to 70%, 40% to 65%, 40% to 60%, 40% to 55%, 40% to 60%, 40% to 55%, 40% to 50%, 40% to 45%, 45% to 99%, 45% to 95%, 45% to 95%, 45% to 90%, 45% to 85%, 45% to 80%, 45% to 75%, 45% to 70%, 45% to 65%, 45% to 60%, 45% to 55%, 45% to 50%, 50% to 99%, 50% to 95%, 50% to 90%, 50% to 85%, 50% to 80%, 50% to 75%, 50% to 70%, 50% to 65%, 50% to 60%, 50% to 55%, 55% to 99%, 55% to 95%, 55% to 90%, 55% to 85%, 55% to 80%, 55% to 75%, 55% to 70%, 55% to 65%, 55% to 60%, 60% to 99%, 60% to 95%, 60% to 90%, 60% to 85%, 60% to 80%, 60% to 75%, 60% to 70%, 60% to 65%, 65% to 99%, 60% to 95%, 60% to 90%, 60% to 85%, 60% to 80%, 60% to 75%, 60% to 70%, 60% to 65%, 70% to 99%, 70% to 95%, 70% to 90%, 70% to 85%, 70% to 80%, 70% to 75%, 75% to 99%, 75% to 95%, 75% to 90%, 75% to 85%, 75% to 80%, 80% to 99%, 80% to 95%, 80% to 90%, 80% to 85%, 85% to 99%, 85% to 95%, 85% to 90%, 90% to 99%, 90% to 95%, or 95% to 100%) reduction in the volume of one or more solid tumors in a patient following treatment with the combination therapy for a period of time between 1 day and 2 years (e.g., between 1 day and 22 months, between 1 day and 20 months, between 1 day and 18 months, between 1 day and 16 months, between 1 day and 14 months, between 1 day and 12 months, between 1 day and 10 months, between 1 day and 9 months, between 1 day and 8 months, between 1 day and 7 months, between 1 day and 6 months, between 1 day and 5 months, between 1 day and 4 months, between 1 day and 3 months, between 1 day and 2 months, between 1 day and 1 month, between one week and 2 years, between 1 week and 22 months, between 1 week and 20 months, between 1 week and 18 months, between 1 week and 16 months, between 1 week and 14 months, between 1 week and 12 months, between 1 week and 10 months, between 1 week and 9 months, between 1 week and 8 months, between 1 week and 7 months, between 1 week and 6 months, between 1 week and 5 months, between 1 week and 4 months, between 1 week and 3 months, between 1 week and 2 months, between 1 week and 1 month, between 2 weeks and 2 years, between 2 weeks and 22 months, between 2 weeks and 20 months, between 2 weeks and 18 months, between 2 weeks and 16 months, between 2 weeks and 14 months, between 2 weeks and 12 months, between 2 weeks and 10 months, between 2 weeks and 9 months, between 2 weeks and 8 months, between 2 weeks and 7 months, between 2 weeks and 6 months, between 2 weeks and 5 months, between 2 weeks and 4 months, between 2 weeks and 3 months, between 2 weeks and 2 months, between 2 weeks and 1 month, between 1 month and 2 years, between 1 month and 22 months, between 1 month and 20 months, between 1 month and 18 months, between 1 month and 16 months, between 1 month and 14 months, between 1 month and 12 months, between 1 month and 10 months, between 1 month and 9 months, between 1 month and 8 months, between 1 month and 7 months, between 1 month and 6 months, between 1 month and 6 months, between 1 month and 5 months, between 1 month and 4 months, between 1 month and 3 months, between 1 month and 2 months, between 2 months and 2 years, between 2 months and 22 months, between 2 months and 20 months, between 2 months and 18 months, between 2 months and 16 months, between 2 months and 14 months, between 2 months and 12 months, between 2 months and 10 months, between 2 months and 9 months, between 2 months and 8 months, between 2 months and 7 months, between 2 months and 6 months, or between 2 months and 5 months, between 2 months and 4 months, between 3 months and 2 years, between 3 months and 22 months, between 3 months and 20 months, between 3 months and 18 months, between 3 months and 16 months, between 3 months and 14 months, between 3 months and 12 months, between 3 months and 10 months, between 3 months and 8 months, between 3 months and 6 months, between 4 months and 2 years, between 4 months and 22 months, between 4 months and 20 months, between 4 months and 18 months, between 4 months and 16 months, between 4 months and 14 months, between 4 months and 12 months, between 4 months and 10 months, between 4 months and 8 months, between 4 months and 6 months, between 6 months and 2 years, between 6 months and 22 months, between 6 months and 20 months, between 6 months and 18 months, between 6 months and 16 months, between 6 months and 14 months, between 6 months and 12 months, between 6 months and 10 months, or between 6 months and 8 months) (e.g., as compared to the size of the one or more solid tumors in the patient prior to treatment).

The phrase “time of survival” means the length of time between the identification or diagnosis of cancer (e.g., any of the cancers described herein) in a mammal by a medical professional and the time of death of the mammal (caused by the cancer). Methods of increasing the time of survival in a mammal having a cancer are described herein.

In some embodiments, any of the methods described herein can result in an increase (e.g., a 1% to 400%, 1% to 380%, 1% to 360%, 1% to 340%, 1% to 320%, 1% to 300%, 1% to 280%, 1% to 260%, 1% to 240%, 1% to 220%, 1% to 200%, 1% to 180%, 1% to 160%, 1% to 140%, 1% to 120%, 1% to 100%, 1% to 95%, 1% to 90%, 1% to 85%, 1% to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 5% to 400%, 5% to 380%, 5% to 360%, 5% to 340%, 5% to 320%, 5% to 300%, 5% to 280%, 5% to 260%, 5% to 240%, 5% to 220%, 5% to 200%, 5% to 180%, 5% to 160%, 5% to 140%, 5% to 120%, 5% to 100%, 5% to 90%, 5% to 80%, 5% to 70%, 5% to 60%, 5% to 50%, 5% to 40%, 5% to 30%, 5% to 20%, 5% to 10%, 10% to 400%, 10% to 380%, 10% to 360%, 10% to 340%, 10% to 320%, 10% to 300%, 10% to 280%, 10% to 260%, 10% to 240%, 10% to 220%, 10% to 200%, 10% to 180%, 10% to 160%, 10% to 140%, 10% to 120%, 10% to 100%, 10% to 90%, 10% to 80%, 10% to 70%, 10% to 60%, 10% to 50%, 10% to 40%, 10% to 30%, 10% to 20%, 20% to 400%, 20% to 380%, 20% to 360%, 20% to 340%, 20% to 320%, 20% to 300%, 20% to 280%, 20% to 260%, 20% to 240%, 20% to 220%, 20% to 200%, 20% to 180%, 20% to 160%, 20% to 140%, 20% to 120%, 20% to 100%, 20% to 90%, 20% to 80%, 20% to 70%, 20% to 60%, 20% to 50%, 20% to 40%, 20% to 30%, 30% to 400%, 30% to 380%, 30% to 360%, 30% to 340%, 30% to 320%, 30% to 300%, 30% to 280%, 30% to 260%, 30% to 240%, 30% to 220%, 30% to 200%, 30% to 180%, 30% to 160%, 30% to 140%, 30% to 120%, 30% to 100%, 30% to 90%, 30% to 80%, 30% to 70%, 30% to 60%, 30% to 50%, 30% to 40%, 40% to 400%, 40% to 380%, 40% to 360%, 40% to 340%, 40% to 320%, 40% to 300%, 40% to 280%, 40% to 260%, 40% to 240%, 40% to 220%, 40% to 200%, 40% to 180%, 40% to 160%, 40% to 140%, 40% to 120%, 40% to 100%, 40% to 90%, 40% to 80%, 40% to 70%, 40% to 60%, 40% to 50%, 50% to 400%, 50% to 380%, 50% to 360%, 50% to 340%, 50% to 320%, 50% to 300%, 50% to 280%, 50% to 260%, 50% to 240%, 50% to 220%, 50% to 200%, 50% to 180%, 50% to 160%, 50% to 140%, 50% to 140%, 50% to 120%, 50% to 100%, 50% to 90%, 50% to 80%, 50% to 70%, 50% to 60%, 60% to 400%, 60% to 380%, 60% to 360%, 60% to 340%, 60% to 320%, 60% to 300%, 60% to 280%, 60% to 260%, 60% to 240%, 60% to 220%, 60% to 200%, 60% to 180%, 60% to 160%, 60% to 140%, 60% to 120%, 60% to 100%, 60% to 90%, 60% to 80%, 60% to 70%, 70% to 400%, 70% to 380%, 70% to 360%, 70% to 340%, 70% to 320%, 70% to 300%, 70% to 280%, 70% to 260%, 70% to 240%, 70% to 220%, 70% to 200%, 70% to 180%, 70% to 160%, 70% to 140%, 70% to 120%, to 100%, 70% to 90%, 70% to 80%, 80% to 400%, 80% to 380%, 80% to 360%, 80% to 340%, 80% to 320%, 80% to 300%, 80% to 280%, 80% to 260%, 80% to 240%, 80% to 220%, 80% to 200%, 80% to 180%, 80% to 160%, 80% to 140%, 80% to 120%, 80% to 100%, 80% to 90%, 90% to 400%, 90% to 380%, 90% to 360%, 90% to 340%, 90% to 320%, 90% to 300%, 90% to 280%, 90% to 260%, 90% to 240%, 90% to 220%, 90% to 200%, 90% to 180%, 90% to 160%, 90% to 140%, 90% to 120%, 90% to 100%, 100% to 400%, 100% to 380%, 100% to 360%, 100% to 340%, 100% to 320%, 100% to 300%, 100% to 280%, 100% to 260%, 100% to 240%, 100% to 220%, 100% to 200%, 100% to 180%, 100% to 160%, 100% to 140%, 100% to 120%, 120% to 400%, 120% to 380%, 120% to 360%, 120% to 340%, 120% to 320%, 120% to 300%, 120% to 280%, 120% to 260%, 120% to 240%, 120% to 220%, 120% to 200%, 120% to 180%, 120% to 160%, 120% to 140%, 140% to 400%, 140% to 380%, 140% to 360%, 140% to 340%, 140% to 320%, 140% to 300%, 140% to 280%, 140% to 260%, 140% to 240%, 140% to 220%, 140% to 200%, 140% to 180%, 140% to 160%, 160% to 400%, 160% to 380%, 160% to 360%, 160% to 340%, 160% to 320%, 160% to 300%, 160% to 280%, 160% to 260%, 160% to 240%, 160% to 220%, 160% to 200%, 160% to 180%, 180% to 400%, 180% to 380%, 180% to 360%, 180% to 340%, 180% to 320%, 180% to 300%, 180% to 280%, 180% to 260%, 180% to 240%, 180% to 220%, 180% to 200%, 200% to 400%, 200% to 380%, 200% to 360%, 200% to 340%, 200% to 320%, 200% to 300%, 200% to 280%, 200% to 260%, 200% to 240%, 200% to 220%, 220% to 400%, 220% to 380%, 220% to 360%, 220% to 340%, 220% to 320%, 220% to 300%, 220% to 280%, 220% to 260%, 220% to 240%, 240% to 400%, 240% to 380%, 240% to 360%, 240% to 340%, 240% to 320%, 240% to 300%, 240% to 280%, 240% to 260%, 260% to 400%, 260% to 380%, 260% to 360%, 260% to 340%, 260% to 320%, 260% to 300%, 260% to 280%, 280% to 400%, 280% to 380%, 280% to 360%, 280% to 340%, 280% to 320%, 280% to 300%, 300% to 400%, 300% to 380%, 300% to 360%, 300% to 340%, or 300% to 320%) in the time of survival of the patient (e.g., as compared to a patient having a similar cancer and administered a different treatment or not receiving a treatment).

In some embodiments of any of the methods described herein, before treatment with the compositions or methods of the invention, the patient was treated with one or more of a chemotherapy, a targeted anticancer agent, radiation therapy, and surgery, and optionally, the prior treatment was unsuccessful; and/or the patient has been administered surgery and optionally, the surgery was unsuccessful; and/or the patient has been treated with a platinum-based chemotherapeutic agent, and optionally, the patient has been previously determined to be non-responsive to treatment with the platinum-based chemotherapeutic agent; and/or the patient has been treated with a kinase inhibitor, and optionally, the prior treatment with the kinase inhibitor was unsuccessful; and/or the patient was treated with one or more other therapeutic agent(s).

### Kits

The present invention also relates to a kit comprising a pan ErbB family inhibitor, or a pharmaceutically acceptable salt or a pharmaceutical composition thereof, and a KRas G12D inhibitor compound of Formula (I), or a pharmaceutically acceptable salt or a pharmaceutical composition thereof. Also provided is a kit comprising a pan ErbB family inhibitor, or a pharmaceutically acceptable salt or a pharmaceutical composition thereof, and a KRas G12D inhibitor compound of Formula (I), or a pharmaceutically acceptable salt or a pharmaceutical composition thereof, for use in treating a hematological cancer.

In a related aspect, the invention provides a kit containing a dose of a pan ErbB family inhibitor, or a pharmaceutically acceptable salt or a pharmaceutical composition thereof, and dose of a KRas G12D inhibitor compound of Formula (I), or a pharmaceutically acceptable salt or a pharmaceutical composition thereof, in an amount effective to inhibit proliferation of cancer cells, particularly KRas G12D-expressing cancer cells, in a subject. The kit in some cases includes an insert with instructions for administration of the a pan ErbB family inhibitor, or a pharmaceutically acceptable salt or a pharmaceutical composition thereof, and a KRas G12D inhibitor compound of Formula (I), or a pharmaceutically acceptable salt or a pharmaceutical composition thereof. The insert may provide a user with one set of instructions for using the a pan ErbB family inhibitor, or a pharmaceutically acceptable salt or a pharmaceutical composition thereof, in combination with a KRas G12D inhibitor compound of Formula (I), or a pharmaceutically acceptable salt or a pharmaceutical composition thereof.

### Example A

### Pan ErbB Family Inhibitors Synergistically Increase the Activity of KRas G12D Inhibitors Against Cell Lines Expressing KRas G12D

This Example illustrates that the combination of exemplary KRas G12D inhibitor compound of Formula I (i.e., MRTX1133) and a pan ErbB family inhibitor synergistically inhibits the growth of tumor cell lines that express KRas G12D.

A panel of colon, pancreatic, gastric and endometrial cell lines harboring KRas G12D mutations was assembled to determine whether combining pan ErbB family inhibitors with exemplary KRas G12D inhibitors disclosed herein results in synergistic activity. The collection included SNU61 (colon, KCLB #00061), LS180 (colon, ATCC #CL-187), Panc 05.04 (pancreas, ATCC #CRL-2557), Panc 02.03 (pancreas ATCC #CRL-2553), SNU-407 (colon, AddexBio #C0009016), LS513 (colon, ATCC #CRL-2134), HPAC (pancreas, ATCC #CRL-2119), AGS (gastric, ATCC #CRL-1739), SNU-1197 (colon, KCLB #01197.1), SNU-1033 (colon, KCLB #01033), SNU-410 (pancreas, KCLB #00410), HEC-1-B (endometrial, ATCC #HTB-113), SU.86.86 (pancreas, ATCC #CRL-1837), SNU-C2B (colon, ATCC #CCL-250), Panc 08.13 (pancreas, ATCC #CRL-2551), SUIT-2 (pancreas, JCRB #JCRB1094), HPAF-II (pancreas, ATCC #CRL-1997), Panc 04.03 (pancreas, ATCC #CRL-2555), HCC-1588 (lung, KCLB #71588), GP2D (colon, SigmaAldrich #95090714), AsPC-1 (pancreas, ATCC CRL-1682), SW 1990 (pancreas, ATCC CRL-2172), and PANC-1 (pancreas, ATCC #CRL-1469).

Assays for determining the synergy score for the pairwise combinations for each cell line were performed in triplicate. Three 96-well plates plus an additional 4 wells of a separate 96-well control plate for determining baseline luminescence were seeded with 2000 cells/well of a particular cell line in a total volume of 90 μl of a suitable growth medium for that cell line, e.g., RPMI 1640 medium supplemented with 10% FBS and any cell line specific reagents need for growth. The plates were incubated overnight at 37° C. in a 5% CO₂ atmosphere.

To each of the designated baseline wells, 30 μl of Cell-Titer Glo reagent (CTG; Promega Corporation) was added to each well and the plates were incubated for 20 min with shaking at room temperature. Baseline luminescence was quantitated using a BMG ClarioStar multimode plate reader according to the manufacturer's instructions.

A series of working stock 1000× drug dilutions in 100% DMSO was prepared that includes an 8 point single agent dilution of MRTX 1133 and a 5-point single agent dilution of the pan ErbB family inhibitor. The dilutions used for MRTX1133 and the pan ErbB family inhibitor varied for each individual compound but were in the range of 3- to 6-fold/serial dilution.

A 10× intermediate dosing plate was prepared in serum free RPMI medium that contains arrayed single agent dilutions MRTX1133 or the pan ErbB family inhibitor. In addition, a matrix of 40 dilution combinations of MRTX1133 and the pan ErbB family inhibitor was prepared as test samples.

To each corresponding well of the three 96-well plates seeded with the appropriate cell line above, 10 μl of each 10× single agent and the 40 combinations of the dose matrix was added and the plates were incubated for 72 hours at 37 C in 5% CO₂ atmosphere. A 30 μl aliquot of Cell-Titer Glo reagent (CTG) was added to each test well, the plates were incubated for 20 min with shaking at room temperature, and luminescence was quantitated using a BMG ClarioStar multimode plate reader according to the manufacturer's instructions.

The raw data and metadata files were used as input files to calculate percent effect for each treatment condition and analyzed using four independent mathematical reference models designed to determine whether the two test compounds demonstrate synergy: Loewe additivity, Bliss independence, Highest Single Agent and ZIP.

The output of the data from each mathematical model is the assignment of a relative synergy score. The data reported in Table 1 are the aggregate sum of the Loewe additivity, Bliss independence, Highest Single Agent and ZIP scores (“Composite Synergy Score”).

TABLE 1
Composite Synergy Scores for Exemplary pan ErbB Family Inhibitors
Combined with MRTX1133 Against KRas G12D Cell Lines

| Pan ErbB | | |
|---|---|---|
| Family | | |
| Inhibitor | | |
| combined | | |
| with | | |
| MRTX1133 | Cetuximab | Afatinib |
| Cell Line | | |
| SNU61 | 52 | 44 |
| LS180 | 65 | 76 |
| Panc0504 | 44 | 24 |
| Panc0203 | 47 | 54 |
| SNU407 | 23 | 23 |
| LS513 | 62 | 69 |
| HPAC | 33 | 33 |
| AGS | 53 | 36 |
| SNU1197 | 6 | 63 |
| SNU1033 | 29 | 30 |
| SNU410 | −2 | −13 |
| Hec1B | 4 | −5 |
| SU8684 | 26 | 42 |
| SNUC2B | 50 | 25 |
| Panc0813 | 32 | 20 |
| SUIT2 | 45 | 45 |
| HPAFII | 31 | 23 |
| Panc0403 | 40 | 40 |
| Panc1005 | 29 | 37 |
| HCC1588 | −7 | 10 |
| GP2D | 48 | 26 |
| ASPC1 | 34 | 28 |
| SW1990 | 16 | 57 |
| Panc1 | −18 | −6 |

A custom R-script was created, integrating open source Bioconductor packages, to batch process metadata files containing experimental parameters and raw data files. Various numerical and graphical outputs were generated to summarize the data. Single agent parameters were generated using GRmetrics Clark N, Hafner M, Kouril M, Muhlich J, Niepel M, Williams E, Sorger P, Medvedovic M (2016). “GRcalculator: an online tool for calculating and mining drug response data.” doi: 10.6084/m9.figshare.4244408.v1, http://www.grcalculator.org/.

The synergyfinder package was used to determine whether the two test compounds demonstrate synergy using four independent mathematical reference models (Loewe additivity, Bliss independence, Highest Single Agent and ZIP) (He L et al) https://bioconductor.statistik.tu-dortmund.de/packages/3.6/bioc/vignettes/synergyfinder/inst/doc/synergyfinder.pdf

The output of the data from each mathematical model is the assignment of a relative synergy score. The data reported in the table are the aggregate sum of the Loewe additivity, Bliss independence, Highest Single Agent and ZIP scores (“Composite Synergy Score”).

A composite score of 22 to 80 was interpreted as a synergistic hit whereas a composite score of 11 to 21 indicates additive effect and score of <0 to 10 indicates no benefit. These results demonstrate that certain members of the panel of KRas G12D cell lines exhibited a synergistic effect for the combination of a panErbB family inhibitor with MRTX1133 warranting further interrogation of the combine efficacy studies in in vivo models.

### Example B

### In Vivo Models for Examining KRas G12D Inhibitor—Pan ErbB Family Inhibitor Combinations

Immunocompromised nude/nude mice are inoculated in the right hind flank with cells harboring a KRas G12D mutation. When tumor volumes reach between 200-400 mm³ in size, the mice are divided into four to five groups of 5 mice each. The first group is administered vehicle only. The second and third groups, depending on cell line, is administered either a twice daily single agent dose of the KRas G12D inhibitor at a concentration that yields a maximal biological effect or a less than maximal biological effect, depending on the cell line and the single agent activity, that does not result in complete tumor regression, or may be administered a twice daily for 2 sequential days followed by 5 days off, the KRas G12D inhibitor at a concentration that yields a maximal biological effect or a less than maximal biological effect, depending on the cell line and single agent activity, that does not result in complete tumor regression. In some cell lines, the third or fourth group is administered a single agent dose of EGFR inhibitors at a concentration that yields a maximal biological effect or a less than maximal biological effect, depending on the cell line and the single agent activity, that also does not result in complete tumor regression. The fourth or last groups are administered the single agent dose of the KRas G12D inhibitor using the twice daily schedule and/or the 2 sequential days followed by 5 days off schedule in combination with the single agent dose of one of the EGFR inhibitors. The treatment period varies from cell line to cell line but typically is between 15-40 days. Tumor volumes are measured using a caliper every two-three days and tumor volumes are calculated by the formula: 0.5×(Length×Width)². A greater degree of tumor growth inhibition for the combination in this model demonstrates that the combination therapy is likely to have a clinically meaningful benefit to treated subjects relative to treatment with only a KRas G12D inhibitor.

20 to 30 nude/nude mice per study were inoculated in the right hind limb with 5×10⁶ LS180 cells, AsPC-1 cells, GP2D cells, Panc 02.03 cells, SW1990 cells, or SNU-1033 cells. When tumor volumes reached ˜200 mm3-400 mm3 (study day 0) 5 mice in each of the groups were administered i.p. vehicle only (10% captisol in 50 mM citrate buffer pH 5.0), 30 mg/kg of Kras G12D inhibitor MRTX-'1133 (10% captisol in 50 mM citrate buffer, pH 5.0) either on the twice daily schedule or the twice daily for 2 consecutive days followed by 5 days off schedule, 12.5 mg/kg once daily of the EGFR inhibitor afatinib (10% captisol in 50 mM citrate buffer pH 5.0) or 0.25 mg/dose once every third day of the EGFR inhibitor cetuximab (saline), or 30 mg/kg of Kras G12D inhibitor on either schedule and either afatinib or cetuximab. Tumor volumes, measured at pre-specified days, for the five mice per group were averaged and are reported for LS180, AsPC-1, GP2D, Panc 02.03, SW1990, and SNU-1033 in Tables 2, 3, 4, 5 and 6, respectively.

### Example C

### KRas G12D Inhibitor MRTX-1133 in Combination with Cetuximab (LS180 Colon Cancer Cell Line)

Experimental Procedures. 25 nude/nude mice were inoculated with LS180 cells in the right hind flank. When the tumors reached ˜250 mm³ five treatment groups were established with five mice per group. The results of this study are provided in Table 2.

TABLE 2
Average Tumor Volumes (mm³) of LS180 Tumor Bearing
Mice Treated with Single Agents and in Combination

| | Vehicle | MRTX1133 | MRTX1133 | Cetuximab | Cetuximab |
|---|---|---|---|---|---|
| Study | BID | 30 mg/kg | 30 mg/kg | 0.25 mg/kg | Q3D + MRTX1133 |
| Day | daily | BID Daily | BID 2x/week | Q3D | BID 2x/week |
| 1 | 268.842 | 268.94 | 272.338 | 274.034 | 274.634 |
| 4 | 601.218 | 418.808 | 570.528 | 502.822 | 345.432 |
| 8 | 1089.326 | 593.71 | 1134.668 | 854.638 | 402.506 |
| 11 | 1738.074 | 808.808 | 1439.99 | 1330.518 | 471.35 |
| 15 | 1965.054 | 1197.106 | 1899.832 | 1715.024 | 607.458 |

As shown in Table 2, the administration of MRTX1133 at 30 mg/kg BID (twice per day) as a single agent exhibited 45% tumor growth inhibition at Day 15 (daily administration) and 4% tumor growth inhibition at Day 15 (twice per week administration). The administration of Cetuximab at 0.25 mg/kg Q3D (every third day as a single agent exhibited 15% tumor growth inhibition at Day 15. The combination of Cetuximab and MRTX1133 administered twice per week resulted in 80% tumor growth inhibition at Day 15.

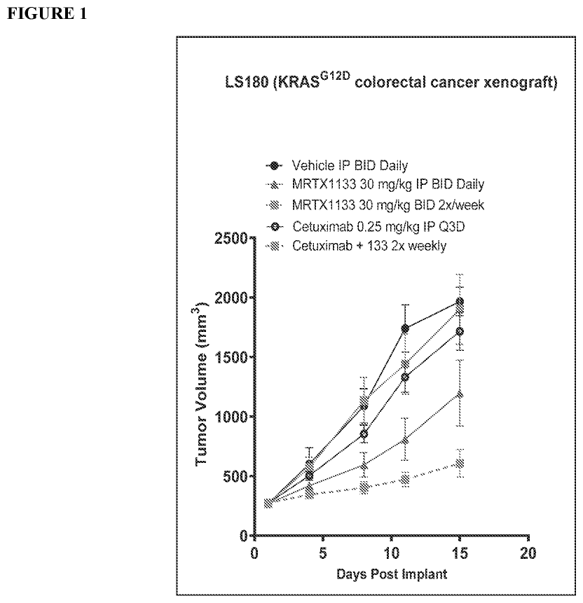

The results are also shown in FIG. 1.

### Example D

### KRas G12D Inhibitor MRTX-1133 in Combination with Afatinib (AsPC-1 TGI-42 Pancreatic Cancer Cell Line)

Experimental Procedures. 30 nude/nude mice were inoculated with AsPC-1 cells in the right hind flank. When the tumors reached ˜300 mm³ six treatment groups were established with five mice per group. The results of this study are provided in Table 3.

TABLE 3
Average Tumor Volumes (mm³) of AsPC-1 TGI-42 Tumor Bearing
Mice Treated with Single Agents and in Combination

| | | | MRTX1133 | | | |
|---|---|---|---|---|---|---|
| | | MRTX1133 | 30 mg/kg | Afatinib | Afatinib + | Afatinib + |
| Study | Vehicle | 30 mg/kg | BID | 12.5 mg/kg | MRTX1133 | MRTX1133 |
| Day | BID daily | BID Daily | 2x/week | QD | BID Daily | 2x/week |
| 1 | 325.54208 | 323.454 | 325.894 | 328.208 | 329.62 | 330.664 |
| 6 | 460.79656 | 356.398 | 394.182 | 463.11 | 342.904 | 390.086 |
| 9 | 545.79776 | 374.684 | 472.586 | 641.776 | 321.136 | 393.998 |
| 13 | 618.26576 | 316.51 | 496.598 | 720.894 | 275.318 | 441.406 |
| 16 | 758.40264 | 291.71 | 554.052 | 768.716 | 246.84 | 450.594 |
| 20 | 857.15704 | 269.246 | 586.5625 | 964.434 | 230.8 | 436.91 |
| 23 | 949.84696 | 213.334 | 640.49 | 1002.602 | 198.5975 | 429.614 |
| 27 | 1047.63768 | 195.354 | 679.96 | 1211.064 | 153.5225 | 424.944 |
| 30 | 1109.212 | 216.072 | 795.11 | 1289.494 | 147.5975 | 464.676 |
| 34 | 1182.71952 | 266.324 | 862.74 | 1252.045 | 194.315 | 516.112 |

As shown in Table 3, the administration of MRTX1133 at 30 mg/kg BID (twice per day) daily as a single agent exhibited −9% tumor regression at Day 34. The administration of Afatinib at 12.5 mg/kg QD (once daily) as a single agent exhibited 11% tumor growth inhibition at Day 34. The combination of Afatinib and MRTX1133 administered BID daily resulted in −44% tumor regression at Day 34, and the combination of Afatinib and MRTX1133 administered twice a week resulted in 77% tumor growth inhibition at Day 34.

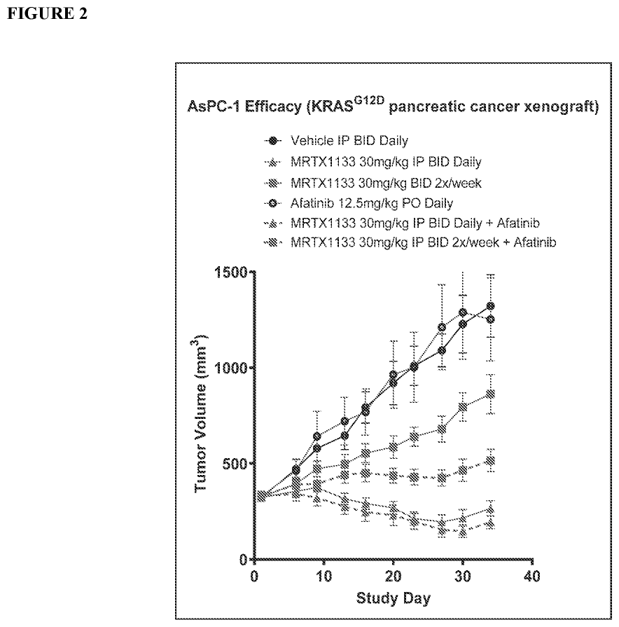

The results are also shown in FIG. 2.

### Example E

### KRas G12D Inhibitor MRTX-1133 in Combination with Cetuximab (GP2D TGI MDS 200108-807 Colorectal Cancer Cell Line)

Experimental Procedures. 20 nude/nude mice were inoculated with GP2D cells in the right hind flank. When the tumors reached ˜300 mm³ four treatment groups were established with five mice per group. The results of this study are provided in Table 4.

TABLE 4
Average Tumor Volumes (mm³) of GP2D TGI MDS 200108-807 Tumor
Bearing Mice Treated with Single Agents and in Combination

| | Vehicle | MRTX1133 | Cetuximab | Cetuximab+ |
|---|---|---|---|---|
| Study | BID | 30 mg/kg | 0.25 mg/kg | MRTX1133 |
| Day | daily | BID Daily | Q3D | BID daily |
| 2 | 332.1 | 341.3 | 359.72 | 334.68 |
| 6 | 543 | 282.98 | 503.18 | 238.62 |
| 9 | 717.96 | 237.18 | 774.9 | 199.38 |
| 13 | 1001.94 | 244.1 | 1022.3 | 187.64 |
| 16 | 1293.4 | 198.725 | 1333.14 | 155.65 |
| 20 | 1487.62 | 288.05 | 1738.12 | 185.83 |
| 23 | 1688.86 | 339 | 1848.12 | 174.13 |
| 27 | 2005.7 | 385.85 | 2033.58 | 193.53 |
| 30 | 2005.7 | 394.05 | 2098.62 | 225.77 |

As shown in Table 4, the administration of MRTX1133 at 30 mg/kg BID (twice per day) as a single agent exhibited 96% tumor growth inhibition at Day 30 (daily administration). The administration of Cetuximab at 0.25 mg/dose Q3D (every third day as a single agent exhibited 0% tumor growth inhibition at Day 30. The combination of Cetuximab and MRTX1133 administered BID daily resulted in −33% tumor regression at Day 30.

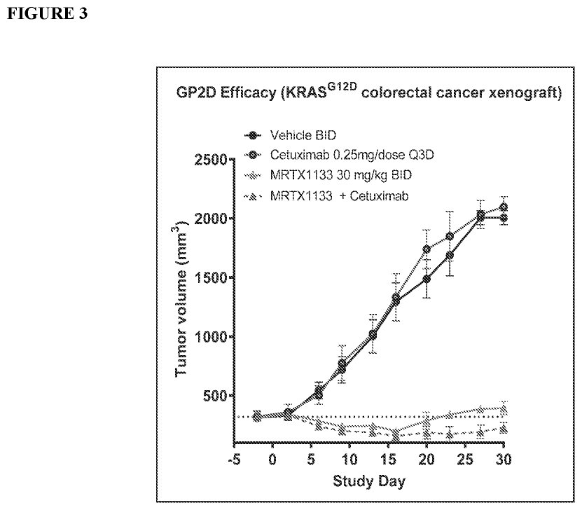

The results are also shown in FIG. 3.

### Example F

### KRas G12D Inhibitor MRTX-1133 in Combination with Afatinib or Cetuximab (Panc 02.03 TGI-43 Pancreatic Cancer Cell Line)

Experimental Procedures. 30 nude/nude mice were inoculated with SW1990 cells in the right hind flank. When the tumors reached ˜300 mm³ six treatment groups were established with five mice per group. The results of this study are provided in Table 5.

TABLE 5
Average Tumor Volumes (mm³) of SW1990 Tumor Bearing
Mice Treated with Single Agents and in Combination

| | | MRTX1133 | | Cetuximab + | Afatinib | Afatinib + |
|---|---|---|---|---|---|---|
| | Vehicle | 30 mg/kg | Cetuximab | MRTX1133 | 12.5 mg/kg | MRTX1133 |
| Study | BID | BID | 0.25 mg/dose | BID | QD (once | BID |
| Day | daily | 2x/week | Q3D | 2x/week | daily) | 2x/week |
| 1 | 328.896 | 329.18 | 330.908 | 330.114 | 331.548 | 329.49 |
| 4 | 446.116 | 265.246 | 382.648 | 258.862 | 361.83 | 296.166 |
| 8 | 544.424 | 386.354 | 456.018 | 212.472 | 398.444 | 275.256 |
| 11 | 647.874 | 392.042 | 489.668 | 184.77 | 470.498 | 285.106 |
| 15 | 786.634 | 480.434 | 589.42 | 161.7 | 660.352 | 331.838 |
| 18 | 927.746 | 496.496 | 730.102 | 130.748 | 784.04 | 368.774 |
| 22 | 1113.464 | 549.618 | 858.32 | 147.574 | 904.724 | 409.548 |

As shown in Table 5, the administration of MRTX1133 at 30 mg/kg BID (twice per week) as a single agent exhibited 72% tumor growth inhibition at Day 22. The administration of cetuximab at 0.25 mg/dose Q3D (every third day) as a single agent exhibited 33% tumor growth inhibition at Day 22. The combination of cetuximab and MRTX1133 administered BID twice per week resulted in −55% tumor regression at Day 22. The administration of afatinib at 12.5 mg/kg daily as a single agent exhibited 27% tumor growth inhibition at Day 22. The combination of afatinib and MRTX1133 administered BID twice weekly resulted in 90% tumor growth inhibition at Day 22.

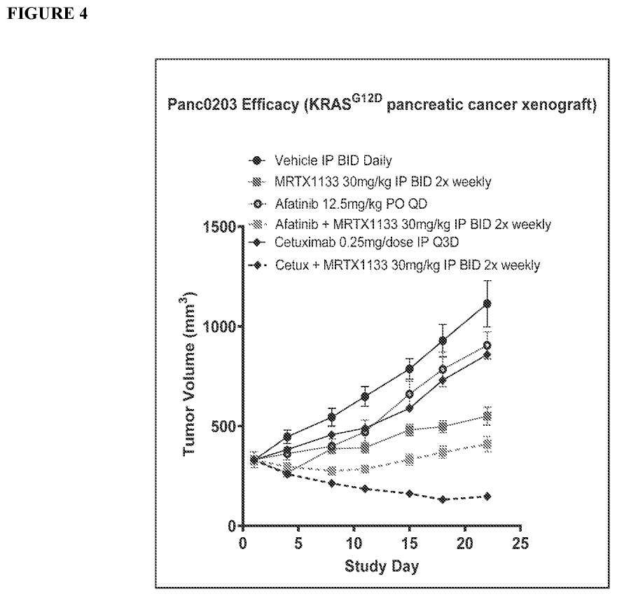

The results are also shown in FIG. 4.

### Example G

### KRas G12D Inhibitor MRTX-1133 in Combination with Afatinib or Cetuximab (SW1990 TGI MDS #200407-807 Pancreatic Cancer Cell Line)

Experimental Procedures. 30 nude/nude mice were inoculated with SW1990 cells in the right hind flank. When the tumors reached ˜300 mm³ six treatment groups were established with five mice per group. The results of this study are provided in Table 6.

TABLE 6
Average Tumor Volumes (mm³) of SW1990 Tumor Bearing
Mice Treated with Single Agents and in Combination

| | | | Afatinib | | Cetuximab | Cetuximab + |
|---|---|---|---|---|---|---|
| | Vehicle | MRTX1133 | 12.5 mg/kg | Afatinib + | 0.25 mg/dose | MRTX1133 |
| Study | BID | 30 mg/kg | QD once | MRTX1133 | Q3D every | BID |
| Day | daily | BID Daily | daily | BID Daily | 3 days | daily |
| −1 | 363.4 | 362.82 | 363.4 | 366.54 | 365.06 | 364.62 |
| 2 | 456.88 | 265.08 | 455.54 | 263.54 | 448.1 | 252.9 |
| 6 | 622.52 | 194.42 | 536.86 | 149.94 | 517.16 | 140.72 |
| 9 | 699.3 | 187.24 | 624.98 | 130.44 | 561.72 | 106.94 |
| 13 | 1054.22 | 241.84 | 698.56 | 118.36 | 641.2 | 82.68 |
| 16 | 1229.54 | 235.32 | 778.74 | 106.425 | 749.78 | 72.94 |
| 20 | 1409.68 | 214.98 | 1141.575 | 79.45 | 933.62 | 53.88 |
| 23 | 1502.06 | 195.04 | 1293.5 | 71.85 | 1065.66 | 52.62 |

As shown in Table 6, the administration of MRTX1133 at 30 mg/kg BID (twice per day) daily as a single agent exhibited −46% tumor regression at Day 23. The administration of Afatinib at 12.5 mg/kg QD daily as a single agent exhibited 20% tumor growth inhibition at Day 23. The combination of Afatinib and MRTX1133 administered BID daily resulted in −80% tumor regression at Day 23. The administration of Cetuximab at 0.25 mg/dose Q3D (every third day) as a single agent exhibited 42% tumor growth inhibition at Day 23. The combination of Cetuximab and MRTX1133 administered BID daily resulted in −86% tumor regression at Day 23.

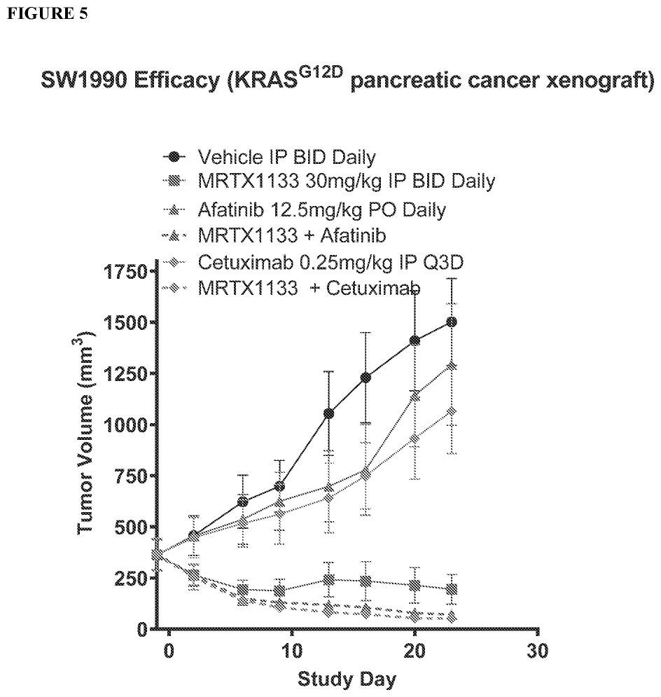

The results are also shown in FIG. 5.

### Example H

### KRas G12D Inhibitor MRTX-1133 in Combination with Cetuximab (SNU-1033 TGI 46 Colorectal Cancer Cell Line)

Experimental Procedures. 20 nude/nude mice were inoculated with SNU-1033 cells in the right hind flank. When the tumors reached ˜300 mm³ four treatment groups were established with five mice per group. The results of this study are provided in Table 7.

TABLE 7
Average Tumor Volumes (mm³) of SNU-1033 TGI 46 Tumor Bearing
Mice Treated with Single Agents and in Combination

| | | | Cetuximab | |
|---|---|---|---|---|
| | | MRTX1133 | 0.25 | Cetuximab + |
| | Vehicle | 30 mg/kg | mg/dose | MRTX1133 |
| Study | BID | BID | Q3D (every | BID |
| Day | daily | 2x/week | 3^{rd} day) | 2x/week |
| 1 | 294.992 | 298.92 | 297.512 | 305.968 |
| 5 | 487.128 | 336.336 | 389.768 | 283.152 |
| 8 | 560.436 | 431.99 | 418.018 | 287.536 |
| 12 | 727.846 | 468.892 | 448.118 | 296.612 |
| 15 | 826.37 | 529.222 | 460.894 | 290.012 |
| 19 | 1054.748 | 643.516 | 512.09 | 260.822 |
| 22 | 1225.776 | 703.368 | 555.326 | 287.638 |
| 26 | 1287.22 | 802.69 | 618.748 | 300.294 |
| 29 | 1350.962 | 838.708 | 636.83 | 295.17 |
| 33 | 1435.752 | 895.918 | 668.412 | 290.256 |

As shown in Table 7, the administration of MRTX1133 at 30 mg/kg twice per week as a single agent exhibited 47% tumor growth inhibition at Day 33. The administration of Cetuximab at 0.25 mg/dose Q3D (every third day) as a single agent exhibited 67% tumor growth inhibition at Day 33. The combination of Cetuximab and MRTX1133 administered twice per week resulted in −5% tumor regression at Day 33.

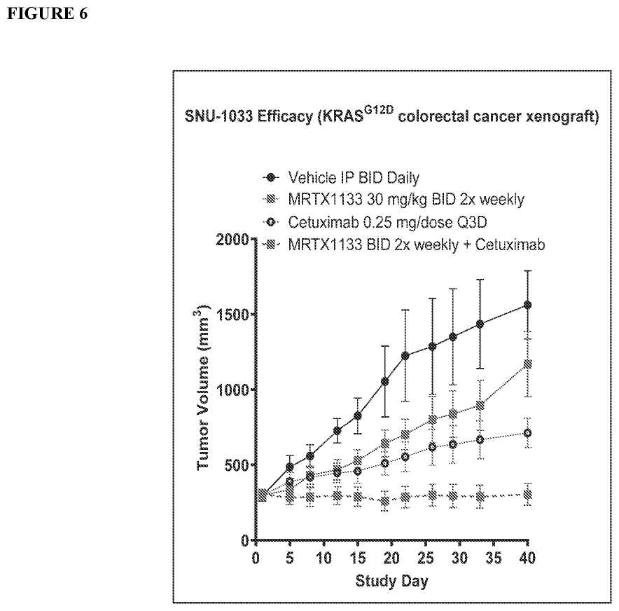

The results are also shown in FIG. 6.

### Example I

### KRas G12D Inhibitor MRTX-1133 in Combination with Cetuximab (AsPC-1 TGI 26 Pancreatic Cancer Cell Line)

Experimental Procedures. 20 nude/nude mice were inoculated with AsPC-1 cells in the right hind flank. When the tumors reached ˜200 mm³ four treatment groups were established with five mice per group. The results of this study are provided in Table 8.

TABLE 8
Average Tumor Volumes (mm³) of AsPC-1 TGI 26 Tumor Bearing
Mice Treated with Single Agents and in Combination

| | | | Cetuximab | |
|---|---|---|---|---|
| | | | 0.25 | |
| | | | mg/dose | |
| | Vehicle | MRTX1133 | IP Q3D | |
| Study | IP BID | 30 mg/kg IP | (every 3^{rd} | Cetuximab + |
| Day | daily | BID daily | day | MRTX1133 |
| 0 | 197.13 | 197.468 | 197.598 | 197.716 |
| 4 | 226.264 | 194.514 | 261.156 | 153.57 |
| 7 | 272.766 | 175.198 | 303.582 | 151.266 |
| 11 | 326.554 | 159.756 | 342.314 | 105.61 |
| 14 | 400.27 | 172.986 | 402.046 | 89.764 |
| 18 | 427.542 | 168.412 | 441.54 | 59.718 |
| 21 | 474.58 | 143.928 | 472.884 | 26.4 |
| 25 | 519.324 | 149.308 | 533.024 | 19 |

As shown in Table 8, the administration of MRTX1133 at 30 mg/kg twice daily as a single agent exhibited −24% tumor regression on Day 25. The administration of Cetuximab at 0.25 mg/dose Q3D (every third day) as a single agent exhibited 0% tumor growth inhibition at Day 25. The combination of Cetuximab and MRTX1133 resulted in −90% tumor regression at Day 25.

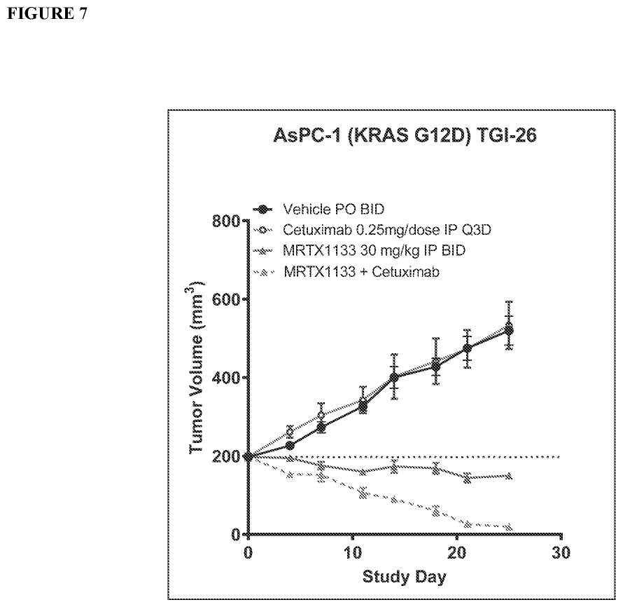

The results are also shown in FIG. 7.

### Example J

### KRas G12D Inhibitor MRTX-1133 in Combination with Erlotinib (HPAC TGI 73 Pancreatic Cancer Cell Line)

Experimental Procedures. 20 nude/nude mice were inoculated with HPAC cells in the right hind flank. When the tumors reached ˜200 mm³ four treatment groups were established with five mice per group. The results of this study are provided in Table 9.

TABLE 9
Average Tumor Volumes (mm³) of HPAC TGI 73 Tumor Bearing
Mice Treated with Single Agents and in Combination

| | Vehicle | MRTX1133 | Erlotinib 50 | |
|---|---|---|---|---|
| Study | IP BID | 30 mg/kg IP | mg/kg PO | Erlotinib + |
| Day | daily | BID daily | daily | MRTX1133 |
| 0 | 190.47 | 196.76 | 197.582 | 197.618 |
| 3 | 309.65 | 149.646 | 320.132 | 147.36 |
| 7 | 447.76 | 107.504 | 421.908 | 93.002 |
| 10 | 686.434 | 99.742 | 587.79 | 76.612 |
| 14 | 828.298 | 91.818 | 705.688 | 75.142 |
| 17 | 1098.288 | 93.348 | 856.214 | 75.488 |
| 21 | 1359.228 | 74.006 | 945.702 | 36.848 |

As shown in Table 9, the administration of MRTX1133 at 30 mg/kg daily as a single agent exhibited −62% tumor regression on Day 21. The administration of Erlotinib at 50 mg/kg daily as a single agent exhibited 35% tumor growth inhibition at Day 21. The combination of Erlotinib and MRTX1133 resulted in −81% tumor regression at Day 21.

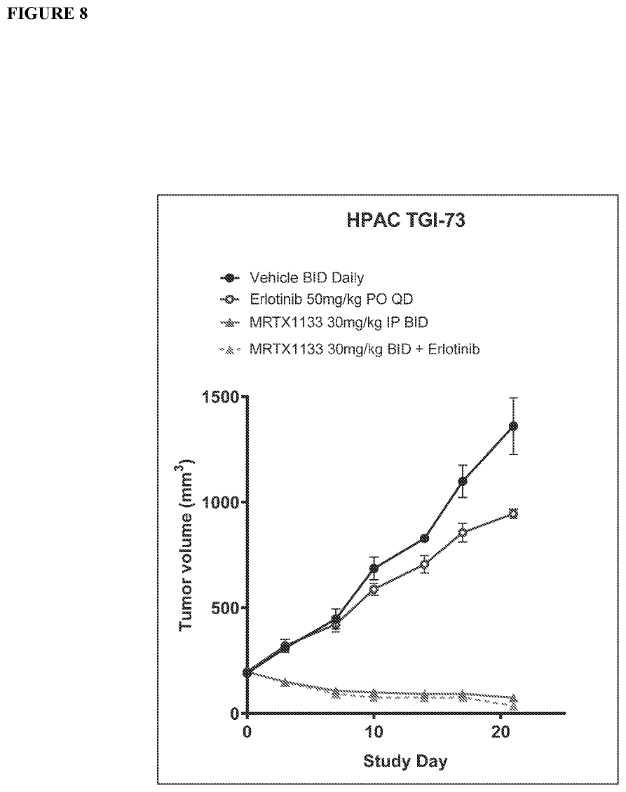

The results are also shown in FIG. 8.

These results demonstrate that the combination therapy resulted in greater amount of tumor growth inhibition compared to either single agent alone demonstrating enhanced in vivo anti-tumor efficacy of the combination against KRas G12D expressing cancer.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A method of treating cancer in a subject in need thereof, comprising administering to the subject a therapeutically effective amount of a combination of a pan ErbB family inhibitor and a KRas G12D inhibitor of formula (I): 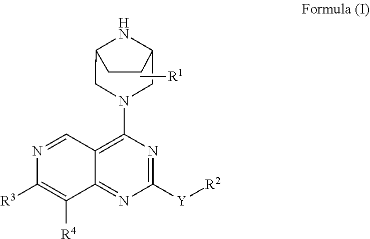
or a pharmaceutically acceptable salt thereof,
wherein:
R¹ is hydrogen, hydroxy, halogen, C1-C3 alkyl, C1-C3 cyanoalkyl, C1-C3 hydroxyalkyl, HC(═O)—, —CO₂R⁵, —CO₂N(R⁵)₂ or a 5-6 membered heteroaryl;
Y is a bond, O or NR⁵;
R² is hydrogen, —N(R⁵)₂, heterocyclyl, C1-C6 alkyl, -L-heterocyclyl, -L-aryl, -L-heteroaryl, -L-cycloalkyl, -L-N(R⁵)₂, -L-NHC(═NH)NH₂, -L-C(O)N(R⁵)₂, -L-C1-C6 haloalkyl, -L-OR⁵, -L-(CH₂OR⁵)(CH₂)ₙOR⁵, -L-NR₅C(O)-aryl, -L-COOH, or -LC(═O)OC1-C6 alkyl, wherein the heterocyclyl and the aryl portion of -L-NR₅C(O)-aryl and the heterocyclyl portion of -L-heterocyclyl and the cycloalkyl portion of the -L-cycloalkyl may be optionally substituted with one or more R⁶, and wherein the aryl or heteroaryl of the -L-aryl and the -L-heteroaryl may be optionally substituted with one or more R⁷;
each L is independently a C1-C4 alkylene optionally substituted with hydroxy, C1-C4 hydroxyalkyl or heteroaryl;
R³ is aryl or heteroaryl, wherein the aryl or the heteroaryl is optionally substituted with one or more R⁸;
R⁴ is hydrogen, halogen or C1-C3 alkyl;
each R⁶ is independently hydrogen or C1-C3 alkyl;
each R⁶ is independently halogen, hydroxy, C1-C3 hydroxyalkyl, C1-C3 alkyl, C1-C3 haloalkyl, C1-C3 alkoxy, cyano, -Q-phenyl, -Q-phenylSO₂F, —NHC(O)phenyl, —NHC(O)phenylSO₂F, C1-C3 alkyl substituted pyrazolyl, araC1-C3 alkyl-, tert-butyldimethylsilyloxyCH₂-, —N(R⁵)₂, (C1-C3 alkoxy)C1-C3 alkyl-, (C1-C3 alkyl)C(═O), oxo, (C1-C3 haloalkyl)C(═O)—, —SO₂F, (C1-C3 alkoxy)C1-C3 alkoxy, —CH₂OC(O)N(R⁵)₂, —CH₂NHC(O)OC1-C6 alkyl, —CH₂NHC(O)N(R⁵)₂, —CH₂NHC(O)C1-C6 alkyl, —CH₂(pyrazolyl), —CH₂NHSO₂C1-C6 alkyl, —CH₂OC(O)heterocyclyl, —OC(O)N(R⁵)₂, —OC(O)NH(C1-C3 alkyl)O(C1-C3 alkyl), —OC(O)NH(C1-C3 alkyl)O(C1-C3 alkyl)phenyl(C1-C3 alkyl)N(CH₃)₂, —OC(O)NH(C1-C3 alkyl)O(C1-C3 alkyl)phenyl or —OC(O)heterocyclyl, —CH₂heterocyclyl, wherein the phenyl of —NHC(O)phenyl or —OC(O)NH(C1-C3 alkyl)O(C1-C3 alkyl)phenyl is optionally substituted with —C(O)H or OH and wherein the heterocyclyl of —CH₂heterocyclyl is optionally substituted with oxo;
Q is a bond or O;
each R⁷ is independently halogen, hydroxy, HC(═O)—, C1-C4 alkyl, C1-C4 alkoxy, C1-C4 haloalkyl, C1-C4 hydroxyalkyl, or —N(R⁵)₂; and
each R⁸ is independently halogen, cyano, hydroxy, C1-C4 alkyl, —S—C1-C3 alkyl, C2-C4 alkenyl, C2-C4 alkynyl, C2-C4 hydroxyalkynyl, C1-C3 cyanoalkyl, triazolyl, C1-C3 haloalkyl, —O—C1-C3 haloalkyl, —S—C1-C3 haloalkyl, C1-C3 alkoxy, hydroxyC1-C3 alkyl, —CH₂C(═O)N(R⁵)₂, —C3-C4 alkynyl(NR⁵)₂, —N(R⁵)₂, deuteroC2-C4 alkynyl, (C1-C3 alkoxy)haloC1-C3 alkyl-, or C3-C6 cycloalkyl wherein said C3-C6 cycloalkyl is optionally substituted with halogen or C1-C3 alkyl.

2. The method of claim 1, wherein R¹ is hydrogen, halogen, hydroxy, C1-C3 alkyl, C1-C3 cyanoalkyl, hydroxyalkyl, HC(═O)—, —CO₂R⁵, or —CO₂N(R⁵)₂.

3. The method of claim 2, wherein R⁵ is hydrogen, C1-C3 alkyl or C1-C3 cyanoalkyl.

4. The method of claim 2, wherein Y is O and R² is C1-C6 alkyl or -L-heterocyclyl optionally substituted with one or more R⁶.

5. The method of claim 4, wherein the C1-C6 alkyl is methyl, ethyl, isopropyl or isobutyl.

6. The method of claim 4, wherein L is methylene and the heterocyclyl is hexahydro-1H-pyrrolizinyl, hexahydro-3H-pyrrolizin-3-one, hexahydro-1H-pyrrolo[2,1-c][1,4]oxazinyl, octahydroindolizinyl, hexahydropyrrolizine 4(1H)-oxide, azetidinyl, pyrrolidinyl, pyrrolidin-2-one, oxetanyl, piperidinyl, 1-azabicyclo[2.2.1]heptanyl, morpholinyl, oxa-5-azabicyclo[2.2.1]heptan-5-yl, thiopyranyl, 6-oxa-2 [symbol] ²-azaspiro[3.4]octanyl, 7-oxa-2 [symbol] ²-azaspiro[3.5]nonanyl, 2′,3′-dihydrospiro[cyclopropane-1,1′-indenyl], (2S)-1-azabicyclo[2.2.1]heptan-2-yl, or tetrahydrofuranyl, each optionally substituted with one or more R⁶.

7. The method of claim 4, wherein L is methylene and the heterocyclyl is hexahydro-1H-pyrrolizinyl.

8. The method of claim 7, wherein heterocyclyl is hexahydro-1H-pyrrolizinyl substituted with one R⁶, wherein R⁶ is halogen, hydroxy, C1-C3 hydroxyalkyl, C1-C3 haloalkyl, C1-C3 alkyl, C1-C3 alkoxy, phenyl or pyrazolyl.

9. The method of claim 8, wherein the halogen is fluorine.

10. The method of claim 6, wherein the heterocyclyl is hexahydro-1H-pyrrolizinyl further substituted with two additional R⁶ groups, wherein the two additional R⁶ groups are independently C1-C3 alkyl.

11. The method of claim 6, wherein the heterocyclyl is azetidinyl substituted with one R⁶, wherein R⁶ is C1-C3 alkyl.

12. The method of claim 6, wherein the heterocyclyl is pyrrolidinyl substituted with one R⁶, wherein R⁶ is hydroxalkyl, haloalkyl, C1-C3 alkyl, alkoxy, araC1-C3 alkyl, -Q-phenyl and —NHC(O)phenyl, and wherein the aryl portion of the araC1-C3 alkyl or the phenyl portion of the -Q-phenyl and —NHC(O)phenyl are each optionally substituted with one or more R⁶.

13. The method of claim 12, wherein the phenyl group of the -Q-phenyl or the —NHC(O)phenyl is substituted with SO₂F.

14. The method of claim 6, wherein the heterocyclyl is pyrrolidinyl substituted with two R⁶ groups wherein one R⁶ is C1-C3 alkyl and the other R⁶ is C1-C3 alkoxy or halogen.

15. The method of claim 6, wherein the heterocyclyl is pyrrolidin-2-one substituted with one R⁶, wherein R⁶ is C1-C3 alkyl.

16. The compound or salt of claim 6, wherein the heterocyclyl is piperidinyl substituted with one R⁶, wherein R⁶ is acetyl, (C1-C3 alkoxy)C1-C3 alkoxy, or —C(O)CH₂Cl.

17. The method of claim 6, wherein Y is O, L is ethylene or propylene and the heterocyclyl is morpholinyl or oxa-5-azabicyclo[2.2.1]heptan-5-yl.

18. The method of claim 2, wherein Y is O and R² is -L-heteroaryl, wherein the heteroaryl portion is optionally substituted with one or more R⁷.

19. The method of claim 18, wherein L is methylene or ethylene and the heteroaryl is pyridyl, pyrazolyl, imidazolyl, triazolyl, 4,5,6,7-tetrahydro-1H-indazolyl, benzimidazolyl, imidazo[1,2-a]pyridinyl, or pyrimidinyl, each optionally substituted with one or more R⁷.

20. The method of claim 19, wherein the heteroaryl is pyridyl substituted with one R⁷, wherein R⁷ is halogen, C1-C4 alkyl, —N(R⁵)₂, or C1-C4 alkoxy.

21. The method of claim 19, wherein the heteroaryl is pyrazolyl substituted with one R⁷, wherein R⁷ is C1-C4 alkyl or —N(R⁵)₂.

22. The method of claim 19, wherein the heteroaryl is imidazolyl substituted with one R⁷, wherein R⁷ is C1-C4 alkyl, C1-C4 haloalkyl, or C1-C4 hydroxyalkyl.

23. The method of claim 19, wherein the heteroaryl is triazolyl substituted with one R⁷, wherein R⁷ is C1-C4 alkyl.

24. The method of claim 2, wherein Y is O and R² is -L-aryl, wherein the aryl portion is optionally substituted with one or more R⁷.

25. The method of claim 2, wherein Y is O and R² is -L-cycloalkyl, wherein the cycloalkyl portion is optionally substituted with one or more R⁷.

26. The method of claim 2, wherein Y is O, and R² is -L-N(R⁵)₂.

27. The method of claim 26, wherein L is ethylene and each R⁵ is an independently selected C1-C3 alkyl.

28. The method of claim 2, wherein Y is O, and R² is -L-NC(═NH)—NH₂.

29. The method of claim 28, wherein L is ethylene or propylene.

30. The method of claim 2, wherein Y is O, and R² is -L-C1-C6 haloalkyl.

31. The method of claim 2, wherein Y is O, and R² is -L-OR⁵.

32. The method of claim 2, wherein Y is O, and R² is -L-(CH₂OR⁵)(CH₂)ₙOR⁵.

33. The method of claim 2, wherein Y is O, and R² is -L-NR₅C(O)-aryl.

34. The method of claim 2, wherein R³ is aryl optionally substituted with one or more R⁸.

35. The method of claim 34, wherein the aryl is phenyl, naphthyl, 1,2,3,4-tetrahydronaphthalenyl and 2,3-dihydro-1H-indenyl, each optionally substituted with one or more R⁸.

36. The method of claim 2, wherein R³ is heteroaryl optionally substituted with one or more R⁸.

37. The method of claim 36, wherein the heteroaryl is isoquinolinyl, indazolyl, or benzo[d][1,3]dioxolyl optionally substituted with one or more R⁸.

38. The method of claim 37, wherein the heteroaryl is isoquinolinyl substituted with one R⁸, wherein R⁸ is halogen or C2-C4 alkynyl.

39. The method of claim 37, wherein the heteroaryl is indazolyl substituted with one R⁸, wherein R⁸ is C1-C3 alkyl.

40. The method of claim 37, wherein the heteroaryl is benzo[d][1,3]dioxolyl substituted with two R⁸ groups, wherein the R⁸ groups are independently selected halogens.

41. The method of claim 2, wherein R⁴ is halogen, or C1-C3 alkyl.

42. The method of claim 41, wherein the halogen is fluorine.

43. The method of claim 41, wherein the C1-C3 alkyl is methyl.

44. The method of claim 1, wherein R¹ is hydrogen.

45. The method of claim 1, wherein the KRas G12D inhibitor is selected from the group consisting of: 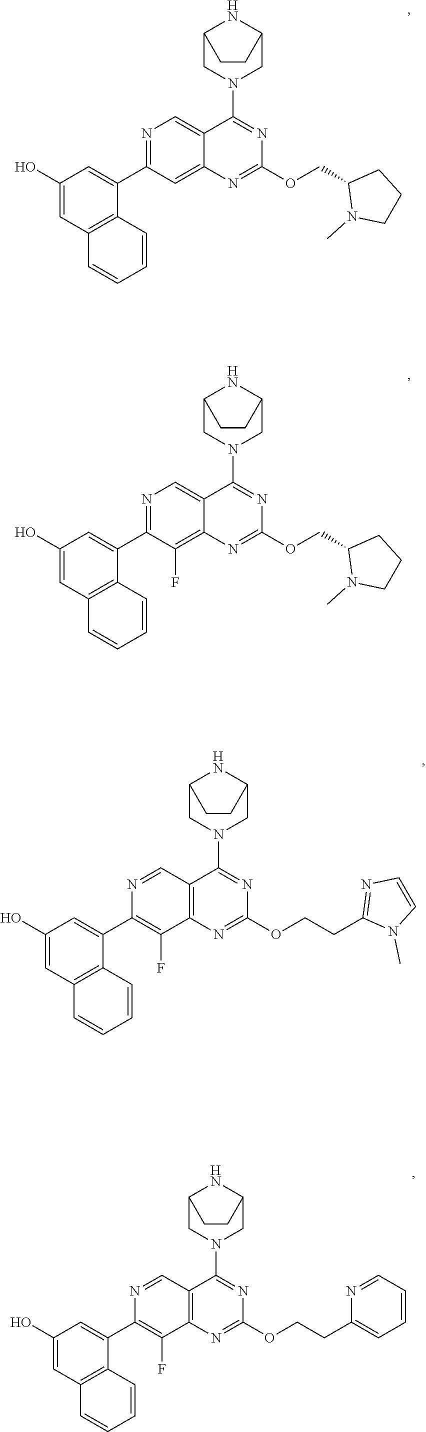 [SMILES: CN1CCC[C@H]1COc1nc(N2CC3CCC(C2)N3)c2cnc(-c3cc(O)cc4ccccc34)cc2n1 ; CN1CCC[C@H]1COc1nc(N2CC3CCC(C2)N3)c2cnc(-c3cc(O)cc4ccccc34)c(F)c2n1 ; Cn1ccnc1CCOc1nc(N2CC3CCC(C2)N3)c2cnc(-c3cc(O)cc4ccccc34)c(F)c2n1 ; Oc1cc(-c2ncc3c(N4CC5CCC(C4)N5)nc(OCCc4ccccn4)nc3c2F)c2ccccc2c1] 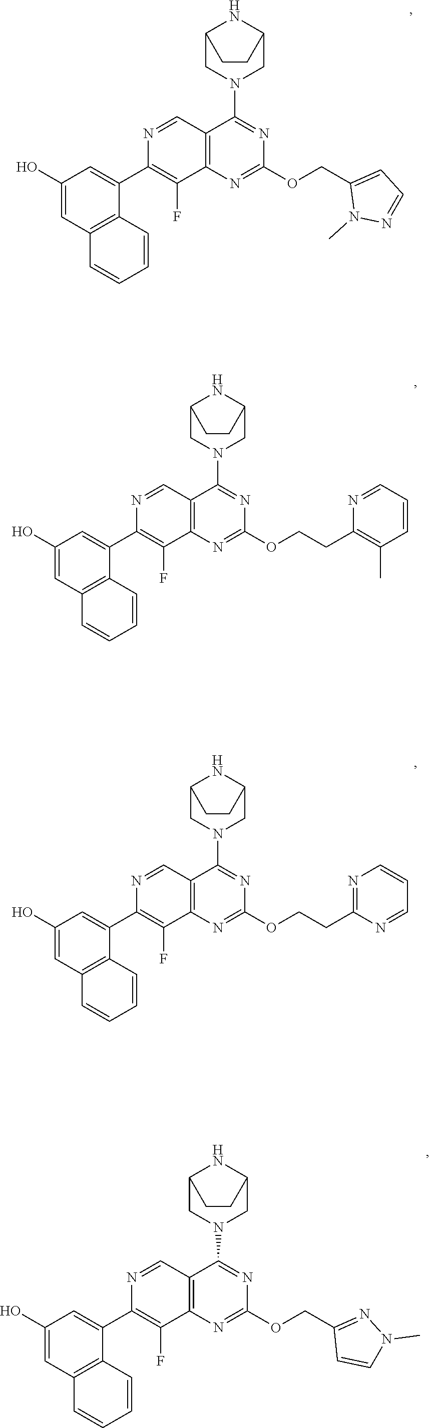 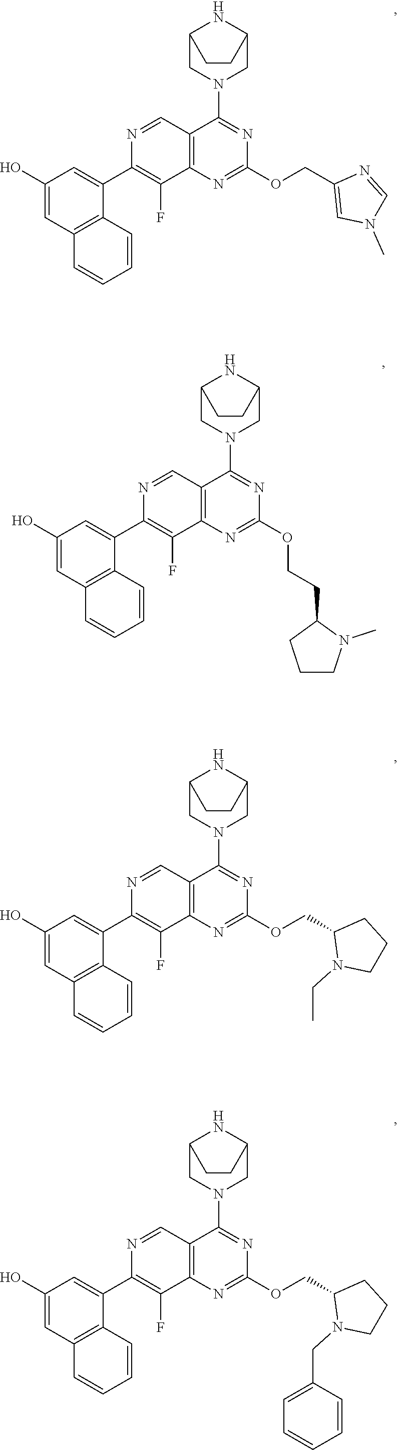 [SMILES: Cn1cnc(COc2nc(N3CC4CCC(C3)N4)c3cnc(-c4cc(O)cc5ccccc45)c(F)c3n2)c1 ; CN1CCC[C@H]1CCOc1nc(N2CC3CCC(C2)N3)c2cnc(-c3cc(O)cc4ccccc34)c(F)c2n1 ; CCN1CCC[C@H]1COc1nc(N2CC3CCC(C2)N3)c2cnc(-c3cc(O)cc4ccccc34)c(F)c2n1 ; Oc1cc(-c2ncc3c(N4CC5CCC(C4)N5)nc(OC[C@@H]4CCCN4Cc4ccccc4)nc3c2F)c2ccccc2c1] 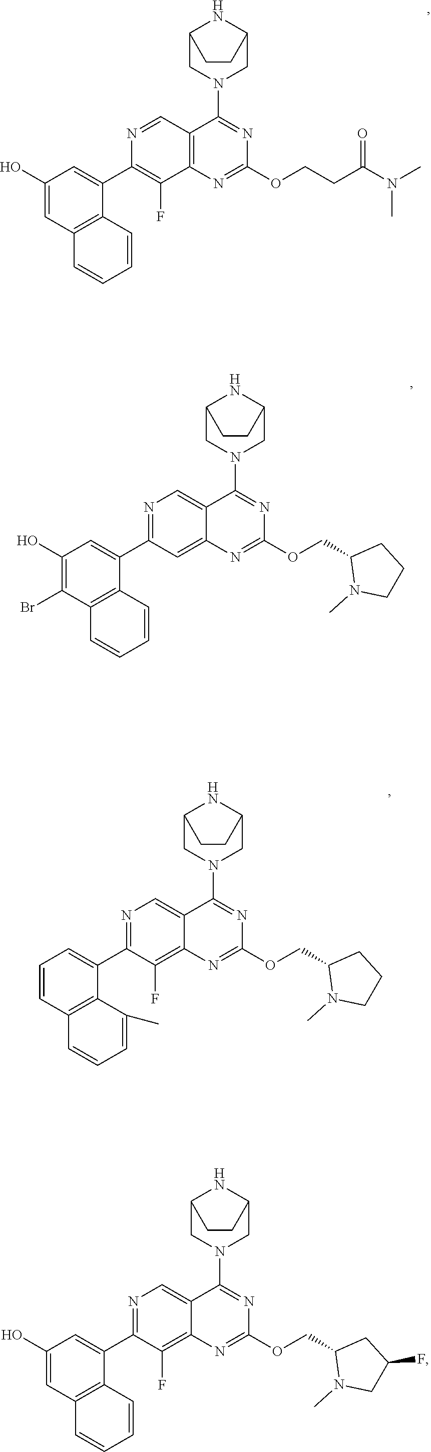 [SMILES: CN(C)C(=O)CCOc1nc(N2CC3CCC(C2)N3)c2cnc(-c3cc(O)cc4ccccc34)c(F)c2n1 ; CN1CCC[C@H]1COc1nc(N2CC3CCC(C2)N3)c2cnc(-c3cc(O)c(Br)c4ccccc34)cc2n1 ; Cc1cccc2cccc(-c3ncc4c(N5CC6CCC(C5)N6)nc(OC[C@@H]5CCCN5C)nc4c3F)c12 ; CN1C[C@H](F)C[C@H]1COc1nc(N2CC3CCC(C2)N3)c2cnc(-c3cc(O)cc4ccccc34)c(F)c2n1] 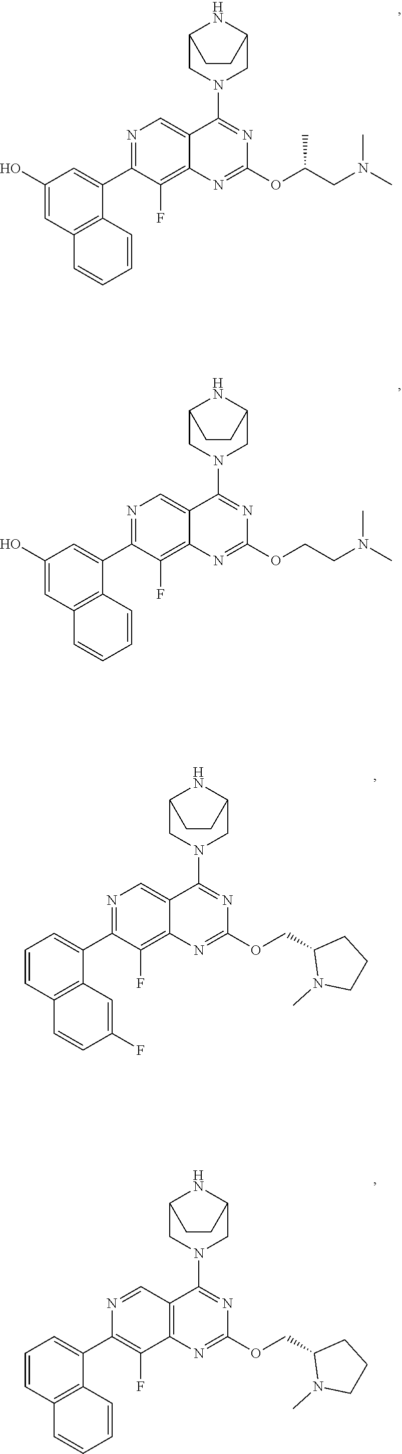 [SMILES: C[C@H](CN(C)C)Oc1nc(N2CC3CCC(C2)N3)c2cnc(-c3cc(O)cc4ccccc34)c(F)c2n1 ; CN(C)CCOc1nc(N2CC3CCC(C2)N3)c2cnc(-c3cc(O)cc4ccccc34)c(F)c2n1 ; CN1CCC[C@H]1COc1nc(N2CC3CCC(C2)N3)c2cnc(-c3cccc4ccc(F)cc34)c(F)c2n1 ; CN1CCC[C@H]1COc1nc(N2CC3CCC(C2)N3)c2cnc(-c3cccc4ccccc34)c(F)c2n1] 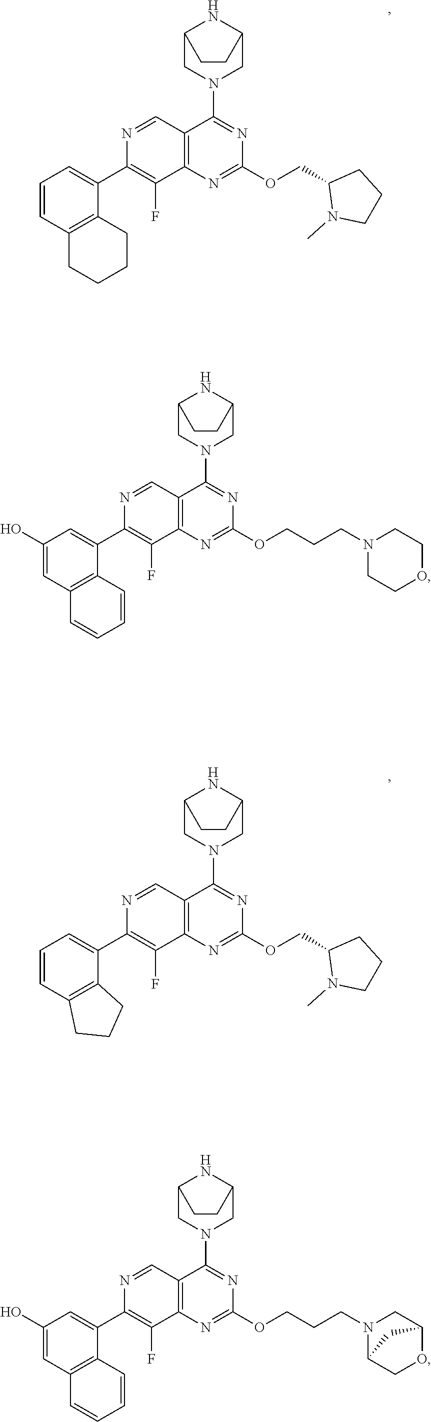 [SMILES: CN1CCC[C@H]1COc1nc(N2CC3CCC(C2)N3)c2cnc(-c3cccc4c3CCCC4)c(F)c2n1 ; Oc1cc(-c2ncc3c(N4CC5CCC(C4)N5)nc(OCCCN4CCOCC4)nc3c2F)c2ccccc2c1 ; CN1CCC[C@H]1COc1nc(N2CC3CCC(C2)N3)c2cnc(-c3cccc4c3CCC4)c(F)c2n1 ; Oc1cc(-c2ncc3c(N4CC5CCC(C4)N5)nc(OCCCN4C[C@@H]5C[C@H]4CO5)nc3c2F)c2ccccc2c1] 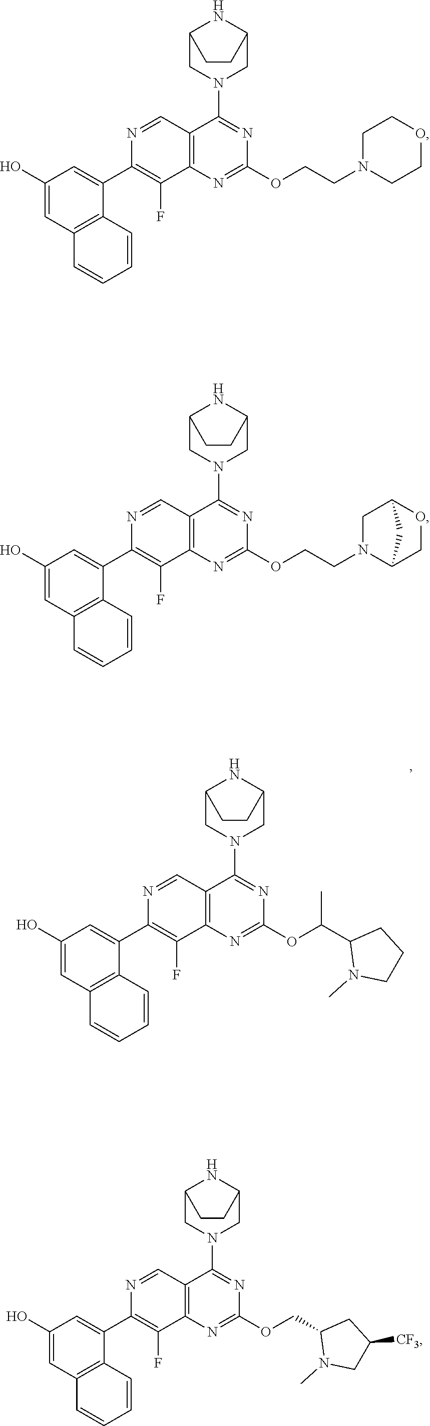 [SMILES: Oc1cc(-c2ncc3c(N4CC5CCC(C4)N5)nc(OCCN4CCOCC4)nc3c2F)c2ccccc2c1 ; Oc1cc(-c2ncc3c(N4CC5CCC(C4)N5)nc(OCCN4C[C@@H]5C[C@H]4CO5)nc3c2F)c2ccccc2c1 ; CC(Oc1nc(N2CC3CCC(C2)N3)c2cnc(-c3cc(O)cc4ccccc34)c(F)c2n1)C1CCCN1C ; CN1C[C@H](C(F)(F)F)C[C@H]1COc1nc(N2CC3CCC(C2)N3)c2cnc(-c3cc(O)cc4ccccc34)c(F)c2n1] 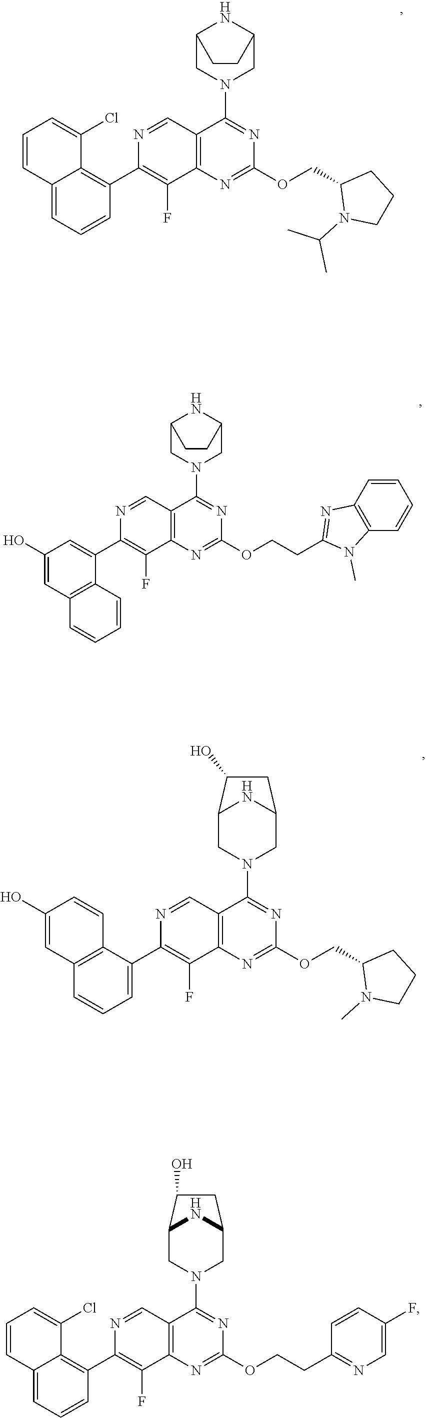 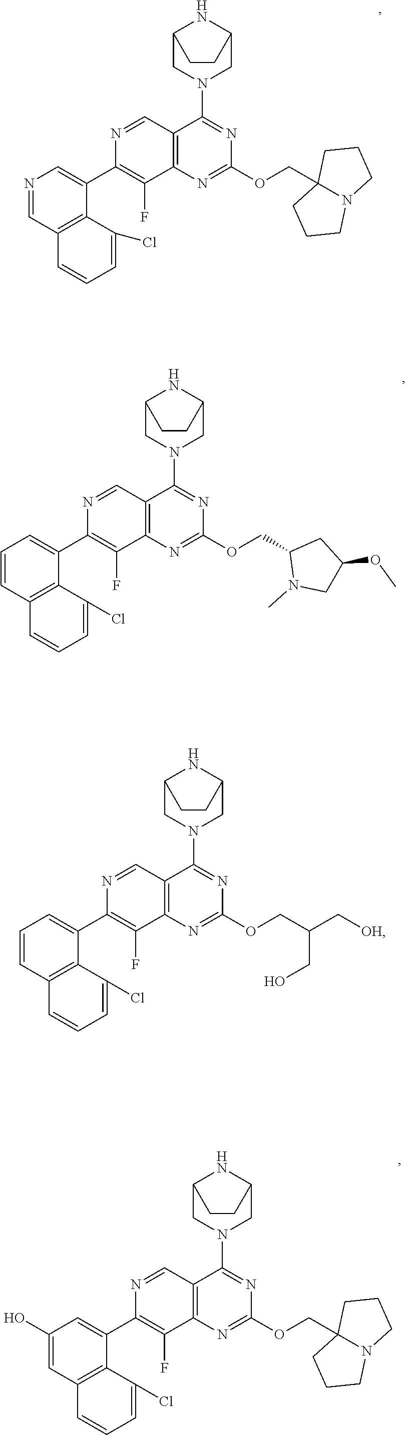 [SMILES: Fc1c(-c2cncc3cccc(Cl)c23)ncc2c(N3CC4CCC(C3)N4)nc(OCC34CCCN3CCC4)nc12 ; CO[C@@H]1C[C@@H](COc2nc(N3CC4CCC(C3)N4)c3cnc(-c4cccc5cccc(Cl)c45)c(F)c3n2)N(C)C1 ; OCC(CO)COc1nc(N2CC3CCC(C2)N3)c2cnc(-c3cccc4cccc(Cl)c34)c(F)c2n1 ; Oc1cc(-c2ncc3c(N4CC5CCC(C4)N5)nc(OCC45CCCN4CCC5)nc3c2F)c2c(Cl)cccc2c1] 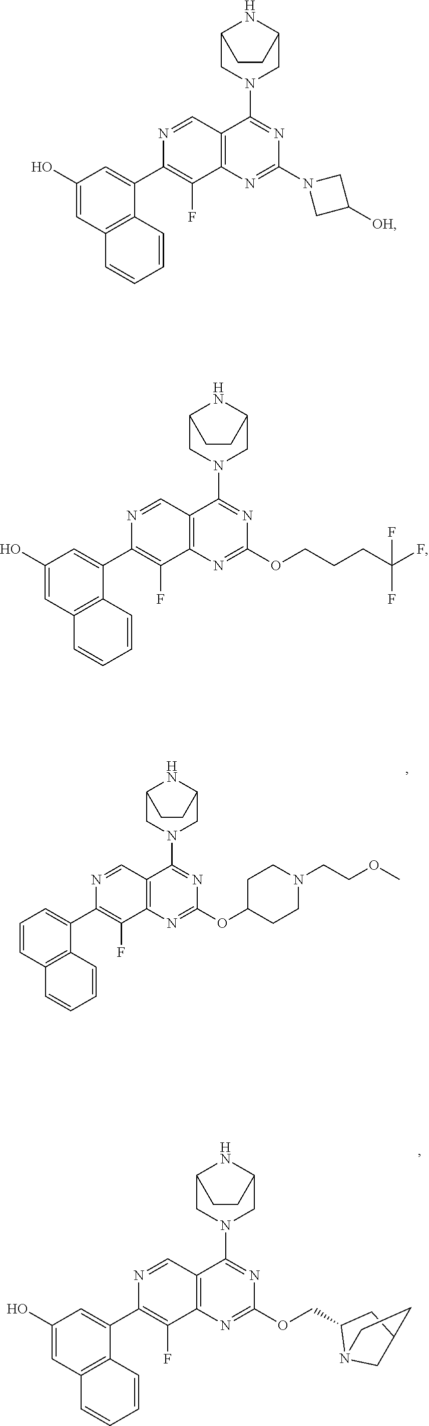 [SMILES: Oc1cc(-c2ncc3c(N4CC5CCC(C4)N5)nc(N4CC(O)C4)nc3c2F)c2ccccc2c1 ; Oc1cc(-c2ncc3c(N4CC5CCC(C4)N5)nc(OCCCC(F)(F)F)nc3c2F)c2ccccc2c1 ; COCCN1CCC(Oc2nc(N3CC4CCC(C3)N4)c3cnc(-c4cccc5ccccc45)c(F)c3n2)CC1 ; Oc1cc(-c2ncc3c(N4CC5CCC(C4)N5)nc(OC[C@@H]4CC5CCN4C5)nc3c2F)c2ccccc2c1] 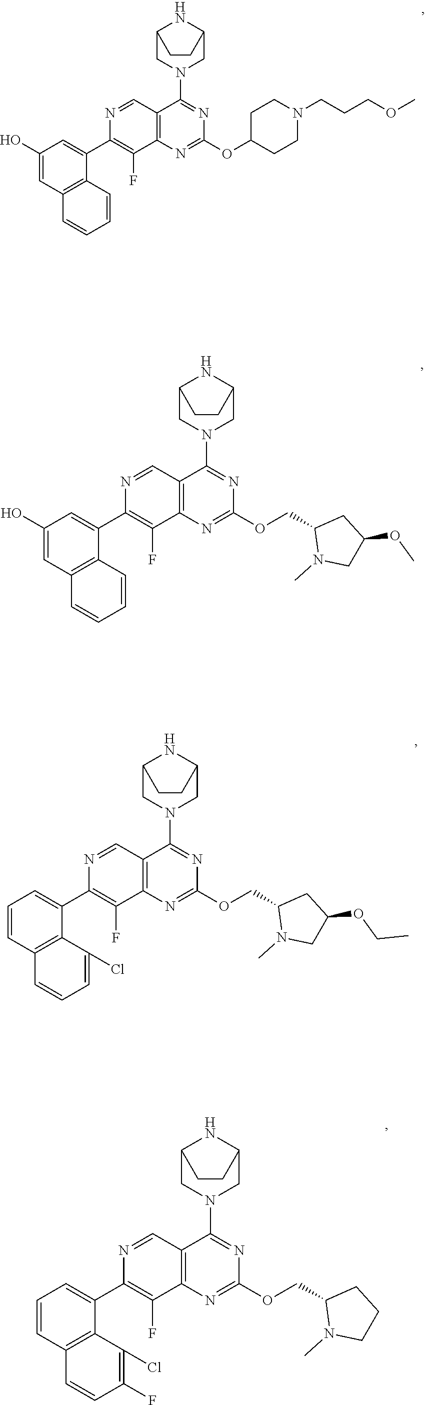 [SMILES: COCCCN1CCC(Oc2nc(N3CC4CCC(C3)N4)c3cnc(-c4cc(O)cc5ccccc45)c(F)c3n2)CC1 ; CO[C@@H]1C[C@@H](COc2nc(N3CC4CCC(C3)N4)c3cnc(-c4cc(O)cc5ccccc45)c(F)c3n2)N(C)C1 ; CCO[C@@H]1C[C@@H](COc2nc(N3CC4CCC(C3)N4)c3cnc(-c4cccc5cccc(Cl)c45)c(F)c3n2)N(C)C1 ; CN1CCC[C@H]1COc1nc(N2CC3CCC(C2)N3)c2cnc(-c3cccc4ccc(F)c(Cl)c34)c(F)c2n1] 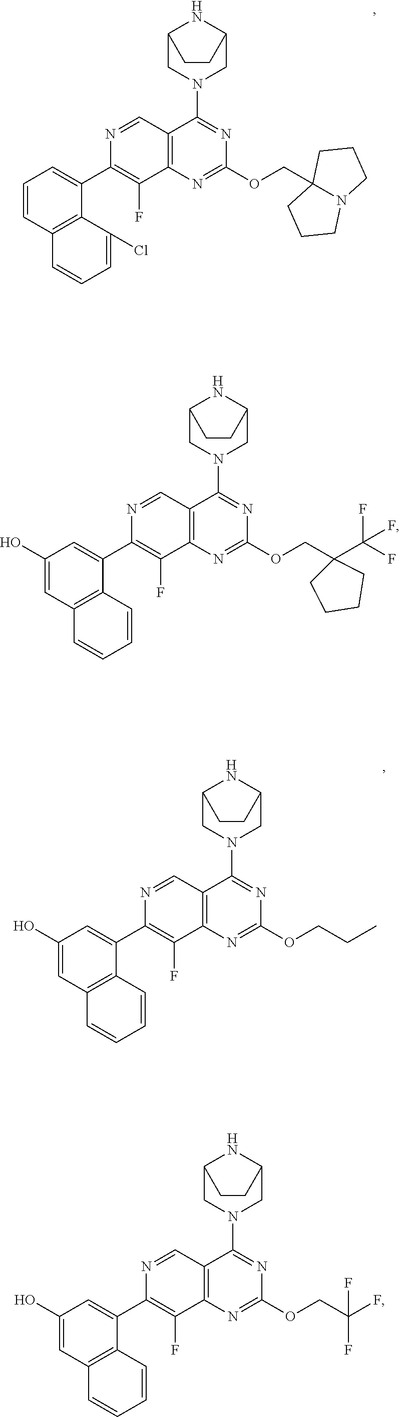 [SMILES: Fc1c(-c2cccc3cccc(Cl)c23)ncc2c(N3CC4CCC(C3)N4)nc(OCC34CCCN3CCC4)nc12 ; Oc1cc(-c2ncc3c(N4CC5CCC(C4)N5)nc(OCC4(C(F)(F)F)CCCC4)nc3c2F)c2ccccc2c1 ; CCCOc1nc(N2CC3CCC(C2)N3)c2cnc(-c3cc(O)cc4ccccc34)c(F)c2n1 ; Oc1cc(-c2ncc3c(N4CC5CCC(C4)N5)nc(OCC(F)(F)F)nc3c2F)c2ccccc2c1] 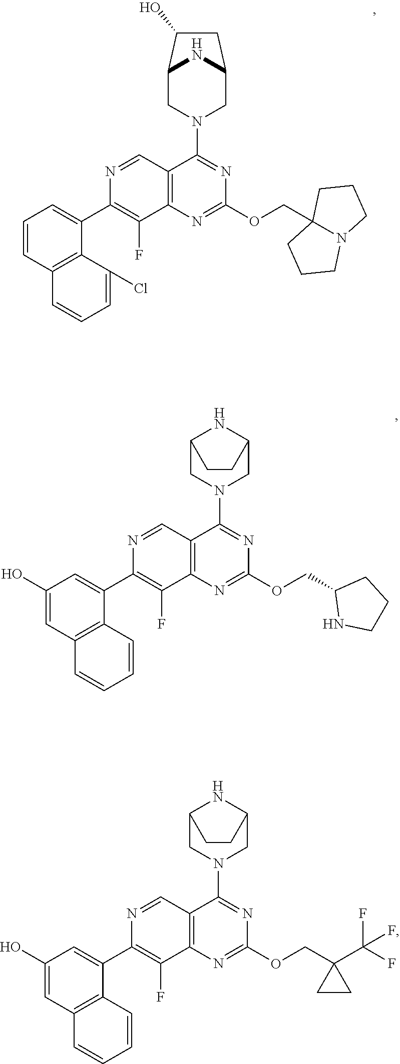 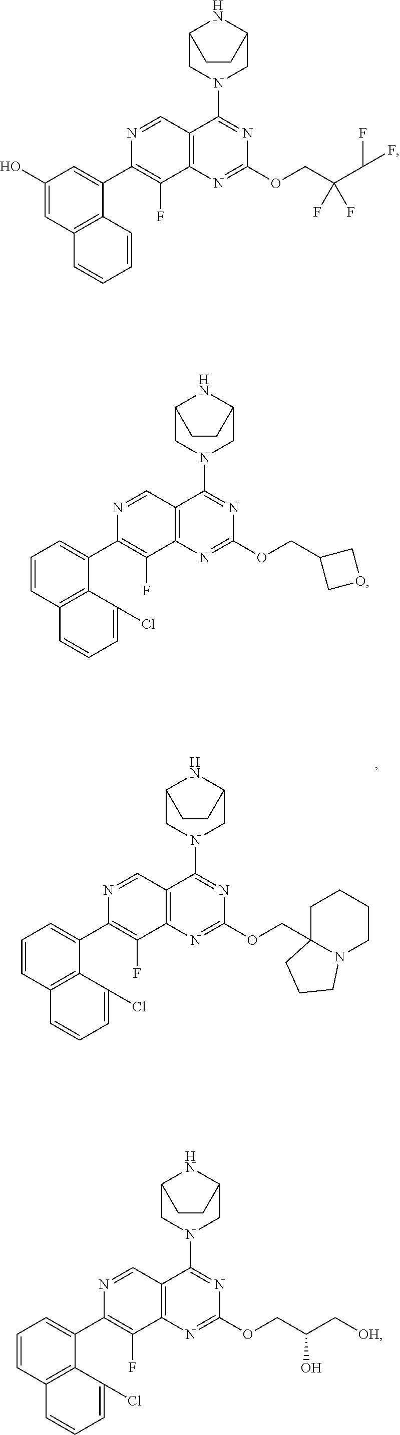 [SMILES: Oc1cc(-c2ncc3c(N4CC5CCC(C4)N5)nc(OCC(F)(F)C(F)F)nc3c2F)c2ccccc2c1 ; Fc1c(-c2cccc3cccc(Cl)c23)ncc2c(N3CC4CCC(C3)N4)nc(OCC3COC3)nc12 ; Fc1c(-c2cccc3cccc(Cl)c23)ncc2c(N3CC4CCC(C3)N4)nc(OCC34CCCCN3CCC4)nc12 ; OC[C@H](O)COc1nc(N2CC3CCC(C2)N3)c2cnc(-c3cccc4cccc(Cl)c34)c(F)c2n1] 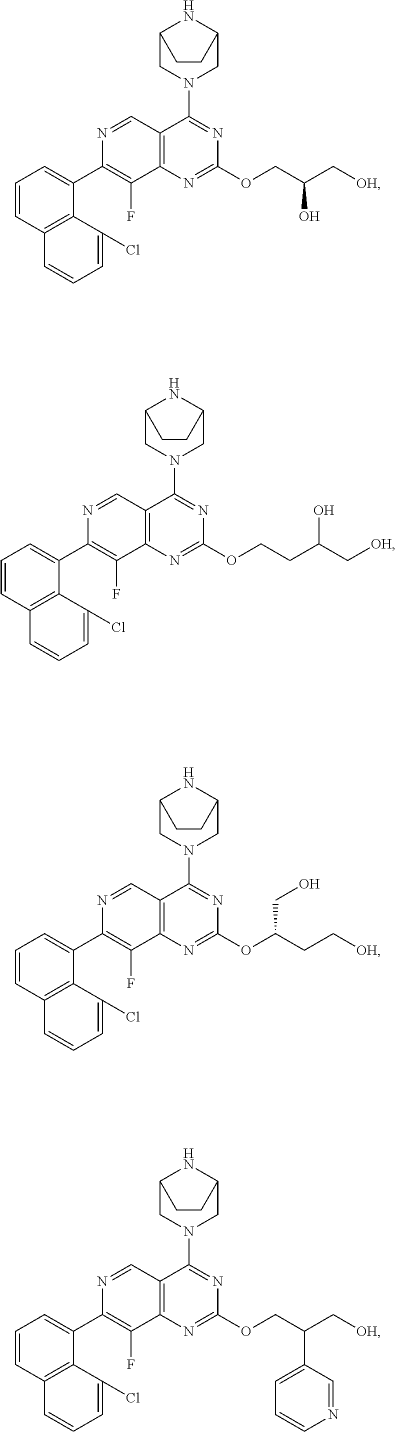 [SMILES: OC[C@@H](O)COc1nc(N2CC3CCC(C2)N3)c2cnc(-c3cccc4cccc(Cl)c34)c(F)c2n1 ; OCC(O)CCOc1nc(N2CC3CCC(C2)N3)c2cnc(-c3cccc4cccc(Cl)c34)c(F)c2n1 ; OCC[C@@H](CO)Oc1nc(N2CC3CCC(C2)N3)c2cnc(-c3cccc4cccc(Cl)c34)c(F)c2n1 ; OCC(COc1nc(N2CC3CCC(C2)N3)c2cnc(-c3cccc4cccc(Cl)c34)c(F)c2n1)c1cccnc1] 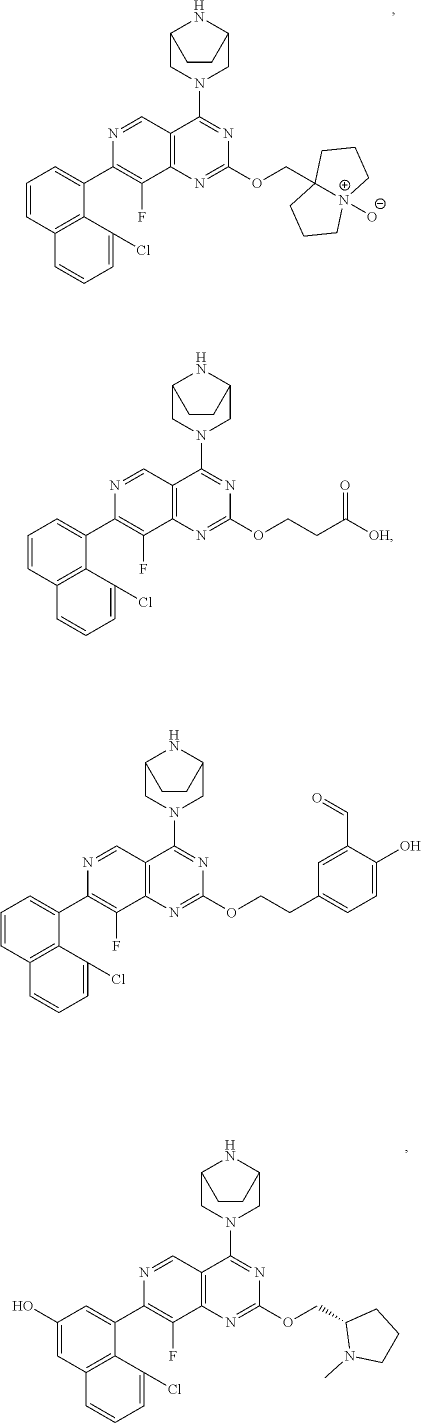 [SMILES: [O-][N+]12CCCC1(COc1nc(N3CC4CCC(C3)N4)c3cnc(-c4cccc5cccc(Cl)c45)c(F)c3n1)CCC2 ; O=C(O)CCOc1nc(N2CC3CCC(C2)N3)c2cnc(-c3cccc4cccc(Cl)c34)c(F)c2n1 ; O=Cc1cc(CCOc2nc(N3CC4CCC(C3)N4)c3cnc(-c4cccc5cccc(Cl)c45)c(F)c3n2)ccc1O ; CN1CCC[C@H]1COc1nc(N2CC3CCC(C2)N3)c2cnc(-c3cc(O)cc4cccc(Cl)c34)c(F)c2n1] 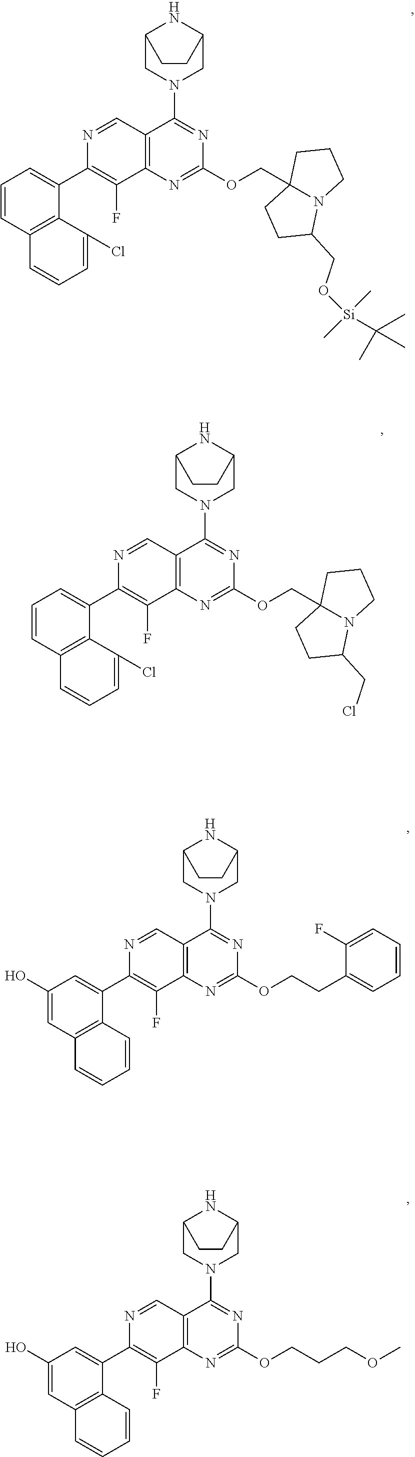 [SMILES: CC(C)(C)[Si](C)(C)OCC1CCC2(COc3nc(N4CC5CCC(C4)N5)c4cnc(-c5cccc6cccc(Cl)c56)c(F)c4n3)CCCN12 ; Fc1c(-c2cccc3cccc(Cl)c23)ncc2c(N3CC4CCC(C3)N4)nc(OCC34CCCN3C(CCl)CC4)nc12 ; Oc1cc(-c2ncc3c(N4CC5CCC(C4)N5)nc(OCCc4ccccc4F)nc3c2F)c2ccccc2c1 ; COCCCOc1nc(N2CC3CCC(C2)N3)c2cnc(-c3cc(O)cc4ccccc34)c(F)c2n1] 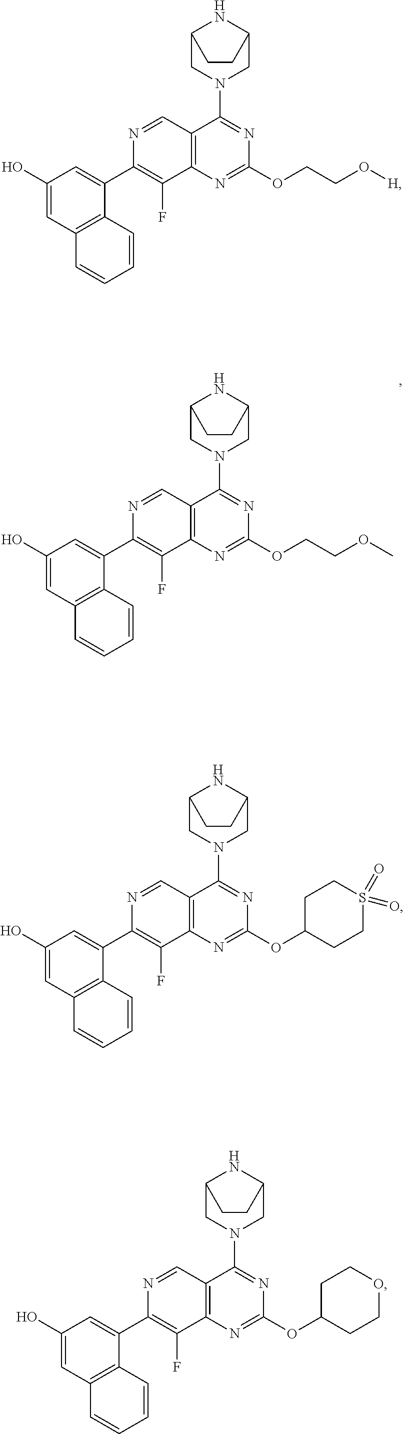 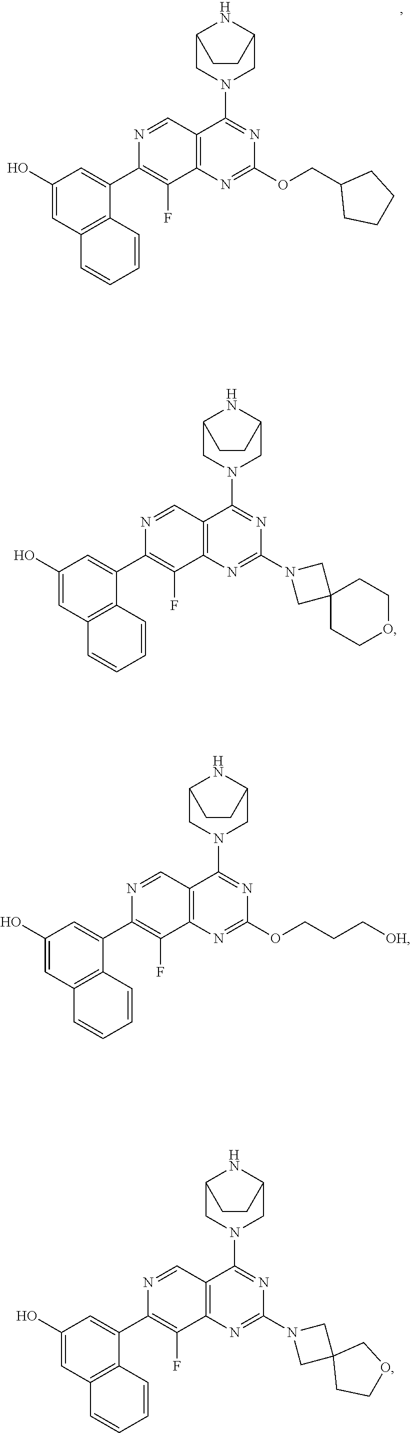 [SMILES: Oc1cc(-c2ncc3c(N4CC5CCC(C4)N5)nc(OCC4CCCC4)nc3c2F)c2ccccc2c1 ; Oc1cc(-c2ncc3c(N4CC5CCC(C4)N5)nc(N4CC5(CCOCC5)C4)nc3c2F)c2ccccc2c1 ; OCCCOc1nc(N2CC3CCC(C2)N3)c2cnc(-c3cc(O)cc4ccccc34)c(F)c2n1 ; Oc1cc(-c2ncc3c(N4CC5CCC(C4)N5)nc(N4CC5(CCOC5)C4)nc3c2F)c2ccccc2c1] 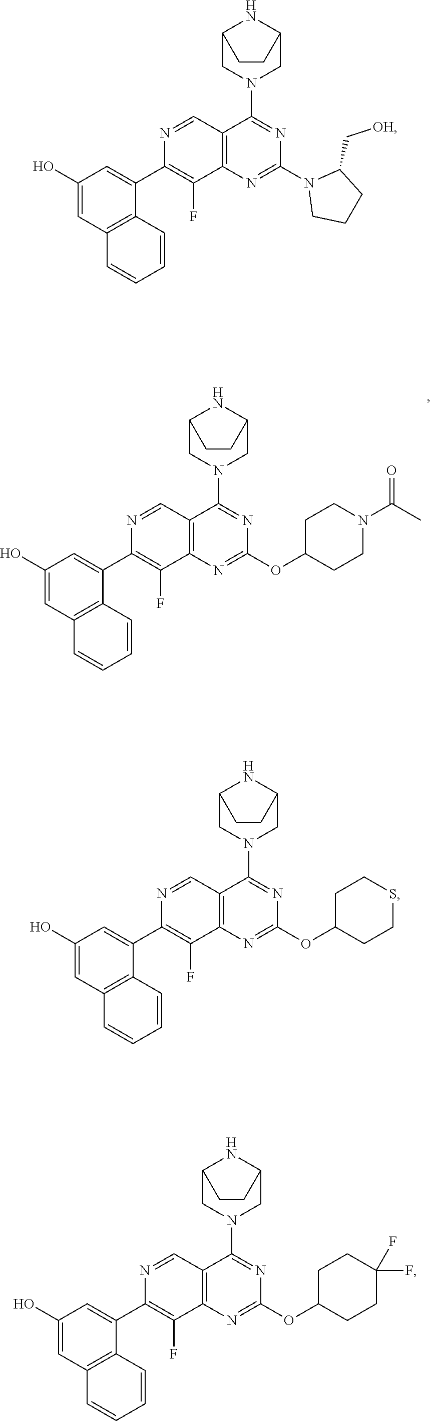 [SMILES: OC[C@@H]1CCCN1c1nc(N2CC3CCC(C2)N3)c2cnc(-c3cc(O)cc4ccccc34)c(F)c2n1 ; CC(=O)N1CCC(Oc2nc(N3CC4CCC(C3)N4)c3cnc(-c4cc(O)cc5ccccc45)c(F)c3n2)CC1 ; Oc1cc(-c2ncc3c(N4CC5CCC(C4)N5)nc(OC4CCSCC4)nc3c2F)c2ccccc2c1 ; Oc1cc(-c2ncc3c(N4CC5CCC(C4)N5)nc(OC4CCC(F)(F)CC4)nc3c2F)c2ccccc2c1] 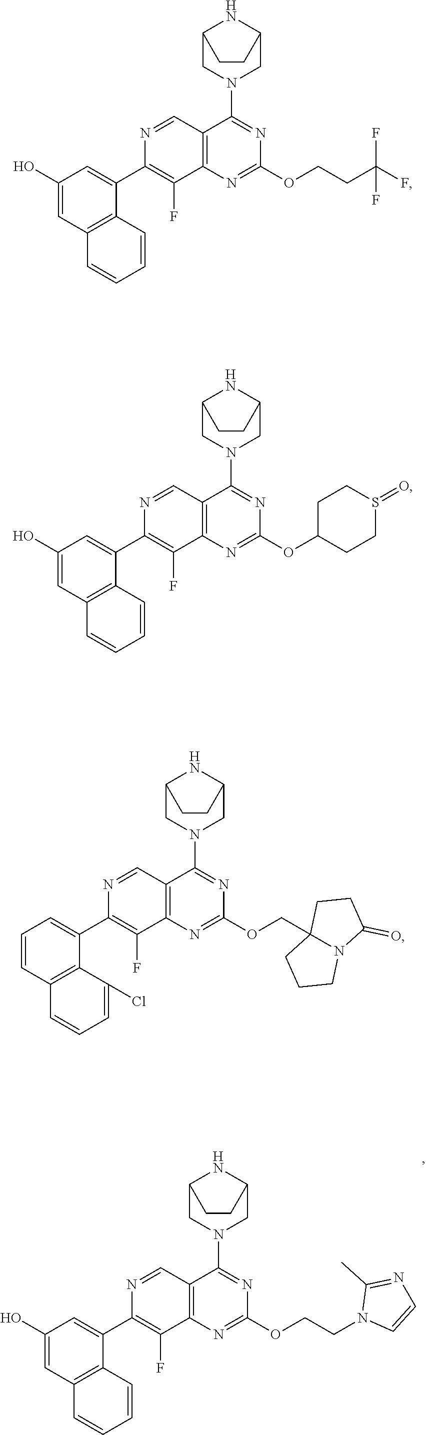 [SMILES: Oc1cc(-c2ncc3c(N4CC5CCC(C4)N5)nc(OCCC(F)(F)F)nc3c2F)c2ccccc2c1 ; O=S1CCC(Oc2nc(N3CC4CCC(C3)N4)c3cnc(-c4cc(O)cc5ccccc45)c(F)c3n2)CC1 ; O=C1CCC2(COc3nc(N4CC5CCC(C4)N5)c4cnc(-c5cccc6cccc(Cl)c56)c(F)c4n3)CCCN12 ; Cc1nccn1CCOc1nc(N2CC3CCC(C2)N3)c2cnc(-c3cc(O)cc4ccccc34)c(F)c2n1] 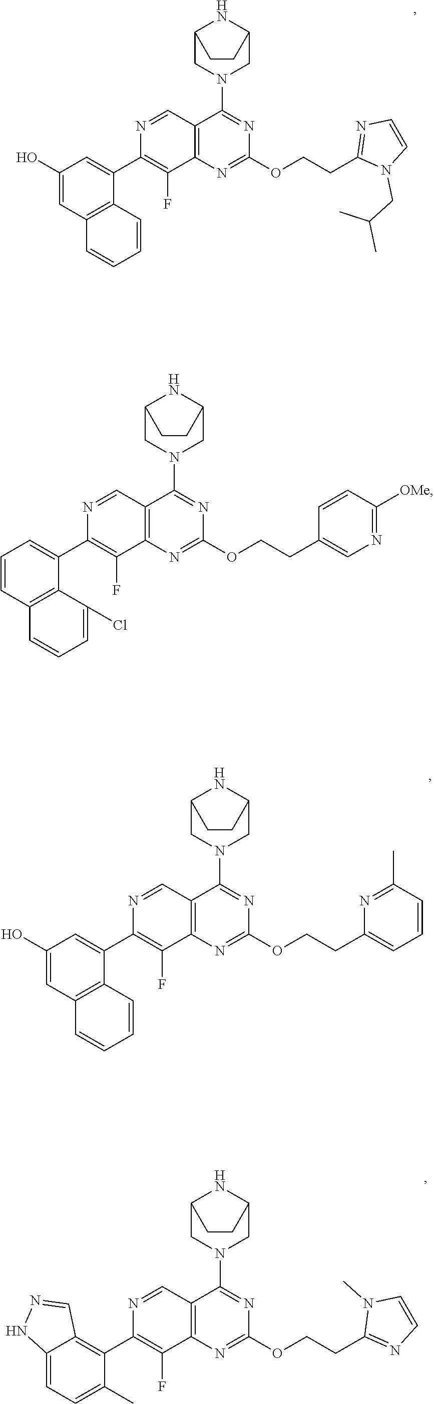 [SMILES: CC(C)Cn1ccnc1CCOc1nc(N2CC3CCC(C2)N3)c2cnc(-c3cc(O)cc4ccccc34)c(F)c2n1 ; COc1ccc(CCOc2nc(N3CC4CCC(C3)N4)c3cnc(-c4cccc5cccc(Cl)c45)c(F)c3n2)cn1 ; Cc1cccc(CCOc2nc(N3CC4CCC(C3)N4)c3cnc(-c4cc(O)cc5ccccc45)c(F)c3n2)n1 ; Cc1ccc2[nH]ncc2c1-c1ncc2c(N3CC4CCC(C3)N4)nc(OCCc3nccn3C)nc2c1F] 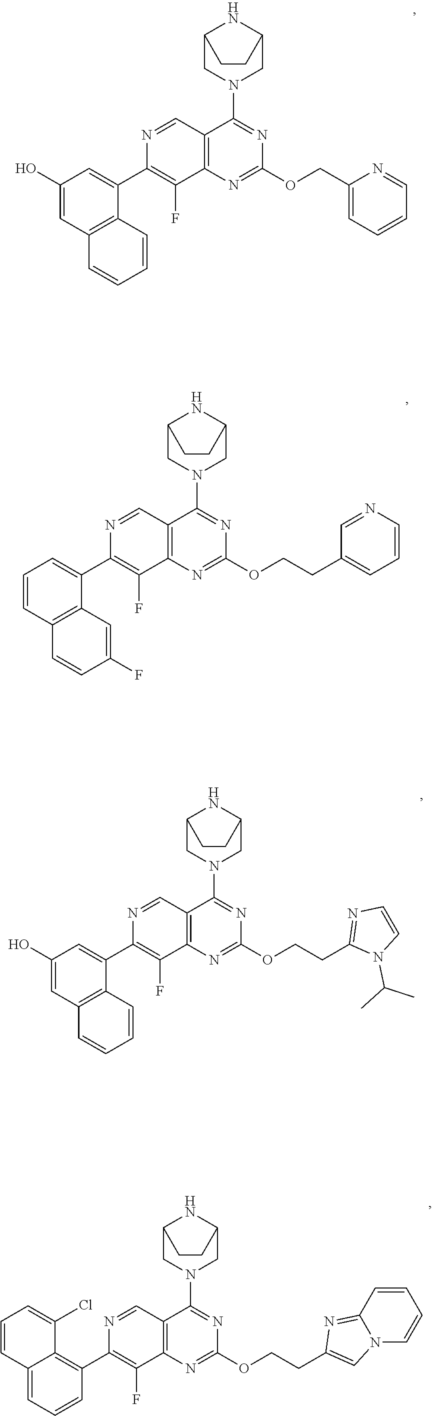 [SMILES: Oc1cc(-c2ncc3c(N4CC5CCC(C4)N5)nc(OCc4ccccn4)nc3c2F)c2ccccc2c1 ; Fc1ccc2cccc(-c3ncc4c(N5CC6CCC(C5)N6)nc(OCCc5cccnc5)nc4c3F)c2c1 ; CC(C)n1ccnc1CCOc1nc(N2CC3CCC(C2)N3)c2cnc(-c3cc(O)cc4ccccc34)c(F)c2n1 ; Fc1c(-c2cccc3cccc(Cl)c23)ncc2c(N3CC4CCC(C3)N4)nc(OCCc3cn4ccccc4n3)nc12] 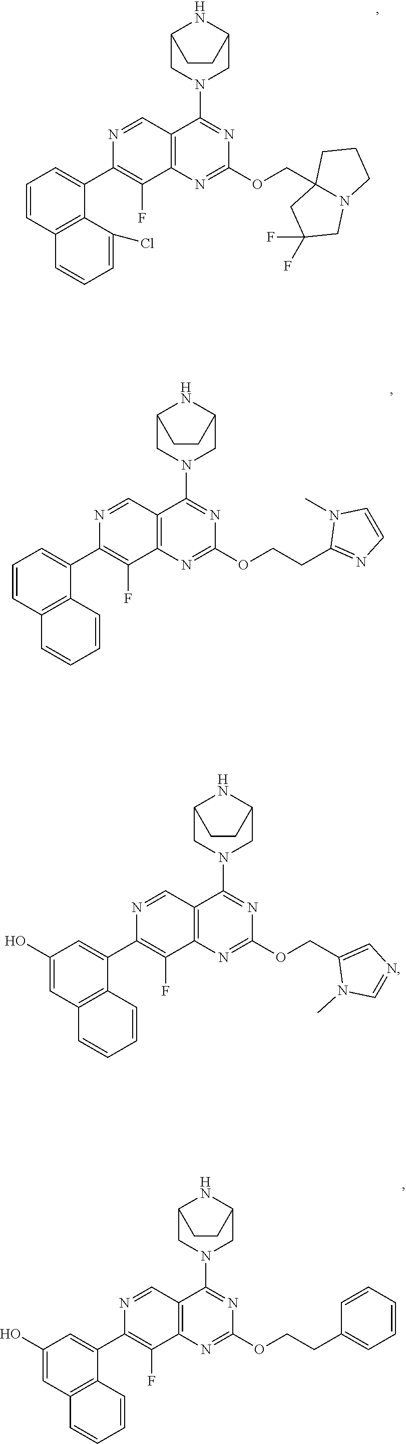 [SMILES: Fc1c(-c2cccc3cccc(Cl)c23)ncc2c(N3CC4CCC(C3)N4)nc(OCC34CCCN3CC(F)(F)C4)nc12 ; Cn1ccnc1CCOc1nc(N2CC3CCC(C2)N3)c2cnc(-c3cccc4ccccc34)c(F)c2n1 ; Cn1cncc1COc1nc(N2CC3CCC(C2)N3)c2cnc(-c3cc(O)cc4ccccc34)c(F)c2n1 ; Oc1cc(-c2ncc3c(N4CC5CCC(C4)N5)nc(OCCc4ccccc4)nc3c2F)c2ccccc2c1] 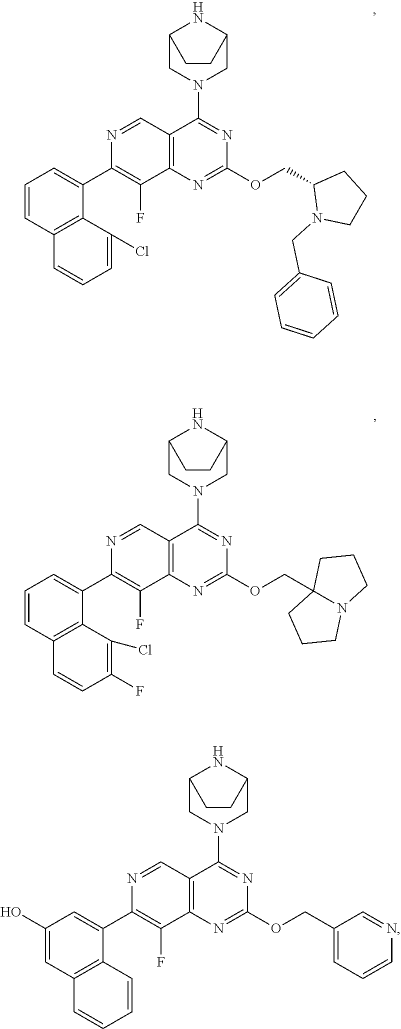 [SMILES: Fc1c(-c2cccc3cccc(Cl)c23)ncc2c(N3CC4CCC(C3)N4)nc(OC[C@@H]3CCCN3Cc3ccccc3)nc12 ; Fc1ccc2cccc(-c3ncc4c(N5CC6CCC(C5)N6)nc(OCC56CCCN5CCC6)nc4c3F)c2c1Cl ; Oc1cc(-c2ncc3c(N4CC5CCC(C4)N5)nc(OCc4cccnc4)nc3c2F)c2ccccc2c1] 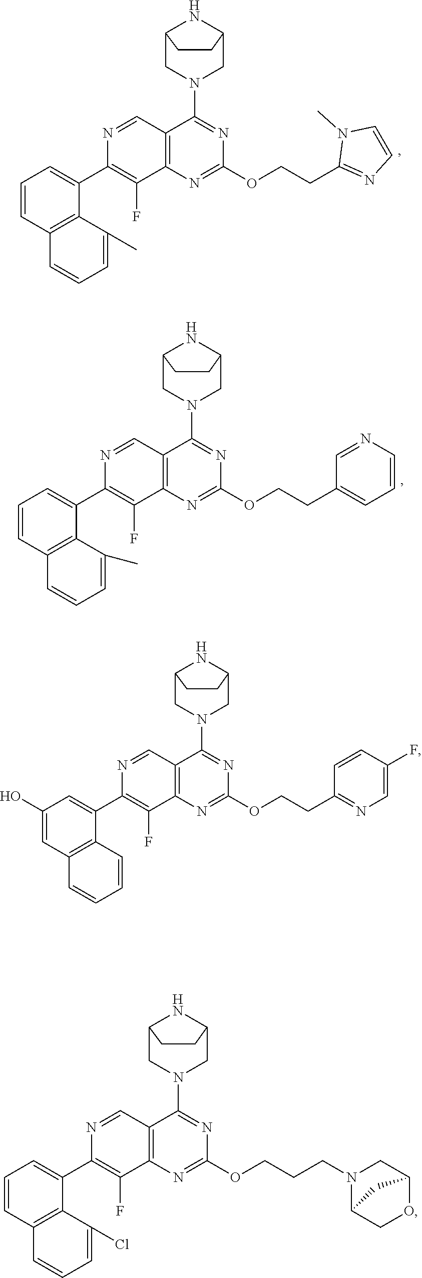 [SMILES: Cc1cccc2cccc(-c3ncc4c(N5CC6CCC(C5)N6)nc(OCCc5nccn5C)nc4c3F)c12 ; Cc1cccc2cccc(-c3ncc4c(N5CC6CCC(C5)N6)nc(OCCc5cccnc5)nc4c3F)c12 ; Oc1cc(-c2ncc3c(N4CC5CCC(C4)N5)nc(OCCc4ccc(F)cn4)nc3c2F)c2ccccc2c1 ; Fc1c(-c2cccc3cccc(Cl)c23)ncc2c(N3CC4CCC(C3)N4)nc(OCCCN3C[C@@H]4C[C@H]3CO4)nc12] 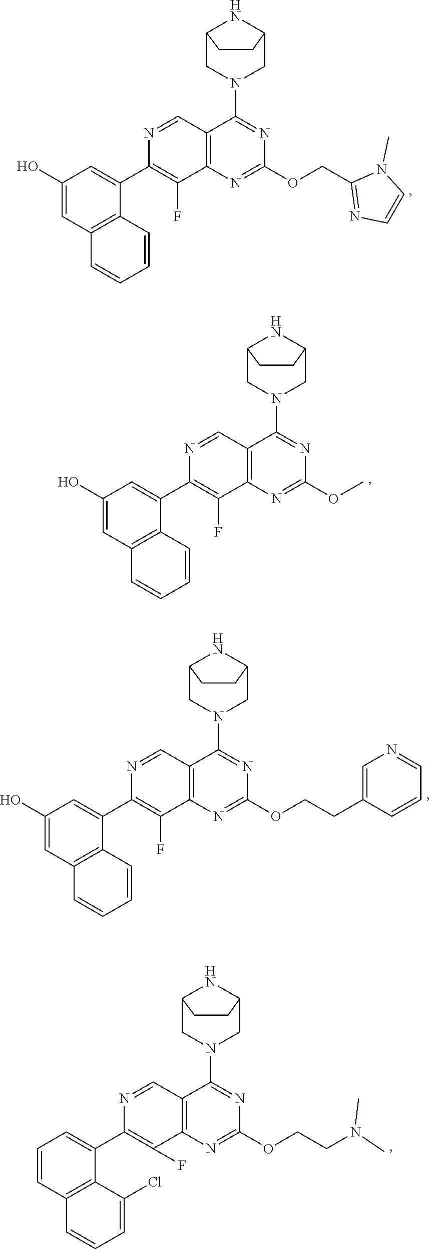 [SMILES: Cn1ccnc1COc1nc(N2CC3CCC(C2)N3)c2cnc(-c3cc(O)cc4ccccc34)c(F)c2n1 ; COc1nc(N2CC3CCC(C2)N3)c2cnc(-c3cc(O)cc4ccccc34)c(F)c2n1 ; Oc1cc(-c2ncc3c(N4CC5CCC(C4)N5)nc(OCCc4cccnc4)nc3c2F)c2ccccc2c1 ; CN(C)CCOc1nc(N2CC3CCC(C2)N3)c2cnc(-c3cccc4cccc(Cl)c34)c(F)c2n1] 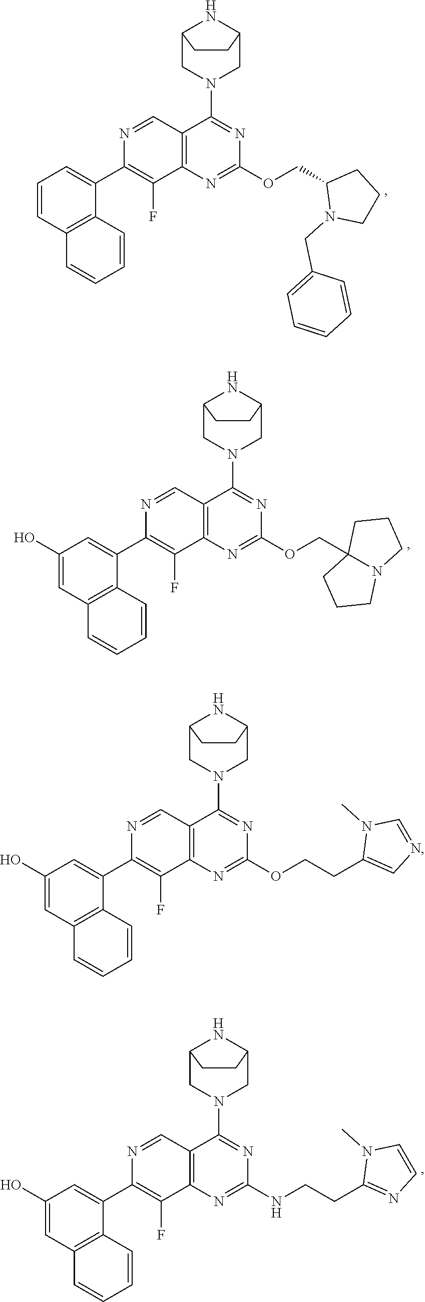 [SMILES: Fc1c(-c2cccc3ccccc23)ncc2c(N3CC4CCC(C3)N4)nc(OC[C@@H]3CCCN3Cc3ccccc3)nc12 ; Oc1cc(-c2ncc3c(N4CC5CCC(C4)N5)nc(OCC45CCCN4CCC5)nc3c2F)c2ccccc2c1 ; Cn1cncc1CCOc1nc(N2CC3CCC(C2)N3)c2cnc(-c3cc(O)cc4ccccc34)c(F)c2n1 ; Cn1ccnc1CCNc1nc(N2CC3CCC(C2)N3)c2cnc(-c3cc(O)cc4ccccc34)c(F)c2n1] 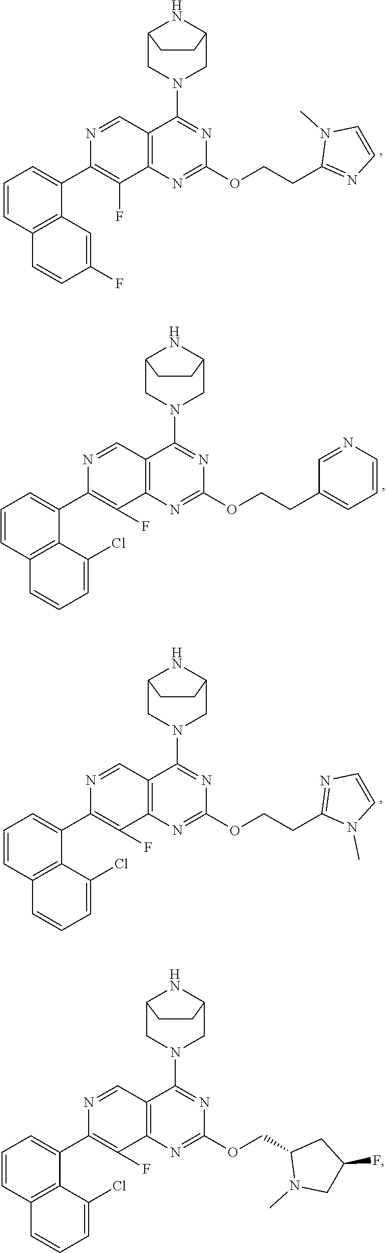 [SMILES: Cn1ccnc1CCOc1nc(N2CC3CCC(C2)N3)c2cnc(-c3cccc4ccc(F)cc34)c(F)c2n1 ; Fc1c(-c2cccc3cccc(Cl)c23)ncc2c(N3CC4CCC(C3)N4)nc(OCCc3cccnc3)nc12 ; Cn1ccnc1CCOc1nc(N2CC3CCC(C2)N3)c2cnc(-c3cccc4cccc(Cl)c34)c(F)c2n1 ; CN1C[C@H](F)C[C@H]1COc1nc(N2CC3CCC(C2)N3)c2cnc(-c3cccc4cccc(Cl)c34)c(F)c2n1] 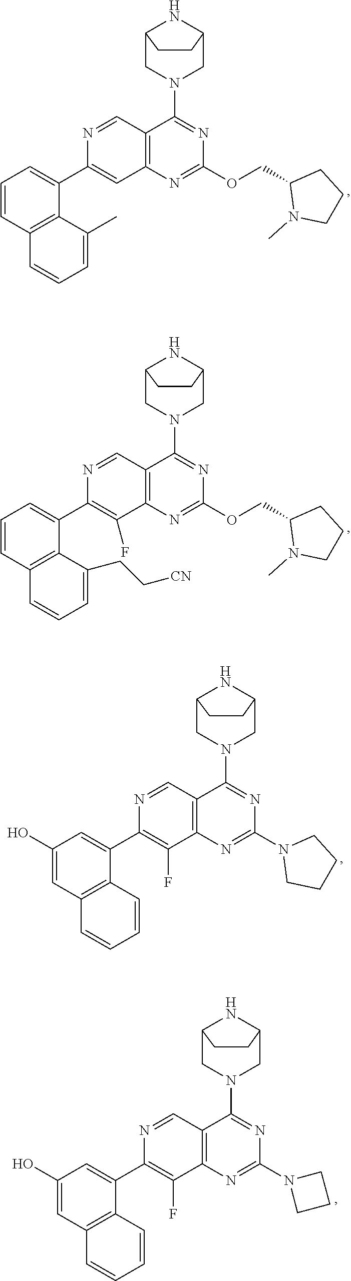 [SMILES: Cc1cccc2cccc(-c3cc4nc(OC[C@@H]5CCCN5C)nc(N5CC6CCC(C5)N6)c4cn3)c12 ; CN1CCC[C@H]1COc1nc(N2CC3CCC(C2)N3)c2cnc(-c3cccc4cccc(CCC#N)c34)c(F)c2n1 ; Oc1cc(-c2ncc3c(N4CC5CCC(C4)N5)nc(N4CCCC4)nc3c2F)c2ccccc2c1 ; Oc1cc(-c2ncc3c(N4CC5CCC(C4)N5)nc(N4CCC4)nc3c2F)c2ccccc2c1] 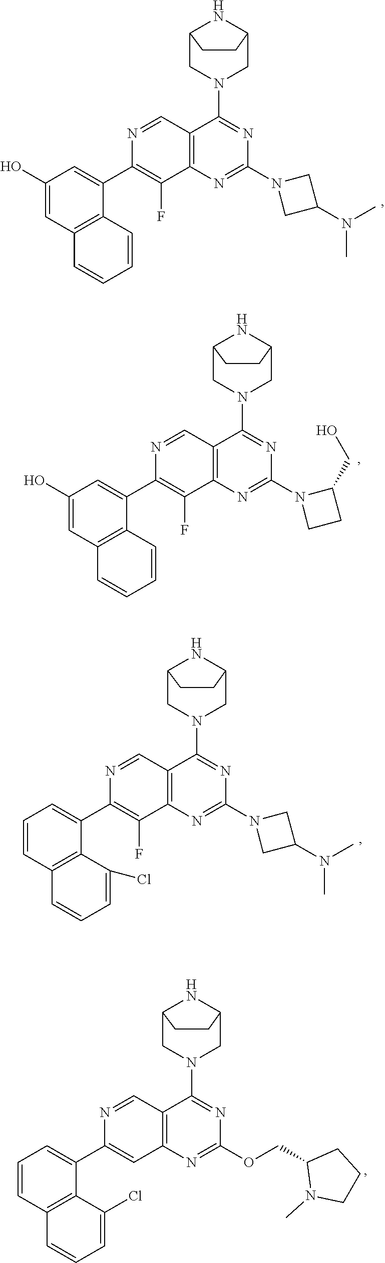 [SMILES: CN(C)C1CN(c2nc(N3CC4CCC(C3)N4)c3cnc(-c4cc(O)cc5ccccc45)c(F)c3n2)C1 ; OC[C@@H]1CCN1c1nc(N2CC3CCC(C2)N3)c2cnc(-c3cc(O)cc4ccccc34)c(F)c2n1 ; CN(C)C1CN(c2nc(N3CC4CCC(C3)N4)c3cnc(-c4cccc5cccc(Cl)c45)c(F)c3n2)C1 ; CN1CCC[C@H]1COc1nc(N2CC3CCC(C2)N3)c2cnc(-c3cccc4cccc(Cl)c34)cc2n1] 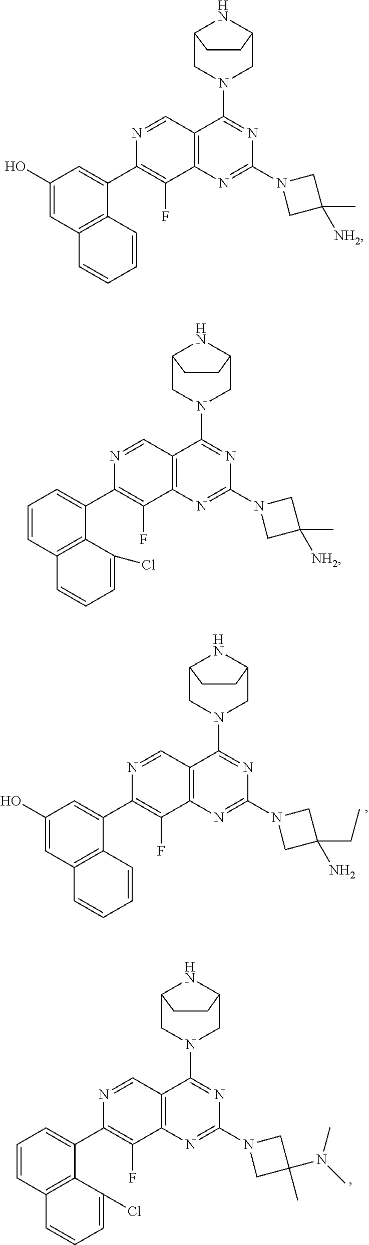 [SMILES: CC1(N)CN(c2nc(N3CC4CCC(C3)N4)c3cnc(-c4cc(O)cc5ccccc45)c(F)c3n2)C1 ; CC1(N)CN(c2nc(N3CC4CCC(C3)N4)c3cnc(-c4cccc5cccc(Cl)c45)c(F)c3n2)C1 ; CCC1(N)CN(c2nc(N3CC4CCC(C3)N4)c3cnc(-c4cc(O)cc5ccccc45)c(F)c3n2)C1 ; CN(C)C1(C)CN(c2nc(N3CC4CCC(C3)N4)c3cnc(-c4cccc5cccc(Cl)c45)c(F)c3n2)C1] 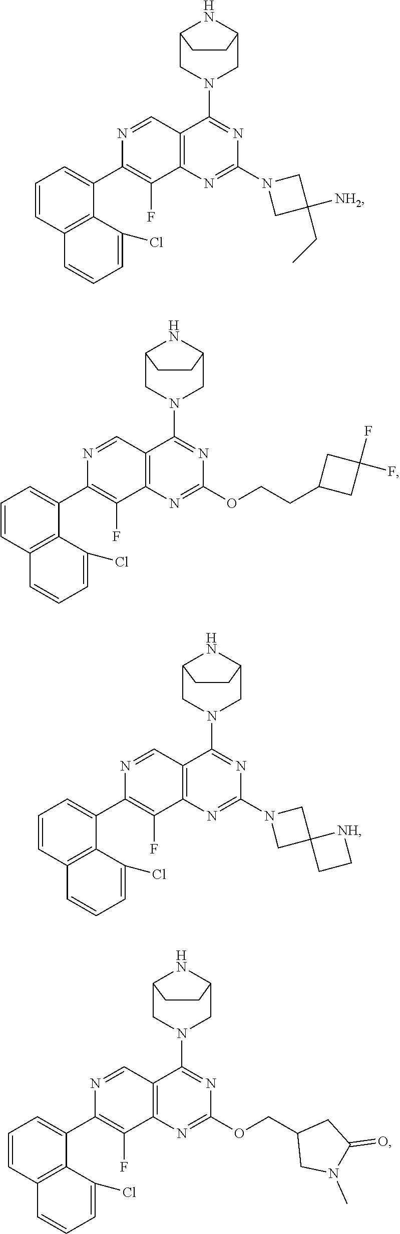 [SMILES: CCC1(N)CN(c2nc(N3CC4CCC(C3)N4)c3cnc(-c4cccc5cccc(Cl)c45)c(F)c3n2)C1 ; Fc1c(-c2cccc3cccc(Cl)c23)ncc2c(N3CC4CCC(C3)N4)nc(OCCC3CC(F)(F)C3)nc12 ; Fc1c(-c2cccc3cccc(Cl)c23)ncc2c(N3CC4CCC(C3)N4)nc(N3CC4(CCN4)C3)nc12 ; CN1CC(COc2nc(N3CC4CCC(C3)N4)c3cnc(-c4cccc5cccc(Cl)c45)c(F)c3n2)CC1=O] 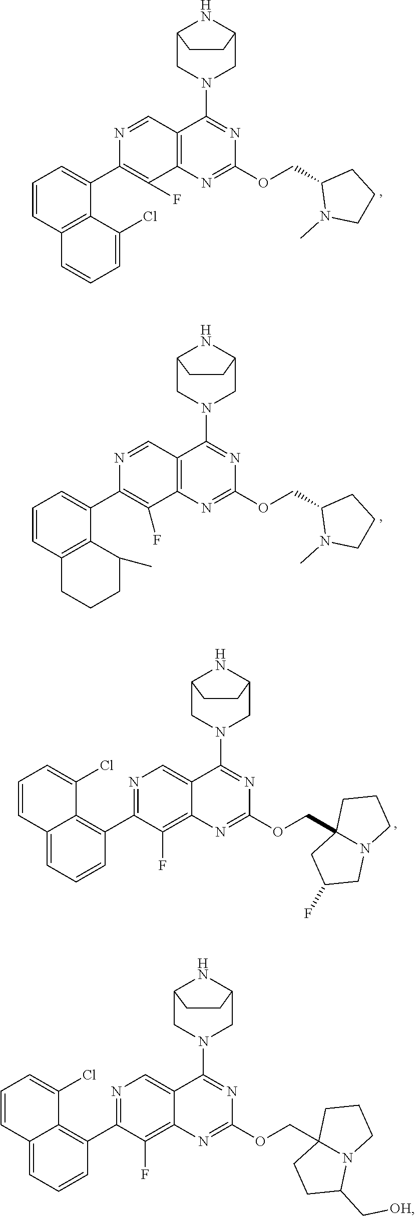 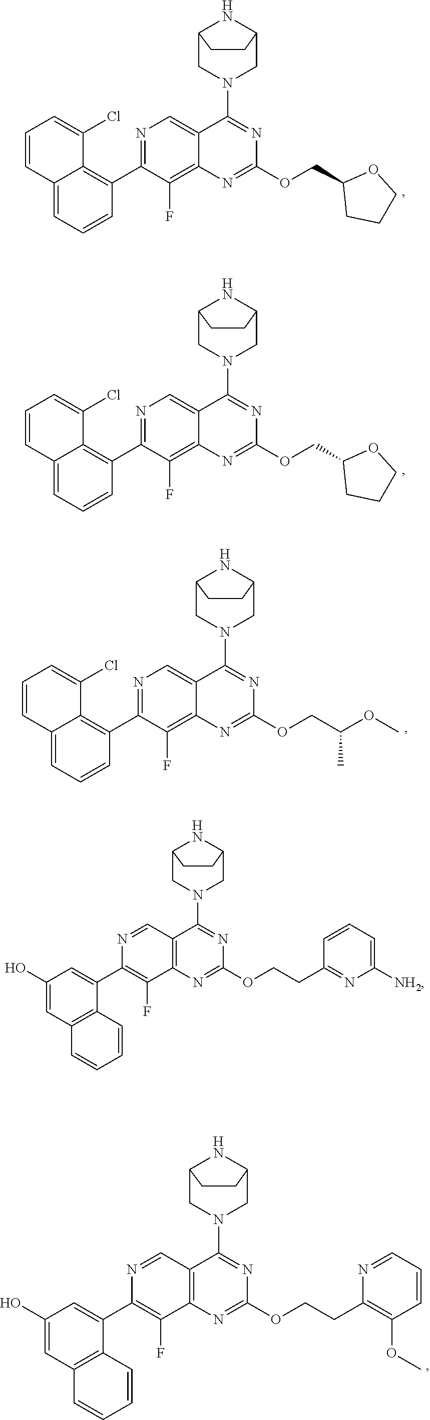 [SMILES: Fc1c(-c2cccc3cccc(Cl)c23)ncc2c(N3CC4CCC(C3)N4)nc(OC[C@@H]3CCCO3)nc12 ; Fc1c(-c2cccc3cccc(Cl)c23)ncc2c(N3CC4CCC(C3)N4)nc(OC[C@H]3CCCO3)nc12 ; CO[C@H](C)COc1nc(N2CC3CCC(C2)N3)c2cnc(-c3cccc4cccc(Cl)c34)c(F)c2n1 ; Nc1cccc(CCOc2nc(N3CC4CCC(C3)N4)c3cnc(-c4cc(O)cc5ccccc45)c(F)c3n2)n1 ; COc1cccnc1CCOc1nc(N2CC3CCC(C2)N3)c2cnc(-c3cc(O)cc4ccccc34)c(F)c2n1] 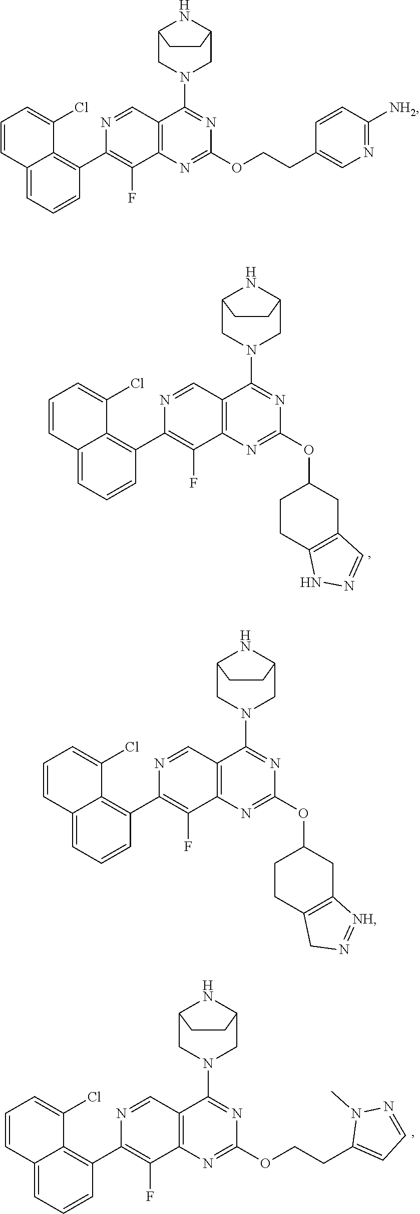 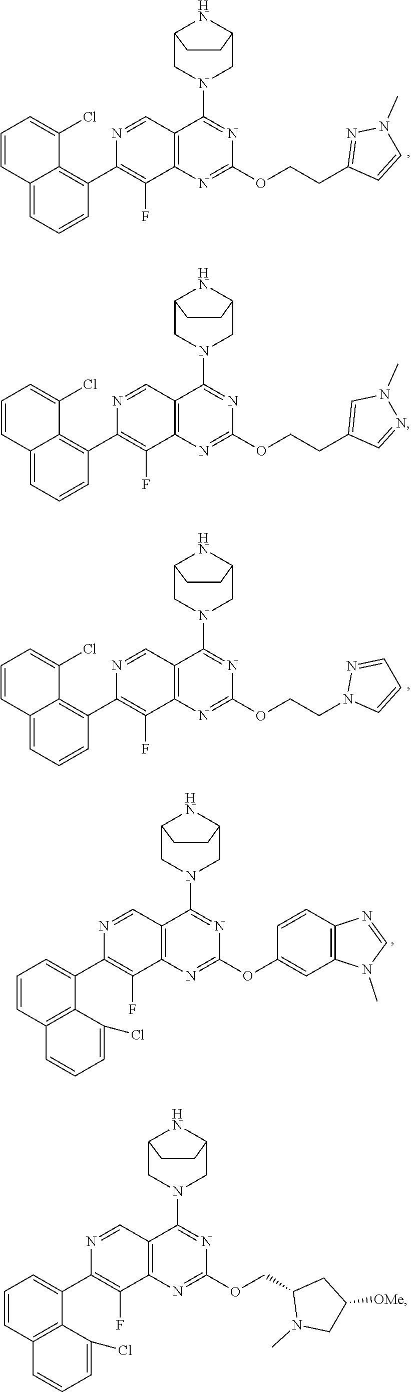 [SMILES: Cn1ccc(CCOc2nc(N3CC4CCC(C3)N4)c3cnc(-c4cccc5cccc(Cl)c45)c(F)c3n2)n1 ; Cn1cc(CCOc2nc(N3CC4CCC(C3)N4)c3cnc(-c4cccc5cccc(Cl)c45)c(F)c3n2)cn1 ; Fc1c(-c2cccc3cccc(Cl)c23)ncc2c(N3CC4CCC(C3)N4)nc(OCCn3cccn3)nc12 ; Cn1cnc2ccc(Oc3nc(N4CC5CCC(C4)N5)c4cnc(-c5cccc6cccc(Cl)c56)c(F)c4n3)cc21 ; CO[C@H]1C[C@@H](COc2nc(N3CC4CCC(C3)N4)c3cnc(-c4cccc5cccc(Cl)c45)c(F)c3n2)N(C)C1] 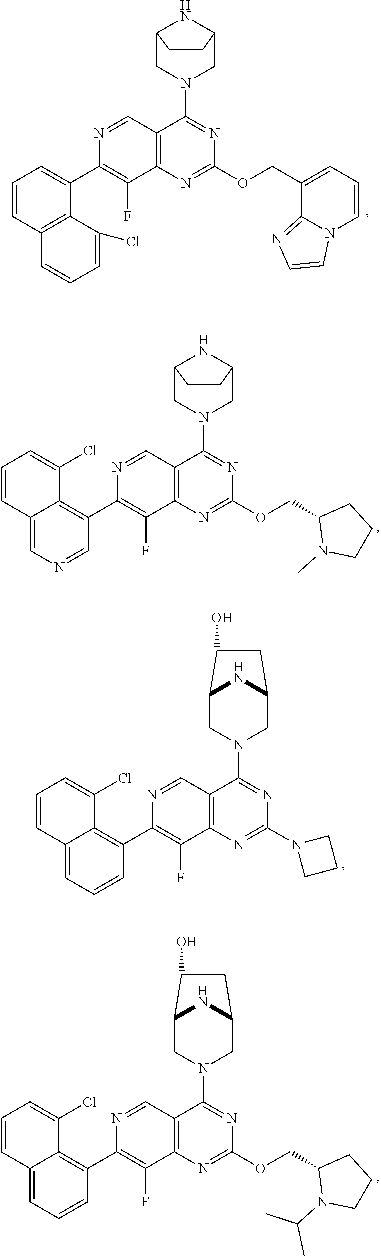 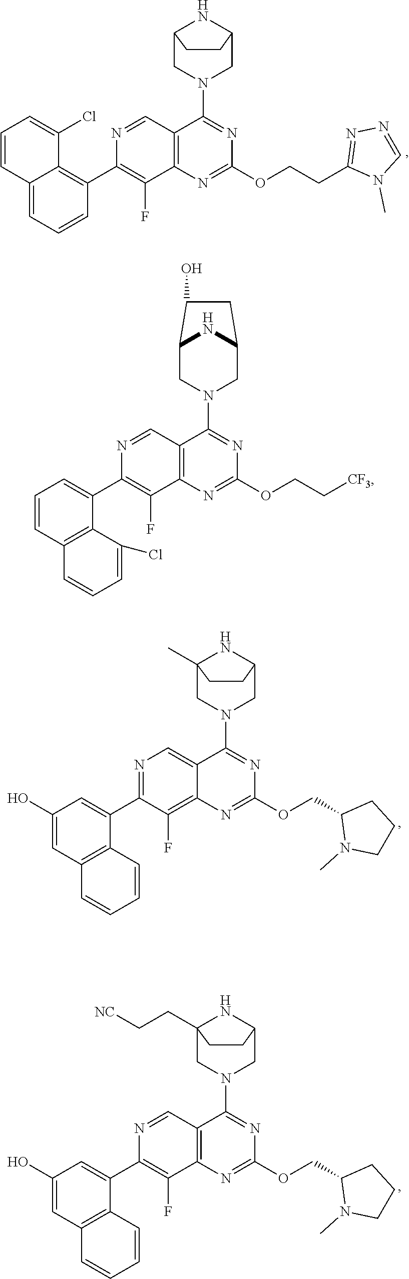 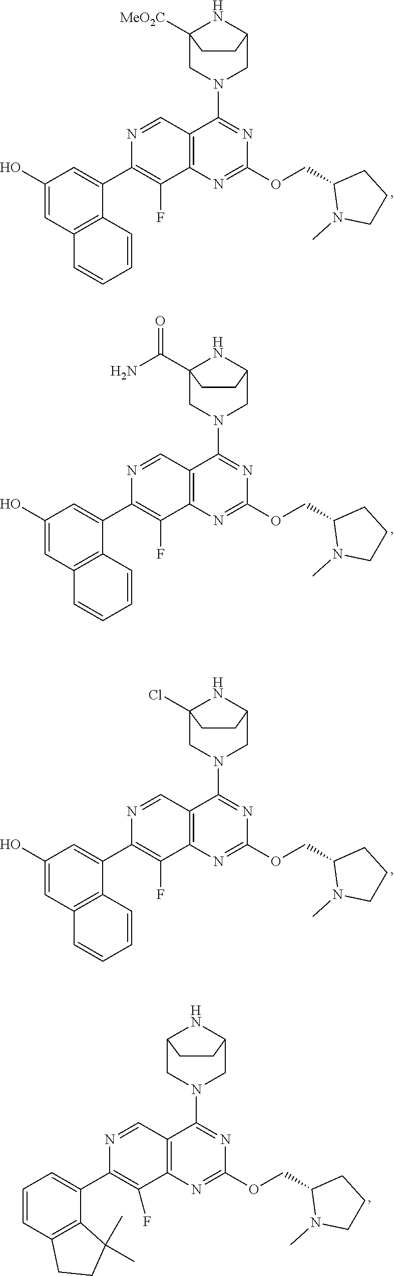 [SMILES: COC(=O)C12CCC(CN(c3nc(OC[C@@H]4CCCN4C)nc4c(F)c(-c5cc(O)cc6ccccc56)ncc34)C1)N2 ; CN1CCC[C@H]1COc1nc(N2CC3CCC(C(N)=O)(C2)N3)c2cnc(-c3cc(O)cc4ccccc34)c(F)c2n1 ; CN1CCC[C@H]1COc1nc(N2CC3CCC(Cl)(C2)N3)c2cnc(-c3cc(O)cc4ccccc34)c(F)c2n1 ; CN1CCC[C@H]1COc1nc(N2CC3CCC(C2)N3)c2cnc(-c3cccc4c3C(C)(C)CC4)c(F)c2n1] 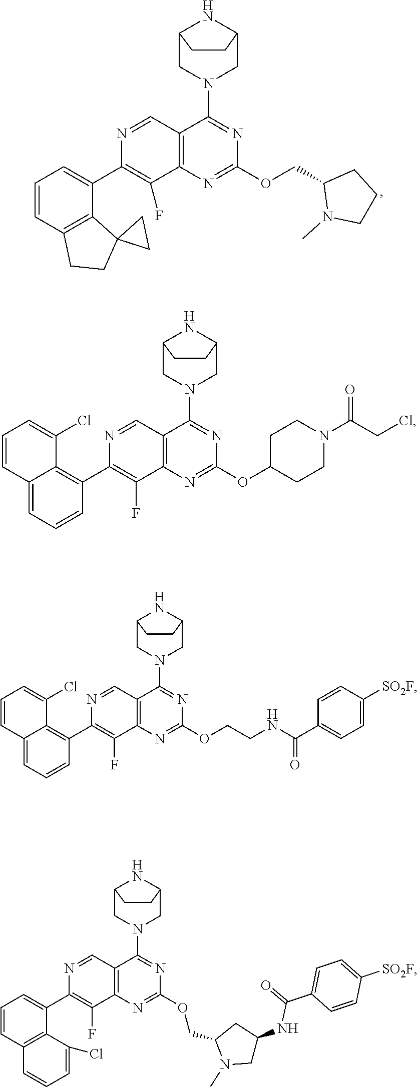 [SMILES: CN1CCC[C@H]1COc1nc(N2CC3CCC(C2)N3)c2cnc(-c3cccc4c3C3(CC4)CC3)c(F)c2n1 ; O=C(CCl)N1CCC(Oc2nc(N3CC4CCC(C3)N4)c3cnc(-c4cccc5cccc(Cl)c45)c(F)c3n2)CC1 ; O=C(NCCOc1nc(N2CC3CCC(C2)N3)c2cnc(-c3cccc4cccc(Cl)c34)c(F)c2n1)c1ccc(S(=O)(=O)F)cc1 ; CN1C[C@H](NC(=O)c2ccc(S(=O)(=O)F)cc2)C[C@H]1COc1nc(N2CC3CCC(C2)N3)c2cnc(-c3cccc4cccc(Cl)c34)c(F)c2n1] 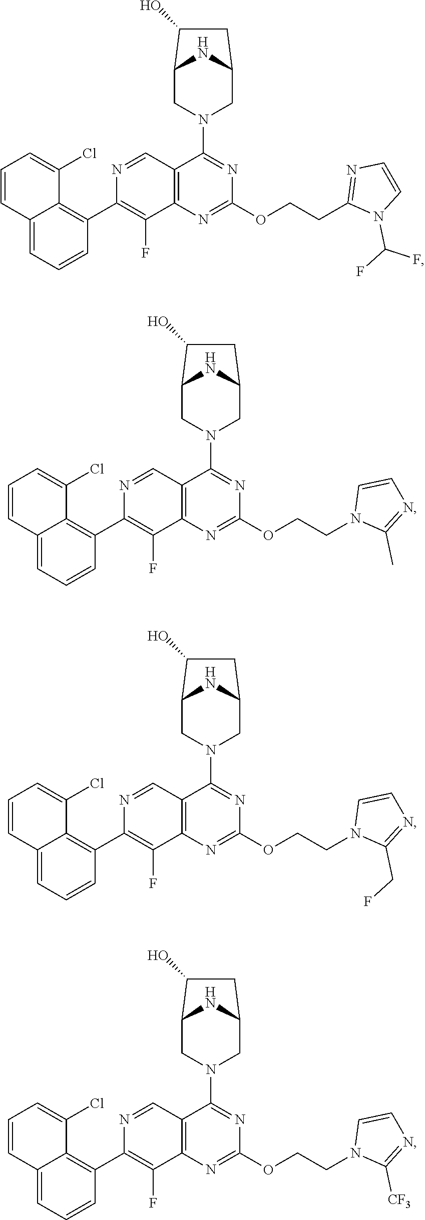 [SMILES: O[C@@H]1C[C@@H]2CN(c3nc(OCCc4nccn4C(F)F)nc4c(F)c(-c5cccc6cccc(Cl)c56)ncc34)C[C@H]1N2 ; Cc1nccn1CCOc1nc(N2C[C@H]3C[C@@H](O)[C@@H](C2)N3)c2cnc(-c3cccc4cccc(Cl)c34)c(F)c2n1 ; O[C@@H]1C[C@@H]2CN(c3nc(OCCn4ccnc4CF)nc4c(F)c(-c5cccc6cccc(Cl)c56)ncc34)C[C@H]1N2 ; O[C@@H]1C[C@@H]2CN(c3nc(OCCn4ccnc4C(F)(F)F)nc4c(F)c(-c5cccc6cccc(Cl)c56)ncc34)C[C@H]1N2] 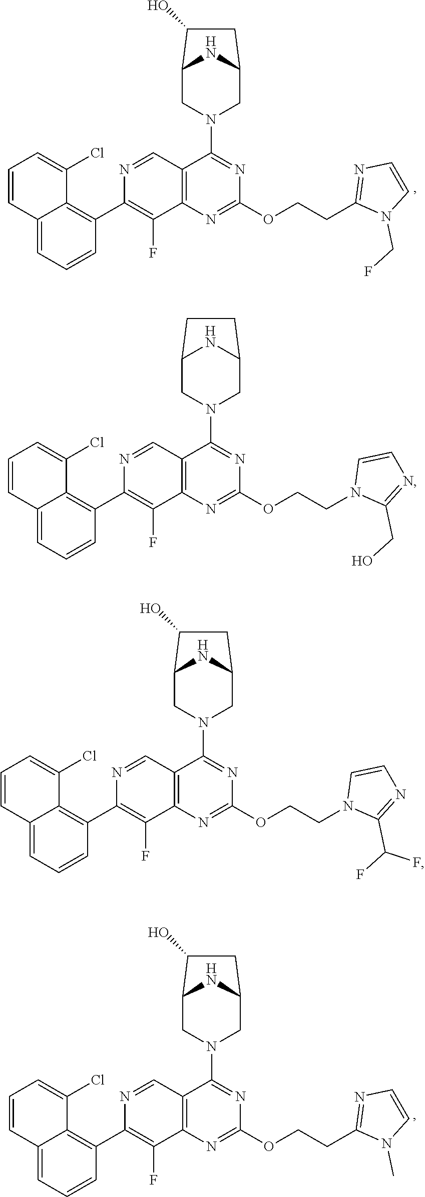 [SMILES: O[C@@H]1C[C@@H]2CN(c3nc(OCCc4nccn4CF)nc4c(F)c(-c5cccc6cccc(Cl)c56)ncc34)C[C@H]1N2 ; OCc1nccn1CCOc1nc(N2CC3CCC(C2)N3)c2cnc(-c3cccc4cccc(Cl)c34)c(F)c2n1 ; O[C@@H]1C[C@@H]2CN(c3nc(OCCn4ccnc4C(F)F)nc4c(F)c(-c5cccc6cccc(Cl)c56)ncc34)C[C@H]1N2 ; Cn1ccnc1CCOc1nc(N2C[C@H]3C[C@@H](O)[C@@H](C2)N3)c2cnc(-c3cccc4cccc(Cl)c34)c(F)c2n1] 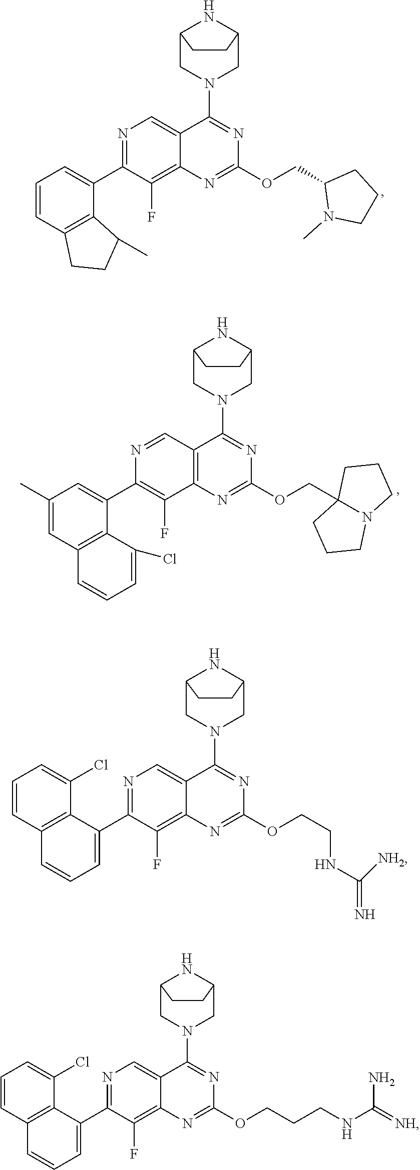 [SMILES: CC1CCc2cccc(-c3ncc4c(N5CC6CCC(C5)N6)nc(OC[C@@H]5CCCN5C)nc4c3F)c21 ; Cc1cc(-c2ncc3c(N4CC5CCC(C4)N5)nc(OCC45CCCN4CCC5)nc3c2F)c2c(Cl)cccc2c1 ; N=C(N)NCCOc1nc(N2CC3CCC(C2)N3)c2cnc(-c3cccc4cccc(Cl)c34)c(F)c2n1 ; N=C(N)NCCCOc1nc(N2CC3CCC(C2)N3)c2cnc(-c3cccc4cccc(Cl)c34)c(F)c2n1] 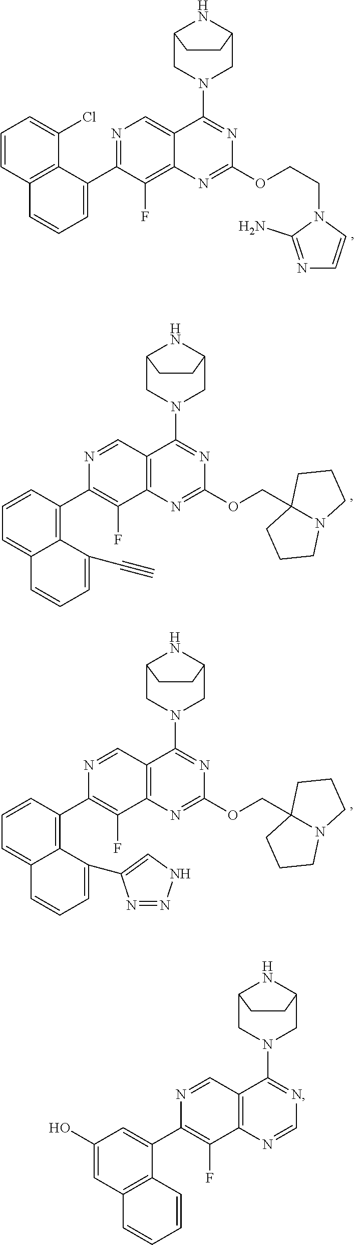 [SMILES: Nc1nccn1CCOc1nc(N2CC3CCC(C2)N3)c2cnc(-c3cccc4cccc(Cl)c34)c(F)c2n1 ; C#Cc1cccc2cccc(-c3ncc4c(N5CC6CCC(C5)N6)nc(OCC56CCCN5CCC6)nc4c3F)c12 ; Fc1c(-c2cccc3cccc(-c4c[nH]nn4)c23)ncc2c(N3CC4CCC(C3)N4)nc(OCC34CCCN3CCC4)nc12 ; Oc1cc(-c2ncc3c(N4CC5CCC(C4)N5)ncnc3c2F)c2ccccc2c1] 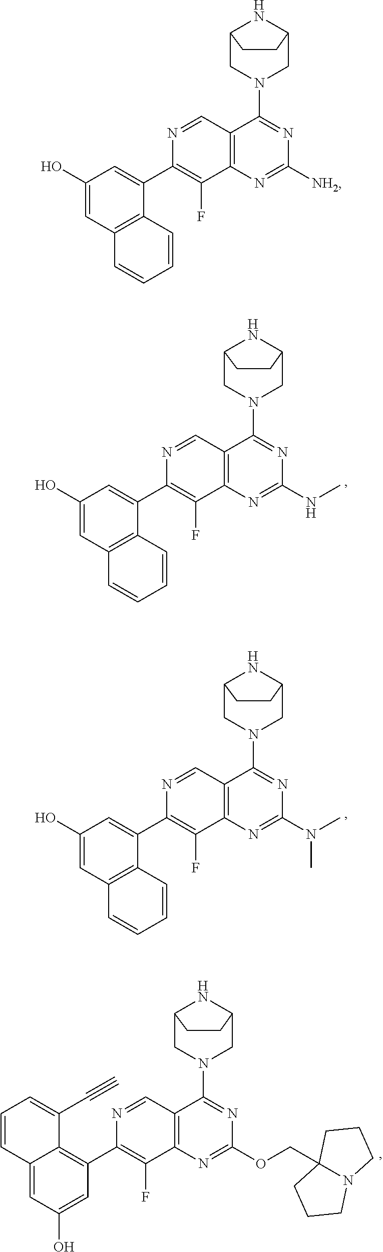 [SMILES: Nc1nc(N2CC3CCC(C2)N3)c2cnc(-c3cc(O)cc4ccccc34)c(F)c2n1 ; CNc1nc(N2CC3CCC(C2)N3)c2cnc(-c3cc(O)cc4ccccc34)c(F)c2n1 ; CN(C)c1nc(N2CC3CCC(C2)N3)c2cnc(-c3cc(O)cc4ccccc34)c(F)c2n1 ; C#Cc1cccc2cc(O)cc(-c3ncc4c(N5CC6CCC(C5)N6)nc(OCC56CCCN5CCC6)nc4c3F)c12] 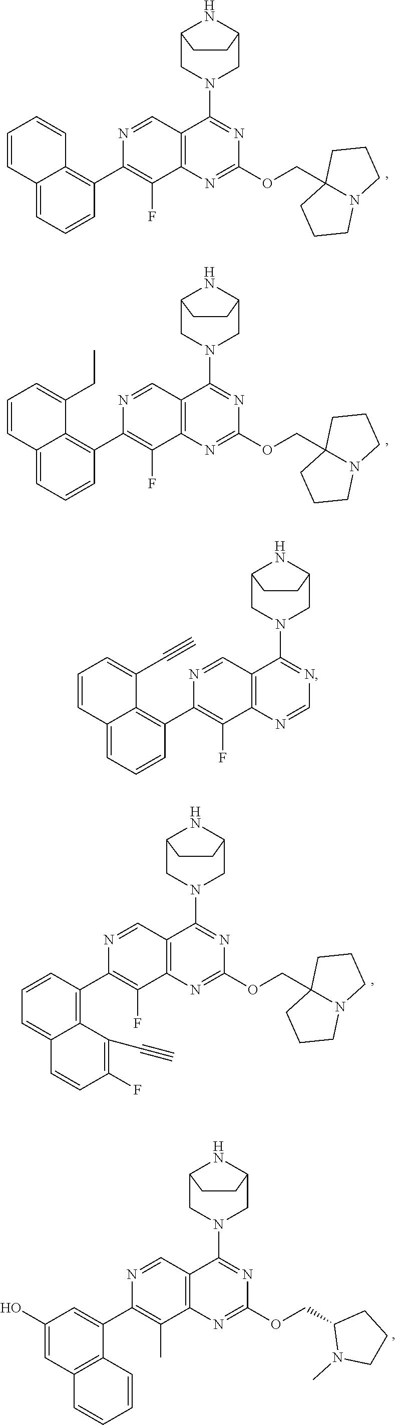 [SMILES: Fc1c(-c2cccc3ccccc23)ncc2c(N3CC4CCC(C3)N4)nc(OCC34CCCN3CCC4)nc12 ; CCc1cccc2cccc(-c3ncc4c(N5CC6CCC(C5)N6)nc(OCC56CCCN5CCC6)nc4c3F)c12 ; C#Cc1cccc2cccc(-c3ncc4c(N5CC6CCC(C5)N6)ncnc4c3F)c12 ; C#Cc1c(F)ccc2cccc(-c3ncc4c(N5CC6CCC(C5)N6)nc(OCC56CCCN5CCC6)nc4c3F)c12 ; Cc1c(-c2cc(O)cc3ccccc23)ncc2c(N3CC4CCC(C3)N4)nc(OC[C@@H]3CCCN3C)nc12] 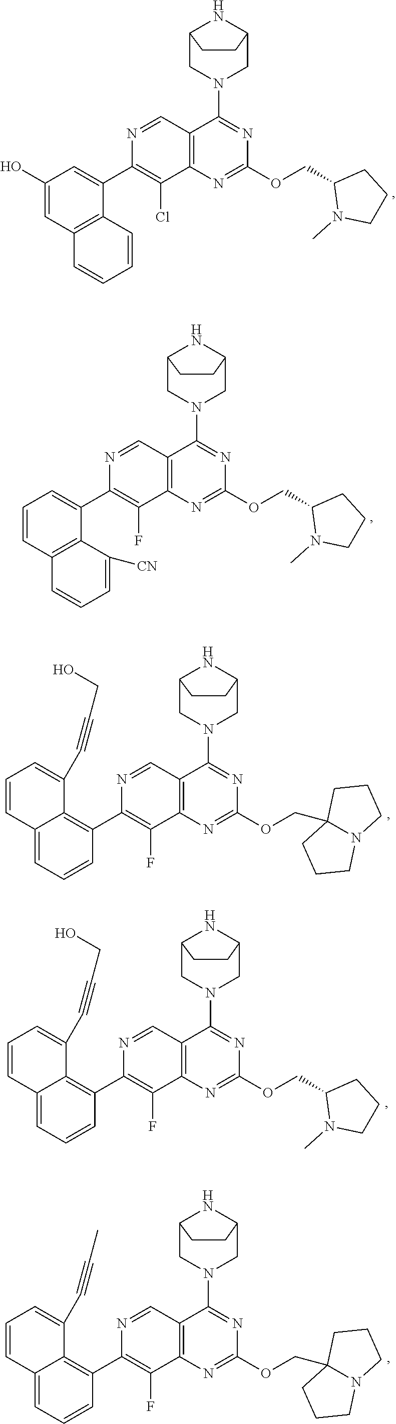 [SMILES: CN1CCC[C@H]1COc1nc(N2CC3CCC(C2)N3)c2cnc(-c3cc(O)cc4ccccc34)c(Cl)c2n1 ; CN1CCC[C@H]1COc1nc(N2CC3CCC(C2)N3)c2cnc(-c3cccc4cccc(C#N)c34)c(F)c2n1 ; OCC#Cc1cccc2cccc(-c3ncc4c(N5CC6CCC(C5)N6)nc(OCC56CCCN5CCC6)nc4c3F)c12 ; CN1CCC[C@H]1COc1nc(N2CC3CCC(C2)N3)c2cnc(-c3cccc4cccc(C#CCO)c34)c(F)c2n1 ; CC#Cc1cccc2cccc(-c3ncc4c(N5CC6CCC(C5)N6)nc(OCC56CCCN5CCC6)nc4c3F)c12] 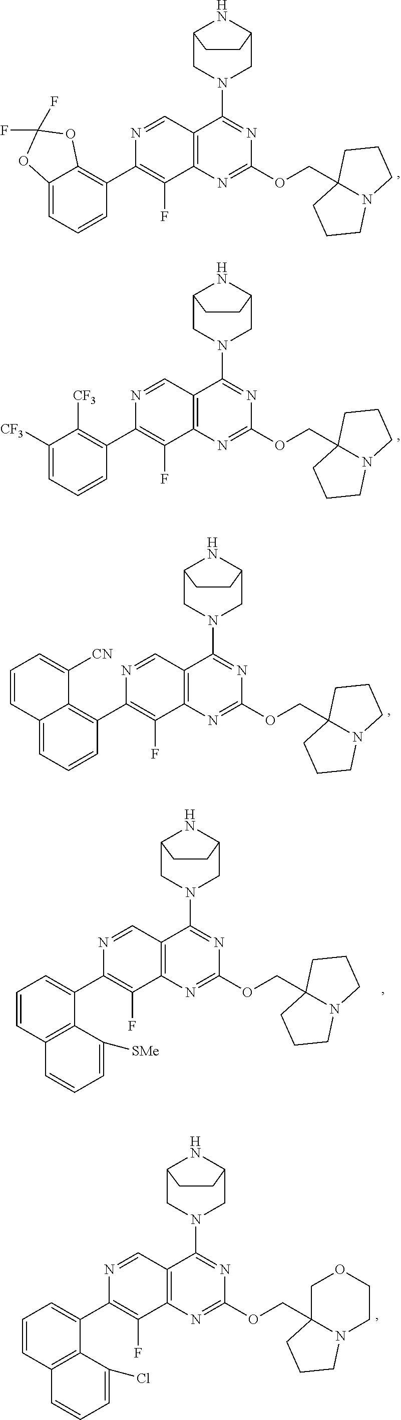 [SMILES: Fc1c(-c2cccc3c2OC(F)(F)O3)ncc2c(N3CC4CCC(C3)N4)nc(OCC34CCCN3CCC4)nc12 ; Fc1c(-c2cccc(C(F)(F)F)c2C(F)(F)F)ncc2c(N3CC4CCC(C3)N4)nc(OCC34CCCN3CCC4)nc12 ; N#Cc1cccc2cccc(-c3ncc4c(N5CC6CCC(C5)N6)nc(OCC56CCCN5CCC6)nc4c3F)c12 ; CSc1cccc2cccc(-c3ncc4c(N5CC6CCC(C5)N6)nc(OCC56CCCN5CCC6)nc4c3F)c12 ; Fc1c(-c2cccc3cccc(Cl)c23)ncc2c(N3CC4CCC(C3)N4)nc(OCC34CCCN3CCOC4)nc12] 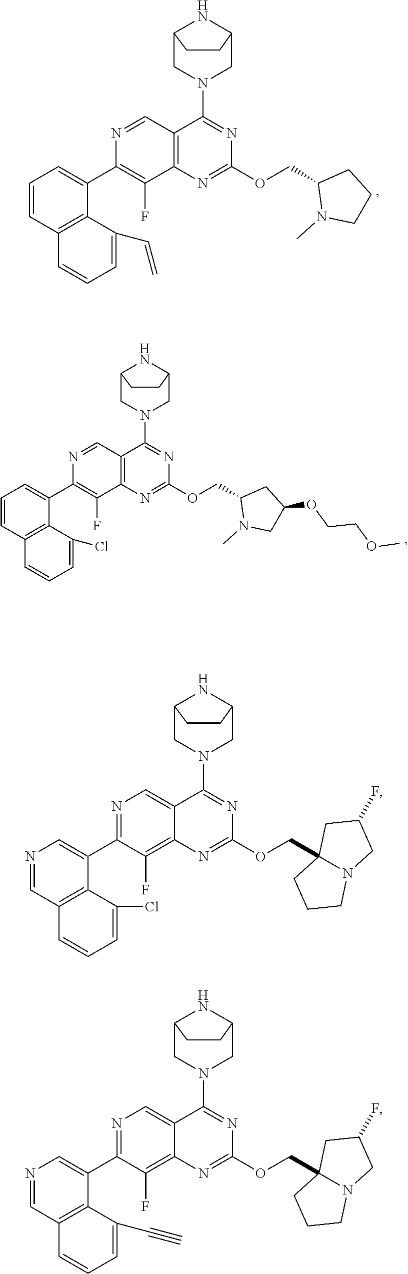 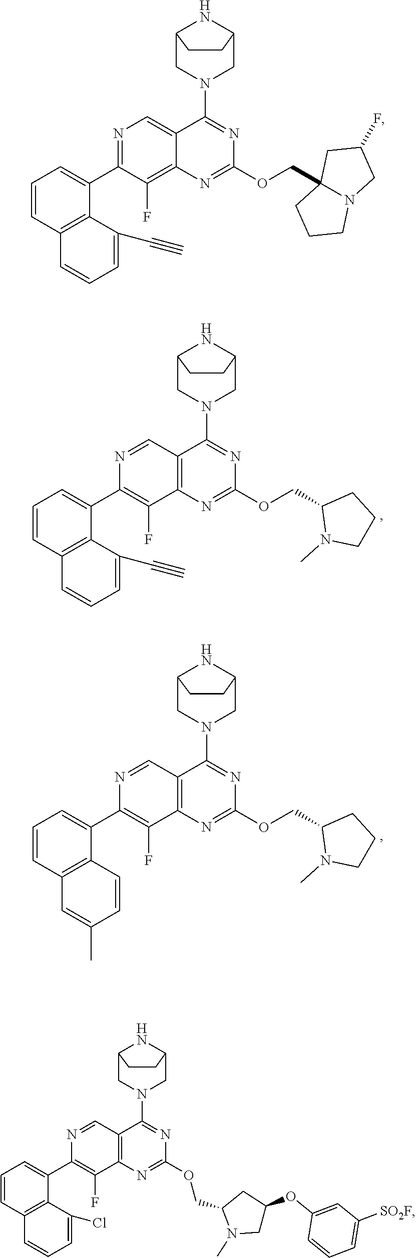 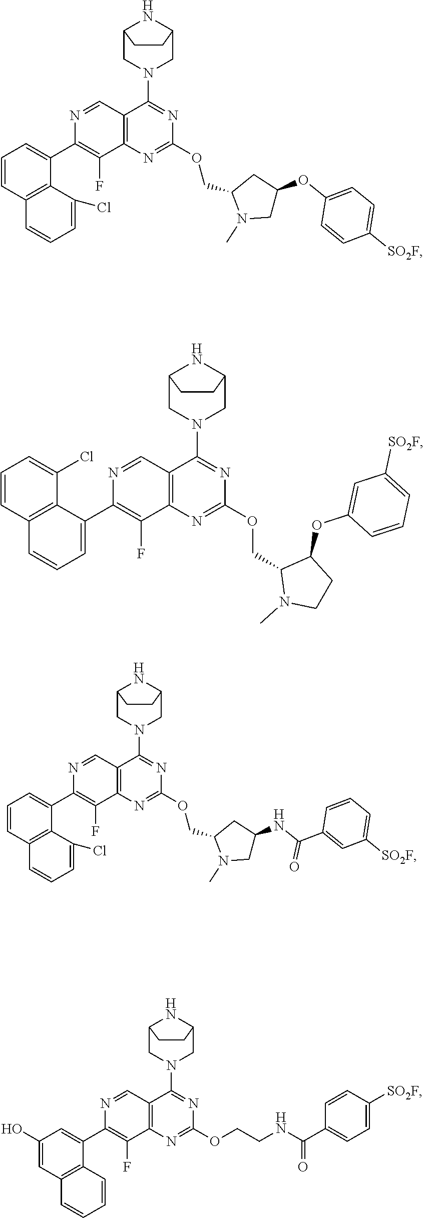 [SMILES: CN1C[C@H](Oc2ccc(S(=O)(=O)F)cc2)C[C@H]1COc1nc(N2CC3CCC(C2)N3)c2cnc(-c3cccc4cccc(Cl)c34)c(F)c2n1 ; CN1CC[C@H](Oc2cccc(S(=O)(=O)F)c2)[C@H]1COc1nc(N2CC3CCC(C2)N3)c2cnc(-c3cccc4cccc(Cl)c34)c(F)c2n1 ; CN1C[C@H](NC(=O)c2cccc(S(=O)(=O)F)c2)C[C@H]1COc1nc(N2CC3CCC(C2)N3)c2cnc(-c3cccc4cccc(Cl)c34)c(F)c2n1 ; O=C(NCCOc1nc(N2CC3CCC(C2)N3)c2cnc(-c3cc(O)cc4ccccc34)c(F)c2n1)c1ccc(S(=O)(=O)F)cc1] 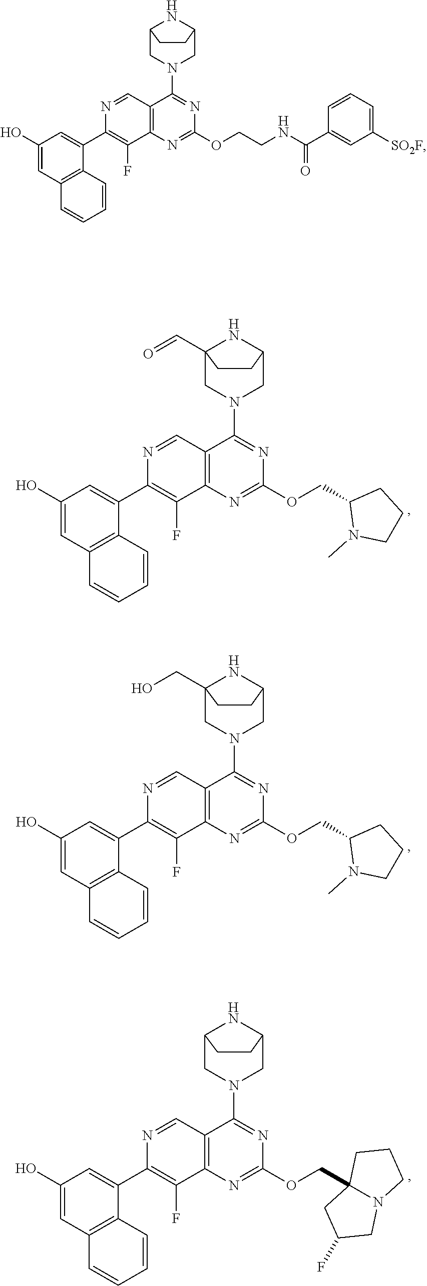 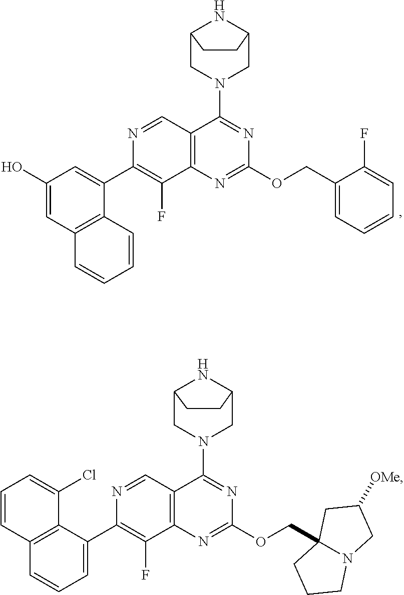 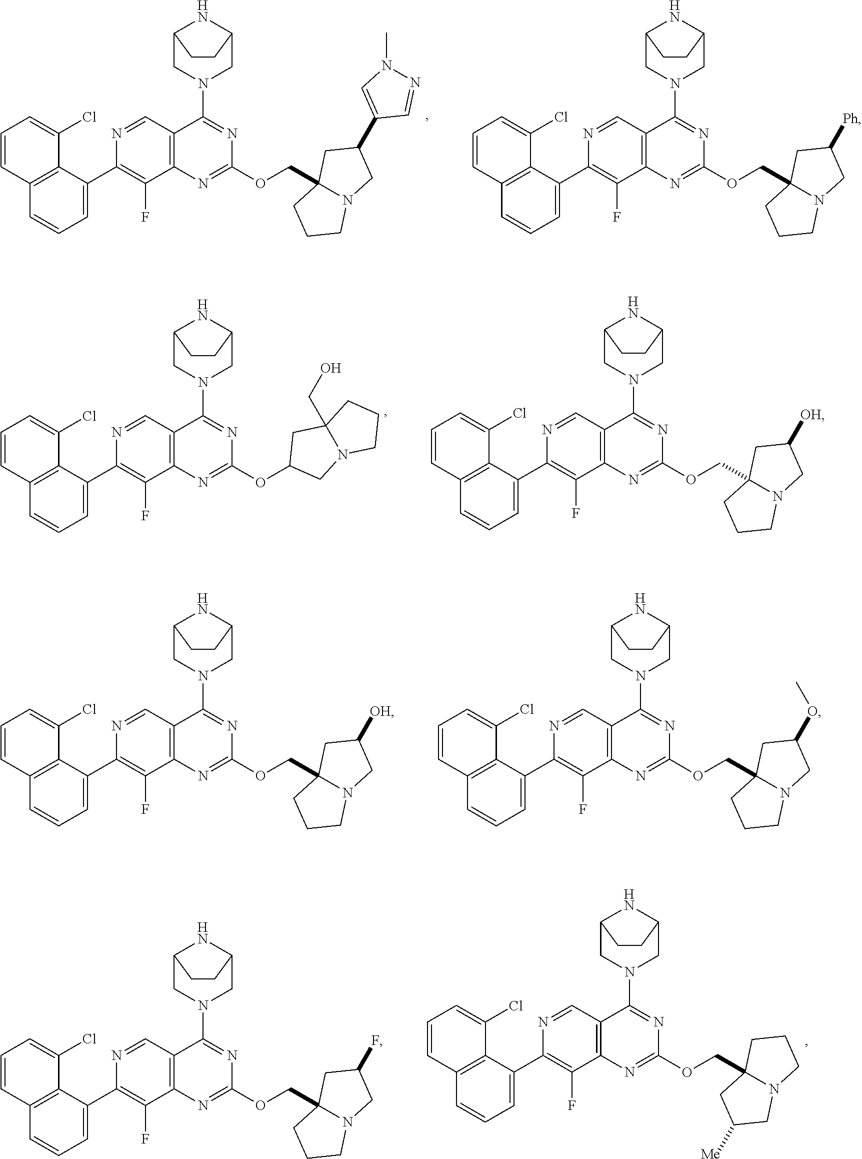 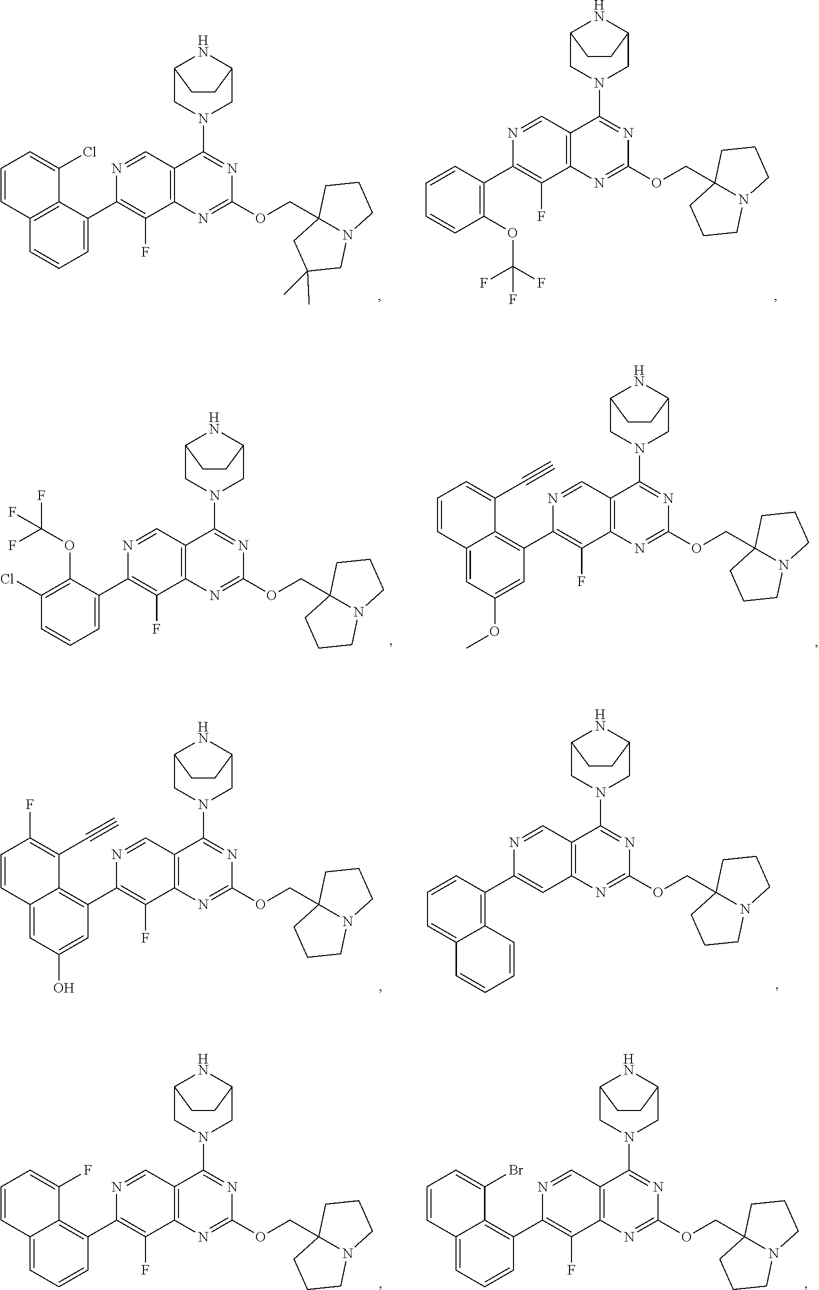 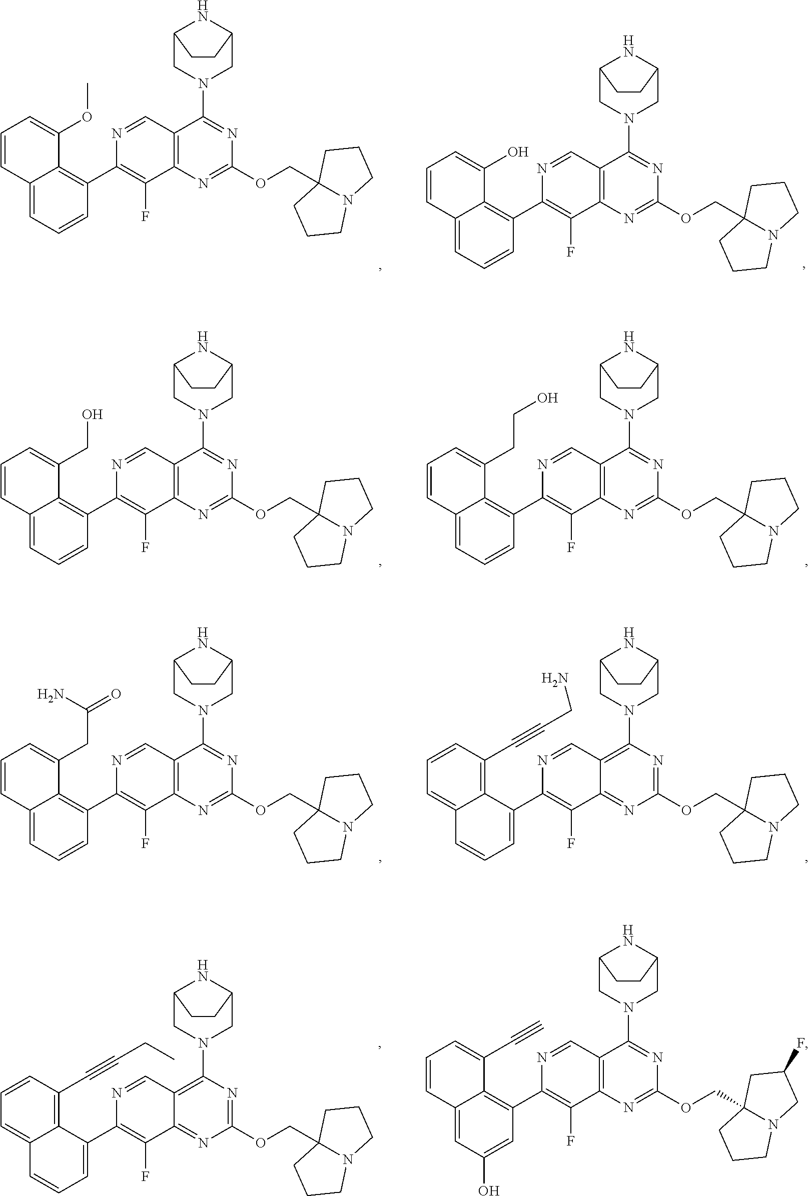 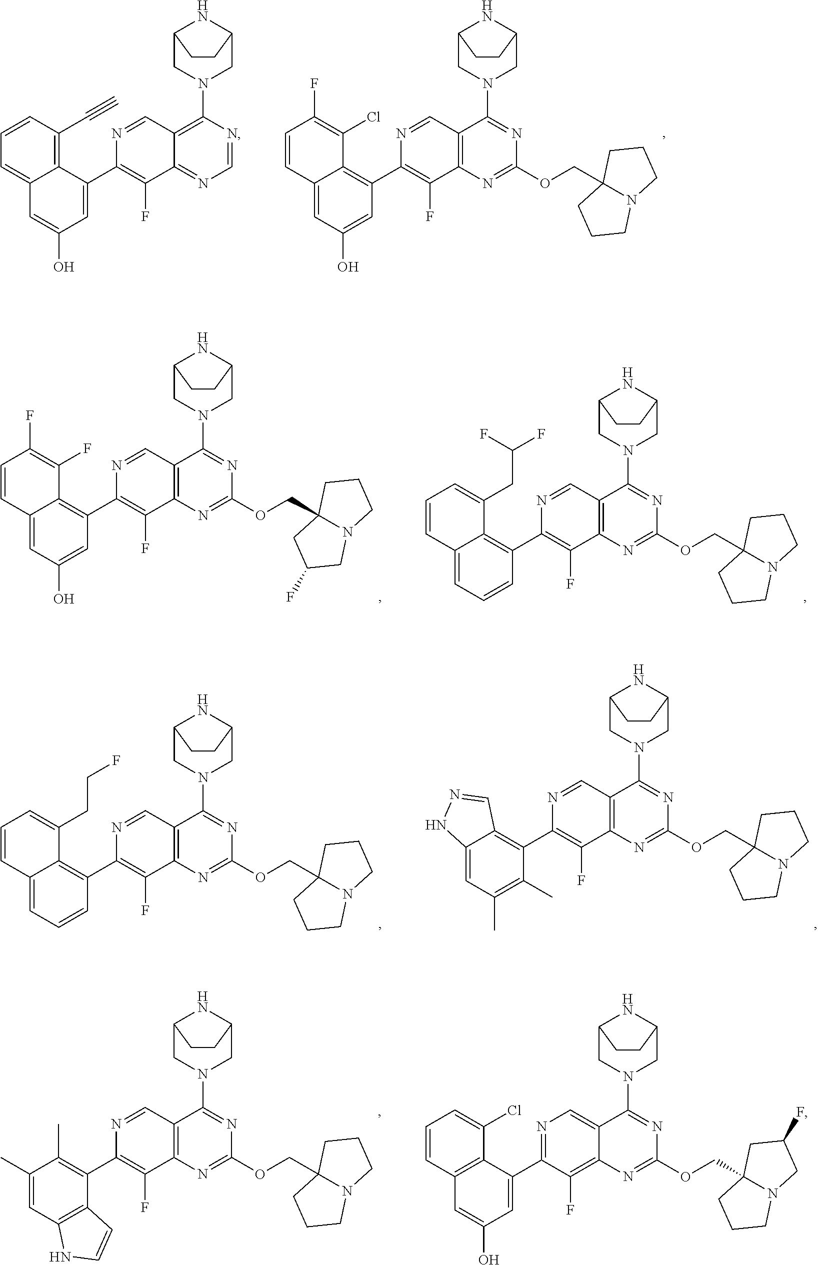 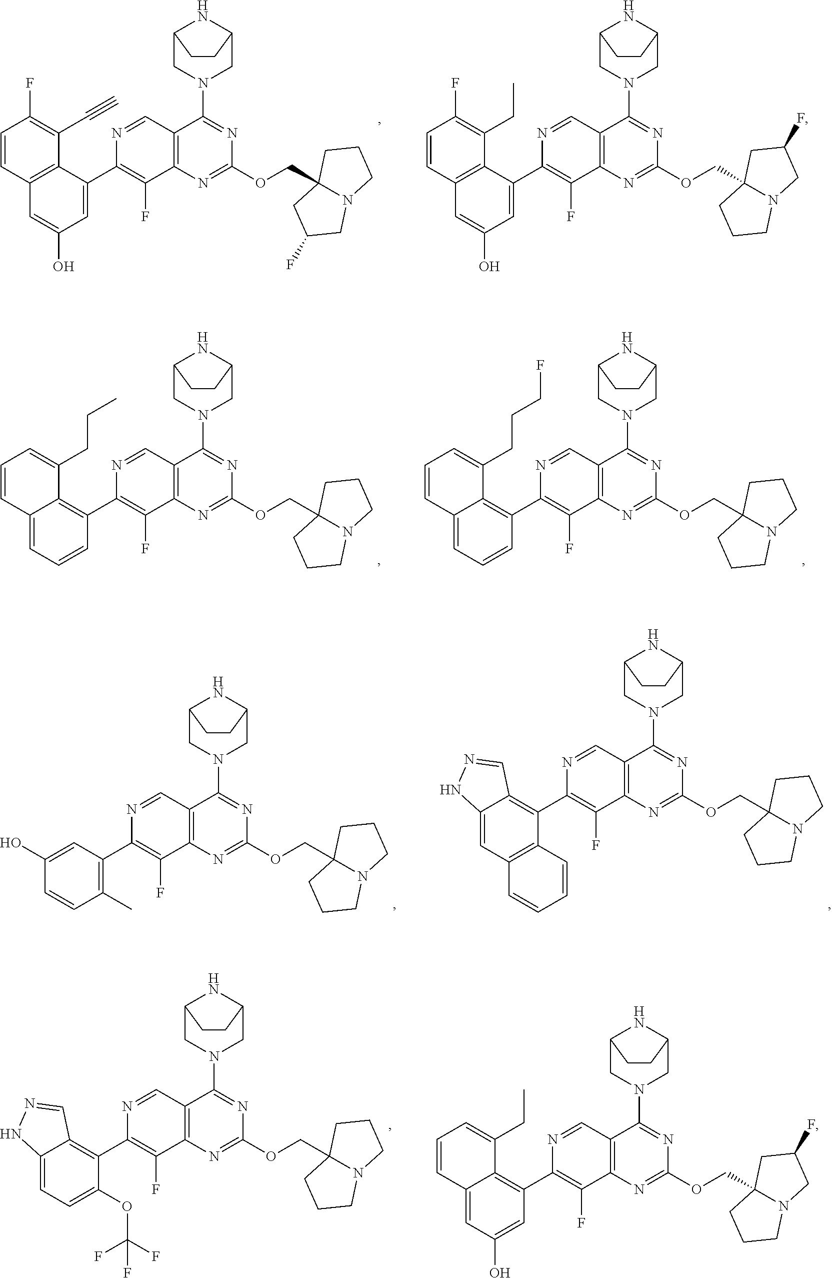 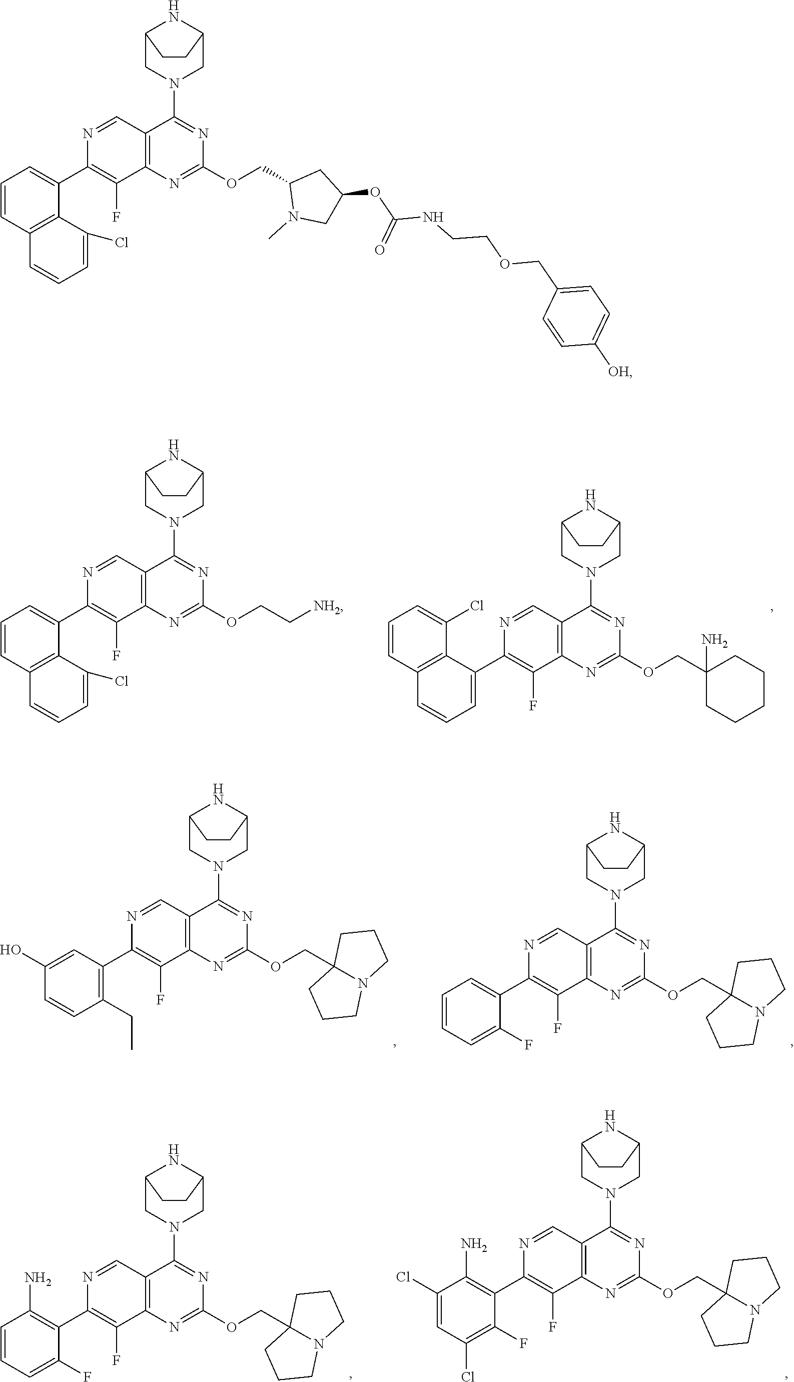 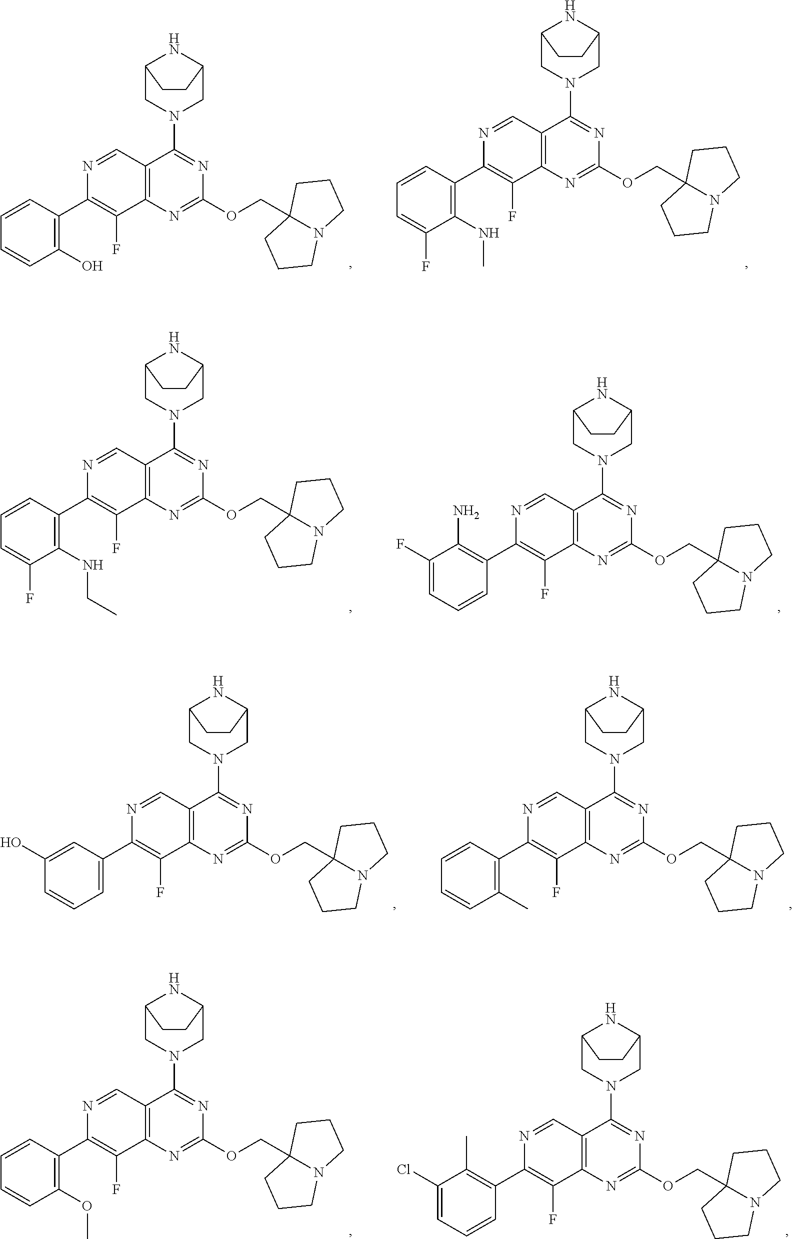 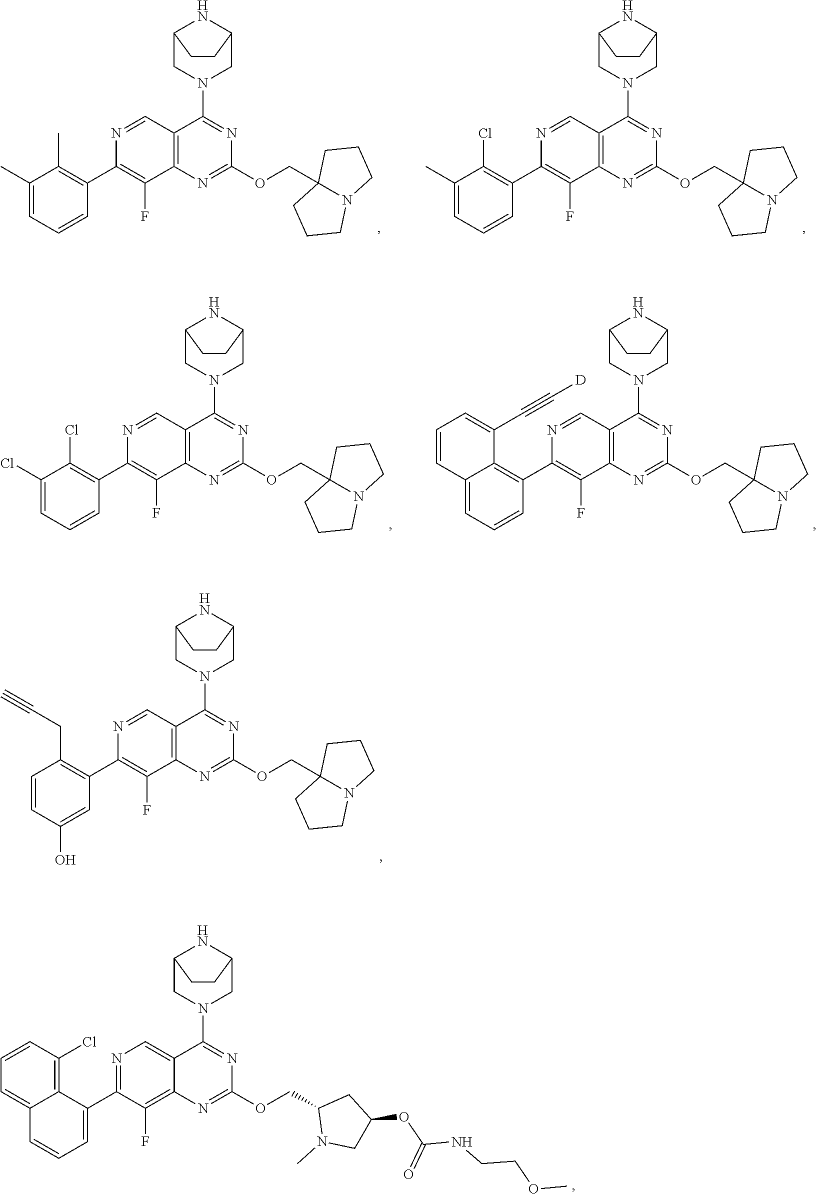 [SMILES: Cc1cccc(-c2ncc3c(N4CC5CCC(C4)N5)nc(OCC45CCCN4CCC5)nc3c2F)c1C ; Cc1cccc(-c2ncc3c(N4CC5CCC(C4)N5)nc(OCC45CCCN4CCC5)nc3c2F)c1Cl ; Fc1c(-c2cccc(Cl)c2Cl)ncc2c(N3CC4CCC(C3)N4)nc(OCC34CCCN3CCC4)nc12 ; [2H]C#Cc1cccc2cccc(-c3ncc4c(N5CC6CCC(C5)N6)nc(OCC56CCCN5CCC6)nc4c3F)c12 ; C#CCc1ccc(O)cc1-c1ncc2c(N3CC4CCC(C3)N4)nc(OCC34CCCN3CCC4)nc2c1F ; COCCNC(=O)O[C@@H]1C[C@@H](COc2nc(N3CC4CCC(C3)N4)c3cnc(-c4cccc5cccc(Cl)c45)c(F)c3n2)N(C)C1] 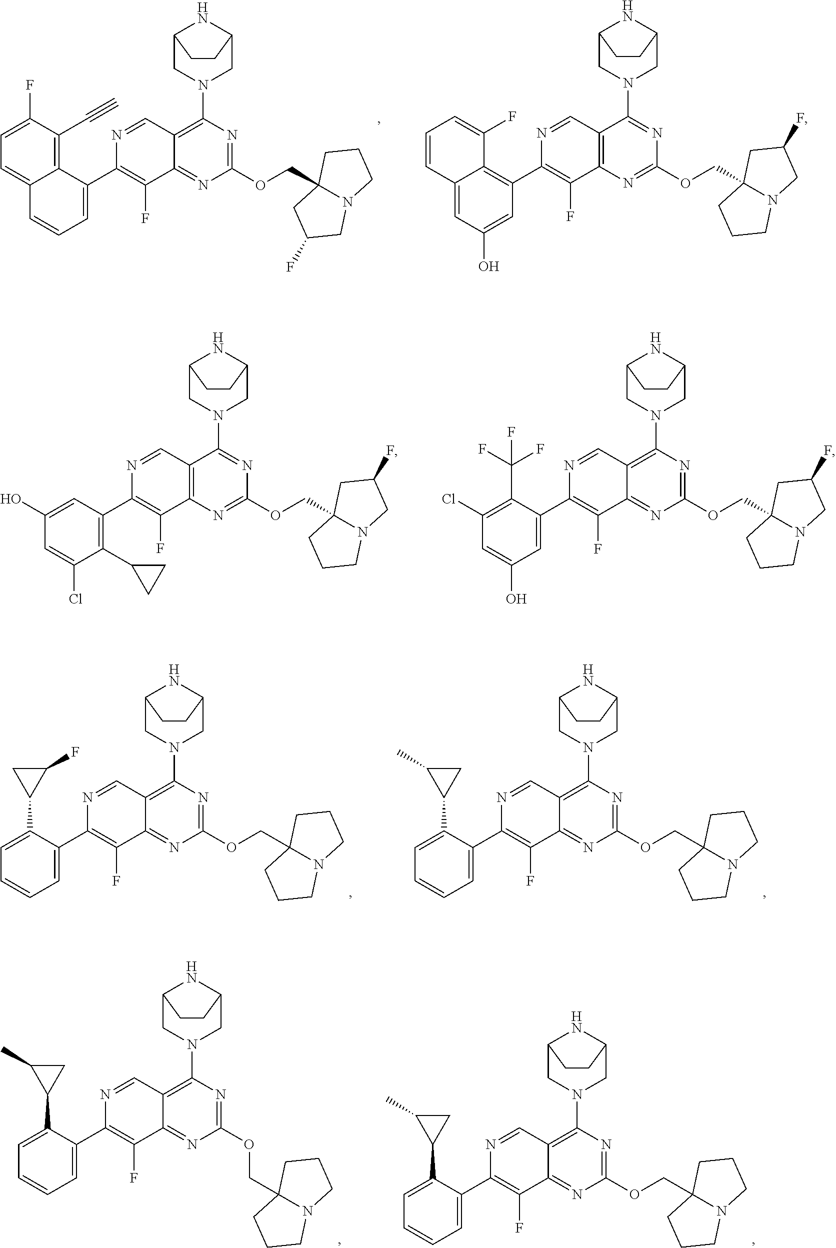 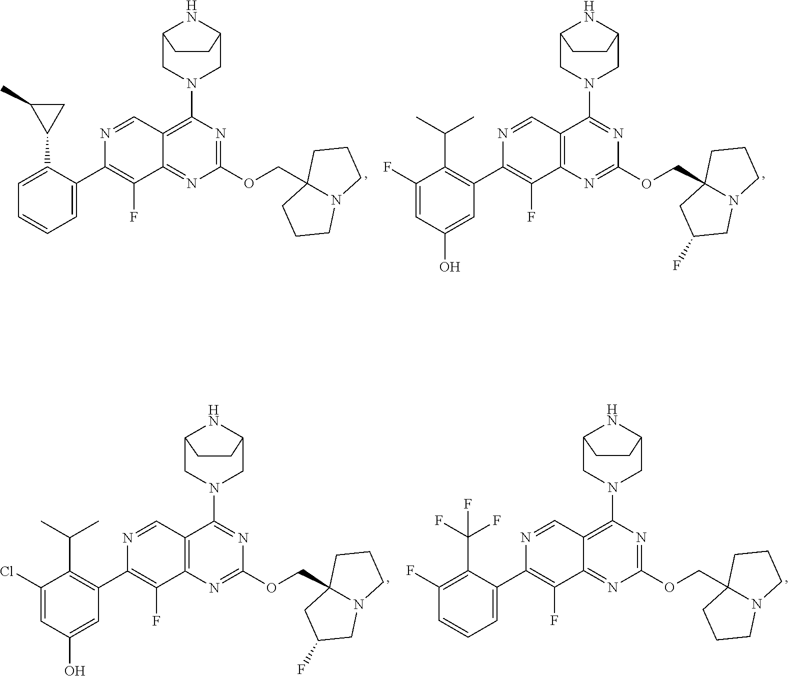 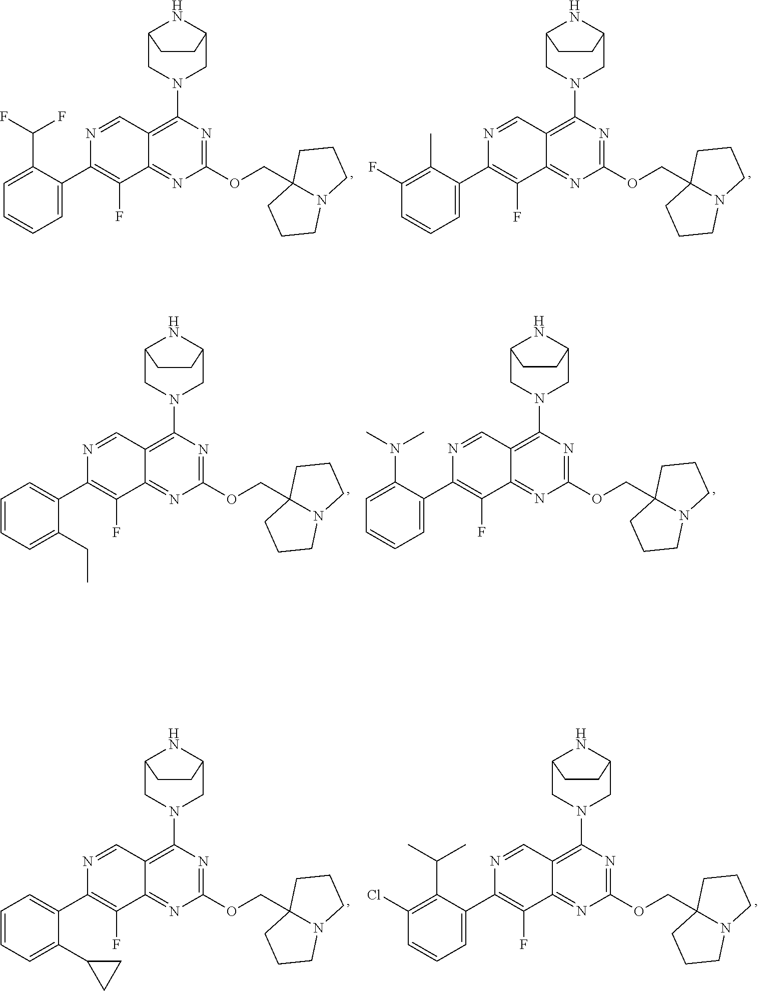 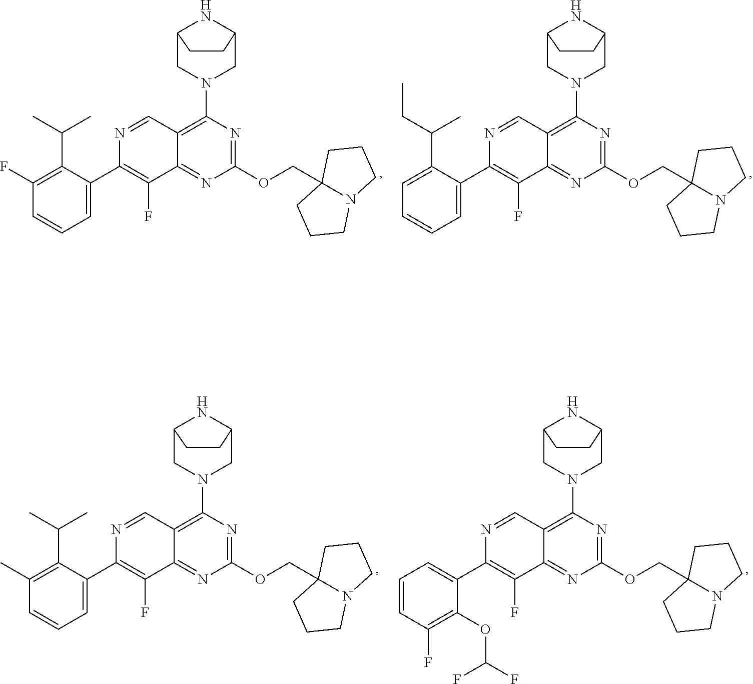 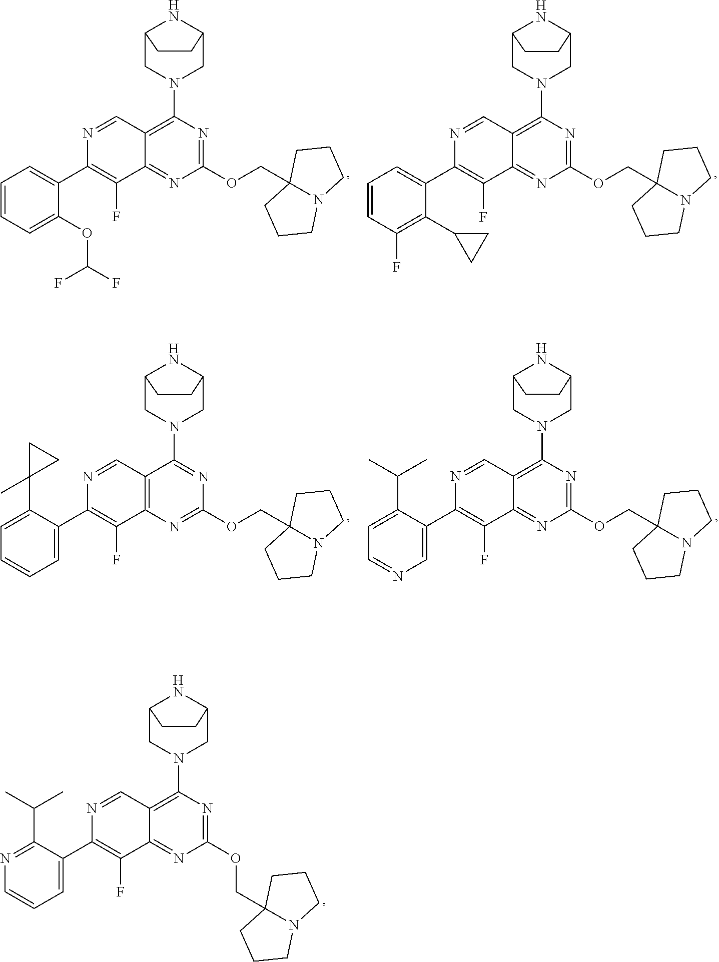 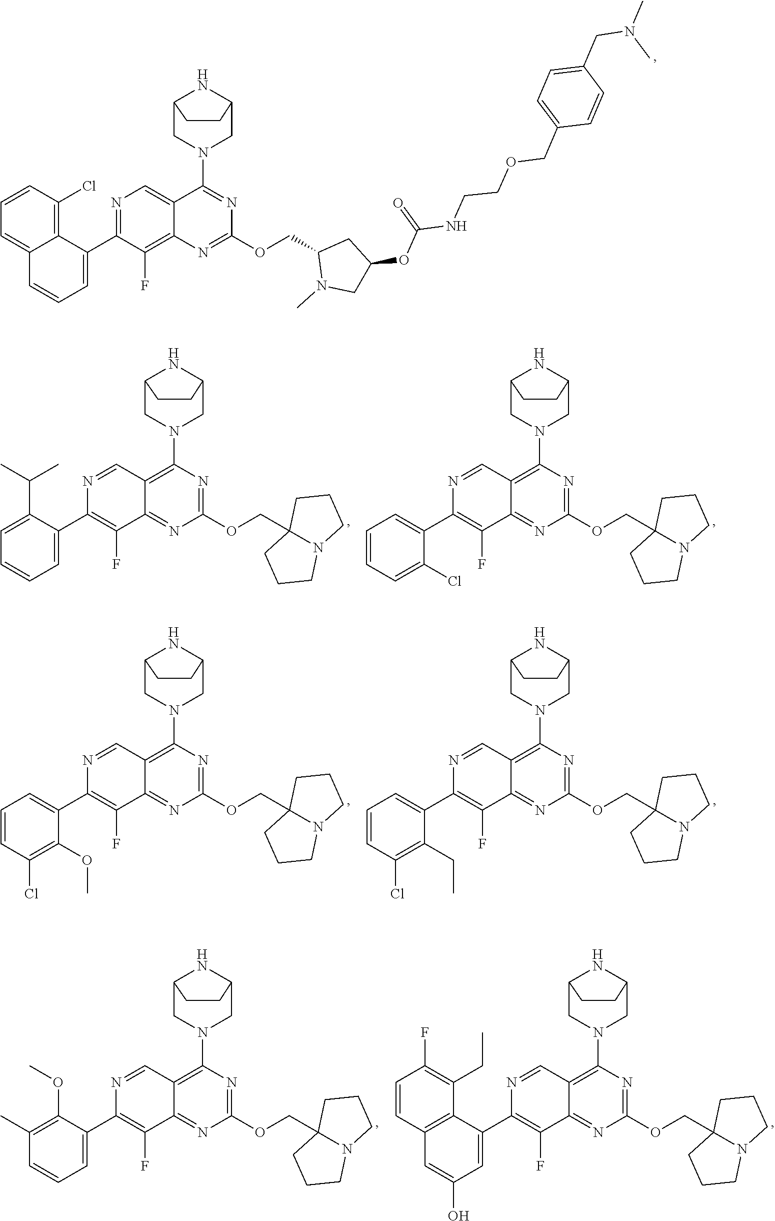 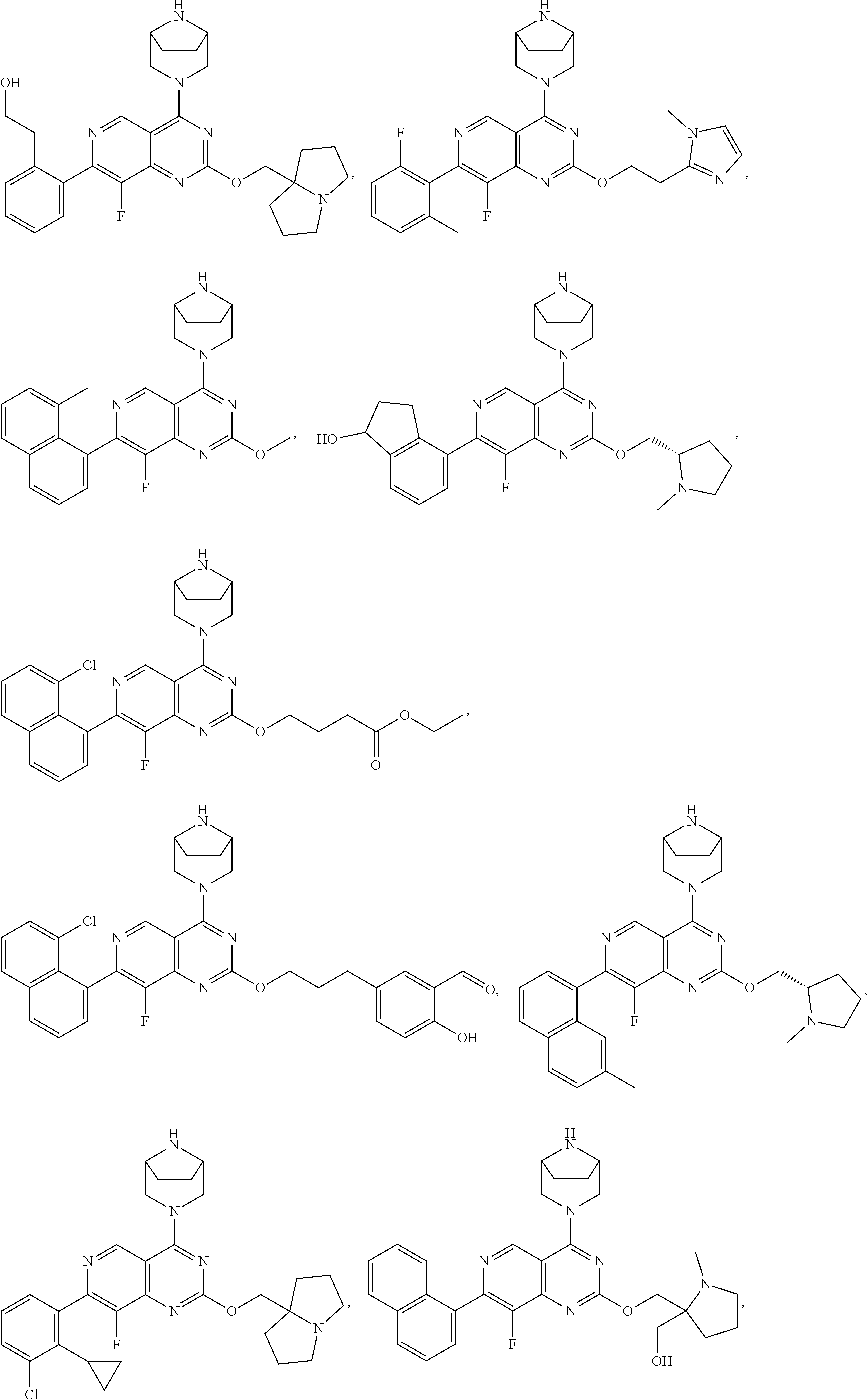 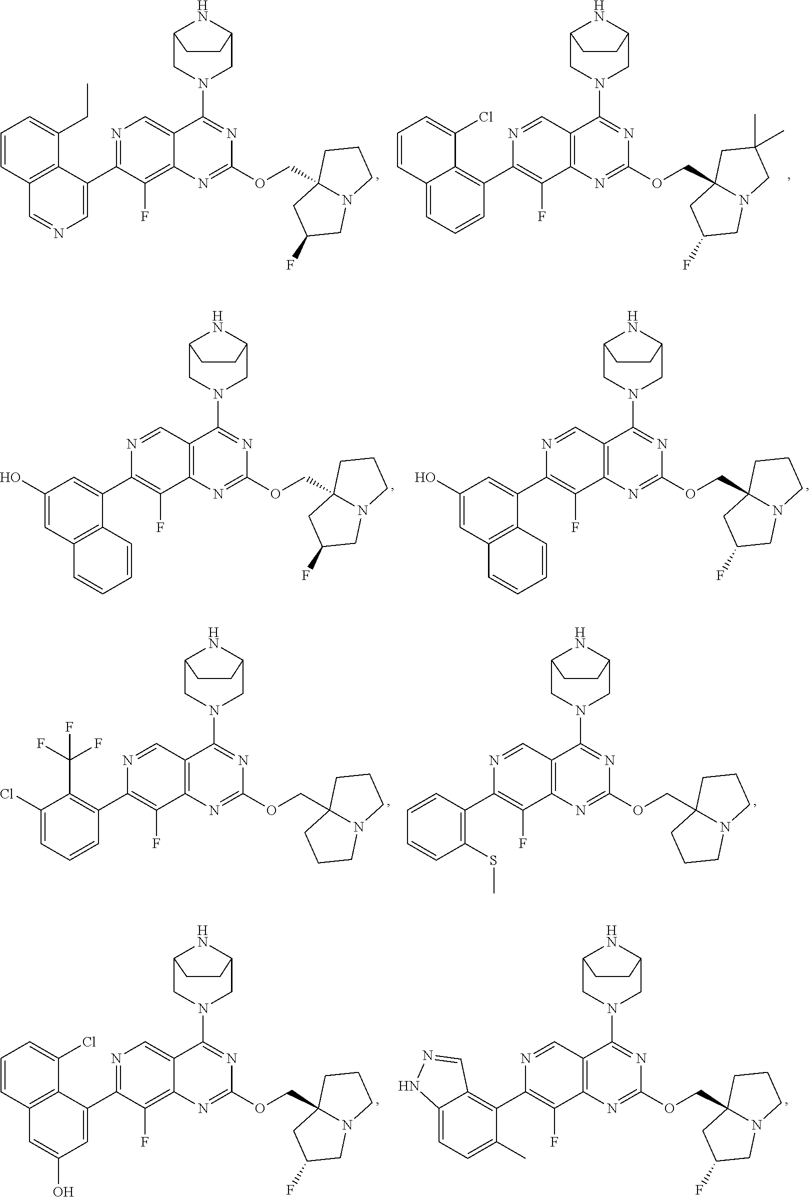 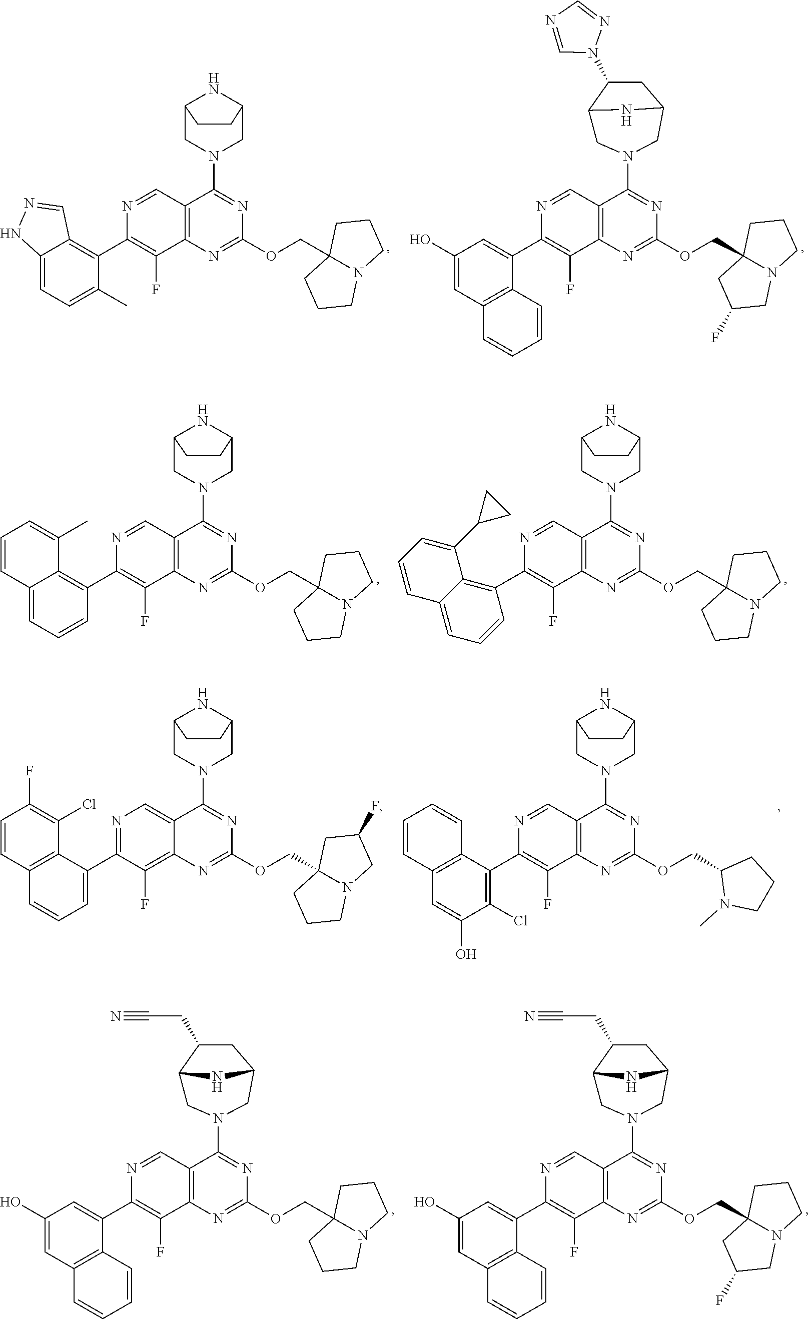 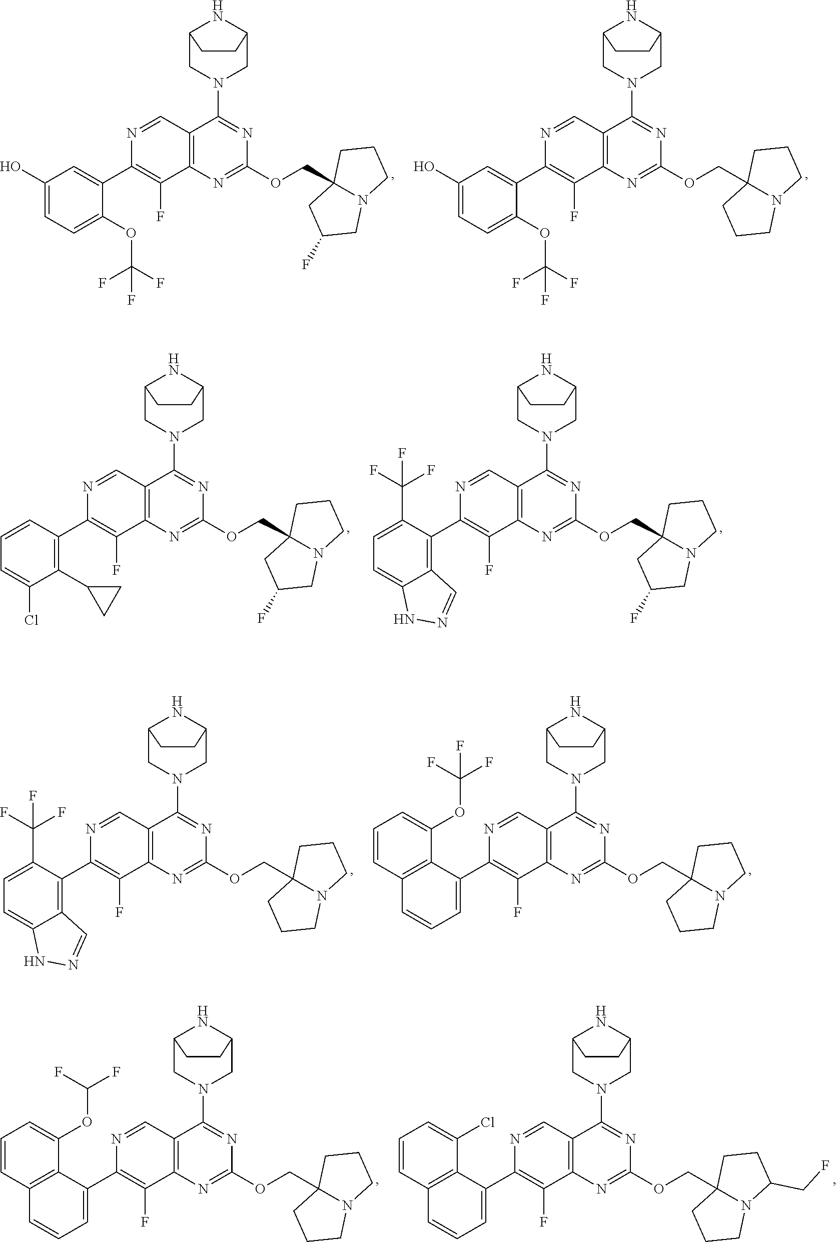 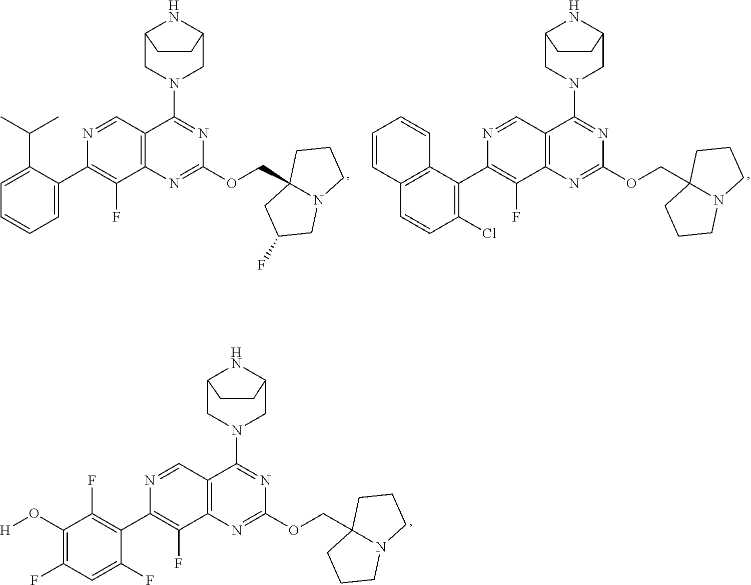 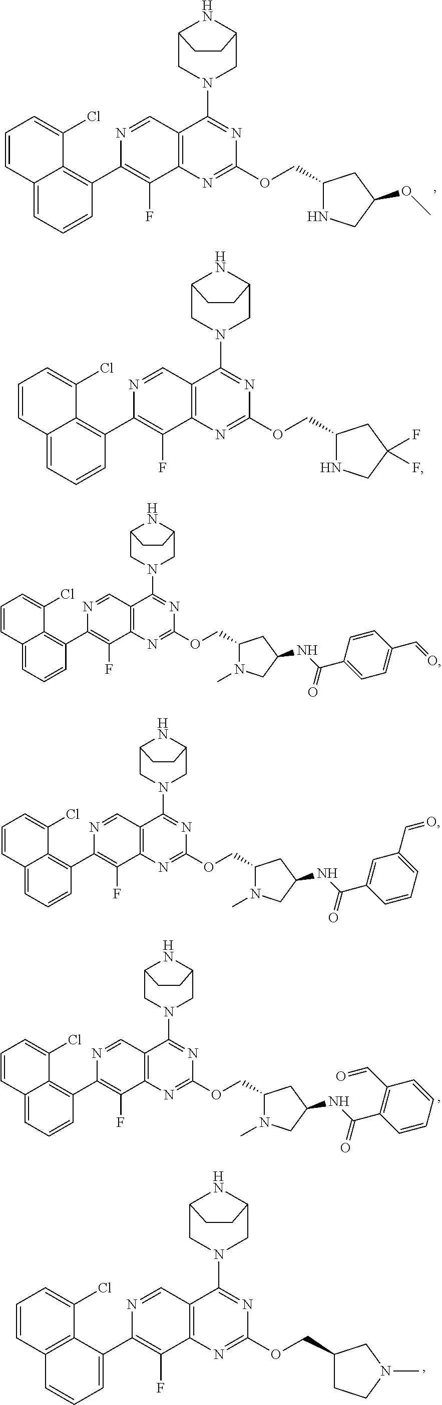 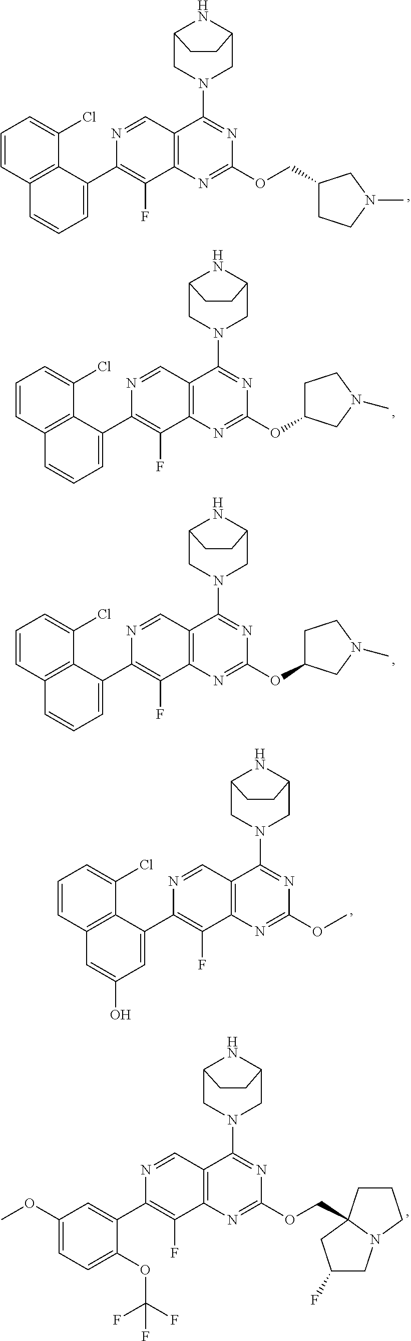 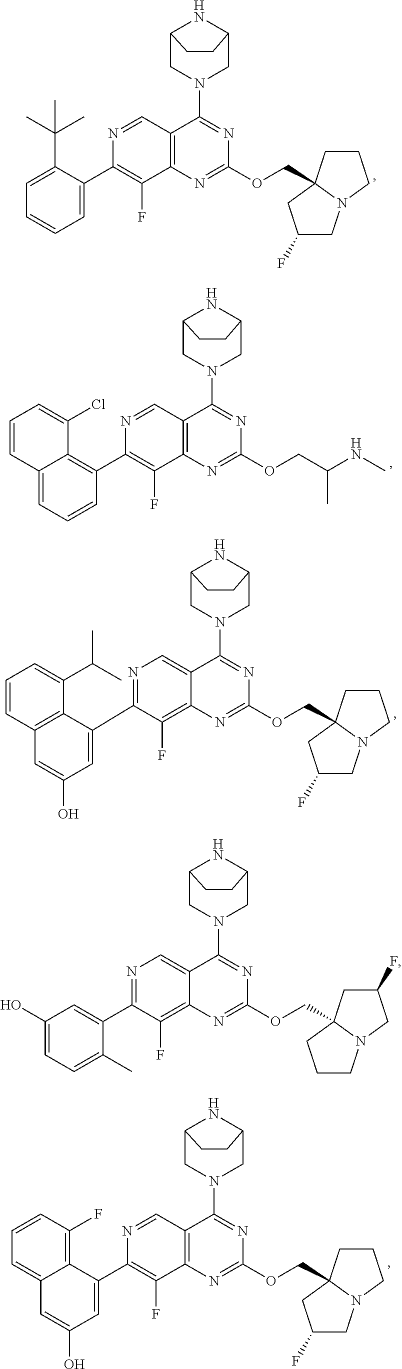 [SMILES: CC(C)(C)c1ccccc1-c1ncc2c(N3CC4CCC(C3)N4)nc(OC[C@@]34CCCN3C[C@H](F)C4)nc2c1F ; CNC(C)COc1nc(N2CC3CCC(C2)N3)c2cnc(-c3cccc4cccc(Cl)c34)c(F)c2n1 ; CC(C)c1cccc2cc(O)cc(-c3ncc4c(N5CC6CCC(C5)N6)nc(OC[C@@]56CCCN5C[C@H](F)C6)nc4c3F)c12 ; Cc1ccc(O)cc1-c1ncc2c(N3CC4CCC(C3)N4)nc(OC[C@@]34CCCN3C[C@H](F)C4)nc2c1F ; Oc1cc(-c2ncc3c(N4CC5CCC(C4)N5)nc(OC[C@@]45CCCN4C[C@H](F)C5)nc3c2F)c2c(F)cccc2c1] 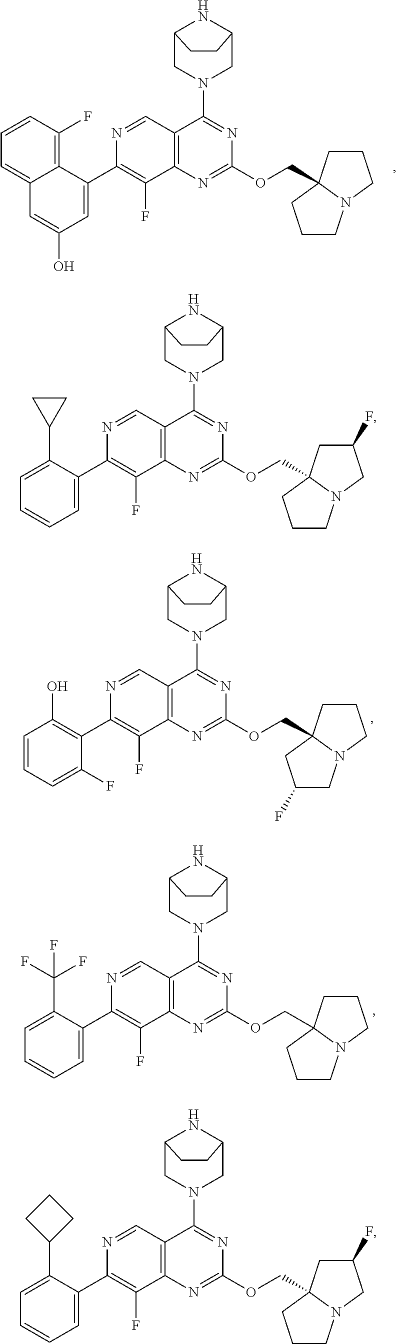 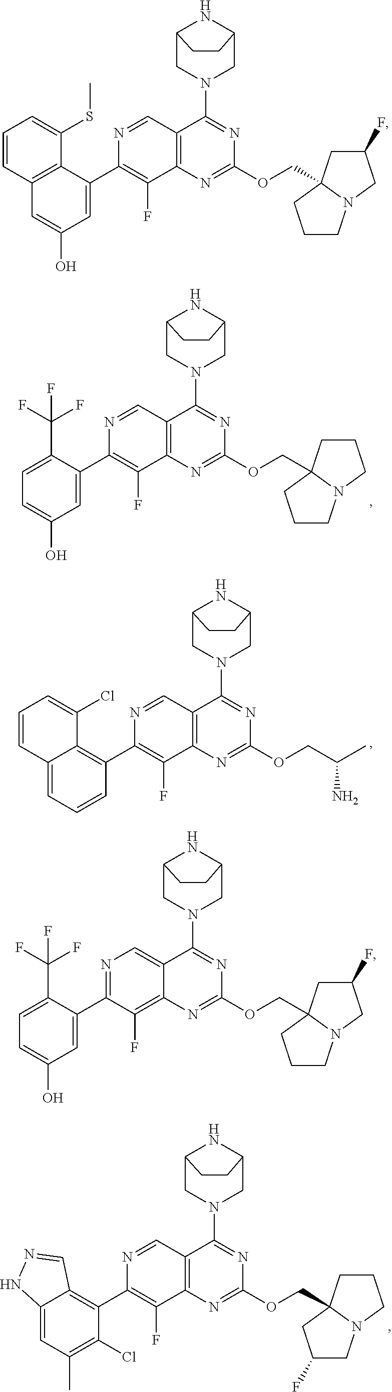 [SMILES: CSc1cccc2cc(O)cc(-c3ncc4c(N5CC6CCC(C5)N6)nc(OC[C@@]56CCCN5C[C@H](F)C6)nc4c3F)c12 ; Oc1ccc(C(F)(F)F)c(-c2ncc3c(N4CC5CCC(C4)N5)nc(OCC45CCCN4CCC5)nc3c2F)c1 ; C[C@H](N)COc1nc(N2CC3CCC(C2)N3)c2cnc(-c3cccc4cccc(Cl)c34)c(F)c2n1 ; Oc1ccc(C(F)(F)F)c(-c2ncc3c(N4CC5CCC(C4)N5)nc(OCC45CCCN4C[C@H](F)C5)nc3c2F)c1 ; Cc1cc2[nH]ncc2c(-c2ncc3c(N4CC5CCC(C4)N5)nc(OC[C@@]45CCCN4C[C@H](F)C5)nc3c2F)c1Cl] 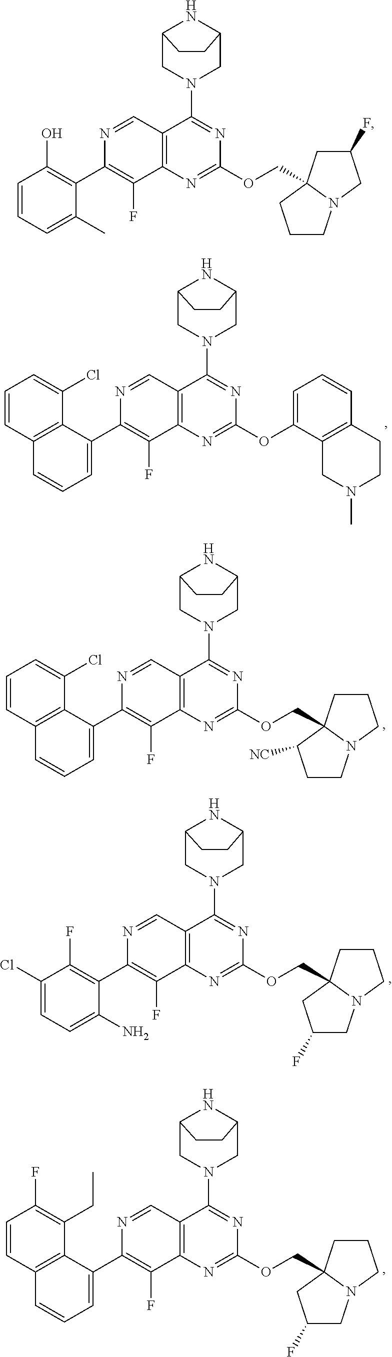 [SMILES: Cc1cccc(O)c1-c1ncc2c(N3CC4CCC(C3)N4)nc(OC[C@@]34CCCN3C[C@H](F)C4)nc2c1F ; CN1CCc2cccc(Oc3nc(N4CC5CCC(C4)N5)c4cnc(-c5cccc6cccc(Cl)c56)c(F)c4n3)c2C1 ; N#C[C@H]1CCN2CCC[C@@]12COc1nc(N2CC3CCC(C2)N3)c2cnc(-c3cccc4cccc(Cl)c34)c(F)c2n1 ; Nc1ccc(Cl)c(F)c1-c1ncc2c(N3CC4CCC(C3)N4)nc(OC[C@@]34CCCN3C[C@H](F)C4)nc2c1F ; CCc1c(F)ccc2cccc(-c3ncc4c(N5CC6CCC(C5)N6)nc(OC[C@@]56CCCN5C[C@H](F)C6)nc4c3F)c12] 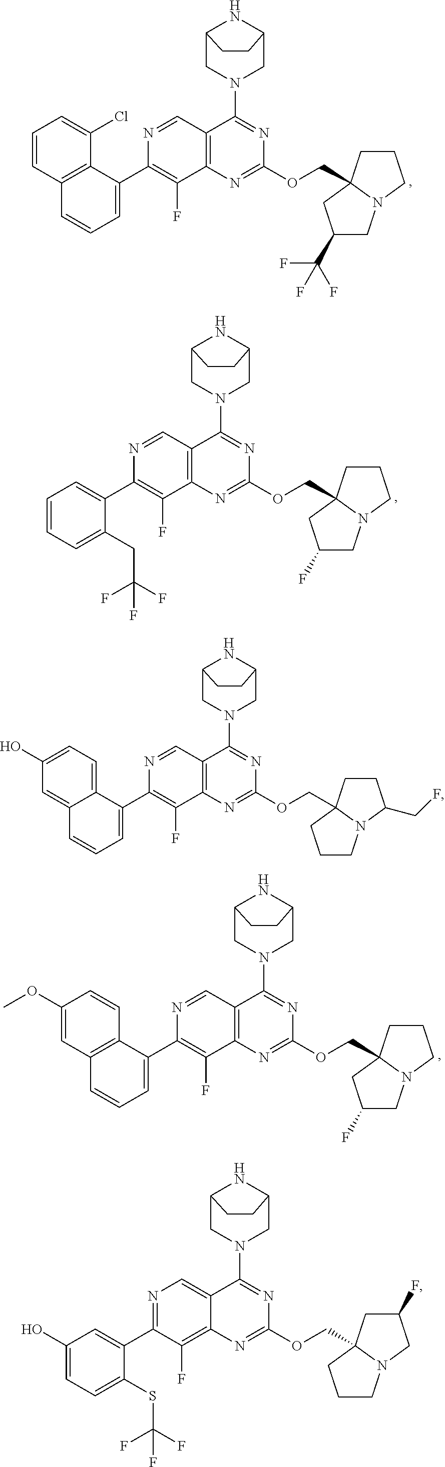 [SMILES: Fc1c(-c2cccc3cccc(Cl)c23)ncc2c(N3CC4CCC(C3)N4)nc(OC[C@@]34CCCN3C[C@@H](C(F)(F)F)C4)nc12 ; Fc1c(-c2ccccc2CC(F)(F)F)ncc2c(N3CC4CCC(C3)N4)nc(OC[C@@]34CCCN3C[C@H](F)C4)nc12 ; Oc1ccc2c(-c3ncc4c(N5CC6CCC(C5)N6)nc(OCC56CCCN5C(CF)CC6)nc4c3F)cccc2c1 ; COc1ccc2c(-c3ncc4c(N5CC6CCC(C5)N6)nc(OC[C@@]56CCCN5C[C@H](F)C6)nc4c3F)cccc2c1 ; Oc1ccc(SC(F)(F)F)c(-c2ncc3c(N4CC5CCC(C4)N5)nc(OC[C@@]45CCCN4C[C@H](F)C5)nc3c2F)c1] 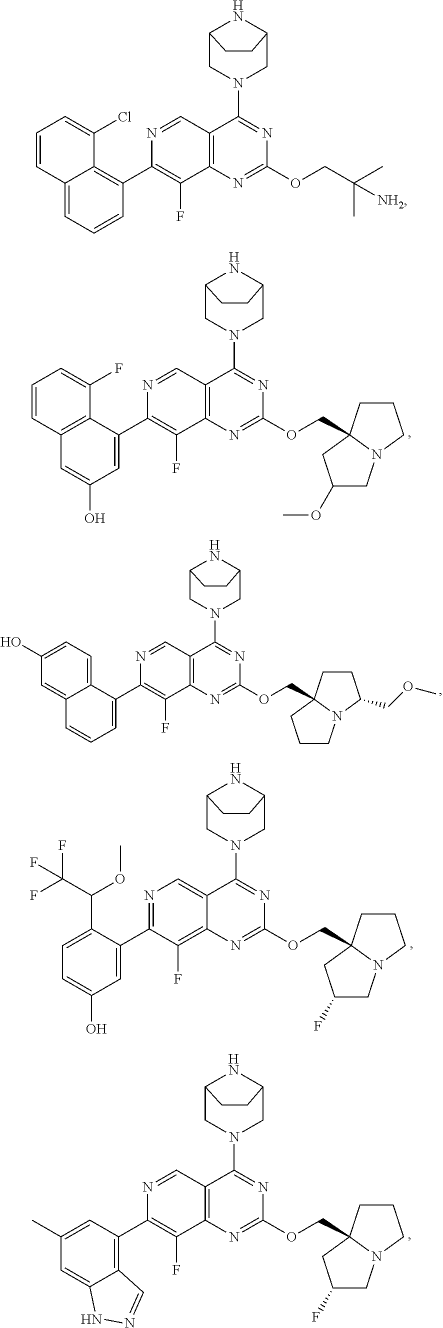 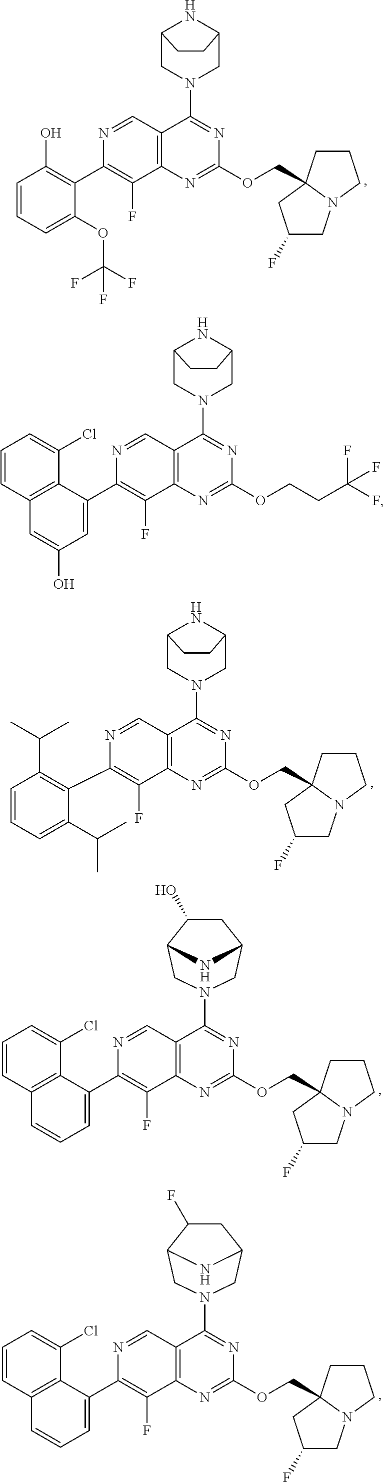 [SMILES: Oc1cccc(OC(F)(F)F)c1-c1ncc2c(N3CC4CCC(C3)N4)nc(OC[C@@]34CCCN3C[C@H](F)C4)nc2c1F ; Oc1cc(-c2ncc3c(N4CC5CCC(C4)N5)nc(OCCC(F)(F)F)nc3c2F)c2c(Cl)cccc2c1 ; CC(C)c1cccc(C(C)C)c1-c1ncc2c(N3CC4CCC(C3)N4)nc(OC[C@@]34CCCN3C[C@H](F)C4)nc2c1F ; O[C@@H]1C[C@@H]2CN(c3nc(OC[C@@]45CCCN4C[C@H](F)C5)nc4c(F)c(-c5cccc6cccc(Cl)c56)ncc34)C[C@H]1N2 ; Fc1c(-c2cccc3cccc(Cl)c23)ncc2c(N3CC4CC(F)C(C3)N4)nc(OC[C@@]34CCCN3C[C@H](F)C4)nc12] 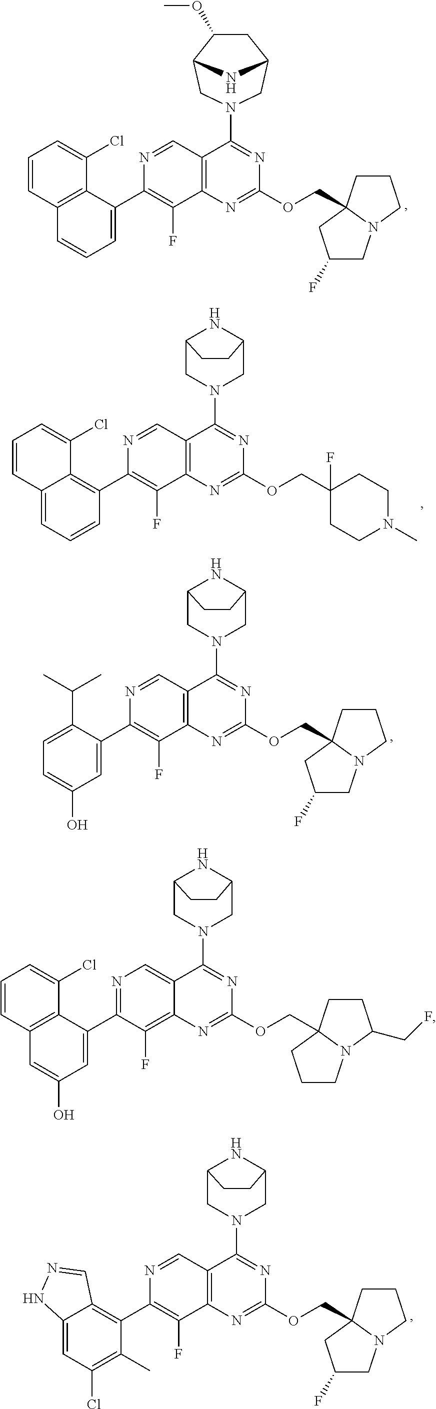 [SMILES: CO[C@@H]1C[C@@H]2CN(c3nc(OC[C@@]45CCCN4C[C@H](F)C5)nc4c(F)c(-c5cccc6cccc(Cl)c56)ncc34)C[C@H]1N2 ; CN1CCC(F)(COc2nc(N3CC4CCC(C3)N4)c3cnc(-c4cccc5cccc(Cl)c45)c(F)c3n2)CC1 ; CC(C)c1ccc(O)cc1-c1ncc2c(N3CC4CCC(C3)N4)nc(OC[C@@]34CCCN3C[C@H](F)C4)nc2c1F ; Oc1cc(-c2ncc3c(N4CC5CCC(C4)N5)nc(OCC45CCCN4C(CF)CC5)nc3c2F)c2c(Cl)cccc2c1 ; Cc1c(Cl)cc2[nH]ncc2c1-c1ncc2c(N3CC4CCC(C3)N4)nc(OC[C@@]34CCCN3C[C@H](F)C4)nc2c1F] 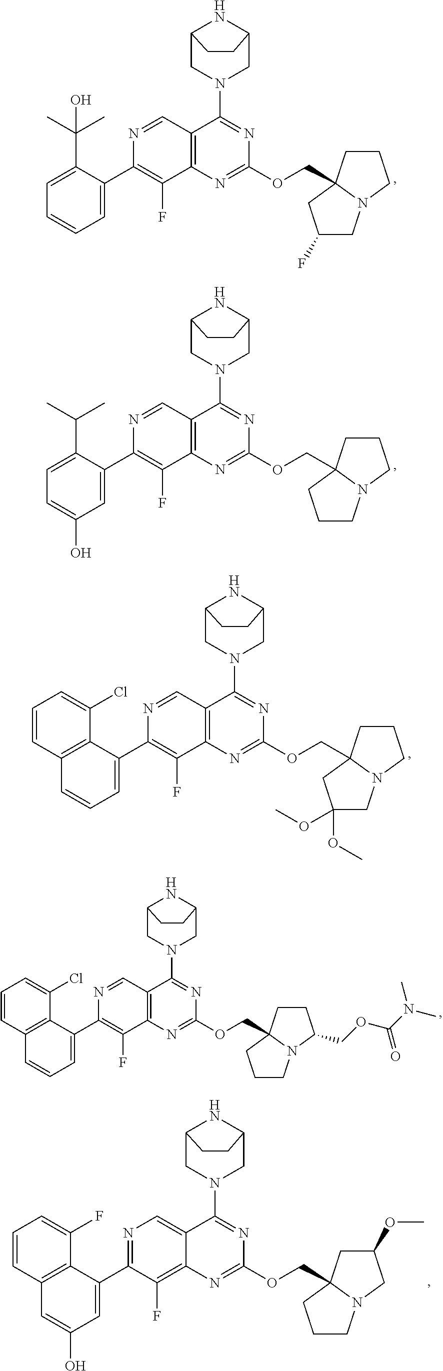 [SMILES: CC(C)(O)c1ccccc1-c1ncc2c(N3CC4CCC(C3)N4)nc(OC[C@@]34CCCN3C[C@H](F)C4)nc2c1F ; CC(C)c1ccc(O)cc1-c1ncc2c(N3CC4CCC(C3)N4)nc(OCC34CCCN3CCC4)nc2c1F ; COC1(OC)CN2CCCC2(COc2nc(N3CC4CCC(C3)N4)c3cnc(-c4cccc5cccc(Cl)c45)c(F)c3n2)C1 ; CN(C)C(=O)OC[C@H]1CC[C@@]2(COc3nc(N4CC5CCC(C4)N5)c4cnc(-c5cccc6cccc(Cl)c56)c(F)c4n3)CCCN12 ; CO[C@H]1CN2CCC[C@]2(COc2nc(N3CC4CCC(C3)N4)c3cnc(-c4cc(O)cc5cccc(F)c45)c(F)c3n2)C1] 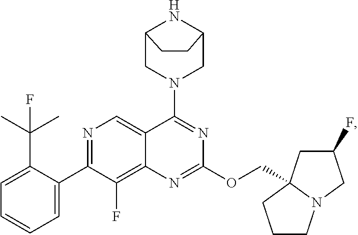 [SMILES: CC(C)(F)c1ccccc1-c1ncc2c(N3CC4CCC(C3)N4)nc(OC[C@@]34CCCN3C[C@H](F)C4)nc2c1F] 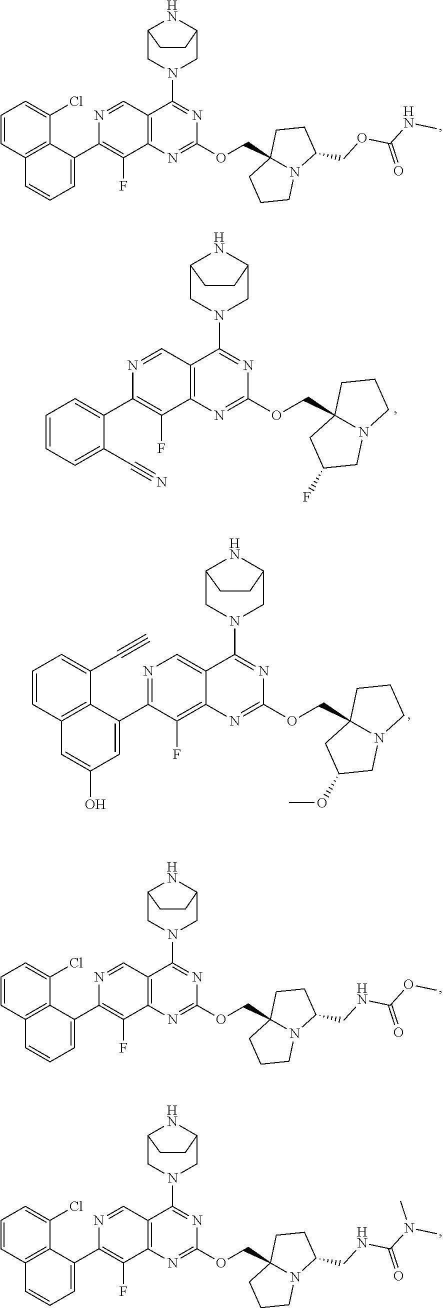 [SMILES: CNC(=O)OC[C@H]1CC[C@@]2(COc3nc(N4CC5CCC(C4)N5)c4cnc(-c5cccc6cccc(Cl)c56)c(F)c4n3)CCCN12 ; N#Cc1ccccc1-c1ncc2c(N3CC4CCC(C3)N4)nc(OC[C@@]34CCCN3C[C@H](F)C4)nc2c1F ; C#Cc1cccc2cc(O)cc(-c3ncc4c(N5CC6CCC(C5)N6)nc(OC[C@@]56CCCN5C[C@H](OC)C6)nc4c3F)c12 ; COC(=O)NC[C@H]1CC[C@@]2(COc3nc(N4CC5CCC(C4)N5)c4cnc(-c5cccc6cccc(Cl)c56)c(F)c4n3)CCCN12 ; CN(C)C(=O)NC[C@H]1CC[C@@]2(COc3nc(N4CC5CCC(C4)N5)c4cnc(-c5cccc6cccc(Cl)c56)c(F)c4n3)CCCN12] 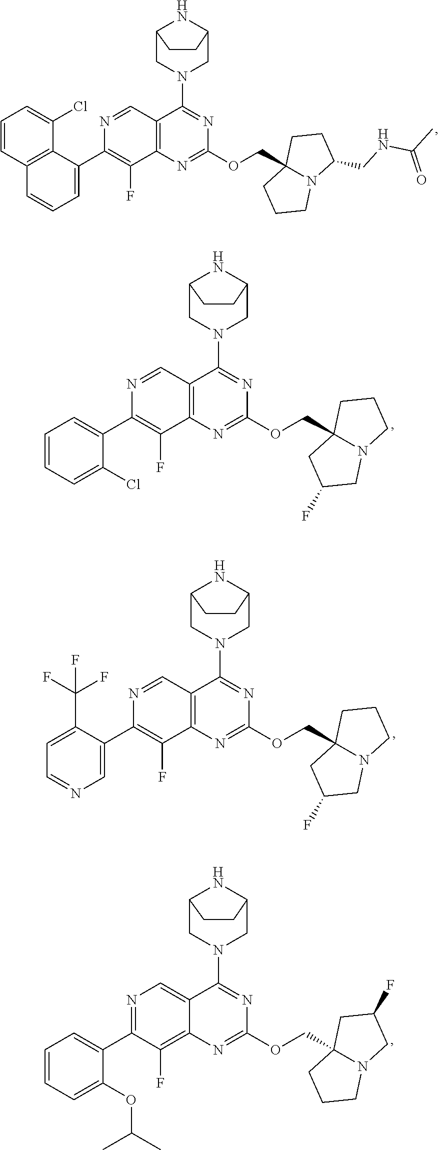 [SMILES: CC(=O)NC[C@H]1CC[C@@]2(COc3nc(N4CC5CCC(C4)N5)c4cnc(-c5cccc6cccc(Cl)c56)c(F)c4n3)CCCN12 ; Fc1c(-c2ccccc2Cl)ncc2c(N3CC4CCC(C3)N4)nc(OC[C@@]34CCCN3C[C@H](F)C4)nc12 ; Fc1c(-c2cnccc2C(F)(F)F)ncc2c(N3CC4CCC(C3)N4)nc(OC[C@@]34CCCN3C[C@H](F)C4)nc12 ; CC(C)Oc1ccccc1-c1ncc2c(N3CC4CCC(C3)N4)nc(OC[C@@]34CCCN3C[C@H](F)C4)nc2c1F] 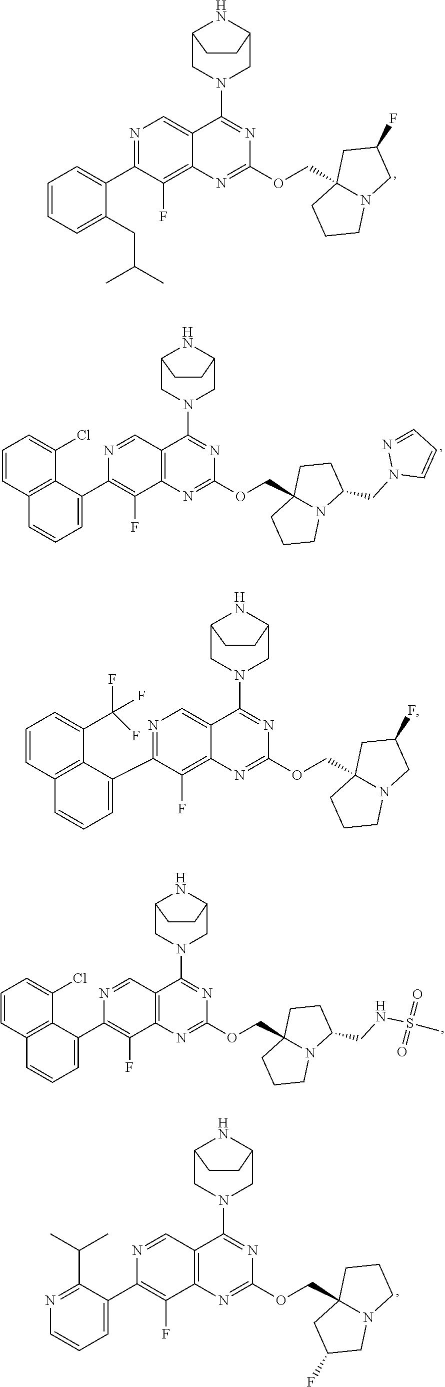 [SMILES: CC(C)Cc1ccccc1-c1ncc2c(N3CC4CCC(C3)N4)nc(OC[C@@]34CCCN3C[C@H](F)C4)nc2c1F ; Fc1c(-c2cccc3cccc(Cl)c23)ncc2c(N3CC4CCC(C3)N4)nc(OC[C@]34CCCN3[C@@H](Cn3cccn3)CC4)nc12 ; Fc1c(-c2cccc3cccc(C(F)(F)F)c23)ncc2c(N3CC4CCC(C3)N4)nc(OC[C@@]34CCCN3C[C@H](F)C4)nc12 ; CS(=O)(=O)NC[C@H]1CC[C@@]2(COc3nc(N4CC5CCC(C4)N5)c4cnc(-c5cccc6cccc(Cl)c56)c(F)c4n3)CCCN12 ; CC(C)c1ncccc1-c1ncc2c(N3CC4CCC(C3)N4)nc(OC[C@@]34CCCN3C[C@H](F)C4)nc2c1F] 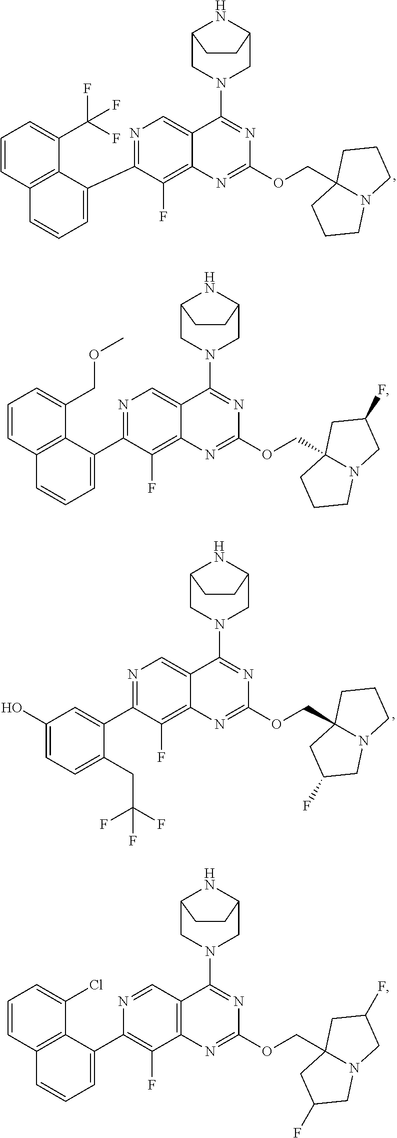 [SMILES: Fc1c(-c2cccc3cccc(C(F)(F)F)c23)ncc2c(N3CC4CCC(C3)N4)nc(OCC34CCCN3CCC4)nc12 ; COCc1cccc2cccc(-c3ncc4c(N5CC6CCC(C5)N6)nc(OC[C@@]56CCCN5C[C@H](F)C6)nc4c3F)c12 ; Oc1ccc(CC(F)(F)F)c(-c2ncc3c(N4CC5CCC(C4)N5)nc(OC[C@@]45CCCN4C[C@H](F)C5)nc3c2F)c1 ; Fc1c(-c2cccc3cccc(Cl)c23)ncc2c(N3CC4CCC(C3)N4)nc(OCC34CC(F)CN3CC(F)C4)nc12] 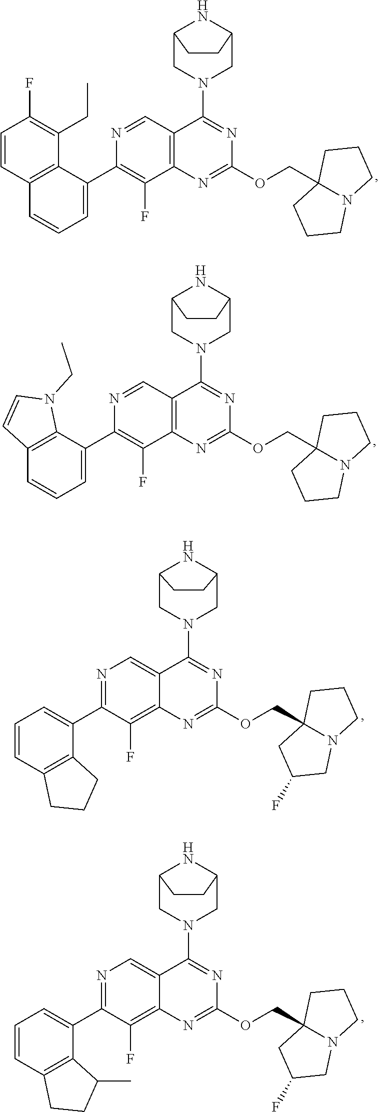 [SMILES: CCc1c(F)ccc2cccc(-c3ncc4c(N5CC6CCC(C5)N6)nc(OCC56CCCN5CCC6)nc4c3F)c12 ; CCn1ccc2cccc(-c3ncc4c(N5CC6CCC(C5)N6)nc(OCC56CCCN5CCC6)nc4c3F)c21 ; Fc1c(-c2cccc3c2CCC3)ncc2c(N3CC4CCC(C3)N4)nc(OC[C@@]34CCCN3C[C@H](F)C4)nc12 ; CC1CCc2cccc(-c3ncc4c(N5CC6CCC(C5)N6)nc(OC[C@@]56CCCN5C[C@H](F)C6)nc4c3F)c21] 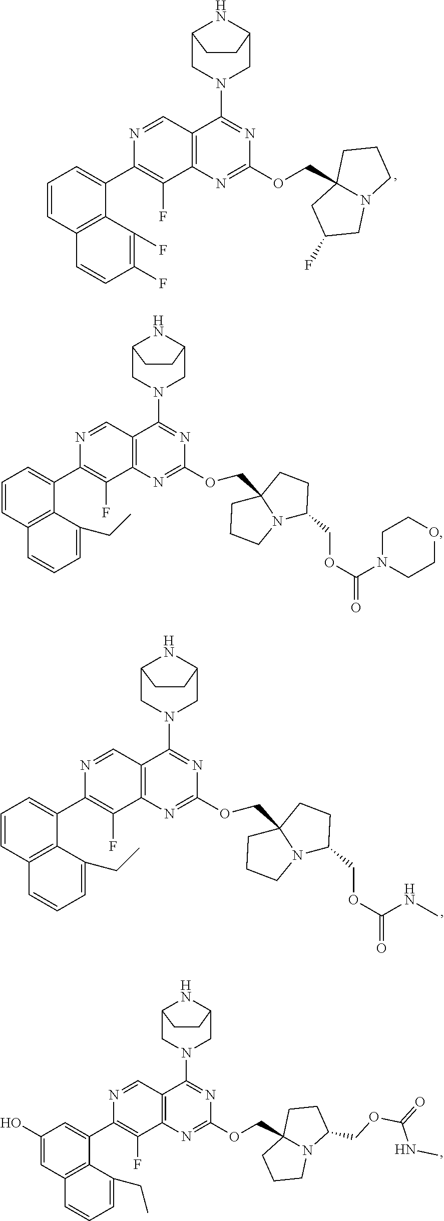 [SMILES: Fc1ccc2cccc(-c3ncc4c(N5CC6CCC(C5)N6)nc(OC[C@@]56CCCN5C[C@H](F)C6)nc4c3F)c2c1F ; CCc1cccc2cccc(-c3ncc4c(N5CC6CCC(C5)N6)nc(OC[C@]56CCCN5[C@@H](COC(=O)N5CCOCC5)CC6)nc4c3F)c12 ; CCc1cccc2cccc(-c3ncc4c(N5CC6CCC(C5)N6)nc(OC[C@]56CCCN5[C@@H](COC(=O)NC)CC6)nc4c3F)c12 ; CCc1cccc2cc(O)cc(-c3ncc4c(N5CC6CCC(C5)N6)nc(OC[C@]56CCCN5[C@@H](COC(=O)NC)CC6)nc4c3F)c12] 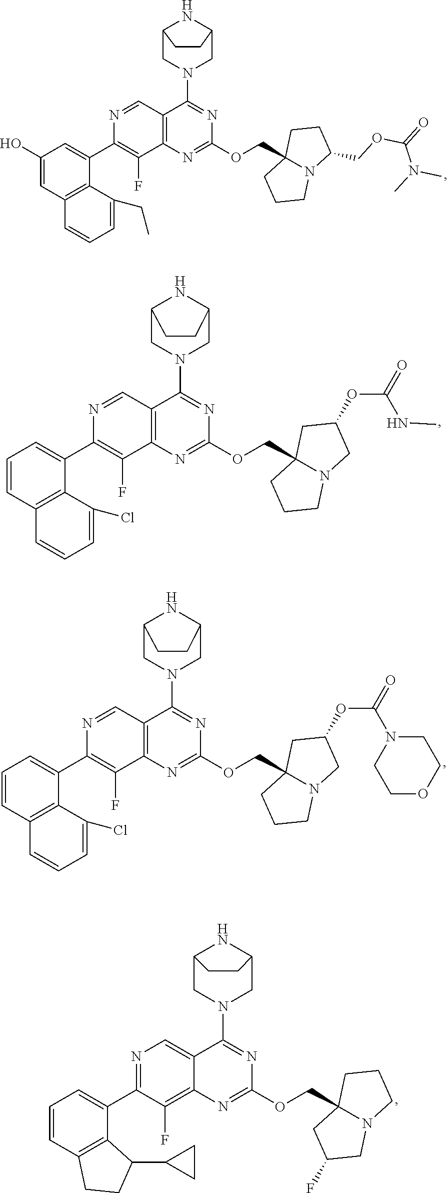 [SMILES: CCc1cccc2cc(O)cc(-c3ncc4c(N5CC6CCC(C5)N6)nc(OC[C@]56CCCN5[C@@H](COC(=O)N(C)C)CC6)nc4c3F)c12 ; CNC(=O)O[C@@H]1CN2CCC[C@]2(COc2nc(N3CC4CCC(C3)N4)c3cnc(-c4cccc5cccc(Cl)c45)c(F)c3n2)C1 ; O=C(O[C@@H]1CN2CCC[C@]2(COc2nc(N3CC4CCC(C3)N4)c3cnc(-c4cccc5cccc(Cl)c45)c(F)c3n2)C1)N1CCOCC1 ; Fc1c(-c2cccc3c2C(C2CC2)CC3)ncc2c(N3CC4CCC(C3)N4)nc(OC[C@@]34CCCN3C[C@H](F)C4)nc12] 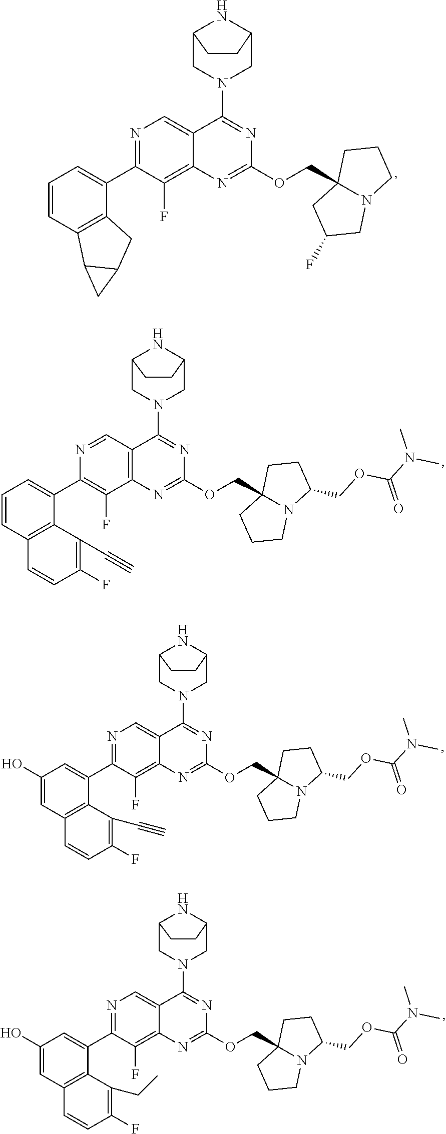 [SMILES: Fc1c(-c2cccc3c2CC2CC32)ncc2c(N3CC4CCC(C3)N4)nc(OC[C@@]34CCCN3C[C@H](F)C4)nc12 ; C#Cc1c(F)ccc2cccc(-c3ncc4c(N5CC6CCC(C5)N6)nc(OC[C@]56CCCN5[C@@H](COC(=O)N(C)C)CC6)nc4c3F)c12 ; C#Cc1c(F)ccc2cc(O)cc(-c3ncc4c(N5CC6CCC(C5)N6)nc(OC[C@]56CCCN5[C@@H](COC(=O)N(C)C)CC6)nc4c3F)c12 ; CCc1c(F)ccc2cc(O)cc(-c3ncc4c(N5CC6CCC(C5)N6)nc(OC[C@]56CCCN5[C@@H](COC(=O)N(C)C)CC6)nc4c3F)c12] 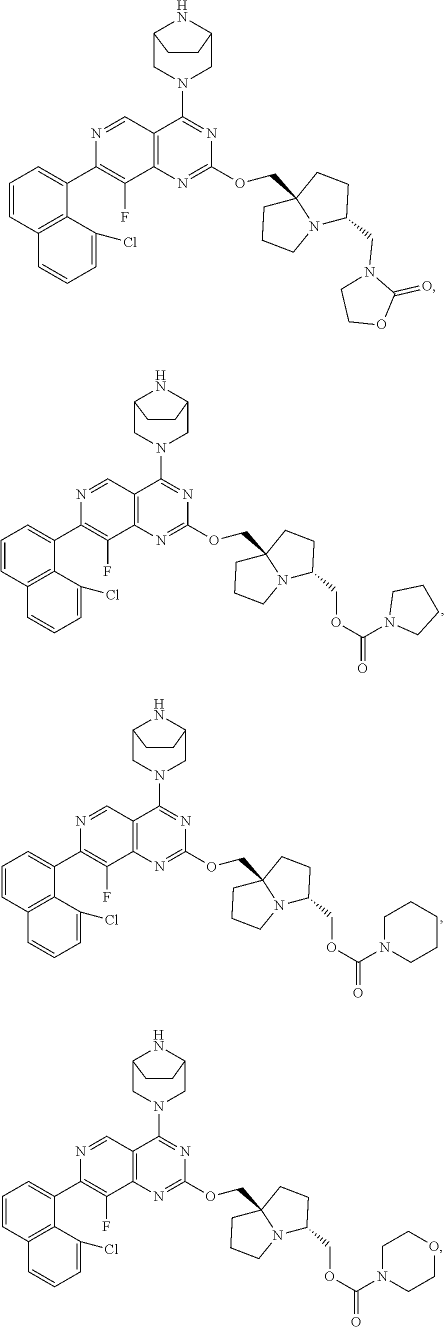 [SMILES: O=C1OCCN1C[C@H]1CC[C@@]2(COc3nc(N4CC5CCC(C4)N5)c4cnc(-c5cccc6cccc(Cl)c56)c(F)c4n3)CCCN12 ; O=C(OC[C@H]1CC[C@@]2(COc3nc(N4CC5CCC(C4)N5)c4cnc(-c5cccc6cccc(Cl)c56)c(F)c4n3)CCCN12)N1CCCC1 ; O=C(OC[C@H]1CC[C@@]2(COc3nc(N4CC5CCC(C4)N5)c4cnc(-c5cccc6cccc(Cl)c56)c(F)c4n3)CCCN12)N1CCCCC1 ; O=C(OC[C@H]1CC[C@@]2(COc3nc(N4CC5CCC(C4)N5)c4cnc(-c5cccc6cccc(Cl)c56)c(F)c4n3)CCCN12)N1CCOCC1] 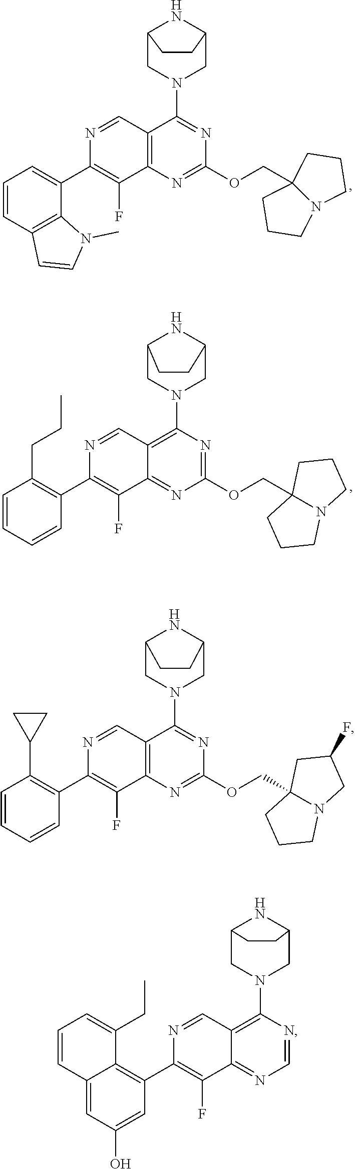 [SMILES: Cn1ccc2cccc(-c3ncc4c(N5CC6CCC(C5)N6)nc(OCC56CCCN5CCC6)nc4c3F)c21 ; CCCc1ccccc1-c1ncc2c(N3CC4CCC(C3)N4)nc(OCC34CCCN3CCC4)nc2c1F ; Fc1c(-c2ccccc2C2CC2)ncc2c(N3CC4CCC(C3)N4)nc(OC[C@@]34CCCN3C[C@H](F)C4)nc12 ; CCc1cccc2cc(O)cc(-c3ncc4c(N5CC6CCC(C5)N6)ncnc4c3F)c12] 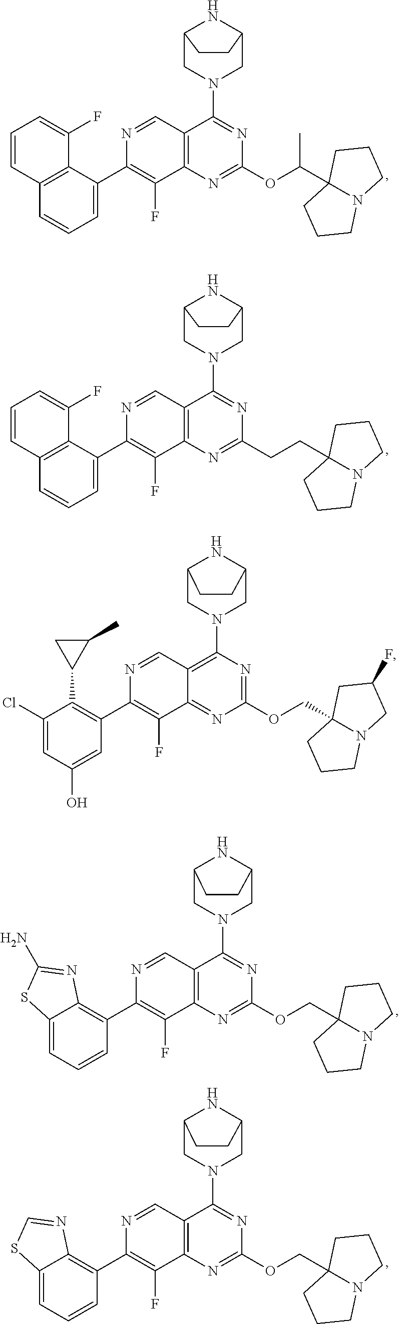 [SMILES: CC(Oc1nc(N2CC3CCC(C2)N3)c2cnc(-c3cccc4cccc(F)c34)c(F)c2n1)C12CCCN1CCC2 ; Fc1c(-c2cccc3cccc(F)c23)ncc2c(N3CC4CCC(C3)N4)nc(CCC34CCCN3CCC4)nc12 ; C[C@@H]1C[C@H]1c1c(Cl)cc(O)cc1-c1ncc2c(N3CC4CCC(C3)N4)nc(OC[C@@]34CCCN3C[C@H](F)C4)nc2c1F ; Nc1nc2c(-c3ncc4c(N5CC6CCC(C5)N6)nc(OCC56CCCN5CCC6)nc4c3F)cccc2s1 ; Fc1c(-c2cccc3scnc23)ncc2c(N3CC4CCC(C3)N4)nc(OCC34CCCN3CCC4)nc12] 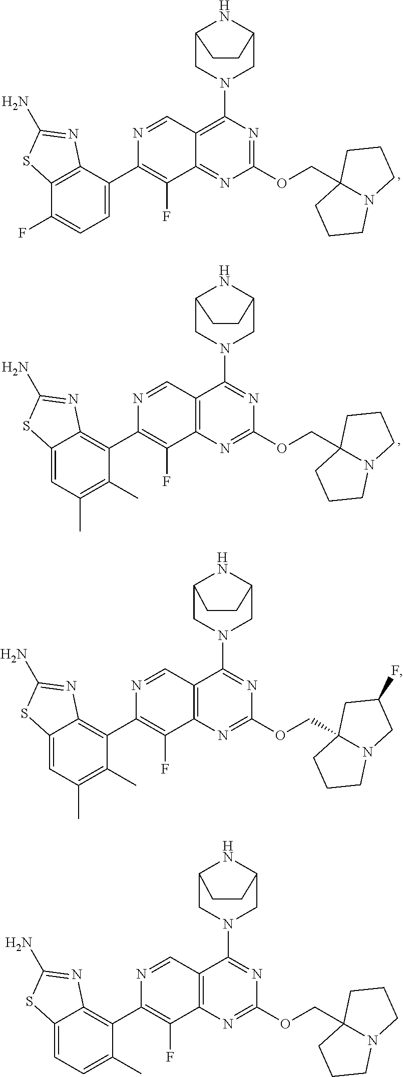 [SMILES: Nc1nc2c(-c3ncc4c(N5CC6CCC(C5)N6)nc(OCC56CCCN5CCC6)nc4c3F)ccc(F)c2s1 ; Cc1cc2sc(N)nc2c(-c2ncc3c(N4CC5CCC(C4)N5)nc(OCC45CCCN4CCC5)nc3c2F)c1C ; Cc1cc2sc(N)nc2c(-c2ncc3c(N4CC5CCC(C4)N5)nc(OC[C@@]45CCCN4C[C@H](F)C5)nc3c2F)c1C ; Cc1ccc2sc(N)nc2c1-c1ncc2c(N3CC4CCC(C3)N4)nc(OCC34CCCN3CCC4)nc2c1F]
and pharmaceutically acceptable salts thereof.

46. The method of claim 1, wherein the KRas G12D inhibitor is: 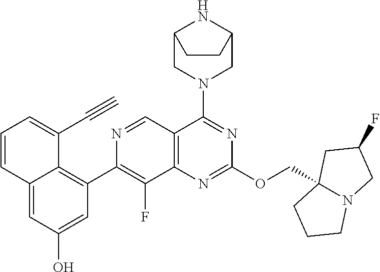 [SMILES: C#Cc1cccc2cc(O)cc(-c3ncc4c(N5CC6CCC(C5)N6)nc(OC[C@@]56CCCN5C[C@H](F)C6)nc4c3F)c12]
or a pharmaceutically acceptable salt thereof.

47. The method of claim 1, wherein the KRas G12D inhibitor is: 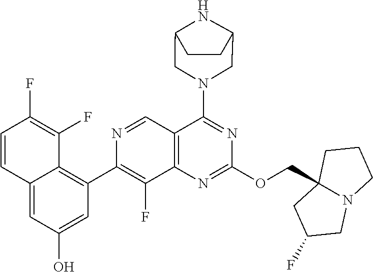 [SMILES: Oc1cc(-c2ncc3c(N4CC5CCC(C4)N5)nc(OC[C@@]45CCCN4C[C@H](F)C5)nc3c2F)c2c(F)c(F)ccc2c1]
or a pharmaceutically acceptable salt thereof.

48. The method of claim 1, wherein the KRas G12D inhibitor is: 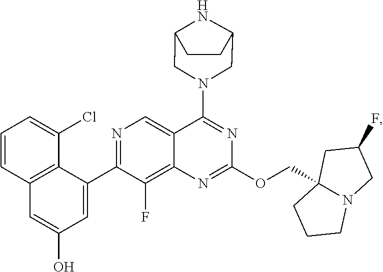 [SMILES: Oc1cc(-c2ncc3c(N4CC5CCC(C4)N5)nc(OC[C@@]45CCCN4C[C@H](F)C5)nc3c2F)c2c(Cl)cccc2c1]
or a pharmaceutically acceptable salt thereof.

49. The method of claim 1, wherein the KRas G12D inhibitor is: 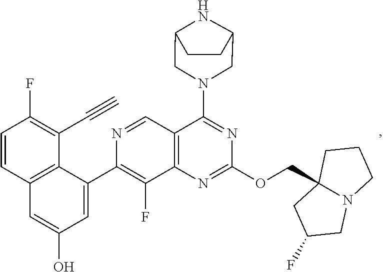 [SMILES: C#Cc1c(F)ccc2cc(O)cc(-c3ncc4c(N5CC6CCC(C5)N6)nc(OC[C@@]56CCCN5C[C@H](F)C6)nc4c3F)c12 ; ","]
or a pharmaceutically acceptable salt thereof.

50. The method of claim 1, wherein the KRas G12D inhibitor is: 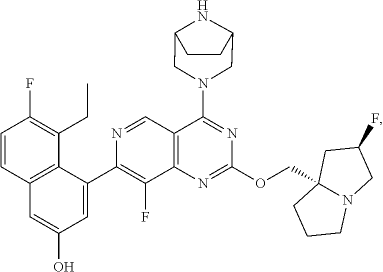 [SMILES: CCc1c(F)ccc2cc(O)cc(-c3ncc4c(N5CC6CCC(C5)N6)nc(OC[C@@]56CCCN5C[C@H](F)C6)nc4c3F)c12]
or a pharmaceutically acceptable salt thereof.

51. The method of claim 1, wherein the KRas G12D inhibitor is: 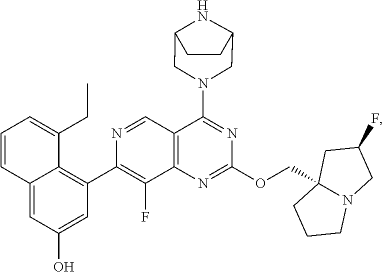 [SMILES: CCc1cccc2cc(O)cc(-c3ncc4c(N5CC6CCC(C5)N6)nc(OC[C@@]56CCCN5C[C@H](F)C6)nc4c3F)c12]
or a pharmaceutically acceptable salt thereof.

52. The method of claim 1, wherein the KRas G12D inhibitor is: 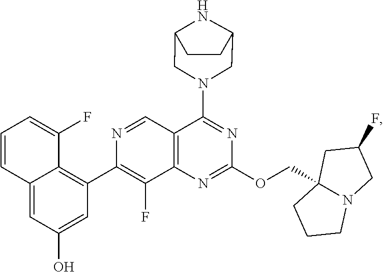 [SMILES: Oc1cc(-c2ncc3c(N4CC5CCC(C4)N5)nc(OC[C@@]45CCCN4C[C@H](F)C5)nc3c2F)c2c(F)cccc2c1]
or a pharmaceutically acceptable salt thereof.

53. The method according to claim 1, wherein the pan ErbB family inhibitor is selected from the group consisting of afatinib, dacomitinib, poziotinib, erlotinib, gefitinib, sapitinib, tarloxotinib, and cetuximab.

54. The method of claim 53, wherein the pan ErbB family inhibitor is afatinib.

55. The method of claim 53, wherein the pan ErbB family inhibitor is cetuximab.

56. The method of claim 46, wherein the pan ErbB family inhibitor is afatinib.

57. The method of claim 46, wherein the pan ErbB family inhibitor is cetuximab.

58. The method of claim 47, wherein the pan ErbB family inhibitor is afatinib.

59. The method of claim 47, wherein the pan ErbB family inhibitor is cetuximab.

60. The method of claim 48, wherein the pan ErbB family inhibitor is afatinib.

61. The method of claim 48, wherein the pan ErbB family inhibitor is cetuximab.

62. The method of claim 49, wherein the pan ErbB family inhibitor is afatinib.

63. The method of claim 49, wherein the pan ErbB family inhibitor is cetuximab.

64. The method of claim 50, wherein the pan ErbB family inhibitor is afatinib.

65. The method of claim 50, wherein the pan ErbB family inhibitor is cetuximab.

66. The method of claim 51, wherein the pan ErbB family inhibitor is afatinib.

67. The method of claim 51, wherein the pan ErbB family inhibitor is cetuximab.

68. The method of claim 52, wherein the pan ErbB family inhibitor is afatinib.

69. The method of claim 52, wherein the pan ErbB family inhibitor is cetuximab

70. The method of according to claim 1, wherein the pan ErbB family inhibitor and the KRAS G12D inhibitor are administered on the same day.

71. The method of according to claim 1, wherein the pan ErbB family inhibitor and the KRAS G12D inhibitor are administered on different days.

72. The method of according to claim 1, wherein the KRas G12D inhibitor is administered at a maximum tolerated dose.

73. The method according to claim 1, wherein the pan ErbB family inhibitor and the KRAS G12D inhibitor are each administered at a maximum tolerated dose.

74. The method according to claim 1, wherein the therapeutically effective amount of the combination of the pan ErbB family inhibitor and the KRAS G12D inhibitor results in an increased duration of overall survival, an increased duration of progression free survival, an increase in tumor growth regression, an increase in tumor growth inhibition or an increased duration of stable disease in the subjects relative to treatment with only the KRas G12D inhibitor.

75. A pharmaceutical composition, comprising a therapeutically effective amount of a combination of a pan ErbB family inhibitor and a KRas G12D inhibitor according to claim 1, and a pharmaceutically acceptable excipient.

76. A method for inhibiting KRas G12D activity in a cancer cell, comprising contacting the cancer cell in which inhibition of KRas G12D activity is desired with an effective amount of a pan ErbB family inhibitor and a KRas G12D inhibitor compound according to claim 1, pharmaceutical compositions or pharmaceutically acceptable salts thereof, wherein the pan ErbB family inhibitor synergistically increases the sensitivity of the cancer cells to the KRas G12D inhibitor.

77. The method according to claim 1, wherein the pan ErbB family inhibitor synergistically increases the sensitivity of the cancer cells to the KRas G12D inhibitor.

78. A method for increasing the sensitivity of a cancer cell to a KRas G12D inhibitor compound of Formula (I), comprising administering to a subject undergoing KRas G12D treatment with a compound according to claim 1, alone or combined with a pharmaceutically acceptable carrier, excipient or diluents, a therapeutically effective amount of a pan ErbB family inhibitor, wherein the pan ErbB family inhibitor synergistically increases the sensitivity of the cancer cell to the KRas G12D inhibitor.

79. The method according to claim 78, wherein the therapeutically effective amount of the KRas G12D inhibitor in the combination is between about 0.01 to 100 mg/kg per day.

80. The method according to claim 79, wherein the therapeutically effective amount of the KRas G12D inhibitor in the combination is between about 0.1 to 50 mg/kg per day.

81. The method according to claim 78, wherein the therapeutically effective amount of the pan ErbB family inhibitor in the combination is between about 0.01 to 100 mg/kg per day.

82. The method according to claim 81, wherein the therapeutically effective amount of the pan ErbB family inhibitor in the combination is between about 0.1 to 50 mg/kg per day.

83. The method according to claim 1, wherein the cancer is selected from the group consisting of Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial, carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma.

84. The method of claim 83, wherein the cancer wherein the cancer is a KRas G12D-associated cancer.

85. The method of claim 83, wherein the cancer is non-small cell lung cancer.

86. A kit comprising the pharmaceutical composition of claim 75 for treating KRas G12D cancer in a subject.

87. A kit comprising: a) a pharmaceutical composition comprising a pan ErbB family inhibitor and b) a pharmaceutical composition comprising a KRas G12D inhibitor of claim 1, for treating a KRas G12D cancer in a subject.

88. The kit according to claim 86, further comprising an insert with instructions for administration of the pharmaceutical composition(s).
